SEQUENCE LISTING (141 sequences)

SEQ ID NO: 1 - protein, 10 aa, synthetic construct
  source 1..10: note = Antibody Clone A3, A2, P12 VH CDR1 (Exemplary, AbM)
GFTFSSYYIH

SEQ ID NO: 2 - protein, 17 aa, synthetic construct
  source 1..17: note = Antibody Clone A3 VH CDR2 (Exemplary, Kabat)
SISPYDGYTDYADSVKG

SEQ ID NO: 3 - protein, 15 aa, synthetic construct
  source 1..15: note = Antibody Clone A3 VH CDR3 (Exemplary, Kabat, AbM)
SSDFSPWTSSGGMDY

SEQ ID NO: 4 - protein, 11 aa, synthetic construct
  source 1..11: note = Antibody Clone A3, A42, P12 VL CDR1 (Exemplary, Kabat, AbM)
RASQSVSSAVA

SEQ ID NO: 5 - protein, 7 aa, synthetic construct
  source 1..7: note = Antibody Clone A3, A42, P12 VL CDR2 (Exemplary, Kabat, AbM)
SASSLYS

SEQ ID NO: 6 - protein, 9 aa, synthetic construct
  source 1..9: note = Antibody Clone A3 VL CDR3 (Exemplary, IMGT, Kabat, AbM)
QQYGSSLRT

SEQ ID NO: 7 - protein, 8 aa, synthetic construct
  source 1..8: note = Antibody Clone A3, A2, P12 VH CDR1 (IMGT)
GFTFSSYY

SEQ ID NO: 8 - protein, 8 aa, synthetic construct
  source 1..8: note = Antibody Clone A3 VH CDR2 (IMGT)
ISPYDGYT

SEQ ID NO: 9 - protein, 17 aa, synthetic construct
  source 1..17: note = Antibody Clone A3 VH CDR3 (IMGT)
ARSSDFSPWTSSGGMDY

SEQ ID NO: 10 - protein, 6 aa, synthetic construct
  source 1..6: note = Antibody Clone A3, A42, P12 VL CDR1 (IMGT)
QSVSSA

SEQ ID NO: 11 - skipped

SEQ ID NO: 12 - protein, 5 aa, synthetic construct
  source 1..5: note = Antibody Clone A3, A2, P12 VH CDR1 (Kabat)
SYYIH

SEQ ID NO: 13 - protein, 7 aa, synthetic construct
  source 1..7: note = Antibody Clone A3, A2, P12 VH CDR1 (Chothia)
GFTFSSY

SEQ ID NO: 14 - protein, 4 aa, synthetic construct
  source 1..4: note = Antibody Clone A3 VH CDR2 (Chothia)
PYDG

SEQ ID NO: 15 - protein, 13 aa, synthetic construct
  source 1..13: note = Antibody Clone A3 VH CDR3 (Chothia)
SDFSPWTSSGGMD

SEQ ID NO: 16 - protein, 7 aa, synthetic construct
  source 1..7: note = Antibody Clone A3, A42, P12 VL CDR1 (Chothia)
SQSVSSA

SEQ ID NO: 17 - protein, 6 aa, synthetic construct
  source 1..6: note = Antibody Clone A3 VL CDR3 (Chothia)
YGSSLR

SEQ ID NO: 18 - protein, 6 aa, synthetic construct
  source 1..6: note = Antibody Clone A3, A2, P12 VH CDR1 (Contact)
SSYYIH

SEQ ID NO: 19 - protein, 13 aa, synthetic construct
  source 1..13: note = Antibody Clone A3 VH CDR2 (Contact)
WVASISPYDGYTD

SEQ ID NO: 20 - protein, 16 aa, synthetic construct
  source 1..16: note = Antibody Clone A3 VH CDR3 (Contact)
ARSSDFSPWTSSGGMD

SEQ ID NO: 21 - protein, 7 aa, synthetic construct
  source 1..7: note = Antibody Clone A3, A42, P12 VL CDR1 (Contact)
SSAVAWY

SEQ ID NO: 22 - protein, 10 aa, synthetic construct
  source 1..10: note = Antibody Clone A3, A42, P12 VL CDR2 (Contact)
LLIYSASSLY

SEQ ID NO: 23 - protein, 8 aa, synthetic construct
  source 1..8: note = Antibody Clone A3 VL CDR3 (Contact)
QQYGSSLR

SEQ ID NO: 24 - protein, 10 aa, synthetic construct
  source 1..10: note = Antibody Clone A3 VH CDR2 (AbM)
SISPYDGYTD

SEQ ID NO: 25 - protein, 124 aa, synthetic construct
  source 1..124: note = Antibody Clone A3 VH Sequence
EVQLVESGGGLVQPGGSLRLSCAASGFTFSSYYIHWVRQAPGKGLEWVASISPYDGYTDYADSVKGRFTISADTSKNTAYLQMNSLRAEDTAVYYCARSSDFSPWTSSGGMDYWGQGTLVTVSS

SEQ ID NO: 26 - protein, 107 aa, synthetic construct
  source 1..107: note = Antibody Clone A3 VL Sequence
DIQMTQSPSSLSASVGDRVTITCRASQSVSSAVAWYQQKPGKAPKLLIYSASSLYSGVPSRFSGSRSGTDFTLTISSLQPEDFATYYCQQYGSSLRTFGQGTKVEIK

SEQ ID NO: 27 - protein, 17 aa, synthetic construct
  source 1..17: note = Antibody Clone A2 VH CDR2 (Exemplary, Kabat)
SYVPEDGYTDYADSVKG

SEQ ID NO: 28 - protein, 15 aa, synthetic construct
  source 1..15: note = Antibody Clone A2 VH CDR3 (Exemplary, Kabat, AbM)
SSDFIPWTSSGGMDY

SEQ ID NO: 29 - protein, 11 aa, synthetic construct
  source 1..11: note = Antibody Clone A2 VL CDR1 (Exemplary, Kabat, AbM)
RASQSVSRAVA

SEQ ID NO: 30 - protein, 7 aa, synthetic construct
  source 1..7: note = Antibody Clone A2 VL CDR2 (Exemplary, Kabat, AbM)
SASSLHS

SEQ ID NO: 31 - protein, 9 aa, synthetic construct
  source 1..9: note = Antibody Clone A2 VL CDR3 (Exemplary, IMGT, Kabat, AbM)
QQYGSSART

SEQ ID NO: 32 - protein, 8 aa, synthetic construct
  source 1..8: note = Antibody Clone A2 VH CDR2 (IMGT)
YVPEDGYT

SEQ ID NO: 33 - protein, 17 aa, synthetic construct
  source 1..17: note = Antibody Clone A2 VH CDR3 (IMGT)
ARSSDFIPWTSSGGMDY

SEQ ID NO: 34 - protein, 6 aa, synthetic construct
  source 1..6: note = Antibody Clone A2 VL CDR1 (IMGT)
QSVSRA

SEQ ID NO: 35 - protein, 4 aa, synthetic construct
  source 1..4: note = Antibody Clone A2 VH CDR2 (Chothia)
PEDG

SEQ ID NO: 36 - protein, 13 aa, synthetic construct
  source 1..13: note = Antibody Clone A2 VH CDR3 (Chothia)
SDFIPWTSSGGMD

SEQ ID NO: 37 - protein, 7 aa, synthetic construct
  source 1..7: note = Antibody Clone A2 VL CDR1 (Chothia)
SQSVSRA

SEQ ID NO: 38 - protein, 6 aa, synthetic construct
  source 1..6: note = Antibody Clone A2 VL CDR3 (Chothia)
YGSSAR

SEQ ID NO: 39 - protein, 13 aa, synthetic construct
  source 1..13: note = Antibody Clone A2 VH CDR2 (Contact)
WVASYVPEDGYTD

SEQ ID NO: 40 - protein, 16 aa, synthetic construct
  source 1..16: note = Antibody Clone A2 VH CDR3 (Contact)
ARSSDFIPWTSSGGMD

SEQ ID NO: 41 - protein, 7 aa, synthetic construct
  source 1..7: note = Antibody Clone A2 VL CDR1 (Contact)
SRAVAWY

SEQ ID NO: 42 - protein, 10 aa, synthetic construct
  source 1..10: note = Antibody Clone A2 VL CDR2 (Contact)
LLIYSASSLH

SEQ ID NO: 43 - protein, 8 aa, synthetic construct
  source 1..8: note = Antibody Clone A2 VL CDR3 (Contact)
QQYGSSAR

SEQ ID NO: 44 - protein, 10 aa, synthetic construct
  source 1..10: note = Antibody Clone A2 VH CDR2 (AbM)
SYVPEDGYTD

SEQ ID NO: 45 - protein, 124 aa, synthetic construct
  source 1..124: note = Antibody Clone A2 VH Sequence
EVQLVESGGGLVQPGGSLRLSCAASGFTFSSYYIHWVRQAPGKGLEWVASYVPEDGYTDYADSVKGRFTISADTSKNTAYLQMNSLRAEDTAVYYCARSSDFIPWTSSGGMDYWGQGTLVTVSS

SEQ ID NO: 46 - protein, 107 aa, synthetic construct
  source 1..107: note = Antibody Clone A2 VL Sequence
DIQMTQSPSSLSASVGDRVTITCRASQSVSRAVAWYQQKPGKAPKLLIYSASSLHSGVPSRFSGSRSGTDFTLTISSLQPEDFATYYCQQYGSSARTFGQGTKVEIK

SEQ ID NO: 47 - protein, 10 aa, synthetic construct
  source 1..10: note = Antibody Clone A42, A11 VH CDR1 (Exemplary, AbM)
GFTFSWYYIH

SEQ ID NO: 48 - protein, 17 aa, synthetic construct
  source 1..17: note = Antibody Clone A42, A11 VH CDR2 (Exemplary, Kabat)
VIDPISSYTYYADSVKG

SEQ ID NO: 49 - protein, 8 aa, synthetic construct
  source 1..8: note = Antibody Clone A42, A11 VH CDR3 (Exemplary, Kabat, AbM)
GFDRAMDY

SEQ ID NO: 50 - protein, 9 aa, synthetic construct
  source 1..9: note = Antibody Clone A42, A11 VL CDR3 (Exemplary, IMGT, Kabat, AbM)
QQYYSSPYT

SEQ ID NO: 51 - protein, 8 aa, synthetic construct
  source 1..8: note = Antibody Clone A42, A11 VH CDR1 (IMGT)
GFTFSWYY

SEQ ID NO: 52 - protein, 8 aa, synthetic construct
  source 1..8: note = Antibody Clone A42, A11 VH CDR2 (IMGT)
IDPISSYT

SEQ ID NO: 53 - protein, 10 aa, synthetic construct
  source 1..10: note = Antibody Clone A42, A11 VH CDR3 (IMGT)
ARGFDRAMDY

SEQ ID NO: 54 - protein, 5 aa, synthetic construct
  source 1..5: note = Antibody Clone A42, A11 VH CDR1 (Kabat)
WYYIH

SEQ ID NO: 55 - protein, 7 aa, synthetic construct
  source 1..7: note = Antibody Clone A42, A11 VH CDR1 (Chothia)
GFTFSWY

SEQ ID NO: 56 - protein, 4 aa, synthetic construct
  source 1..4: note = Antibody Clone A42, A11 VH CDR2 (Chothia)
PISS

SEQ ID NO: 57 - protein, 6 aa, synthetic construct
  source 1..6: note = Antibody Clone A42, A11 VH CDR3 (Chothia)
FDRAMD

SEQ ID NO: 58 - protein, 6 aa, synthetic construct
  source 1..6: note = Antibody Clone A42, A11 VL CDR3 (Chothia)
YYSSPY

SEQ ID NO: 59 - protein, 6 aa, synthetic construct
  source 1..6: note = Antibody Clone A42, A11 VH CDR1 (Contact)
SWYYIH

SEQ ID NO: 60 - protein, 13 aa, synthetic construct
  source 1..13: note = Antibody Clone A42, A11 VH CDR2 (Contact)
WVAVIDPISSYTY

SEQ ID NO: 61 - protein, 9 aa, synthetic construct
  source 1..9: note = Antibody Clone A42, A11 VH CDR3 (Contact)
ARGFDRAMD

SEQ ID NO: 62 - protein, 8 aa, synthetic construct
  source 1..8: note = Antibody Clone A42, A11 VL CDR3 (Contact)
QQYYSSPY

SEQ ID NO: 63 - protein, 10 aa, synthetic construct
  source 1..10: note = Antibody Clone A42, A11 VH CDR2 (AbM)
VIDPISSYTY

SEQ ID NO: 64 - protein, 117 aa, synthetic construct
  source 1..117: note = Antibody Clone A42, A11 VH Sequence
EVQLVESGGGLVQPGGSLRLSCAASGFTFSWYYIHWVRQAPGKGLEWVAVIDPISSYTYYADSVKGRFTISADTSKNTAYLQMNSLRAEDTAVYYCARGFDRAMDYWGQGTLVTVSS

SEQ ID NO: 65 - protein, 107 aa, synthetic construct
  source 1..107: note = Antibody Clone A42 VL Sequence
DIQMTQSPSSLSASVGDRVTITCRASQSVSSAVAWYQQKPGKAPKLLIYSASSLYSGVPSRFSGSRSGTDFTLTISSLQPEDFATYYCQQYYSSPYTFGQGTKVEIK

SEQ ID NO: 66 - protein, 11 aa, synthetic construct
  source 1..11: note = Antibody Clone A11 VL CDR1 (Exemplary, Kabat, AbM)
RASKSVSSALA

SEQ ID NO: 67 - protein, 7 aa, synthetic construct
  source 1..7: note = Antibody Clone A11 VL CDR2 (Exemplary, Kabat, AbM)
VGSSRYS

SEQ ID NO: 68 - protein, 6 aa, synthetic construct
  source 1..6: note = Antibody Clone A11 VL CDR1 (IMGT)
KSVSSA

SEQ ID NO: 69 - skipped

SEQ ID NO: 70 - protein, 7 aa, synthetic construct
  source 1..7: note = Antibody Clone A11 VL CDR1 (Chothia)
SKSVSSA

SEQ ID NO: 71 - protein, 7 aa, synthetic construct
  source 1..7: note = Antibody Clone A11 VL CDR1 (Contact)
SSALAWY

SEQ ID NO: 72 - protein, 10 aa, synthetic construct
  source 1..10: note = Antibody Clone A11 VL CDR2 (Contact)
LLIYVGSSRY

SEQ ID NO: 73 - protein, 107 aa, synthetic construct
  source 1..107: note = Antibody Clone A11 VL Sequence
DIQMTQSPSSLSASVGDRVTITCRASKSVSSALAWYQQKPGKAPKLLIYVGSSRYSGVPSRFSGSRSGTDFTLTISSLQPEDFATYYCQQYYSSPYTFGQGTKVEIK

SEQ ID NO: 74 - protein, 140 aa, Homo sapiens
  source 1..140: note = Exemplary amino acid sequence of extracellular domain of human NKG2A
PSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 75 - protein, 138 aa, Homo sapiens
  source 1..138: note = Exemplary extracellular domain of human NKG2C
IPFLEQNNSSPNTRTQKARHCGHCPEEWITYSNSCYYIGKERRTWEESLLACTSKNSSLLSIDNEEEMKFLASILPSSWIGVFRNSSHHPWVTINGLAFKHKIKDSDNAELNCAVLQVNRLKSAQCGSSMIYHCKHKL

SEQ ID NO: 76 - protein, 148 aa, Homo sapiens
  source 1..148: note = Exemplary extracellular domain of human CD94
KNSFTKLSIEPAFTPGPNIELQKDSDCCSCQEKWVGYRCNCYFISSEQKTWNESRHLCASQKSSLLQLQNTDELDFMSSSQQFYWIGLSYSEEHTAWLWENGSALSQYLFPSFETFNTKNCIAYNPNGNALDESCEDKNRYICKQQLI

SEQ ID NO: 77 - protein, 140 aa, Macaca fascicularis
  source 1..140: note = Exemplary extracellular domain of cynomolgus NKG2A
PSTLTQKHNNSSLNTRTQKARHCGHCPEEWITYSNSCYYIGKEKRTWAESLLACTLKNSSLLSIDNEEEMKFLTAISPSTWTGVFRDSSQHPWVTINGLTFKHEIKDSDNAEHNCAMLHARGLKSDRCGSSKIYHCKHKL

SEQ ID NO: 78 - protein, 148 aa, Macaca fascicularis
  source 1..148: note = Exemplary extracellular domain of cynomolgus CD94
KNSFTKLSVEPAYTPGPNIELQKDSDCCSCHEKWVGYRCNCYFISSEEKTWNESRHFCASQKSSLLQLQNRDELDFMSSSQHFYWIGLSYSEEHTAWLWENGSALSQYLFPSFETFKPKNCIAYNSKGNALDESCETKNRYICKQQLI

SEQ ID NO: 79 - protein, 226 aa, synthetic construct
  source 1..226: note = Phage display heavy chain
  VARIANT 31..35: note = X is any amino acid:
  VARIANT 50..66: note = X is any amino acid
  VARIANT 99..112: note = X is any amino acid
EVQLVESGGGLVQPGGSLRLSCAASGFTFSXXXXXWVRQAPGKGLEWVAXXXXXXXXXXXXXXXXXRFTISADTSKNTAYLQMNSLRAEDTAVYYCARXXXXXXXXXXXXXXWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 80 - protein, 214 aa, synthetic construct
  source 1..214: note = Phage display light chain
  VARIANT 89..97: note = X is any amino acid
DIQMTQSPSSLSASVGDRVTITCRASQSVSSAVAWYQQKPGKAPKLLIYSASSLYSGVPSRFSGSRSGTDFTLTISSLQPEDFATYYCXXXXXXXXXFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 330 aa, synthetic construct [sequence omitted]

SEQ ID NO: 82 - protein, 107 aa, synthetic construct
  source 1..107: note = Exemplary light chain constant domain (CL)
RTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 222 aa, synthetic construct
  source 1..222: note = Exemplary Fc region sequence
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 222 aa, synthetic construct
  source 1..222: note = Exemplary variant Fc region (silent Fc) sequence
CPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 330 aa, synthetic construct
  source 1..330: note = Exemplary IgG heavy chain comprises CH1, Hinge, CH2, and CH3 amino acid sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 17 aa, synthetic construct
  source 1..17: note = Antibody Clone P12 VH CDR2 (Exemplary, Kabat)
TINSRGGFTLYADSVKG

SEQ ID NO: 87 - protein, 10 aa, synthetic construct
  source 1..10: note = Antibody Clone P12 VH CDR3 (Exemplary, Kabat, AbM)
GYTLTPVLDY

SEQ ID NO: 88 - protein, 9 aa, synthetic construct
  source 1..9: note = Antibody Clone P12 VL CDR3 (Exemplary, IMGT, Kabat, AbM)
QQFMLEPIT

SEQ ID NO: 89 - protein, 8 aa, synthetic construct
  source 1..8: note = Antibody Clone P12 VH CDR2 (IMGT)
INSRGGFT

SEQ ID NO: 90 - protein, 12 aa, synthetic construct
  source 1..12: note = Antibody Clone P12 VH CDR3 (IMGT)
ARGYTLTPVLDY

SEQ ID NO: 91 - protein, 4 aa, synthetic construct
  source 1..4: note = Antibody Clone P12 VH CDR2 (Chothia)
SRGG

SEQ ID NO: 92 - protein, 8 aa, synthetic construct
  source 1..8: note = Antibody Clone P12 VH CDR3 (Chothia)
YTLTPVLD

SEQ ID NO: 93 - protein, 6 aa, synthetic construct
  source 1..6: note = Antibody Clone P12 VL CDR3 (Chothia)
FMLEPI

SEQ ID NO: 94 - protein, 13 aa, synthetic construct
  source 1..13: note = Antibody Clone P12 VH CDR2 (Contact)
WVATINSRGGFTL

SEQ ID NO: 95 - protein, 11 aa, synthetic construct
  source 1..11: note = Antibody Clone P12 VH CDR3 (Contact)
ARGYTLTPVLD

SEQ ID NO: 96 - protein, 8 aa, synthetic construct
  source 1..8: note = Antibody Clone P12 VL CDR3 (Contact)
QQFMLEPI

SEQ ID NO: 97 - protein, 10 aa, synthetic construct
  source 1..10: note = Antibody Clone P12 VH CDR2 (AbM)
TINSRGGFTL

SEQ ID NO: 98 - protein, 119 aa, synthetic construct
  source 1..119: note = Antibody Clone P12 VH Sequence
EVQLVESGGGLVQPGGSLRLSCAASGFTFSSYYIHWVRQAPGKGLEWVATINSRGGFTLYADSVKGRFTISADTSKNTAYLQMNSLRAEDTAVYYCARGYTLTPVLDYWGQGTLVTVSS

SEQ ID NO: 99 - protein, 107 aa, synthetic construct
  source 1..107: note = Antibody Clone P12 VL Sequence
DIQMTQSPSSLSASVGDRVTITCRASQSVSSAVAWYQQKPGKAPKLLIYSASSLYSGVPSRFSGSRSGTDFTLTISSLQPEDFATYYCQQFMLEPITFGQGTKVEIK

SEQ ID NO: 100 - protein, 290 aa, Homo sapiens
  source 1..290: note = Exemplary full-length amino acid sequence of human PD-L1
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 101 - protein, 288 aa, Homo sapiens
  source 1..288: note = Exemplary full-length amino acid sequence of human PD-1
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 102 - protein, 107 aa, synthetic construct
  source 1..107: note = Exemplary CH3 sequence (aa 116 to aa 222 of SEQ ID NO:83)
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 107 aa, synthetic construct
  source 1..107: note = Exemplary CH3 sequence (Y349C and K370R)
GQPREPQVCTLPPSRDELTKNQVSLTCLVRGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 107 aa, synthetic construct
  source 1..107: note = Exemplary CH3 sequence (N-terminal amino acid residue swapped with CH1, Y349C and K370R)
AQPREPQVCTLPPSRDELTKNQVSLTCLVRGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 107 aa, synthetic construct
  source 1..107: note = Exemplary CH3 sequence (S354C and E357W)
GQPREPQVYTLPPCRDWLTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 107 aa, synthetic construct
  source 1..107: note = Exemplary CH3 sequence (N-terminal amino acid residue swapped with CH1, S354C and E357W)
AQPREPQVYTLPPCRDWLTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 107 aa, synthetic construct
  source 1..107: note = Exemplary CH3 sequence (D356E, L358M, T366S, L368A and Y407V)
GQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 108 - protein, 107 aa, synthetic construct
  source 1..107: note = Exemplary CH3 sequence (T366W)
GQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 110 aa, synthetic construct
  source 1..110: note = Exemplary CH2 sequence (aa 6 to aa 115 of SEQ ID NO:83, L234, L235, and P329)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 110 - protein, 110 aa, synthetic construct
  source 1..110: note = Exemplary CH2 sequence (aa 6 to aa 115 of SEQ ID NO:84, L234A, L235A, and P329K)
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAK

SEQ ID NO: 111 - protein, 107 aa, synthetic construct [sequence omitted]

SEQ ID NO: 112 - protein, 103 aa, synthetic construct
  source 1..103: note = Exemplary CH1 sequence (aa 1 to aa 103 of SEQ ID NO:85)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 113 - protein, 103 aa, synthetic construct
  source 1..103: note = Exemplary CH1 sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 114 - protein, 103 aa, synthetic construct
  source 1..103: note = Exemplary CH1 sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDRKVEPKSC

SEQ ID NO: 115 - protein, 10 aa, synthetic construct
  source 1..10: note = Hinge domain
DKTHTCPPCP

SEQ ID NO: 116 - protein, 4 aa, synthetic construct
  source 1..4: note = Linker
  REPEAT 1..4: note = SSSG can be repeated n times, wherein n is any positive integer, such as 1, 2, 3, 4, 5, 6, 7, 8, 9, 10 or more
SSSG

SEQ ID NO: 117 - protein, 10 aa, synthetic construct
  source 1..10: note = Linker
TASSGGSSSG

SEQ ID NO: 118 - protein, 441 aa, synthetic construct
  source 1..441: note = P12 Chain 1 - VL-CH3 (Y349C and K370R)-Hinge-CH2 (L234, L235, P329, wt)-CH3 (D356E, L358M, T366S, L368A and Y407V, hole)
DIQMTQSPSSLSASVGDRVTITCRASQSVSSAVAWYQQKPGKAPKLLIYSASSLYSGVPSRFSGSRSGTDFTLTISSLQPEDFATYYCQQFMLEPITFGQGTKVEIKGQPREPQVCTLPPSRDELTKNQVSLTCLVRGFYPSDIAVEWESNGQPENNYKTTPPVLDSDGSFFLYSKLTVDKSRWQQGNVFSCSVMHEALHNHYTQKSLSLSPGKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 119 - protein, 665 aa, synthetic construct
  source 1..665: note = P12P12 Chain 1 - VL-CH3 (Y349C and K370R)-Linker-VL-CH3 (Y349C and K370R)-Hinge-CH2 (L234, L235, and P329, wt)-CH3 (D356E, L358M, T366S, L368A and Y407V, hole)
DIQMTQSPSSLSASVGDRVTITCRASQSVSSAVAWYQQKPGKAPKLLIYSASSLYSGVPSRFSGSRSGTDFTLTISSLQPEDFATYYCQQFMLEPITFGQGTKVEIKGQPREPQVCTLPPSRDELTKNQVSLTCLVRGFYPSDIAVEWESNGQPENNYKTTPPVLDSDGSFFLYSKLTVDKSRWQQGNVFSCSVMHEALHNHYTQKSLSLSPGKTASSGGSSSGDIQMTQSPSSLSASVGDRVTITCRASQSVSSAVAWYQQKPGKAPKLLIYSASSLYSGVPSRFSGSRSGTDFTLTISSLQPEDFATYYCQQFMLEPITFGQGTKVEIKGQPREPQVCTLPPSRDELTKNQVSLTCLVRGFYPSDIAVEWESNGQPENNYKTTPPVLDSDGSFFLYSKLTVDKSRWQQGNVFSCSVMHEALHNHYTQKSLSLSPGKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 120 - protein, 226 aa, synthetic construct
  source 1..226: note = P12 Chain 2 - VH-CH3 (G341A, S354C and E357W)
EVQLVESGGGLVQPGGSLRLSCAASGFTFSSYYIHWVRQAPGKGLEWVATINSRGGFTLYADSVKGRFTISADTSKNTAYLQMNSLRAEDTAVYYCARGYTLTPVLDYWGQGTLVTVSSAQPREPQVYTLPPCRDWLTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 441 aa, synthetic construct
  source 1..441: note = A2 Chain 3 - VL-CL-Hinge-CH2 (L234, L235, and P329, wt)-CH3 (T366W, knob)
DIQMTQSPSSLSASVGDRVTITCRASQSVSRAVAWYQQKPGKAPKLLIYSASSLHSGVPSRFSGSRSGTDFTLTISSLQPEDFATYYCQQYGSSARTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 227 aa, synthetic construct
  source 1..227: note = A2 Chain 4 - VH-CH1
EVQLVESGGGLVQPGGSLRLSCAASGFTFSSYYIHWVRQAPGKGLEWVASYVPEDGYTDYADSVKGRFTISADTSKNTAYLQMNSLRAEDTAVYYCARSSDFIPWTSSGGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 123 - protein, 665 aa, synthetic construct
  source 1..665: note = A2A2 Chain 3 - VL-CL-Linker-VL-CL- Hinge-CH2 (L234, L235, and P329, wt)-CH3 (T366W, knob)
DIQMTQSPSSLSASVGDRVTITCRASQSVSRAVAWYQQKPGKAPKLLIYSASSLHSGVPSRFSGSRSGTDFTLTISSLQPEDFATYYCQQYGSSARTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTASSGGSSSGDIQMTQSPSSLSASVGDRVTITCRASQSVSRAVAWYQQKPGKAPKLLIYSASSLHSGVPSRFSGSRSGTDFTLTISSLQPEDFATYYCQQYGSSARTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 665 aa, synthetic construct
  source 1..665: note = A11A11 Chain 3 - VL-CL-Linker-VL-CL-Hinge-CH2 (L234, L235, and P329, wt)-CH3 (T366W, knob)
DIQMTQSPSSLSASVGDRVTITCRASKSVSSALAWYQQKPGKAPKLLIYVGSSRYSGVPSRFSGSRSGTDFTLTISSLQPEDFATYYCQQYYSSPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTASSGGSSSGDIQMTQSPSSLSASVGDRVTITCRASKSVSSALAWYQQKPGKAPKLLIYVGSSRYSGVPSRFSGSRSGTDFTLTISSLQPEDFATYYCQQYYSSPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 441 aa, synthetic construct
  source 1..441: note = A11 Chain 3 - VL-CL-Hinge-CH2 (L234, L235, and P329, wt)-CH3 (T366W, knob)
DIQMTQSPSSLSASVGDRVTITCRASKSVSSALAWYQQKPGKAPKLLIYVGSSRYSGVPSRFSGSRSGTDFTLTISSLQPEDFATYYCQQYYSSPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 220 aa, synthetic construct
  source 1..220: note = A11 Chain 4 - VH-CH1
EVQLVESGGGLVQPGGSLRLSCAASGFTFSWYYIHWVRQAPGKGLEWVAVIDPISSYTYYADSVKGRFTISADTSKNTAYLQMNSLRAEDTAVYYCARGFDRAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 127 - protein, 441 aa, synthetic construct
  source 1..441: note = P12 Chain 1 - VL-CH3 (Y349C and K370R)-Hinge-CH2 (L234A, L235A, and P329K, silent)-CH3 (D356E, L358M, T366S, L368A and Y407V, hole)
DIQMTQSPSSLSASVGDRVTITCRASQSVSSAVAWYQQKPGKAPKLLIYSASSLYSGVPSRFSGSRSGTDFTLTISSLQPEDFATYYCQQFMLEPITFGQGTKVEIKGQPREPQVCTLPPSRDELTKNQVSLTCLVRGFYPSDIAVEWESNGQPENNYKTTPPVLDSDGSFFLYSKLTVDKSRWQQGNVFSCSVMHEALHNHYTQKSLSLSPGKDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 128 - protein, 665 aa, synthetic construct
  source 1..665: note = P12P12 chain 1 - VL-CH3 (Y349C and K370R)-Linker-VL-CH3 (Y349C and K370R)-Hinge-CH2 (L234A, L235A, and P329K, silent)-CH3 (D356E, L358M, T366S, L368A and Y407V, hole)
DIQMTQSPSSLSASVGDRVTITCRASQSVSSAVAWYQQKPGKAPKLLIYSASSLYSGVPSRFSGSRSGTDFTLTISSLQPEDFATYYCQQFMLEPITFGQGTKVEIKGQPREPQVCTLPPSRDELTKNQVSLTCLVRGFYPSDIAVEWESNGQPENNYKTTPPVLDSDGSFFLYSKLTVDKSRWQQGNVFSCSVMHEALHNHYTQKSLSLSPGKTASSGGSSSGDIQMTQSPSSLSASVGDRVTITCRASQSVSSAVAWYQQKPGKAPKLLIYSASSLYSGVPSRFSGSRSGTDFTLTISSLQPEDFATYYCQQFMLEPITFGQGTKVEIKGQPREPQVCTLPPSRDELTKNQVSLTCLVRGFYPSDIAVEWESNGQPENNYKTTPPVLDSDGSFFLYSKLTVDKSRWQQGNVFSCSVMHEALHNHYTQKSLSLSPGKDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 129 - protein, 441 aa, synthetic construct
  source 1..441: note = A2 Chain 3 - VL-CL-Hinge-CH2 (L234A, L235A, and P329K, silent)-CH3 (T366W, knob)
DIQMTQSPSSLSASVGDRVTITCRASQSVSRAVAWYQQKPGKAPKLLIYSASSLHSGVPSRFSGSRSGTDFTLTISSLQPEDFATYYCQQYGSSARTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 665 aa, synthetic construct
  source 1..665: note = A2A2 Chain 3 - VL-CL-Linker-VL-CL-Hinge-CH2 (L234A, L235A, and P329K, silent)-CH3 (T366W, knob)
DIQMTQSPSSLSASVGDRVTITCRASQSVSRAVAWYQQKPGKAPKLLIYSASSLHSGVPSRFSGSRSGTDFTLTISSLQPEDFATYYCQQYGSSARTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTASSGGSSSGDIQMTQSPSSLSASVGDRVTITCRASQSVSRAVAWYQQKPGKAPKLLIYSASSLHSGVPSRFSGSRSGTDFTLTISSLQPEDFATYYCQQYGSSARTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 665 aa, synthetic construct
  source 1..665: note = A11A11 Chain 3 - VL-CL-Linker-VL-CL-Hinge-CH2 (L234A, L235A, and P329K, silent)-CH3 (T366W, knob)
DIQMTQSPSSLSASVGDRVTITCRASKSVSSALAWYQQKPGKAPKLLIYVGSSRYSGVPSRFSGSRSGTDFTLTISSLQPEDFATYYCQQYYSSPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTASSGGSSSGDIQMTQSPSSLSASVGDRVTITCRASKSVSSALAWYQQKPGKAPKLLIYVGSSRYSGVPSRFSGSRSGTDFTLTISSLQPEDFATYYCQQYYSSPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 441 aa, synthetic construct
  source 1..441: note = A11 Chain 3 - VL-CL-Hinge-CH2 (L234A, L235A, and P329K, silent)-CH3 (T366W, knob)
DIQMTQSPSSLSASVGDRVTITCRASKSVSSALAWYQQKPGKAPKLLIYVGSSRYSGVPSRFSGSRSGTDFTLTISSLQPEDFATYYCQQYYSSPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 9 aa, synthetic construct
GILGFVFTL

SEQ ID NO: 134 - protein, 6 aa, synthetic construct
  source 1..6: note = Exemplary NKG2A polypeptide fragment
TWEESL

SEQ ID NO: 135 - protein, 11 aa, synthetic construct
  source 1..11: note = Exemplary NKG2A polypeptide fragment
SIISPSSWIGV

SEQ ID NO: 136 - protein, 9 aa, synthetic construct
  source 1..9: note = Exemplary NKG2A polypeptide fragment
FRNSSHHPW

SEQ ID NO: 137 - protein, 9 aa, synthetic construct
  source 1..9: note = Exemplary NKG2A polypeptide fragment
IKDSDNAEL

SEQ ID NO: 138 - protein, 5 aa, synthetic construct
  source 1..5: note = Exemplary NKG2A polypeptide fragment
LQVNR

SEQ ID NO: 139 - protein, 7 aa, synthetic construct
  source 1..7: note = Exemplary NKG2A polypeptide fragment
AQCGSSI

SEQ ID NO: 140 - protein, 146 aa, Homo sapiens
  source 1..146: note = human CD94
SFTKLSIEPAFTPGPNIELQKDSDCCSCQEKWVGYRCNCYFISSEQKTWNESRHLCASQKSSLLQLQNTDELDFMSSSQQFYWIGLSYSEEHTAWLWENGSALSQYLFPSFETFNTKNCIAYNPNGNALDESCEDKNRYICKQQLI

SEQ ID NO: 141 - protein, 134 aa, Homo sapiens
  source 1..134: note = human NKG2A
RHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

(19) United States
(12) Patent Application Publication
(10) Pub. No.: US 2026/0226161 A1
(43) Pub. Date: Aug. 6, 2026

(54) AGENTS THAT BIND TO NKG2A AND PD-L1 AND USES THEREOF

(71) Applicant: EXELIXIS, INC., Alameda, CA (US)

(72) Inventors: Bonnie HAMMER, Madison, WI (US); Jeffrey N. HIGAKI, San Mateo, CA (US); Seema KANTAK, Pacifica, CA (US); Hanying LI, Alameda, CA (US); Bee-Cheng SIM, Mountain View, CA (US)

(73) Assignee: EXELIXIS, INC., Alameda, CA (US)

(21) Appl. No.: 19/151,275

(22) PCT Filed: Jul. 7, 2023
(86) PCT No.: PCT/US2023/069758

§ 371 Date: Jul. 25, 2025

Related U.S. Application Data
(60) Provisional application No. 63/441,713, filed on Jan. 27, 2023.

(51) Int. Cl. C07K 16/28; A61K 39/00; A61P 35/00; A61P 37/04

(52) U.S. Cl. CPC C07K 16/2803; A61P 35/00; A61P 37/04; C07K 16/2827; A61K 2039/505; C07K 2317/31; C07K 2317/32; C07K 2317/515; C07K 2317/522; C07K 2317/524; C07K 2317/526; C07K 2317/565; C07K 2317/567; C07K 2317/76; C07K 2317/92

(57) ABSTRACT

The present disclosure provides multispecific binding agents that bind to NKG2A and PD-L1 (e.g., multispecific antibodies such as bispecific antibodies) and uses thereof.

### CROSS-REFERENCE TO RELATED APPLICATIONS

This application claims the benefit of U.S. Provisional Application No. 63/441,713, filed on Jan. 27, 2023, which is incorporated by reference herein in its entirety.

### REFERENCE TO SEQUENCE LISTING SUBMITTED ELECTRONICALLY

This application contains a computer readable Sequence Listing which has been submitted in XML file format with this application, the entire content of which is incorporated by reference herein in its entirety. The Sequence Listing XML file submitted with this application is entitled “14529-109-228_SEQ_LISTING.xml”, was created on Jul. 6, 2023, and is 164,926 bytes in size.

### 1. FIELD

The present disclosure relates generally to binding agents, such as antibodies (including fragments thereof) that bind to both NKG2A and PD-L1, including human NKG2A and human PD-L1, and methods of use thereof.

### 2. BACKGROUND

NKG2A is a cell surface molecule that is typically expressed on NK cells and may also be expressed on T cells, especially on CD8⁺ T cells. Thus, NKG2A is a potential target for removing suppressions of immune cells and enhancing anti-tumor responses by immune cells. However, therapeutic success with binding agents targeting NKG2A has not yet been achieved.

Programmed death ligand 1 (PD-L1) is a cell surface glycoprotein ligand that specifically binds to programmed death receptor 1 (PD-1), a key immune checkpoint receptor. PD-1 is upregulated on activated T cells, B cells, and monocytes and mediates immunosuppression. While PD-L2, the other PD-1 ligand, is expressed primarily on activated antigen-presenting cells (APCs), PD-L1 is broadly expressed, including in cells of hematopoietic lineage, such as activated T cells, B cells, monocytes, dendritic cells and macrophages, and peripheral tissues such as heart, skeletal, muscle, placenta, lung, kidney and liver tissues. The binding of PD-L1 to PD-1 is a negative checkpoint that can activate the downstream signaling of PD-1 receptor in T cells, thus inhibiting the proliferation, cytokine generation and release, and cytotoxicity of T cells. This inhibition of T cell activation and secretion of effector cytokines can prevent autoimmunity and chronic infection. However, many tumor cells use this mechanism to protect themselves from immune attack, resulting in tumor immune evasion. Many cancers overexpress PD-L1, and its overexpression is often associated with poor prognosis. In cancer, the PD-1/PD-L1 interaction stimulates the downstream signals to suppress T cell activation, resulting in tumor cell survival. Although the blockade of PD-1 interaction with its ligands has been proposed as an immunotherapeutic method of enhancing T cell immune responses against tumor cells, there remains an urgent need in the art for agents that can inhibit or prevent PD-1/PD-L1 interaction.

Accordingly, there remains a need in the art for agents enhancing immune responses and treating diseases or disorders such as cancer. The multispecific binding agents, compositions and methods provided herein satisfy this need and provide related advantages.

### 3. SUMMARY

The present disclosure provides multispecific binding agents (e.g., bispecific antibodies) that have a first binding domain that binds to NKG2A (or a complex comprising NKG2A and CD94 or extracellular domain of each thereof), including human NKG2A, and one or more additional binding domains that bind to one or more targets that are not NKG2A (e.g., PD-L1). Such agents include multispecific antibodies (e.g., bispecific antibodies) that bind to NKG2A and one or more additional targets that are not NKG2A (e.g., PD-L1), for example, multispecific antibodies that have a first binding domain that binds to NKG2A (or a complex comprising NKG2A and CD94 or extracellular domain of each thereof), including human NKG2A, and one or more additional binding domains that bind to one or more targets that are not NKG2A (e.g., PD-L1). Such agents, in some embodiments, include multispecific antibodies (e.g., bispecific antibodies) that bind to NKG2A (or a complex comprising NKG2A and CD94 or extracellular domain of each thereof) and one or more additional targets that are not NKG2A (e.g., PD-L1), for example, multispecific antibodies that have a first binding domain that binds to NKG2A (or a complex comprising NKG2A and CD94 or extracellular domain of each thereof), including human NKG2A, and one or more additional binding domains that bind to one or more targets that are not NKG2A (e.g., PD-L1), wherein the first binding domain comprises: a heavy chain variable (VH) region comprising a VH CDR1, a VH CDR2, and a VH CDR3 amino acid sequence set forth in any one of Tables 1-4; and a light chain variable (VL) region comprising a VL CDR1, a VL CDR2, and a VL CDR3 amino acid sequence set forth in any one of Tables 1-4, or wherein the multispecific antibodies compete for the binding of NKG2A (or a complex comprising NKG2A and CD94 or extracellular domain of each thereof), including human NKG2A, with an antibody having a heavy chain variable region and a light chain variable region described herein (e.g., Tables 1-4). In some embodiments, the first binding domain specifically binds to one, two, three, four, five, or all of the following NKG2A polypeptide fragments: an NKG2A polypeptide fragment comprising the amino acid sequence of TWEESL (SEQ ID NO:134), an NKG2A polypeptide fragment comprising the amino acid sequence of SIISPSSWIGV (SEQ ID NO: 135), an NKG2A polypeptide fragment comprising the amino acid sequence of FRNSSHHPW (SEQ ID NO:136), an NKG2A polypeptide fragment comprising the amino acid sequence of IKDSDNAEL (SEQ ID NO:137), an NKG2A polypeptide fragment comprising the amino acid sequence of LQVNR (SEQ ID NO:138), and an NKG2A polypeptide fragment comprising the amino acid sequence of AQCGSSI (SEQ ID NO:139). In some embodiments, the first binding domain specifically binds to a conformational epitope formed by a group of amino acid residues comprising at least one amino acid residue (1) from one of the following amino acid sequences: TWEESL (SEQ ID NO:134), SIISPSSWIGV (SEQ ID NO:135), FRNSSHHPW (SEQ ID NO:136), IKDSDNAEL (SEQ ID NO:137), LQVNR (SEQ ID NO:138), and AQCGSSI (SEQ ID NO:139), or (2) from each one of two, three, four, five or all of the following amino acid sequences: TWEESL (SEQ ID NO:134), SIISPSSWIGV (SEQ ID NO:135), FRNSSHHPW (SEQ ID NO:136), IKDSDNAEL (SEQ ID NO: 137), LQVNR (SEQ ID NO:138), and AQCGSSI (SEQ ID NO:139). In some embodiments, the first binding domain specifically binds to a conformational epitope formed by a group of amino acid residues comprising at least one amino acid residue (1) from one of the following amino acid sequences located on the surface of NKG2A: TWEESL (SEQ ID NO: 134), SIISPSSWIGV (SEQ ID NO:135), FRNSSHHPW (SEQ ID NO:136), IKDSDNAEL (SEQ ID NO:137), LQVNR (SEQ ID NO:138), and AQCGSSI (SEQ ID NO: 139), or (2) from each one of two, three, four, five or all of the following amino acid sequences located on the surface of NKG2A: TWEESL (SEQ ID NO:134), SIISPSSWIGV (SEQ ID NO:135), FRNSSHHPW (SEQ ID NO:136), IKDSDNAEL (SEQ ID NO:137), LQVNR (SEQ ID NO:138), and AQCGSSI (SEQ ID NO:139). In some embodiments, the additional binding domain binds to PD-L1 (e.g., human PD-L1), wherein the additional binding domain comprises: a heavy chain variable (VH) region comprising a VH CDR1, a VH CDR2, and a VH CDR3 amino acid sequence set forth in Table 5; and a light chain variable (VL) region comprising a VL CDR1, a VL CDR2, and a VL CDR3 amino acid sequence set forth in Table 5, or wherein the multispecific antibodies compete for the binding of PD-L1 (including human PD-L1) with an antibody having a heavy chain variable region and a light chain variable region described in Table 5.

The present disclosure also provides nucleic acids encoding a multispecific binding agent provided herein (e.g., an antibody or fragment thereof), vectors comprising one or more of such nucleic acids, and cells comprising the nucleic acid, the vector, or both (such as cells expressing the multispecific binding agent).

The present disclosure also provides compositions comprising a multispecific binding agent described herein. Such compositions, in some embodiments, include multispecific antibodies (e.g., bispecific antibodies) that bind to NKG2A (or a complex comprising NKG2A and CD94 or extracellular domain of each thereof) and one or more additional targets that are not NKG2A (e.g., PD-L1), for example, multispecific antibodies that have a first binding domain that binds to NKG2A (or a complex comprising NKG2A and CD94 or extracellular domain of each thereof), including human NKG2A, and one or more additional binding domains that bind to one or more targets that are not NKG2A (e.g., PD-L1). Such compositions, in some embodiments, include multispecific antibodies (e.g., bispecific antibodies) that bind to NKG2A (or a complex comprising NKG2A and CD94 or extracellular domain of each thereof) and one or more additional targets that are not NKG2A (e.g., PD-L1), for example, multispecific antibodies that have a first binding domain that binds to NKG2A (or a complex comprising NKG2A and CD94 or extracellular domain of each thereof), including human NKG2A, and one or more additional binding domains that bind to one or more targets that are not NKG2A (e.g., PD-L1), wherein the first binding domain comprises: a heavy chain variable (VH) region comprising a VH CDR1, a VH CDR2, and a VH CDR3 amino acid sequence set forth in any one of Tables 1-4; and a light chain variable (VL) region comprising a VL CDR1, a VL CDR2, and a VL CDR3 amino acid sequence set forth in any one of Tables 1-4, or wherein the multispecific antibodies compete for the binding of NKG2A (e.g., human NKG2A) (or a complex comprising NKG2A and CD94 or extracellular domain of each thereof) with an antibody having a heavy chain variable region and a light chain variable region described herein (e.g., Tables 1-4). In some embodiments, the first binding domain specifically binds to one, two, three, four, five, or all of the following NKG2A polypeptide fragments: an NKG2A polypeptide fragment comprising the amino acid sequence of TWEESL (SEQ ID NO:134), an NKG2A polypeptide fragment comprising the amino acid sequence of SIISPSSWIGV (SEQ ID NO:135), an NKG2A polypeptide fragment comprising the amino acid sequence of FRNSSHHPW (SEQ ID NO: 136), an NKG2A polypeptide fragment comprising the amino acid sequence of IKDSDNAEL (SEQ ID NO:137), an NKG2A polypeptide fragment comprising the amino acid sequence of LQVNR (SEQ ID NO:138), and an NKG2A polypeptide fragment comprising the amino acid sequence of AQCGSSI (SEQ ID NO:139). In some embodiments, the first binding domain specifically binds to a conformational epitope formed by a group of amino acid residues comprising at least one amino acid residue (1) from one of the following amino acid sequences: TWEESL (SEQ ID NO:134), SIISPSSWIGV (SEQ ID NO:135), FRNSSHHPW (SEQ ID NO:136), IKDSDNAEL (SEQ ID NO:137), LQVNR (SEQ ID NO: 138), and AQCGSSI (SEQ ID NO:139), or (2) from each one of two, three, four, five or all of the following amino acid sequences: TWEESL (SEQ ID NO:134), SIISPSSWIGV (SEQ ID NO:135), FRNSSHHPW (SEQ ID NO:136), IKDSDNAEL (SEQ ID NO:137), LQVNR (SEQ ID NO:138), and AQCGSSI (SEQ ID NO:139). In some embodiments, the first binding domain specifically binds to a conformational epitope formed by a group of amino acid residues comprising at least one amino acid residue (1) from one of the following amino acid sequences located on the surface of NKG2A: TWEESL (SEQ ID NO:134), SIISPSSWIGV (SEQ ID NO:135), FRNSSHHPW (SEQ ID NO:136), IKDSDNAEL (SEQ ID NO: 137), LQVNR (SEQ ID NO:138), and AQCGSSI (SEQ ID NO:139), or (2) from each one of two, three, four, five or all of the following amino acid sequences located on the surface of NKG2A: TWEESL (SEQ ID NO:134), SIISPSSWIGV (SEQ ID NO:135), FRNSSHHPW (SEQ ID NO:136), IKDSDNAEL (SEQ ID NO:137), LQVNR (SEQ ID NO: 138), and AQCGSSI (SEQ ID NO:139). In some embodiments, the additional binding domain binds to PD-L1 (e.g., human PD-L1), wherein the additional binding domain comprises: a heavy chain variable (VH) region comprising a VH CDR1, a VH CDR2, and a VH CDR3 amino acid sequence set forth in Table 5; and a light chain variable (VL) region comprising a VL CDR1, a VL CDR2, and a VL CDR3 amino acid sequence set forth in Table 5, or wherein the multispecific antibodies compete for the binding of PD-L1 (e.g., human PD-L1) with an antibody having a heavy chain variable region and a light chain variable region described in Table 5.

The present disclosure further provides various uses of the present binding agents and compositions, including, for example, methods for inhibiting the interaction between HLA-E and NKG2A (such as an NKG2A expressed on an immune cell), and methods of preventing suppression of an immune cell or activating a response mediated by an immune cell. Other aspects provided herein include methods for treating a disease or disorder in a subject with a multispecific binding agent or a composition provided herein. Such compositions include multispecific antibodies that have a first binding domain that binds to NKG2A (or a complex comprising NKG2A and CD94 or extracellular domain of each thereof), including human NKG2A, and one or more additional binding domains that bind to one or more targets that are not NKG2A (e.g., PD-L1), wherein the first binding domain VH CDR3 amino acid sequence set forth in any one of Tables 1-4; and a light chain variable (VL) region comprising a VL CDR1, a VL CDR2, and a VL CDR3 amino acid sequence set forth in any one of Tables 1-4, or wherein the multispecific antibodies compete for the binding of NKG2A (e.g., human NKG2A) (or a complex comprising NKG2A and CD94 or extracellular domain of each thereof) with an antibody having a heavy chain variable region and a light chain variable region described herein (e.g., Tables 1-4). In some embodiments, the first binding domain specifically binds to one, two, three, four, five, or all of the following NKG2A polypeptide fragments: an NKG2A polypeptide fragment comprising the amino acid sequence of TWEESL (SEQ ID NO:134), an NKG2A polypeptide fragment comprising the amino acid sequence of SIISPSSWIGV (SEQ ID NO:135), an NKG2A polypeptide fragment comprising the amino acid sequence of FRNSSHHPW (SEQ ID NO: 136), an NKG2A polypeptide fragment comprising the amino acid sequence of IKDSDNAEL (SEQ ID NO:137), an NKG2A polypeptide fragment comprising the amino acid sequence of LQVNR (SEQ ID NO:138), and an NKG2A polypeptide fragment comprising the amino acid sequence of AQCGSSI (SEQ ID NO:139). In some embodiments, the first binding domain specifically binds to a conformational epitope formed by a group of amino acid residues comprising at least one amino acid residue (1) from one of the following amino acid sequences: TWEESL (SEQ ID NO:134), SIISPSSWIGV (SEQ ID NO:135), FRNSSHHPW (SEQ ID NO:136), IKDSDNAEL (SEQ ID NO:137), LQVNR (SEQ ID NO: 138), and AQCGSSI (SEQ ID NO:139), or (2) from each one of two, three, four, five or all of the following amino acid sequences: TWEESL (SEQ ID NO:134), SIISPSSWIGV (SEQ ID NO:135), FRNSSHHPW (SEQ ID NO:136), IKDSDNAEL (SEQ ID NO:137), LQVNR (SEQ ID NO:138), and AQCGSSI (SEQ ID NO:139). In some embodiments, the first binding domain specifically binds to a conformational epitope formed by a group of amino acid residues comprising at least one amino acid residue (1) from one of the following amino acid sequences located on the surface of NKG2A: TWEESL (SEQ ID NO:134), SIISPSSWIGV (SEQ ID NO:135), FRNSSHHPW (SEQ ID NO:136), IKDSDNAEL (SEQ ID NO: 137), LQVNR (SEQ ID NO:138), and AQCGSSI (SEQ ID NO:139), or (2) from each one of two, three, four, five or all of the following amino acid sequences located on the surface of NKG2A: TWEESL (SEQ ID NO:134), SIISPSSWIGV (SEQ ID NO:135), FRNSSHHPW (SEQ ID NO:136), IKDSDNAEL (SEQ ID NO:137), LQVNR (SEQ ID NO: 138), and AQCGSSI (SEQ ID NO:139). In some embodiments, the additional binding domain binds to PD-L1 (e.g., human PD-L1), wherein the additional binding domain VH CDR3 amino acid sequence set forth in Table 5; and a light chain variable (VL) region comprising a VL CDR1, a VL CDR2, and a VL CDR3 amino acid sequence set forth in Table 5, or wherein the multispecific antibodies compete for the binding of PD-L1 (e.g., human PD-L1) with an antibody having a heavy chain variable region and a light chain variable region described in Table 5.

In some embodiments, the binding domain that binds to NKG2A (or a complex comprising NKG2A and CD94 or extracellular domain of each thereof) does not bind to NKG2C. Additionally or alternatively, the binding domain that binds to NKG2A (or a first complex comprising NKG2A and CD94 or extracellular domain of each thereof) does not bind to a second complex comprising NKG2C and CD94. Additionally or alternatively, the binding domain that binds to NKG2A (or a first complex comprising NKG2A and CD94 or extracellular domain of each thereof) does not bind to a second complex comprising the extracellular domain of NKG2C and the extracellular domain of CD94.

The present disclosure further provides binding agents that bind to PD-L1 (e.g., human PD-L1), including those that comprise: a heavy chain variable (VH) region comprising a VH CDR1, a VH CDR2, and a VH CDR3 amino acid sequence set forth in Table 5; and a light chain variable (VL) region comprising a VL CDR1, a VL CDR2, and a VL CDR3 amino acid sequence set forth in Table 5, or those competing for the binding of PD-L1 (e.g., PD-L1) with an antibody having a heavy chain variable region and a light chain variable region described in Table 5. The present disclosure also provides multispecific binding agents (e.g., multispecific antibodies) that have a first binding domain that binds to PD-L1, including human PD-L1, and one or more additional binding domains that bind to one or more targets that are not PD-L1 (e.g., NKG2A). In addition, the present disclosure provides nucleic acids encoding such a binding agent provided herein (e.g., an antibody or fragment thereof), vectors comprising one or more of such nucleic acids, and cells expressing the same, and compositions comprising any one or more of the above. Methods or uses of such binding agents, nucleic acids, vectors, cells, and compositions are also provided herein.

### 4. BRIEF DESCRIPTION OF THE DRAWINGS

FIG. 1 illustrates exemplary results for A3 from cell binding assays, further described in Examples 3 and 6.

FIG. 2 illustrates exemplary results for A3 from HLA-E/NKG2A inhibiting assays, further described in Examples 4 and 6.

FIGS. 3A-3C illustrate exemplary results for A3 from developability assays, further described in Examples 5 and 6.

FIG. 4 illustrates exemplary results for A42 from biolayer interferometry (BLI) binding assays (i.e., Octet binding assays), further described in Example and 6.

FIG. 5 illustrates exemplary results for A42 from HLA-E/NKG2A inhibiting assays, further described in Examples 4 and 6.

FIGS. 6A-6C illustrate exemplary results for A42 from developability assays, further described in Examples 5 and 6.

FIG. 7 illustrates exemplary results for A2 from BLI binding assays, further described in Example 6.

FIG. 8 illustrates exemplary results for A2 from HLA-E/NKG2A inhibiting assays, further described in Examples 4 and 6.

FIGS. 9A-9C illustrate exemplary results for A2 from developability assays, further described in Examples 5 and 6.

FIG. 10 illustrates exemplary results for A11 from BLI binding assays, further described in Example 6.

FIG. 11 illustrates exemplary results for A11 from HLA-E/NKG2A inhibiting assays, further described in Examples 4 and 6.

FIGS. 12A-12C illustrate exemplary results for A11 from developability assays, further described in Examples 5 and 6.

FIGS. 13A-13F illustrate exemplary results from cell binding assays, further described in Example 8.

FIGS. 14A-14F illustrate exemplary results from HLA-E/NKG2A inhibiting assays, further described in Example 9.

FIGS. 15A-15F illustrate exemplary results from PD-L1/PD1 inhibiting assays, further described in Example 10.

FIGS. 16A-16F illustrate exemplary results from cytotoxicity assays, further described in Example 11.

FIGS. 17A-17E provide exemplary results (FIG. 17A) of in vitro functional evaluation assays and statistical analysis thereof (FIG. 17B plotting NK degranulation data shown by differences with 95% CI; FIG. 17C plotting NK cytotoxicity data shown by differences with 95% CI; FIG. 17D plotting NK degranulation data shown by percentages of CD107a/b+ NK cells; and FIG. 17E plotting NK cytotoxicity data shown by percentages of live tumor cells), further described in Example 12.

FIGS. 18A-18B provide exemplary results (FIG. 18A) from a flu-stimulated CD8+ T cell assay and statistical analysis thereof (FIG. 18B), further described in Example 12.

FIGS. 19A-19F illustrate exemplary results from SEC chromatography, further described in Example 13.

FIGS. 20A-20F illustrate exemplary results from HIC chromatography, further described in Example 13.

FIGS. 21A-21F illustrate exemplary results from SMAC chromatography, further described in Example 13.

FIG. 22 illustrates exemplary bispecific antibody formats used in constructing the exemplary bispecific antibodies described herein.

FIGS. 23A-23C provide exemplary results of the CellTrace Far Red and CellTrace Violet labeling experiment, further described in Example 14. FIG. 23A compares the P12×A11, sFc data with various controls at the same tested dose (2 nM). FIG. 23B shows a dose-dependent co-engagement of tumor and NK cells by P12×A11, sFc. FIG. 23C compares various target-to-effector (T:E) ratios used in the experiments.

FIGS. 24A-24B provide exemplary results showing expressions of human HLA-E (hHLA-E) and human PD-L1 (hPD-L1) on engineered MC38 cells (B-hHLA-E plus/hPD-L1 MC38 cells) compared to the unengineered MC38 ones in vitro (FIG. 24A) and ex vivo (FIG. 24B), further described in Example 15.

FIGS. 25A-25C provide exemplary results of in vivo functional evaluation further described in Example 15. FIG. 24A plots body weight changes in all groups, while FIG. 25B shows changes in tumor volume. FIG. 25C focuses on treatments at 10 mg/kg body weight.

FIG. 26: surface rendering of NKG2a and CD94 highlights P12×A11, sFc epitope.

FIG. 27 shows a list of regions significantly protected from Deuterium exchange.

### 5. DETAILED DESCRIPTION

The present disclosure is based, at least in part, on novel multispecific binding agents that bind to both NKG2A and PD-L1 and their properties. Such agents include antibodies (e.g., bispecific antibodies) that bind to NKG2A (e.g., human NKG2A) (or a complex comprising NKG2A and CD94 or extracellular domain of each thereof) and PD-L1 (e.g., human PD-L1). In certain aspects, such binding agents are useful in compositions and in methods for inhibiting the interaction between HLA-E and NKG2A and/or between PD-1 and PD-L1, thereby preventing suppression of an immune cell or activating an anti-tumor response mediated by an immune cell. In addition, the multispecific binding agents provided herein are useful for the killing and/or removal of tumor cells. The binding agents provided herein are useful in compositions and in methods for treating a disease or disorder such as cancer.

As it would be understood, the section or subsection headings as used herein is for organizational purposes only and are not to be construed as limiting and/or separating the subject matter described.

### 5.1. Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010). Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “NKG2A” refers to a polypeptide (“polypeptide” and “protein” are used interchangeably herein) or any native NKG2A from any vertebrate source, including mammals such as primates (e.g., humans, cynomolgus monkey (cyno)), dogs, and rodents (e.g., mice and rats), unless otherwise indicated. NKG2A is also known as, for example, NK cell receptor A, NKG2A-activating NK receptor, NKG2-A/B-activating NK receptor, killer cell lectin like receptor C1 (CD159a), CD159 antigen-like family member A, or NKG2-A/NKG2-B type II integral membrane protein. NKG2A belongs to a family of lectins, which forms a heterodimer with CD94 (or KLRD1) another NK cell-expressed C-type lectin. The NKG2A/CD94 complex binds to HLA-E, a non-classical MHC I molecule, in humans and transduces inhibitory signals, which suppress NK and CD8⁺ T cell activities. NKG2A is a protein encoded by the NKG2A gene (or KLRC1). The term NKG2A encompasses “full-length” NKG2A, as well as any form of NKG2A or any fragment thereof that results from processing in a cell. In some embodiments, an exemplary amino acid sequence of a full-length NKG2A is provided below (see, e.g., gene access no. P26715-1 in the Example section below). In some embodiments, the NKG2A comprises a signal sequence. In some embodiments, the NKG2A does not include a signal sequence. In some embodiments, the term NKG2A refers to a fragment of the full-length NKG2A, which comprises an NKG2A extracellular domain. The term NKG2A also encompasses naturally occurring variants of NKG2A, such as SNP variants, splice variants and allelic variants. An exemplary amino acid sequence of extracellular domain of human NKG2A is provided below: PSTLIQRHNNSSLNTRTQKARHCGHCPEEWITYSNSCYYIGKERRTWEESLLACTSKN SSLLSIDNEEEMKFLSIISPSSWIGVFRNSSHHPWVTMNGLAFKHEIKDSDNAELNCA VLQVNRLKSAQCGSSIIYHCKHKL (SEQ ID NO:74). An exemplary amino acid sequence of human NKG2A is provided below: RHNNSSLNTRTQKARHCGHCPEEWITYSNSCYYIGKERRTWEESLLACTSKNSSLLSI DNEEEMKFLSIISPSSWIGVFRNSSHHPWVTMNGLAFKHEIKDSDNAELNCAVLQVN RLKSAQCGSSIIYHCKHKL (SEQ ID NO:141). An exemplary amino acid sequence of extracellular domain of cynomolgus monkey (cyno) NKG2A is provided below:

(SEQ ID NO: 77)
PSTLTQKHNNSSLNTRTQKARHCGHCPEEWITYSNSCYYIGKEKRTWAES
LLACTLKNSSLLSIDNEEEMKFLTAISPSTWTGVFRDSSQHPWVTINGLT
FKHEIKDSDNAEHNCAMLHARGLKSDRCGSSKIYHCKHKL.

In some embodiments, the term NKG2A as used herein refers to an NKG2A epitope. Additionally or alternatively, the term NKG2A as used herein refers to an epitope of a complex comprising NKG2A and CD94, or a complex comprising the extracellular domains of NKG2A and CD94. In further embodiments, the term NKG2A as used herein refers to an epitope of a complex comprising NKG2A and CD94, or a complex comprising the extracellular domains of NKG2A and CD94, but not an epitope solely on CD94 itself. Additionally or alternatively, the term NKG2A as used herein refers to an epitope of a complex comprising NKG2A and CD94, or a complex comprising the extracellular domains of NKG2A and CD94, but not an epitope solely on NKG2A itself. In some embodiments, the term NKG2A as used herein refers to an epitope of a complex comprising NKG2A and CD94, or a complex comprising the extracellular domains of NKG2A and CD94, but not an epitope solely on NKG2A itself or solely on CD94 itself.

In some embodiments, the term NKG2A as used herein refers to an NKG2A epitope. Additionally or alternatively, the term NKG2A as used herein refers to an NKG2A epitope located on the surface of an NKG2A stabilized by complexing with a CD94. In some embodiments, the term NKG2A as used herein refers to an NKG2A epitope located on the surface of a complex comprising NKG2A and CD94 and solely on NKG2A itself. Additionally or alternatively, the term NKG2A as used herein refers to an NKG2A epitope located on the surface of an NKG2A extracellular domain stabilized by complexing with a CD94 extracellular domain. In some embodiments, the term NKG2A as used herein refers to an NKG2A epitope located on the surface of a complex comprising the extracellular domains of NKG2A and CD94 and solely on the extracellular domain of NKG2A itself.

The term “NKG2C” refers to a polypeptide (“polypeptide” and “protein” are used interchangeably herein) or any native NKG2C from any vertebrate source, including mammals such as primates (e.g., humans, cynomolgus monkey (cyno)), dogs, and rodents (e.g., mice and rats), unless otherwise indicated. NKG2C is also known as, for example, KLRC2, CD159c, NKG2-C, NKG2C, killer cell lectin like receptor C2. NKG2C is a protein encoded by the NKG2C gene (or KLRC2). The term NKG2C encompasses “full-length” NKG2C, as well as any form of NKG2C or any fragment thereof that results from processing in a cell. In some embodiments, the NKG2C comprises a signal sequence. In some embodiments, the NKG2C does not include a signal sequence. In some embodiments, the term NKG2C refers to a fragment of the full-length NKG2C, which comprises an NKG2C extracellular domain. The term NKG2C also encompasses naturally occurring variants of NKG2C, such as SNP variants, splice variants and allelic variants. The NKG2C gene is described in various databases with the following ID numbers: HGNC 6375; NCBI Entrez Gene 3822; Ensembl ENSG00000205809; OMIM® 602891; and UniProtKB/Swiss-Prot P26717. An exemplary extracellular domain of human NKG2C is shown in the Example section below (see SEQ ID NO:75).

In some embodiments, the term NKG2C as used herein refers to an NKG2C epitope. Additionally or alternatively, the term NKG2C as used herein refers to an epitope of a complex comprising NKG2C and CD94, or a complex comprising the extracellular domains of NKG2C and CD94. In further embodiments, the term NKG2C as used herein refers to an epitope of a complex comprising NKG2C and CD94, or a complex comprising the extracellular domains of NKG2C and CD94, but not an epitope solely on CD94 itself. Additionally or alternatively, the term NKG2C as used herein refers to an epitope of a complex comprising NKG2C and CD94, or a complex comprising the extracellular domains of NKG2C and CD94, but not an epitope solely on NKG2C itself. In some embodiments, the term NKG2C as used herein refers to an epitope of a complex comprising NKG2C and CD94, or a complex comprising the extracellular domains of NKG2C and CD94, but not an epitope solely on NKG2C itself or soley on CD94 itself.

The term “HLA-E” refers to a polypeptide (“polypeptide” and “protein” are used interchangeably herein) or any native HLA-E or an ortholog thereof from any vertebrate source, including mammals such as primates (e.g., humans, cynomolgus monkey (cyno)), dogs, and rodents (e.g., mice and rats), unless otherwise indicated. HLA-E is also known as, for example, major histocompatibility complex Class I, E; HLA Class I histocompatibility antigen, alpha chain E; MHC Class I Antigen E; HLA-6.2; MHC Class Ib antigen; HLAE; or QA1. HLA-E is a protein that in humans is encoded by the HLA-E gene. HLA-E belongs to the HLA class I heavy chain paralogues, and is approximately 45 kDa and anchored in the membrane. The term HLA-E encompasses “full-length” HLA-E, as well as any form of HLA-E or any fragment thereof that results from processing in a cell. In some embodiments, the HLA-E comprises a signal sequence. In some embodiments, the HLA-E does not include a signal sequence. In some embodiments, the term HLA-E refers to a fragment of the full-length HLA-E, which comprises an HLA-E extracellular domain. The term HLA-E also encompasses naturally occurring variants of HLA-E, such as SNP variants, splice variants and allelic variants. The HLA-E gene is described in various databases with the following ID numbers: HGNC 4962; NCBI Entrez Gene 3133; Ensembl ENSG00000204592; OMIM® 143010; and UniProtKB/Swiss-Prot P13747.

The term “CD94” refers to a polypeptide (“polypeptide” and “protein” are used interchangeably herein) or any native CD94 from any vertebrate source, including mammals such as primates (e.g., humans, cynomolgus monkey (cyno)), dogs, and rodents (e.g., mice and rats), unless otherwise indicated. CD94 is also known as, for example, Killer Cell Lectin Like Receptor D1; Killer Cell Lectin-like Receptor Subfamily D, Member 1; Natural Killer Cells Antigen CD94; NK cell Receptor; or KP43. CD94 is a protein encoded by the KLRD1 gene. CD94 is an immune receptor involved in self-non-self discrimination. It is in complex with NKG2A or NKG2C on cytotoxic and regulatory lymphocyte subsets, recognizes non-classical major histocompatibility (MHC) class Ib molecule HLA-E loaded with self-peptides derived from the signal sequence of classical MHC class Ia and non-classical MHC class Ib molecules. CD94-NKG2A acts as an immune inhibitory receptor, and it is a key inhibitory receptor on natural killer (NK) cells that regulates their activation and effector functions. CD94-NKG2C acts as an immune activating receptor, and it is on cytotoxic lymphocyte subsets that recognizes HLA-E loaded with signal sequence-derived peptides from non-classical MHC class Ib HLA-G molecules. The term CD94 encompasses “full-length” CD94, as well as any form of CD94 or any fragment thereof that results from processing in a cell. In some embodiments, the CD94 comprises a signal sequence. In some embodiments, the CD94 does not include a signal sequence. In some embodiments, the term CD94 refers to a fragment of the full-length CD94, which comprises a CD94 extracellular domain. The term CD94 also encompasses naturally occurring variants of CD94, such as SNP variants, splice variants and allelic variants. The CD94 gene is described in various databases with the following ID numbers: HGNC 6378; NCBI Entrez Gene 3824; Ensembl ENSG00000134539; OMIM® 602894; and UniProtKB/Swiss-Prot Q13241. An exemplary amino acid sequence of human CD94 is provided below:

(SEQ ID NO: 140)
SFTKLSIEPAFTPGPNIELQKDSDCCSCQEKWVGYRCNCYFISSEQKTWN
ESRHLCASQKSSLLQLQNTDELDFMSSSQQFYWIGLSYSEEHTAWLWENG
SALSQYLFPSFETFNTKNCIAYNPNGNALDESCEDKNRYICKQQLI.

The term “Programmed Cell Death Ligand-1 (PD-L1),” “Programmed Death Ligand-1,” “PD-1 ligand 1” or similar terms refers to a polypeptide (“polypeptide” and “protein” are used interchangeably herein) or any native PD-L1 from any vertebrate source, including mammals such as primates (e.g., humans, cynomolgus monkey (cyno)), dogs, and rodents (e.g., mice and rats), unless otherwise indicated. PD-L1, also known as cluster of differentiation 274 (CD274) or B7 homolog 1 (B7-H1), is a protein that in humans is encoded by the CD274 gene. PD-L1 is one of two naturally-occurring cell surface glycoprotein ligands for PD-1 (the other is PD-L2). Like PD-1, PD-L1 belongs to the immunoglobulin superfamily and consists of two extracellular Ig domains, an N-terminal V domain, and a C-terminal constant domain. PD-L1 is known in the art to downregulate T cell activation and cytokine secretion upon binding to PD-1. The term PD-L1 encompasses “full-length” PD-L1, as well as any form of PD-L1 or any fragment thereof that results from processing in the cell. The term PD-L1 also encompasses naturally occurring variants of PD-L1, such as SNP variants, splice variants and allelic variants. An exemplary full-length amino acid sequence of human PD-L1 is provided below (exemplary extracellular domain=underline text): MRIFAVFIFMTYWHLLNAFTVTVPKDLYVVEYGSNMTIECKFPVEKQLDLAALIVY WEMEDKNIIQFVHGEEDLKVQHSSYRQRARLLKDQLSLGNAALQITDVKLQDAGVY RCMISYGGADYKRITVKVNAPYNKINQRILVVDPVTSEHELTCQAEGYPKAEVIWTS SDHQVLSGKTTTTNSKREEKLFNVTSTLRINTTTNEIFYCTFRRLDPEENHTAELVIPE LPLAHPPNERTHL VILGAILLCLGVALTFIFRLRKGRMMDVKKCGIQDTNSKKQSDT HLEET (SEQ ID NO:100). Other related PD-L1 polypeptides that are also encompassed by the term PD-L1 include fragments, derivatives (e.g., substitution, deletion, truncations, and insertion variants), fusion polypeptides, and interspecies homologs that retain PD-L1 activity and/or are sufficient to generate an anti-PD-L1 immune response. As those skilled in the art will appreciate, a PD-L1 binding agent (e.g., an antibody) described herein can bind to a PD-L1 polypeptide, a PD-L1 polypeptide fragment, a PD-L1 antigen, and/or a PD-L1 epitope. An epitope may be part of a larger PD-L1 antigen, which may be part of a larger PD-L1 polypeptide fragment, which, in turn, may be part of a larger PD-L1 polypeptide. PD-L1 may exist in a native or denatured form. PD-L1 polypeptides described herein may be isolated from a variety of sources, such as from human tissue types or from another source, or prepared by recombinant or synthetic methods. A PD-L1 polypeptide may comprise a polypeptide having the same amino acid sequence as a corresponding PD-L1 polypeptide derived from nature. Orthologs to the PD-L1 polypeptide are also well known in the art.

The term “Programmed Cell Death-1 (PD-1),” “Programmed Death-1,” “PD-1 receptor” or similar terms refers to a polypeptide (“polypeptide” and “protein” are used interchangeably herein) or any native PD-1 from any vertebrate source, including mammals such as primates (e.g., humans, cynomolgus monkey (cyno)), dogs, and rodents (e.g., mice and rats), unless otherwise indicated. PD-1, also known as CD279 (cluster of differentiation 279), is an immunoinhibitory receptor belonging to the CD28 family. PD-1 is expressed predominantly on previously activated T cells in vivo, and binds to two ligands, PD-L1 and PD-L2. PD-1 belongs to the immunoglobulin superfamily and consists of two extracellular Ig domains, an N-terminal V domain, and a C-terminal constant domain. PD-1 further contains two cytoplasmic tyrosine-based signaling motifs, an immunoreceptor tyrosine-based inhibition motif (ITIM) and an immunoreceptor tyrosine-based switch motif (ITSM). The term PD-1 encompasses “full-length” PD-1, as well as any form of PD-1 or any fragment thereof that results from processing in the cell. The term PD-1 also encompasses naturally occurring variants of PD-1, such as SNP variants, splice variants and allelic variants. Following T cell stimulation, PD-1 is known in the art to recruit the tyrosine phosphatase SHP-2 to the ITSM motif within its cytoplasmic tail, leading to, among other things, the dephosphorylation of effector molecules such as CD3 Zeta, PKC theta and ZAP70 that are involved in the CD3 T cell signaling cascade (Carter et al. (2002) Eur J Immunol 32:634-43). An exemplary full-length amino acid sequence of human PD-1 is provided below:

(SEQ ID NO: 101)
MQIPQAPWPVVWAVLQLGWRPGWFLDSPDRPWNPPTFSPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQFQTLVVGVVGGLLGSLVLLVWVLAVICSRAARGTI
GARRTGQPLKEDPSAVPVFSVDYGELDFQWREKTPEPPVPCVPEQTEYAT
IVFPSGMGTSSPARRGSADGPRSAQPLRPEDGHCSWPL.

As used herein, the term “binding agent” or a grammatical equivalent thereof refers to a molecule (e.g., antibody) with one or more antigen-binding sites that binds an antigen. In some embodiments, a binding agent as described herein is an antibody (including a multispecific antibody and an antibody fragment, such as an antigen-binding fragment or an epitope-binding fragment) or other peptide-based molecule as well as a conjugate of an antibody, antibody fragment, or peptide-based molecule (e.g., an antibody-drug conjugate) that binds to NKG2A (such as human NKG2A) and/or PD-L1 (such as human PD-L1).

The terms “antibody,” “immunoglobulin,” and “Ig” are used interchangeably herein, and are used in the broadest sense and specifically cover, for example polyclonal antibodies, monoclonal antibodies (including agonist, antagonist, neutralizing antibodies, full-length monoclonal antibodies), antibody compositions with polyepitopic or monoepitopic specificity, recombinantly produced antibodies, single domain (e.g., VHH) antibodies, monospecific antibodies, multispecific antibodies (including bispecific antibodies), synthetic antibodies, chimeric antibodies, humanized antibodies, or human versions of antibodies having full-length heavy and/or light chains. VHH as used herein refers to a domain antibody derived from a variable region of a heavy chain only antibody. Exemplary single domain antibodies include, but are not limited to, antibodies naturally devoid of light chains such as those from Camelidae species (e.g., llama), single domain antibodies derived from conventional 4-chain antibodies, engineered antibodies and single domain scaffolds other than those derived from antibodies. Single domain antibodies may be derived from any species including, but not limited to mouse, human, camel, llama, goat, rabbit, and bovine. VHH can also be derived from other species besides Camelidae that may produce heavy chain antibodies naturally devoid of light chain. Antibodies also include antibody fragments (and/or polypeptides that comprise antibody fragments) that retain NKG2A and/or PD-L1 binding characteristics. Non-limiting examples of antibody fragments include antigen-binding regions and/or effector regions of the antibody, e.g., Fab, Fab′, F(ab′)₂, Fv, scFv, (scFv)₂, single chain antibody molecule, dual variable domain antibody, single variable domain, linear antibody, V region, a multispecific antibody formed from antibody fragments, F(ab)₂, Fd, Fc, diabody, di-diabody, disulfide-linked Fvs (dsFv), single-domain antibody (e.g., nanobody) or other fragments (e.g., fragments consisting of the variable regions of the heavy and light chains that are non-covalently coupled). In general terms, a variable (V) region domain may be any suitable arrangement of immunoglobulin heavy (VH) and/or light (VL) variable domains. For example, antibodies also include tetrameric antibodies comprising two heavy chain and two light chain molecules, an antibody light chain monomer, and an antibody heavy chain monomer. Thus, for example, the V region domain may be dimeric and contain VHH-VHH, VH-VH, VH-VL, or VL-VL dimers that bind NKG2A and/or PD-L1. If desired, the VH and VL may be covalently coupled either directly or through a linker to form a single chain Fv (scFv). For ease of reference, scFv proteins are referred to herein as included in the category “antibody fragments.” Another form of an antibody fragment is a peptide comprising one or more complementarity determining regions (CDRs) of an antibody. CDRs (also termed “minimal recognition units” or “hypervariable regions”) can be obtained by constructing polynucleotides that encode one or more CDRs of interest. Such polynucleotides are prepared, for example, by using the polymerase chain reaction to synthesize the variable region using mRNA of antibody-producing cells as a template (see, for example, Larrick et al., Methods: A Companion to Methods in Enzymology, 2:106 (1991); Courtenay-Luck, “Genetic Manipulation of Monoclonal Antibodies,” in Monoclonal Antibodies Production, Engineering and Clinical Application, Ritter et al. (eds.), page 166, Cambridge University Press (1995); and Ward et al., “Genetic Manipulation and Expression of Antibodies,” in Monoclonal Antibodies: Principles and Applications, Birch et al., (eds.), page 137, Wiley-Liss, Inc. (1995)). Antibody fragments may be incorporated, for example, into single domain antibodies, maxibodies, minibodies, intrabodies, diabodies, triabodies, tetrabodies, variable domains of new antigen receptors (v-NAR), and bis-single chain Fv regions (see, e.g., Hollinger and Hudson, Nature Biotechnology, 23(9):1126-1136, 2005). In some embodiments, antibodies comprising a VH and/or VL further contain a light chain and/or a heavy chain constant region, such as one or more constant regions, including one or more IgG1, IgG2, IgG3 and/or IgG4 constant regions. In some embodiments, antibodies can include epitope-binding fragments of any of the above. The antibodies described herein can be of any class (e.g., IgG, IgE, IgM, IgD, and IgA) or any subclass (e.g., IgG1, IgG2, IgG3, IgG4, IgA1, and IgA2) of immunoglobulin molecule.

The term “monospecific” when used in reference to a binding agent (e.g., an antibody) as used herein denotes a binding agent that has one or more binding sites each of which binds to the same epitope of the same antigen.

The term “multispecific” when used in reference to a binding agent (e.g., an antibody) means that the binding agent is able to specifically bind to at least two distinct epitopes, for example two binding sites each formed by a pair of an antibody heavy chain variable domain (VH) and an antibody light chain variable domain (VL) or each formed by a pair of VHH domains binding to different antigens or to different epitopes on the same antigen. Such a bispecific binding agent (e.g., an antibody) may have a 1+1 format (comprising one binding site for a first antigen or epitope and one binding site for a second antigen or epitope). Other bispecific binding agent (e.g., an antibody) formats may be 2+1 or 1+2 formats (comprising two binding sites for a first antigen or epitope and one binding site for a second antigen or epitope) or 2+2 format (comprising two binding sites for a first antigen or epitope and two binding sites for a second antigen or epitope). When a bispecific binding agent (e.g., an antibody) comprises two antigen-binding sites, each may bind to a different epitope. Such a bispecific binding agent (e.g., an antibody) may bind to two different epitopes on the same antigen (e.g., epitopes on NKG2A or PD-L1).

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides are substantially identical, meaning they have at least 70%, at least 75%, at least 80%, at least 85%, or at least 90%, and in some embodiments at least 95%, 96%, 97%, 98%, or 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full-length of the sequences being compared, such as a nucleotide sequence encoding a protein of interest.

A “conservative amino acid substitution” is one in which one amino acid residue is replaced with another amino acid residue having a side chain with similar chemical characteristics. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Generally, conservative substitutions in the sequences of the polypeptides, soluble proteins, and/or antibodies of the disclosure do not abrogate the binding of the polypeptide, soluble protein, or antibody containing the amino acid sequence, to the target binding site. Methods of identifying amino acid conservative substitutions which do not eliminate binding are well-known in the art.

The term “polypeptide” refers to polymers of amino acids of any length. The polymer can be linear or branched, it can comprise modified amino acids, and it can include (e.g., be interrupted by) non-amino acids. The terms also encompass an amino acid polymer that has been modified naturally or by intervention; for example, disulfide bond formation, glycosylation, lipidation, acetylation, phosphorylation, or any other manipulation or modification, such as linkage to or conjugation with (directly or indirectly) a moiety such as a labeling component or a drug (e.g., toxin). Also included within the definition are, for example, polypeptides containing one or more analogs of an amino acid (including, for example, unnatural amino acids), as well as other modifications known in the art. It is understood that, because the polypeptides of this disclosure can be based upon antibodies or other members of the immunoglobulin superfamily, in some embodiments, the polypeptides can occur as single chains or dimers of single chains.

As used herein, an “antigen” is a moiety or molecule that contains an epitope to which a binding agent (e.g., an antibody) can bind. As such, an antigen can be bound by an antibody. In some embodiments, the antigen, to which a binding agent (e.g., an antibody) described herein binds, is NKG2A (e.g., human NKG2A), or a fragment thereof, including a fragment that comprises one or more domains of NKG2A. In some embodiments, the antigen, to which a binding agent (e.g., an antibody) described herein binds, is PD-L1 (e.g., human PD-L1), or a fragment thereof, including a fragment that comprises one or more domains of PD-L1.

As used herein, an “epitope” is a term in the art and refers to a localized region of an antigen to which an antibody can bind. An epitope can be a linear epitope or a conformational, non-linear, or discontinuous, epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope), e.g., human NKG2A or human PD-L1. It will be appreciated by one of skill in the art that, in general, a linear epitope may or may not be dependent on secondary, tertiary, or quaternary structure. For example, in some embodiments, an antibody binds to a group of amino acids regardless of whether they are folded in a natural three-dimensional protein structure. In other embodiments, an antibody requires amino acid residues making up the epitope to exhibit a particular conformation (e.g., bend, twist, turn or fold) in order to recognize and bind the epitope.

An antibody binds “an epitope” or “essentially the same epitope” or “the same epitope” as a reference antibody, when the two antibodies recognize identical, overlapping or adjacent epitopes in a three-dimensional space. The most widely used and rapid methods for determining whether two antibodies bind to identical, overlapping or adjacent epitopes in a three-dimensional space are competition assays, which can be configured in a number of different formats, for example, using either labeled antigen or labeled antibody. In some assays, the antigen is immobilized on a 96-well plate, or expressed on a cell surface, and the ability of unlabeled antibodies to block the binding of labeled antibodies is measured using radioactive, fluorescent or enzyme labels.

“Epitope binning” is the process of grouping antibodies based on the epitopes they recognize. More particularly, epitope binning comprises methods and systems for discriminating the epitope recognition properties of different antibodies, using competition assays combined with computational processes for clustering antibodies based on their epitope recognition properties and identifying antibodies having distinct binding specificities.

As used herein, the terms “specifically binds,” “specifically recognizes,” “immunospecifically binds,” “selectively binds,” “immunospecifically recognizes” and “immunospecific” are analogous terms in the context of antibodies and refer to molecules that bind to an antigen (e.g., epitope) as such binding is understood by one skilled in the art. In some embodiments, “specifically binds” means, for instance that a polypeptide or molecule interacts more frequently, more rapidly, with greater duration, with greater affinity, or with some combination of the above to the epitope, protein, or target molecule than with alternative substances, including related and unrelated proteins. For example, a molecule that specifically binds to an antigen may bind to other peptides or polypeptides, generally with lower affinity as determined by, e.g., immunoassays, BIACORE™, KinExA 3000 instrument (Sapidyne Instruments, Boise, ID), the OctetQK384 system (ForteBio, Menlo Park, CA), or other assays known in the art. In some embodiments, an antibody or antigen-binding domain binds to or specifically binds to an antigen when it binds to the antigen with higher affinity than to any cross-reactive antigen as determined using experimental techniques, such as radioimmunoassays (RIAs) and enzyme linked immunosorbent assays (ELISAs). Typically a specific or selective reaction will be at least twice background signal or noise and may be more than 10 times background. See, e.g., Fundamental Immunology 332-36 (Paul ed., 2d ed. 1989) for a discussion regarding binding specificity. In some embodiments, the extent of binding of an antibody or antigen-binding domain to a “non-target” protein is less than about 10% of the binding of the antibody or antigen-binding domain to its particular target antigen, for example, as determined by fluorescence activated cell sorting (FACS) analysis or RIAs. In some embodiments, molecules that specifically bind to an antigen bind to the antigen with a Ka that is at least 2 logs, 2.5 logs, 3 logs, 4 logs or greater than the Ka when the molecules bind to another antigen. In some embodiments, molecules that specifically bind to an antigen do not cross react with other proteins. In another specific embodiment, molecules that specifically bind to an antigen do not cross react with other non-NKG2A proteins. In some embodiments “specifically binds” means, for instance, that a polypeptide or molecule binds a protein or target with a K_{D} of about 0.1 mM or less, but more usually less than about 1 μM. In some embodiments, “specifically binds” means that a polypeptide or molecule binds a target with a K_{D} of at least about 0.1 μM or less, at least about 0.01 μM or less, or at least about 1 nM or less. Because of the sequence identity between homologous proteins in different species, specific binding can include a polypeptide or molecule that recognizes a protein or target in more than one species. Likewise, because of homology within certain regions of polypeptide sequences of different proteins, specific binding can include a polypeptide or molecule that recognizes more than one protein or target. It is understood that, in some embodiments, a polypeptide or molecule that specifically binds a first target may or may not specifically bind a second target. As such, “specific binding” does not necessarily require (although it can include) exclusive binding, e.g., binding to a single target. Thus, a polypeptide or molecule can, in some embodiments, specifically bind more than one target. In some embodiments, multiple targets can be bound by the same antigen-binding site on the polypeptide or molecule. For example, an antibody can, in certain instances, comprise two identical antigen-binding sites, each of which specifically binds the same epitope on two or more proteins. In certain alternative embodiments, an antibody can be bispecific and comprise at least two antigen-binding sites with differing specificities. Generally, but not necessarily, reference to “binding” means “specific binding”.

“Binding affinity” generally refers to the strength of the sum total of noncovalent interactions between a single binding site of a molecule (e.g., a binding agent such as an antibody) and its binding partner (e.g., an antigen such as NKG2A). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. In one embodiment, the “K_{D}” or “K_{D} value” may be measured by biolayer interferometry (BLI) using, for example, the OctetQK384 system (ForteBio, Menlo Park, CA). Alternatively, the K_{D} may also be measured in a radiolabeled antigen-binding assay (RIA), for example, performed with the Fab version of an antibody of interest and its antigen (Chen, et al., (1999) J. Mol Biol 293:865-881) or using surface plasmon resonance (SPR) assays by BIACORE™, using, for example, a BIACORE™-2000 or a BIACORE™-3000 (BIACORE™, Inc., Piscataway, NJ). An “on-rate” or “rate of association” or “association rate” or “kₒₙ,” as well as an “off-rate” or “rate of dissociation” or “dissociation rate” or “k_{off},” can also be determined with the same SPR or BLI techniques described above using, for example, the OctetQK384 system (ForteBio, Menlo Park, CA) or a BIACORE™-2000 or a BIACORE™-3000 (BIACORE™, Inc., Piscataway, NJ), respectively.

The term “compete” or any grammatical variation thereof when used in the context of binding agents (e.g., antibodies) means binding agents that compete for the same epitope or binding site on a target, which includes competition between such binding agents as determined by an assay in which the binding agent under study prevents or inhibits the specific binding of a reference molecule (e.g., a reference ligand, or reference antigen-binding protein, such as a reference antibody) to a common antigen (e.g., NKG2A or PD-L1). Numerous types of competitive binding assays can be used to determine if a test binding agent competes with a reference molecule for binding to NKG2A (e.g., human NKG2A) or PD-L1 (e.g., human PD-L1). Examples of assays that can be employed include solid phase direct or indirect radioimmunoassay (RIA); solid phase direct or indirect enzyme immunoassay (EIA), sandwich competition assay (see, e.g., Stahli et al., (1983) Methods in Enzymology 9:242-253); solid phase direct biotin-avidin EIA (see, e.g., Kirkland et al., (1986) J. Immunol. 137:3614-3619 or Cheung, et al., (1990) Virology 176:546-552); solid phase direct labeled assay; solid phase direct labeled sandwich assay (see, e.g., Harlow and Lane, (1988) Antibodies, A Laboratory Manual, Cold Spring Harbor Press); solid phase direct label RIA using I-125 label (see, e.g., Morel et al., (1988) Molec. Immunol. 25:7-15); and direct labeled RIA (Moldenhauer et al., (1990) Scand. J. Immunol. 32:77-82). Typically, such an assay involves the use of a purified antigen (e.g., NKG2A, such as human NKG2A, or PD-L1, such as human PD-L1) bound to a solid surface or cells bearing either of an unlabelled test antigen-binding protein (e.g., test NKG2A antibody or test PD-L1 antibody) or a labeled reference antigen-binding protein (e.g., reference NKG2A antibody or reference PD-L1 antibody). Competitive inhibition may be measured by determining the amount of label bound to the solid surface or cells in the presence of the test antigen-binding protein. Usually, the test antigen-binding protein is present in excess. Antibodies identified by competition assay (competing antibodies) include antibodies binding to the same epitope as the reference antibody and/or antibodies binding to an adjacent epitope sufficiently proximal to the epitope bound by the reference for antibodies steric hindrance to occur (e.g., similar epitope or overlapping epitope). Usually, when a competing antibody is present in excess, it will inhibit specific binding of a reference antibody to a common antigen by at least 20%, for example, at least 20%, 25%, 30%, 35%, 40%, 45%, 50%, 55%, 60%, 65%, 70% or 75%. In some instance, binding is inhibited by at least 80%, 85%, 90%, 95%, 96%, 97%, 98%, 99% or more.

As used herein, the term “constant region” or “constant domain” is a well-known antibody term of art and refers to an antibody portion, for example, a carboxyl terminal portion of a light and/or heavy chain which is not directly involved in binding of an antibody to an antigen but which can exhibit various effector functions, such as interaction with an Fc receptor. The term includes the portion of an immunoglobulin molecule having a generally more conserved amino acid sequence relative to an immunoglobulin variable domain.

Antibody “effector functions” refer to those biological activities attributable to the Fc region (e.g., a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226 (according to the EU numbering system), or from Pro230 (according to the EU numbering system), to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. An exemplary Fc region sequence is provided below (CH2 domain=bold text; CH3 domain=underline text):

(SEQ ID NO: 83)
CPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSC
SVMHEALHNHYTQKSLSLSPGK.

A “functional Fc region” possesses an “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis (such as antibody-dependent cellular phagocytosis, i.e., ADCP); down regulation of cell surface receptors (e.g., B cell receptor; BCR), etc. Such effector functions generally require the Fc region to be combined with a binding region or binding domain (e.g., an antibody variable region or domain) and can be assessed using various assays as disclosed.

A “native sequence Fc region” comprises an amino acid sequence identical to the amino acid sequence of an Fc region found in nature, and not manipulated, modified, and/or changed (e.g., isolated, purified, selected, including or combining with other sequences such as variable region sequences) by a human. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification (e.g., substituting, addition, or deletion), preferably one or more amino acid substitution(s). In some embodiments, the variant Fc region has at least one amino acid substitution compared to a native sequence Fc region or to the Fc region of a parent polypeptide, for example, from about one to about ten amino acid substitutions, and preferably from about one to about five amino acid substitutions in a native sequence Fc region or in the Fc region of the parent polypeptide. The variant Fc region described herein can possess at least about 80% homology with a native sequence Fc region and/or with an Fc region of a parent polypeptide, or at least about 90% homology therewith, for example, at least about 95% homology therewith. The variant Fc region herein described herein may have a loss of an effector function (e.g., silent Fc). An exemplary variant Fc region (“silent Fc”) sequence is provided below (CH2 domain=bold text with amino acid changes underlined; CH3 domain=underline text):

(SEQ ID NO: 84)
CPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVSN
KALKAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSC
SVMHEALHNHYTQKSLSLSPGK

As used herein, the term “heavy chain” when used in reference to an antibody refers to a polypeptide chain of about 50-70 kDa, wherein the amino-terminal portion includes a variable region of about 120 to 130 or more amino acids, and a carboxy-terminal portion includes one or more constant regions. The “heavy chain” can refer to any distinct types, e.g., for example, alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG1, IgG2, IgG3 and IgG4.

As used herein, the term “light chain” when used in reference to an antibody can refer to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, e.g., kappa (κ) or lambda (2) based on the amino acid sequence of the constant domains. Light chain amino acid sequences are well known in the art.

The terms “antigen-binding fragment,” “antigen-binding domain,” “antigen-binding region,” and similar terms refer to that portion of an antibody, which comprises the amino acid residues that interact with an antigen and confer on the binding fragment, domain, or region its specificity and affinity for the antigen (e.g., the CDRs). “Antigen-binding fragment” as used herein includes “antibody fragment,” which comprises a portion of an antibody including one or more CDRs, such as the antigen-binding or variable region of the antibody.

Antibodies described herein include, but are not limited to, synthetic antibodies, monoclonal antibodies, recombinantly produced antibodies, multispecific antibodies (e.g., bispecific antibodies), human antibodies, humanized antibodies, chimeric antibodies, intrabodies, single-chain Fvs (scFv) (e.g., including monospecific, bispecific, etc.), camelized antibodies, Fab fragments, F(ab′) fragments, disulfide-linked Fvs (sdFv), anti-idiotypic (anti-Id) antibodies, and epitope-binding fragments of any of the above.

In some embodiments, antibodies described herein include immunoglobulin molecules and immunologically active portions of immunoglobulin molecules, including molecules that contain one or more antigen-binding sites that bind to an NKG2A antigen and/or a PD-L1 antigen.

Antibodies can be of any type (e.g., IgG, IgE, IgM, IgD, IgA or IgY), any class (e.g., IgG1, IgG2, IgG3, IgG4, IgAQ1 or IgA2), or any subclass (e.g., IgG2a or IgG2b) of immunoglobulin molecule. In some embodiments, antibodies described herein are IgG antibodies (e.g., human IgG), or a class (e.g., human IgG1, IgG2, IgG3 or IgG4) or a subclass thereof.

In some embodiments, an antibody is a 4-chain antibody unit comprising two heavy (H) chain/light (L) chain pairs. In further embodiments, the amino acid sequences of the H chains are identical and the amino acid sequences of the L chains are identical. In other embodiments, the amino acid sequences of the H chains are different from each other. Additionally or alternatively, the amino acid sequences of the L chains are different from each other. For example, an antibody comprises a first H/L chain pair and a second H/L chain pair, wherein the first H/L chain pair binds to an NKG2A antigen and the second H/L chain pair binds to a non-NKG2A (such as PD-L1) antigen. In some embodiments, an antibody is a 2-chain antibody unit comprising a VHH-VHH pair. In further embodiments, the amino acid sequences of the VHH are identical. In other embodiments, the amino acid sequence of the VHH are different from each other. For example, an antibody comprises a first VHH and a second VHH, wherein the first VHH binds to an NKG2A antigen and the second VHH binds to a non-NKG2A (such as PD-L1) antigen. In some embodiments, the H and/or L chains comprise constant regions, for example, human constant regions. In some embodiments, the L chain constant region of such antibodies is a kappa or lambda light chain constant region, for example, a human kappa or lambda light chain constant region. In some embodiments, the H chain constant region of such antibodies comprises a gamma heavy chain constant region, for example, a human gamma heavy chain constant region. In some embodiments, such antibodies comprise IgG constant regions, for example, human IgG constant regions (e.g., IgG1, IgG2, IgG3, and/or IgG4 constant regions).

An antibody or fragment thereof may preferentially bind to NKG2A (such as human NKG2A) and/or PD-L1 (such as human PD-L1), meaning that the antibody or fragment thereof binds NKG2A and/or PD-L1 with greater affinity than it binds to a control protein (e.g., unrelated control proteins such as hen egg white lysozyme, or NKG2C) and/or binds human NKG2A and/or PD-L1 with greater affinity than it binds to an unrelated control protein. For example, the antibody or fragment thereof may specifically recognize and bind NKG2A and/or PD-L1, or a portion of each thereof. “Specific binding” means that the antibody or fragment thereof binds to NKG2A and/or PD-L1 with an affinity that is at least 5, 10, 15, 20, 25, 50, 100, 250, 500, 1000, or 10,000 times greater than the affinity for an unrelated control protein (e.g., hen egg white lysozyme). In some embodiments, the antibody or fragment thereof may bind NKG2A and/or PD-L1 substantially exclusively (e.g., is able to distinguish NKG2A and/or PD-L1 from other known polypeptides, for example, by virtue of measurable differences in binding affinity). In some embodiments, an NKG2A binding agent (e.g., an antibody) may react with NKG2A sequences other than human NKG2A sequences (e.g., cynomolgus monkey NKG2A sequences such as A42 described herein). In other embodiments, an NKG2A binding agent (e.g., an antibody) does not react with non-human (such as cynomolgus monkey) NKG2A sequences such as A2, A3 and A11 provided herein. In some embodiments, a PD-L1 binding agent (e.g., an antibody) may react with PD-L1 sequences other than human PD-L1 sequences (e.g., cynomolgus monkey PD-L1 sequences). In other embodiments, a PD-L1 binding agent (e.g., an antibody) does not react with non-human (such as cynomolgus monkey) PD-L1 sequences.

The term “variable region” or “variable domain” refers to a portion of the light or heavy chains of an antibody that is generally located at the amino-terminal of the light or heavy chain, has a length of about 120 to 130 amino acids in the heavy chain and about 100 to 110 amino acids in the light chain, and is used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” or alternatively called “complementarity determining regions (CDRs).” The variable regions of heavy and light chains each comprise four frameworks (FR1, FR2, FR3 and FR4), largely adopting a β sheet configuration, connected by three hypervariable regions, which form loops connecting, and in some cases forming part of, the β sheet structure. The hypervariable regions in each chain are held together in close proximity by the frameworks and, with the hypervariable regions from the other chain, contribute to the formation of the antigen-binding site of antibodies (see, e.g., Kabat et al., Sequences of Proteins of Immunological Interest, 5th Ed. Public Health Service, National Institutes of Health, Bethesda, MD, (1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. The variability in sequence is concentrated in the CDRs while the less variable portions in the variable region are referred to as framework regions (FR). The CDRs of the light and heavy chains are primarily responsible for the interaction of the antibody with its antigen. In specific embodiments, the variable region is a human variable region.

The term “hypervariable region,” “HVR,” “HV,” “complementarity determining region,” or “CDR” when used herein refers to the regions of an antibody variable region that are hypervariable in sequence and/or form structurally defined loops. Generally, antibodies comprise six hypervariable regions: three in the VH (H1 or VH CDR1, H2 or VH CDR2, and H3 or VH CDR3), and three in the VL (L1 or VL CDR1, L2 or VL CDR2, and L3 or VL CDR3). A number of hypervariable region delineations are in use and are encompassed herein. The Kabat CDRs are based on sequence variability and are the most commonly used (see, e.g., Kabat et al., Sequences of Proteins of Immunological Interest, 5th Ed. Public Health Service, National Institutes of Health, Bethesda, MD. (1991)). Chothia refers instead to the location of the structural loops (see, e.g., Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Martin, in Antibody Engineering, Vol. 2, Chapter 3, Springer Verlag). The “contact” hypervariable regions are based on an analysis of the available complex crystal structures. The residues from each of these hypervariable regions or CDRs are noted below.

A universal numbering system has been developed and widely adopted, ImMunoGeneTics (IMGT®) Information System (Lefranc et al., Dev. Comp. Immunol. 27(1):55-77 (2003)). IMGT is an integrated information system specializing in immunoglobulins (IG), T cell receptors (TR) and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues and are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309:657-670 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra) and is also illustrated below. Various systems known in the art or described herein represent different ways of delineating CDRs, and when they are used to define the same antibody, they are often considered equivalent. An Exemplary system, shown herein, combines Kabat and Chothia. The residues from each of these hypervariable regions or CDRs are exemplified in the table below.

### Exemplary CDRs According to Various Numbering Systems

| | Exemplary | IMGT | Kabat | AbM | Chothia | Contact |
|---|---|---|---|---|---|---|
| V_{H} CDR1 | 26-35 | 27-38 | 31-35 | 26-35 | 26-32 | 30-35 |
| V_{H} CDR2 | 50-65 | 56-65 | 50-65 | 50-58 | 53-55 | 47-58 |
| V_{H} CDR3 | 95-102 | 105-117 | 95-102 | 95-102 | 96-101 | 93-101 |
| V_{L} CDR1 | 24-34 | 27-38 | 24-34 | 24-34 | 26-32 | 30-36 |
| V_{L} CDR2 | 50-56 | 56-65 | 50-56 | 50-56 | 50-52 | 46-55 |
| V_{L} CDR3 | 89-97 | 105-117 | 89-97 | 89-97 | 91-96 | 89-96 |

Hypervariable regions may comprise “extended hypervariable regions” as follows: 24-36 or 24-34 (L1), 46-56 or 50-56 (L2) and 89-97 or 89-96 (L3) in the VL and 26-35 or 26-35A (H1), 50-65 or 49-65 (H2) and 93-102, 94-102, or 95-102 (H3) in the VH. As used herein, the terms “hypervariable region,” “HVR,” “HV,” “complementarity determining region,” or “CDR” are used interchangeably.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. A cell that produces a binding molecule of the present disclosure may include a parent hybridoma cell, as well as bacterial and eukaryotic host cells into which nucleic acids encoding the antibodies have been introduced. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

The term “vector” refers to a substance that is used to carry or include a nucleic acid sequence, including for example, in order to introduce a nucleic acid sequence into a host cell. Vectors applicable for use include, for example, expression vectors, plasmids, phage vectors, viral vectors, episomes and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and/or inducible promoters, transcription enhancers, transcription terminators, and the like which are well known in the art. When two or more nucleic acid molecules are to be co-expressed (e.g., both an antibody heavy and light chain or an antibody VH and VL) both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product (e.g., a binding agent as described herein), and it is further understood that expression levels can be optimized to obtain sufficient expression using methods well known in the art.

The term “pharmaceutically acceptable” as used herein means being approved by a regulatory agency of the federal or a state government, or listed in the U.S. Pharmacopeia, European Pharmacopeia or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete)) or vehicle. In some embodiments, excipients are pharmaceutically acceptable excipients. Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. Other examples of pharmaceutically acceptable excipients are described in Remington and Gennaro, Remington's Pharmaceutical Sciences (18th ed. 1990). In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution. In some embodiments, excipients are sterile liquids, such as water and oils, including those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. An excipient can also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard excipients such as pharmaceutical grades of mannitol, lactose, starch, magnesium stearate, sodium saccharine, cellulose, magnesium carbonate, etc. Compositions, including pharmaceutical compounds, may contain a prophylactically or therapeutically effective amount of an NKG2A and/or PD-L1 binding agent (e.g., an antibody), for example, in isolated or purified form, together with a suitable amount of excipient so as to provide the form for proper administration to the subject (e.g., patient). The formulation should suit the mode of administration.

An “effective amount” is generally an amount sufficient to reduce the severity and/or frequency of symptoms, eliminate the symptoms and/or underlying cause, prevent or delay the occurrence of symptoms and/or their underlying cause, and/or improve or remediate the damage that results from or is associated with a disease, disorder, or condition. In some embodiments, the effective amount is a therapeutically effective amount or a prophylactically effective amount.

The term “therapeutically effective amount” as used herein refers to the amount of an agent (e.g., an antibody described herein or any other agent described herein) that is sufficient to reduce and/or ameliorate the severity and/or duration of a given disease, disorder or condition, and/or a symptom related thereto. A therapeutically effective amount of an agent, including a therapeutic agent, can be an amount necessary for (i) reduction, delay or amelioration of the advancement or progression of a given disease, disorder, or condition, (ii) reduction, delay or amelioration of the recurrence, development or onset of a given disease, disorder or conditions, and/or (iii) to improve or enhance the prophylactic or therapeutic effect of another therapy (e.g., a therapy other than the administration of an agent described herein). A “therapeutically effective amount” of a substance/molecule/agent of the present disclosure (e.g., a bispecific antibody) may vary according to factors such as the disease state, age, sex, and weight of the individual, and the ability of the substance/molecule/agent, to elicit a desired response in the individual. A therapeutically effective amount encompasses an amount in which any toxic or detrimental effects of the substance/molecule/agent are outweighed by the therapeutically beneficial effects. In certain embodiments, the term “therapeutically effective amount” refers to an amount of a multispecific binding agent effective to “treat” a disease, disorder, or condition, in a subject or mammal.

The term “treating” or any grammatical variation thereof refers to reducing and/or ameliorating the severity and/or duration of a given disease, disorder or condition, and/or a symptom related thereto, such as (i) reduction, delay or amelioration of the advancement or progression of a given disease, disorder, or condition, (ii) reduction, delay or amelioration of the recurrence, development or onset of a given disease, disorder or conditions, and/or (iii) to improve or enhance the prophylactic or therapeutic effect of another therapy (e.g., a therapy other than the administration of a multispecific binding agent described herein).

A “prophylactically effective amount” is an amount of a pharmaceutical composition that, when administered to a subject, will have the intended prophylactic effect, e.g., preventing or delaying the onset (or reoccurrence) of a disease, disorder or condition, or reducing the likelihood of the onset (or reoccurrence) of a disease, disorder, or condition or associated symptom(s).

The full therapeutic or prophylactic effect does not necessarily occur by administration of one dose, and may occur only after administration of a series of doses. Thus, a therapeutically or prophylactically effective amount may be administered in one or more administrations.

The terms “about” and “approximately” mean within 20%, within 15%, within 10%, within 9%, within 8%, within 7%, within 6%, within 5%, within 4%, within 3%, within 2%, within 1%, or less variation of a given value or range.

As used herein, comparative terms as used herein, such as reduce, decrease, increase, or any grammatical variation thereof, can refer to certain variation from the reference. In some embodiments, such variation can refer to about 10%, or about 20%, or about 30%, or about 40%, or about 50%, or about 60%, or about 70%, or about 80%, or about 90%, or about 1 fold, or about 2 fold, or about 3 fold, or about 4 fold, or about 5 fold, or about 10 fold, or about 20 fold, or about 30 fold, or about 40 fold, or about 100 fold or higher than the reference. In some embodiments, such variation can refer to about 1%, or about 2%, or about 3%, or about 4%, or about 5%, or about 6%, or about 7%, or about 8%, or about 9%, or about 10%, or about 20%, or about 30%, or about 40%, or about 50%, or about 60%, or about 70%, or about 80%, or about 90%, or about 95%, or about 96%, or about 97%, or about 98%, or about 99% of the reference.

As used in the present disclosure and claims, the singular forms “a”, “an” and “the” include plural forms unless the context clearly dictates otherwise.

In some embodiments, the terms “first,” “second,” “third,” “fourth” and similar in a component name are used to distinguish and identify more than one component sharing certain identity in their names. For example, “first antibody” and “second antibody” are used to distinguish two antibodies.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “optional” or “optionally” means that the subsequently described circumstance may or may not occur, so that the description includes instances wherein the circumstance occurs, and the instances wherein the circumstance does not occur.

### 5.2. Multispecific Binding Agents

### 5.2.1. NKG2A Binding Domains

The multispecific binding agents provided herein comprise one or more NKG2A binding domains. In some embodiments, described herein are multispecific binding agents (e.g., bispecific antibodies) that bind to NKG2A. Asused herein, NKG2A refers to an NKG2A polypeptide, an NKG2A polypeptide fragment, an NKG2A peptide or an NKG2A epitope. In some embodiments, the NKG2A binding domains are derived from human or humanized antibodies (e.g., comprising human framework regions) that bind NKG2A, including an NKG2A polypeptide, an NKG2A polypeptide fragment, an NKG2A peptide or an NKG2A epitope. In some embodiments, the multispecific binding agent (e.g., a bispecific antibody) can bind to NKG2A expressed on the surface of a mammalian (e.g., human) cell, including an NKG2A expressing immune cell (e.g., an NK cell or a T cell). In some embodiments, the multispecific binding agent (e.g., a bispecific antibody) provided herein binds an NKG2A extracellular epitope exposed on a cell such as an immune cell. In some embodiments, described herein is a multispecific binding agent (e.g., a bispecific antibody) that binds to NKG2A, such as human NKG2A or a portion thereof. In some embodiments, NKG2A is a human NKG2A. In some embodiments, the multispecific binding agent provided herein is a human NKG2A binding agent (e.g., an antibody that binds to human NKG2A). In some embodiments, multispecific binding agents (e.g., bispecific antibodies) that bind to NKG2A as disclosed herein bind to both human and cyno NKG2A. In other embodiments, multispecific binding agents (e.g., bispecific antibodies) that bind to NKG2A as disclosed herein bind to human NKG2A but not to cyno NKG2A. In some embodiments, described herein is a multispecific binding agent (e.g., a bispecific antibody) that binds to a complex comprising NKG2A and CD94, or a complex comprising extracellular domains of NKG2A and CD94.

In some embodiments, the multispecific binding agent (e.g., a bispecific antibody) provided herein binds to NKG2A (e.g., human NKG2A) with a dissociation constant (K_{D}) of ≤1μ, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹M to 10⁻¹³ M). A variety of methods of measuring binding affinity are known in the art, any of which can be used for purposes of the present disclosure, including by RIA, for example, performed with the Fab version of an antibody of interest and its antigen (Chen et al., 1999, J. Mol Biol 293:865-81); by biolayer interferometry (BLI) or surface plasmon resonance (SPR) assays by OCTET®, using, for example, an OCTET®Red96 system, or by BIACORE®, using, for example, a BIACORE®TM-2000 or a BIACORE®TM-3000. An “on-rate” or “rate of association” or “association rate” or “kon” may also be determined with the same biolayer interferometry (BLI) or surface plasmon resonance (SPR) techniques described above using, for example, the OCTET®Red96, the BIACORE®TM-2000, the BIACORE®TM-3000 system, the BIACORE®TM-8K, or the BIACORE®TM-8K+ system.

In some embodiments, the multispecific binding agent (e.g., a bispecific antibody) provided herein does not bind to NKG2C (or a complex comprising NKG2C and CD94 or extracellular domain of each thereof) (e.g., human NKG2C and/or cyno NKG2C). In some embodiments, the multispecific binding agent (e.g., a bispecific antibody) provided herein does not bind to human NKG2C. In some embodiments, the multispecific binding agent (e.g., a bispecific antibody) provided herein does not bind to human NKG2C or cyno NKG2C. In other embodiments, the multispecific binding agent (e.g., a bispecific antibody) provided herein binds to NKG2A (e.g., human NKG2A) (or a complex comprising NKG2A and CD94 or extracellular domain of each thereof) with higher affinity than to NKG2C (e.g., human NKG2C) (or a complex comprising NKG2C and CD94 or extracellular domain of each thereof). In some embodiments, the binding affinity of the multispecific binding agent (e.g., a bispecific antibody) provided herein to NKG2A (e.g., human NKG2A) (or a complex comprising NKG2A and CD94 or extracellular domain of each thereof) is at least 2 fold of that to NKG2C (e.g., human NKG2C) (or a complex comprising NKG2C and CD94 or extracellular domain of each thereof). In some embodiments, the binding affinity of the multispecific binding agent (e.g., a bispecific antibody) provided herein to NKG2A (e.g., human NKG2A) (or a complex comprising NKG2A and CD94 or extracellular domain of each thereof) is at least 5 fold of that to NKG2C (e.g., human NKG2C) (or a complex comprising NKG2C and CD94 or extracellular domain of each thereof). In some embodiments, the binding affinity of the multispecific binding agent (e.g., a bispecific antibody) provided herein to NKG2A (e.g., human NKG2A) (or a complex comprising NKG2A and CD94 or extracellular domain of each thereof) is at least 10 fold of that to NKG2C (e.g., human NKG2C) (or a complex comprising NKG2C and CD94 or extracellular domain of each thereof). In some embodiments, the binding affinity of the multispecific binding agent (e.g., a bispecific antibody) provided herein to NKG2A (e.g., human NKG2A) (or a complex comprising NKG2A and CD94 or extracellular domain of each thereof) is at least 100 fold of that to NKG2C (e.g., human NKG2C) (or a complex comprising NKG2C and CD94 or extracellular domain of each thereof). In some embodiments, the binding affinity of the multispecific binding agent (e.g., a bispecific antibody) provided herein to NKG2A (e.g., human NKG2A) (or a complex comprising NKG2A and CD94 or extracellular domain of each thereof) is at least 1000 fold of that to NKG2C (e.g., human NKG2C) (or a complex comprising NKG2C and CD94 or extracellular domain of each thereof).

In some embodiments, the multispecific binding agents (e.g., bispecific antibodies) described herein comprise a VH region, VL region, VH CDR1, VH CDR2, VH CDR3, VL CDR1, VL CDR2, and/or VL CDR3 of any one of the antibodies described herein, such as an amino acid sequence of a VH region, VL region, VH CDR1, VH CDR2, VH CDR3, VL CDR1, VL CDR2, and/or VL CDR3 depicted in Tables 1-4. Accordingly, in some embodiments, a multispecific binding agent (e.g., a bispecific antibody) described herein comprises any one, any two, and/or all three heavy chain CDRs and/or any one, any two, and/or all three light chain CDRs from: (a) the antibody designated A2; (b) the antibody designated A3; (c) the antibody designated A11; and (d) the antibody designated A42, as shown in Tables 1-4. In some embodiments, a multispecific binding agent (e.g., a bispecific antibody) described herein comprises any one, any two, and/or all three heavy chain CDRs and any one, any two, and/or all three light chain CDRs from: (a) the antibody designated A2; (b) the antibody designated A3; (c) the antibody designated A11; and (d) the antibody designated A42, as shown in Tables 1-4.

In some embodiments, a multispecific binding agent (e.g., a bispecific antibody) comprises a VH region, which comprises a VH CDR1, a VH CDR2, and/or a VH CDR3, and/or a VL region, which comprises a VL CDR1, a VL CDR2, and/or a VL CDR3, of any one of the binding agents described herein (see, e.g., any one of Tables 1-4). Accordingly, in some embodiments, a multispecific binding agent (e.g., a bispecific antibody) described herein comprises any one, any two, and/or all three heavy chain CDRs and/or any one, any two, and/or all three light chain CDRs from Table 1. In some embodiments, a multispecific binding agent (e.g., a bispecific antibody) described herein comprises any one, any two, and/or all three heavy chain CDRs and/or any one, any two, and/or all three light chain CDRs from Table 2. In some embodiments, a multispecific binding agent (e.g., a bispecific antibody) described herein comprises any one, any two, and/or all three heavy chain CDRs and/or any one, any two, and/or all three light chain CDRs from Table 3. In some embodiments, a multispecific binding agent (e.g., a bispecific antibody) described herein comprises any one, any two, and/or all three heavy chain CDRs and/or one, two, and/or three light chain CDRs from Table 4.

In some embodiments, the multispecific binding agent provided herein (e.g., a bispecific antibody) comprises (i) a VH CDR1, a VH CDR2, and a VH CDR3 as set forth in a VH comprising the amino acid sequence of SEQ ID NO:25, SEQ ID NO:45, or SEQ ID NO: 64 and/or (ii) a VL CDR1, a VL CDR2, and a VL CDR3 as set forth in a VL comprising the amino acid sequence of SEQ ID NO:26, SEQ ID NO:46, SEQ ID NO:65, or SEQ ID NO: 73.

In some embodiments, the multispecific binding agent provided herein (e.g., a bispecific antibody) comprises a VH CDR1, a VH CDR2, and/or a VH CDR3 as set forth in a VH comprising the amino acid sequence of SEQ ID NO:25 and/or a VL CDR1, a VL CDR2, and/or a VL CDR3 as set forth in a VL comprising the amino acid sequence of SEQ ID NO: 26. In some embodiments, the multispecific binding agent provided herein (e.g., a bispecific antibody) comprises a VH CDR1, a VH CDR2, and a VH CDR3 as set forth in a VH comprising the amino acid sequence of SEQ ID NO:25 and a VL CDR1, a VL CDR2, and a VL CDR3 as set forth in a VL comprising the amino acid sequence of SEQ ID NO:26. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDR sequences are determined according to a combination of any two or more of the above-mentioned numbering systems, for example, a combination of Kabat and Chothia. Various exemplary CDR numbering systems are described and illustrated above in Section 5.1.

In some embodiments, the multispecific binding agent provided herein (e.g., a bispecific antibody) comprises (a) a VH region comprising a VH CDR1 having an amino acid sequence selected from the group consisting of SEQ ID NOs: 1, 7, 12, 13, and 18; a VH CDR2 having an amino acid sequence selected from the group consisting of SEQ ID NOs: 2, 8, 14, 19, and 24; and a VH CDR3 having an amino acid sequence selected from the group consisting of SEQ ID NOs: 3, 9, 15, and 20; and/or (b) a VL region comprising a VL CDR1 having an amino acid sequence selected from the group consisting of SEQ ID NOs: 4, 10, 16, and 21; a VL CDR2 having an amino acid sequence selected from the group consisting of SEQ ID NOs: 5, 11, and 22; and a VL CDR3 having an amino acid sequence selected from the group consisting of SEQ ID NOs: 6, 17, and 23.

In some embodiments, the multispecific binding agent provided herein (e.g., a bispecific antibody) comprises a VH region comprising a VH CDR1 comprising the amino acid sequence of SEQ ID NO:1, a VH CDR2 comprising the amino acid sequence of SEQ ID NO: 2, and a VH CDR3 comprising the amino acid sequence of SEQ ID NO:3; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6.

In some embodiments, the multispecific binding agent provided herein (e.g., a bispecific antibody) comprises a VH region comprising a VH CDR1 comprising the amino acid sequence of SEQ ID NO:7, a VH CDR2 comprising the amino acid sequence of SEQ ID NO: 8, and a VH CDR3 comprising the amino acid sequence of SEQ ID NO:9; and a VL region comprising a VL CDR1 comprising the amino acid sequence of SEQ ID NO:10, a VL CDR2 comprising the amino acid sequence of SEQ ID NO:11, and a VL CDR3 comprising the amino acid sequence of SEQ ID NO:6.

In some embodiments, the multispecific binding agent provided herein (e.g., a bispecific antibody) comprises a VH region comprising a VH CDR1 comprising the amino acid sequence of SEQ ID NO:12, a VH CDR2 comprising the amino acid sequence of SEQ ID NO: 2, and a VH CDR3 comprising the amino acid sequence of SEQ ID NO:3; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6.

In some embodiments, the multispecific binding agent provided herein (e.g., a bispecific antibody) comprises a VH region comprising a VH CDR1 comprising the amino acid sequence of SEQ ID NO:13, a VH CDR2 comprising the amino acid sequence of SEQ ID NO: 14, and a VH CDR3 comprising the amino acid sequence of SEQ ID NO: 15; and a VL region comprising a VL CDR1 comprising the amino acid sequence of SEQ ID NO:16, a VL CDR2 comprising the amino acid sequence of SEQ ID NO:11, and a VL CDR3 comprising the amino acid sequence of SEQ ID NO:17.

In some embodiments, the multispecific binding agent provided herein (e.g., a bispecific antibody) comprises a VH region comprising a VH CDR1 comprising the amino acid sequence of SEQ ID NO:18, a VH CDR2 comprising the amino acid sequence of SEQ ID NO: 19, and a VH CDR3 comprising the amino acid sequence of SEQ ID NO:20; and a VL region comprising a VL CDR1 comprising the amino acid sequence of SEQ ID NO:21, a VL CDR2 comprising the amino acid sequence of SEQ ID NO:22, and a VL CDR3 comprising the amino acid sequence of SEQ ID NO:23.

In some embodiments, the multispecific binding agent provided herein (e.g., a bispecific antibody) comprises a VH region comprising a VH CDR1 comprising the amino acid sequence of SEQ ID NO:1, a VH CDR2 comprising the amino acid sequence of SEQ ID NO: 24, and a VH CDR3 comprising the amino acid sequence of SEQ ID NO:3; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6.

In some embodiments, the multispecific binding agent provided herein (e.g., a bispecific antibody) comprises a VH CDR1, a VH CDR2, and/or a VH CDR3 as set forth in a VH comprising the amino acid sequence of SEQ ID NO:45 and/or a VL CDR1, a VL CDR2, and/or a VL CDR3 as set forth in a VL comprising the amino acid sequence of SEQ ID NO: 46. In some embodiments, the multispecific binding agent provided herein (e.g., a bispecific antibody) comprises a VH CDR1, a VH CDR2, and a VH CDR3 as set forth in a VH comprising the amino acid sequence of SEQ ID NO:45 and a VL CDR1, a VL CDR2, and a VL CDR3 as set forth in a VL comprising the amino acid sequence of SEQ ID NO:46. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDR sequences are determined according to a combination of any two or more of the above-mentioned numbering systems, for example, a combination of Kabat and Chothia.

In some embodiments, the multispecific binding agent provided herein (e.g., a bispecific antibody) comprises (a) a VH region comprising a VH CDR1 having an amino acid sequence selected from the group consisting of SEQ ID NOs: 1, 7, 12, 13, and 18; a VH CDR2 having an amino acid sequence selected from the group consisting of SEQ ID NOs: 27, 32, 35, 39, and 44; and a VH CDR3 having an amino acid sequence selected from the group consisting of SEQ ID NOs: 28, 33, 36, and 40; and/or (b) a VL region comprising a VL CDR1 having an amino acid sequence selected from the group consisting of SEQ ID NOs: 29, 34, 37, and 41; a VL CDR2 having an amino acid sequence selected from the group consisting of SEQ ID NOs: 30, 11, and 42; and a VL CDR3 having an amino acid sequence selected from the group consisting of SEQ ID NOs: 31, 38, and 43.

In some embodiments, the multispecific binding agent provided herein (e.g., a bispecific antibody) comprises a VH region comprising a VH CDR1 comprising the amino acid sequence of SEQ ID NO:1, a VH CDR2 comprising the amino acid sequence of SEQ ID NO: 27, and a VH CDR3 comprising the amino acid sequence of SEQ ID NO:28; and a VL region comprising a VL CDR1 comprising the amino acid sequence of SEQ ID NO:29, a VL CDR2 comprising the amino acid sequence of SEQ ID NO:30, and a VL CDR3 comprising the amino acid sequence of SEQ ID NO:31.

In some embodiments, the multispecific binding agent provided herein (e.g., a bispecific antibody) comprises a VH region comprising a VH CDR1 comprising the amino acid sequence of SEQ ID NO:7, a VH CDR2 comprising the amino acid sequence of SEQ ID NO: 32, and a VH CDR3 comprising the amino acid sequence of SEQ ID NO:33; and a VL region comprising a VL CDR1 comprising the amino acid sequence of SEQ ID NO:34, a VL CDR2 comprising the amino acid sequence of SEQ ID NO:11, and a VL CDR3 comprising the amino acid sequence of SEQ ID NO:31.

In some embodiments, the multispecific binding agent provided herein (e.g., a bispecific antibody) comprises a VH region comprising a VH CDR1 comprising the amino acid sequence of SEQ ID NO:12, a VH CDR2 comprising the amino acid sequence of SEQ ID NO: 27, and a VH CDR3 comprising the amino acid sequence of SEQ ID NO:28; and a VL region comprising a VL CDR1 comprising the amino acid sequence of SEQ ID NO:29, a VL CDR2 comprising the amino acid sequence of SEQ ID NO:30, and a VL CDR3 comprising the amino acid sequence of SEQ ID NO:31.

In some embodiments, the multispecific binding agent provided herein (e.g., a bispecific antibody) comprises a VH region comprising a VH CDR1 comprising the amino acid sequence of SEQ ID NO:13, a VH CDR2 comprising the amino acid sequence of SEQ ID NO: 35, and a VH CDR3 comprising the amino acid sequence of SEQ ID NO:36; and a VL region comprising a VL CDR1 comprising the amino acid sequence of SEQ ID NO:37, a VL CDR2 comprising the amino acid sequence of SEQ ID NO:11, and a VL CDR3 comprising the amino acid sequence of SEQ ID NO:38.

In some embodiments, the multispecific binding agent provided herein (e.g., a bispecific antibody) comprises a VH region comprising a VH CDR1 comprising the amino acid sequence of SEQ ID NO:18, a VH CDR2 comprising the amino acid sequence of SEQ ID NO: 39, and a VH CDR3 comprising the amino acid sequence of SEQ ID NO:40; and a VL region comprising a VL CDR1 comprising the amino acid sequence of SEQ ID NO:41, a VL CDR2 comprising the amino acid sequence of SEQ ID NO:42, and a VL CDR3 comprising the amino acid sequence of SEQ ID NO:43.

In some embodiments, the multispecific binding agent provided herein (e.g., a bispecific antibody) comprises a VH region comprising a VH CDR1 comprising the amino acid sequence of SEQ ID NO:1, a VH CDR2 comprising the amino acid sequence of SEQ ID NO: 44, and a VH CDR3 comprising the amino acid sequence of SEQ ID NO:28; and a VL region comprising a VL CDR1 comprising the amino acid sequence of SEQ ID NO:29, a VL CDR2 comprising the amino acid sequence of SEQ ID NO:30, and a VL CDR3 comprising the amino acid sequence of SEQ ID NO:31.

In some embodiments, the multispecific binding agent provided herein (e.g., a bispecific antibody) comprises a VH CDR1, a VH CDR2, and/or a VH CDR3 as set forth in a VH comprising the amino acid sequence of SEQ ID NO:64 and/or a VL CDR1, a VL CDR2, and/or a VL CDR3 as set forth in a VL comprising the amino acid sequence of SEQ ID NO: 65. In some embodiments, the multispecific binding agent provided herein (e.g., a bispecific antibody) comprises a VH CDR1, a VH CDR2, and a VH CDR3 as set forth in a VH comprising the amino acid sequence of SEQ ID NO:64 and/or a VL CDR1, a VL CDR2, and a VL CDR3 as set forth in a VL comprising the amino acid sequence of SEQ ID NO:65. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDR sequences are determined according to a combination of any two or more of the above-mentioned numbering systems, for example, a combination of Kabat and Chothia.

In some embodiments, the multispecific binding agent provided herein (e.g., a bispecific antibody) comprises (a) a VH region comprising a VH CDR1 having an amino acid sequence selected from the group consisting of SEQ ID NOs: 47, 51, 54, 55, and 59; a VH CDR2 having an amino acid sequence selected from the group consisting of SEQ ID NOs: 48, 52, 56, 60, and 63; and a VH CDR3 having an amino acid sequence selected from the group consisting of SEQ ID NOs: 49, 53, 57, and 61; and (b) a VL region comprising a VL CDR1 having an amino acid sequence selected from the group consisting of SEQ ID NOs: 4, 10, 16, and 21; a VL CDR2 having an amino acid sequence selected from the group consisting of SEQ ID NOs: 5, 11, and 22; and a VL CDR3 having an amino acid sequence selected from the group consisting of SEQ ID NOs: 50, 58, and 62.

In some embodiments, the multispecific binding agent provided herein (e.g., a bispecific antibody) comprises a VH region comprising a VH CDR1 comprising the amino acid sequence of SEQ ID NO:47, a VH CDR2 comprising the amino acid sequence of SEQ ID NO: 48, and a VH CDR3 comprising the amino acid sequence of SEQ ID NO:49; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:50.

In some embodiments, the multispecific binding agent provided herein (e.g., a bispecific antibody) comprises a VH region comprising a VH CDR1 comprising the amino acid sequence of SEQ ID NO:51, a VH CDR2 comprising the amino acid sequence of SEQ ID NO: 52, and a VH CDR3 comprising the amino acid sequence of SEQ ID NO:53; and a VL region comprising a VL CDR1 comprising the amino acid sequence of SEQ ID NO:10, a VL CDR2 comprising the amino acid sequence of SEQ ID NO:11, and a VL CDR3 comprising the amino acid sequence of SEQ ID NO:50.

In some embodiments, the multispecific binding agent provided herein (e.g., a bispecific antibody) comprises a VH region comprising a VH CDR1 comprising the amino acid sequence of SEQ ID NO:54, a VH CDR2 comprising the amino acid sequence of SEQ ID NO: 48, and a VH CDR3 comprising the amino acid sequence of SEQ ID NO:49; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:50.

In some embodiments, the multispecific binding agent provided herein (e.g., a bispecific antibody) comprises a VH region comprising a VH CDR1 comprising the amino acid sequence of SEQ ID NO:55, a VH CDR2 comprising the amino acid sequence of SEQ ID NO: 56, and a VH CDR3 comprising the amino acid sequence of SEQ ID NO:57; and a VL region comprising a VL CDR1 comprising the amino acid sequence of SEQ ID NO:16, a VL CDR2 comprising the amino acid sequence of SEQ ID NO:11, and a VL CDR3 comprising the amino acid sequence of SEQ ID NO:58.

In some embodiments, the multispecific binding agent provided herein (e.g., a bispecific antibody) comprises a VH region comprising a VH CDR1 comprising the amino acid sequence of SEQ ID NO:59, a VH CDR2 comprising the amino acid sequence of SEQ ID NO: 60, and a VH CDR3 comprising the amino acid sequence of SEQ ID NO:61; and a VL region comprising a VL CDR1 comprising the amino acid sequence of SEQ ID NO:21, a VL CDR2 comprising the amino acid sequence of SEQ ID NO:22, and a VL CDR3 comprising the amino acid sequence of SEQ ID NO:62.

In some embodiments, the multispecific binding agent provided herein (e.g., a bispecific antibody) comprises a VH region comprising a VH CDR1 comprising the amino acid sequence of SEQ ID NO:47, a VH CDR2 comprising the amino acid sequence of SEQ ID NO: 63, and a VH CDR3 comprising the amino acid sequence of SEQ ID NO:49; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:50.

In some embodiments, the multispecific binding agent provided herein (e.g., a bispecific antibody) comprises a VH CDR1, a VH CDR2, and/or a VH CDR3 as set forth in a VH comprising the amino acid sequence of SEQ ID NO:64 and/or a VL CDR1, a VL CDR2, and/or a VL CDR3 as set forth in a VL comprising the amino acid sequence of SEQ ID NO: 73. In some embodiments, the multispecific binding agent provided herein (e.g., a bispecific antibody) comprises a VH CDR1, a VH CDR2, and a VH CDR3 as set forth in a VH comprising the amino acid sequence of SEQ ID NO:64 and/or a VL CDR1, a VL CDR2, and a VL CDR3 as set forth in a VL comprising the amino acid sequence of SEQ ID NO:73. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDR sequences are determined according to a combination of any two or more of the above-mentioned numbering systems, for example, a combination of Kabat and Chothia.

In some embodiments, the multispecific binding agent provided herein (e.g., a bispecific antibody) comprises (a) a VH region comprising a VH CDR1 having an amino acid sequence selected from the group consisting of SEQ ID NOs: 47, 51, 54, 55, and 59; a VH CDR2 having an amino acid sequence selected from the group consisting of SEQ ID NOs: 48, 52, 56, 60, and 63; and a VH CDR3 having an amino acid sequence selected from the group consisting of SEQ ID NOs: 49, 53, 57, and 61; and (b) a VL region comprising a VL CDR1 having an amino acid sequence selected from the group consisting of SEQ ID NOs: 66, 68, 70, and 71; a VL CDR2 having an amino acid sequence selected from the group consisting of SEQ ID NOs: 67, 69, and 72; and a VL CDR3 having an amino acid sequence selected from the group consisting of SEQ ID NOs: 50, 58, and 62.

In some embodiments, the multispecific binding agent provided herein (e.g., a bispecific antibody) comprises a VH region comprising a VH CDR1 comprising the amino acid sequence of SEQ ID NO:47, a VH CDR2 comprising the amino acid sequence of SEQ ID NO: 48, and a VH CDR3 comprising the amino acid sequence of SEQ ID NO:49; and a VL region comprising a VL CDR1 comprising the amino acid sequence of SEQ ID NO:66, a VL CDR2 comprising the amino acid sequence of SEQ ID NO:67, and a VL CDR3 comprising the amino acid sequence of SEQ ID NO:50.

In some embodiments, the multispecific binding agent provided herein (e.g., a bispecific antibody) comprises a VH region comprising a VH CDR1 comprising the amino acid sequence of SEQ ID NO:51, a VH CDR2 comprising the amino acid sequence of SEQ ID NO: 52, and a VH CDR3 comprising the amino acid sequence of SEQ ID NO:53; and a VL region comprising a VL CDR1 comprising the amino acid sequence of SEQ ID NO:68, a VL CDR2 comprising the amino acid sequence of SEQ ID NO:69, and a VL CDR3 comprising the amino acid sequence of SEQ ID NO:50.

In some embodiments, the multispecific binding agent provided herein (e.g., a bispecific antibody) comprises a VH region comprising a VH CDR1 comprising the amino acid sequence of SEQ ID NO:54, a VH CDR2 comprising the amino acid sequence of SEQ ID NO: 48, and a VH CDR3 comprising the amino acid sequence of SEQ ID NO:49; and a VL region comprising a VL CDR1 comprising the amino acid sequence of SEQ ID NO:66, a VL CDR2 comprising the amino acid sequence of SEQ ID NO:67, and a VL CDR3 comprising the amino acid sequence of SEQ ID NO:50.

In some embodiments, the multispecific binding agent provided herein (e.g., a bispecific antibody) comprises a VH region comprising a VH CDR1 comprising the amino acid sequence of SEQ ID NO:55, a VH CDR2 comprising the amino acid sequence of SEQ ID NO: 56, and a VH CDR3 comprising the amino acid sequence of SEQ ID NO:57; and a VL region comprising a VL CDR1 comprising the amino acid sequence of SEQ ID NO:70, a VL CDR2 comprising the amino acid sequence of SEQ ID NO:69, and a VL CDR3 comprising the amino acid sequence of SEQ ID NO:58.

In some embodiments, the multispecific binding agent provided herein (e.g., a bispecific antibody) comprises a VH region comprising a VH CDR1 comprising the amino acid sequence of SEQ ID NO:59, a VH CDR2 comprising the amino acid sequence of SEQ ID NO: 60, and a VH CDR3 comprising the amino acid sequence of SEQ ID NO:61; and a VL region comprising a VL CDR1 comprising the amino acid sequence of SEQ ID NO:71, a VL CDR2 comprising the amino acid sequence of SEQ ID NO:72, and a VL CDR3 comprising the amino acid sequence of SEQ ID NO:62.

In some embodiments, the multispecific binding agent provided herein (e.g., a bispecific antibody) comprises a VH region comprising a VH CDR1 comprising the amino acid sequence of SEQ ID NO:47, a VH CDR2 comprising the amino acid sequence of SEQ ID NO: 63, and a VH CDR3 comprising the amino acid sequence of SEQ ID NO:49; and a VL region comprising a VL CDR1 comprising the amino acid sequence of SEQ ID NO:66, a VL CDR2 comprising the amino acid sequence of SEQ ID NO:67, and a VL CDR3 comprising the amino acid sequence of SEQ ID NO:50.

In some embodiments, the NKG2A binding domain provided herein further comprises one or more framework regions of SEQ ID NOs: 25, 26, 45, 46, 64, 65, and/or 73. In some embodiments, the NKG2A binding domain further comprises a framework 1 (FR1), a framework 2 (FR2), a framework 3 (FR3) and/or a framework 4 (FR4) sequence as set forth in any one of SEQ ID NOs: 25, 26, 45, 46, 64, 65, and 73. In some embodiments, the NKG2A binding domain provided herein is derived from a humanized antibody. Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FR1 is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system.

In some embodiments, the NKG2A binding domains described herein comprise a VH region or VH domain. Additionally or alternatively, in some embodiments, the NKG2A binding domains described herein comprise a VL region or VL domain. In some embodiments, the NKG2A binding domains described herein have a combination of (i) a VH domain or VH region; and (ii) a VL domain or VL region.

In some embodiments, the multispecific binding agent provided herein (e.g., a bispecific antibody) comprises a VH comprising the amino acid sequence of SEQ ID NO:25. In some embodiments, the multispecific binding agent provided herein (e.g., a bispecific antibody) comprises a VL comprising the amino acid sequence of SEQ ID NO:26. In some embodiments, the multispecific binding agent provided herein (e.g., a bispecific antibody) comprises a VH comprising the amino acid sequence of SEQ ID NO:25 and a VL comprising the amino acid sequence of SEQ ID NO:26.

In some embodiments, the multispecific binding agent provided herein (e.g., a bispecific antibody) comprises a VH comprising the amino acid sequence of SEQ ID NO:45. In some embodiments, the multispecific binding agent provided herein (e.g., a bispecific antibody) comprises a VL comprising the amino acid sequence of SEQ ID NO:46. In some embodiments, the multispecific binding agent provided herein (e.g., a bispecific antibody) comprises a VH comprising the amino acid sequence of SEQ ID NO:45 and a VL comprising the amino acid sequence of SEQ ID NO:46.

In some embodiments, the multispecific binding agent provided herein (e.g., a bispecific antibody) comprises a VH comprising the amino acid sequence of SEQ ID NO:64. In some embodiments, the multispecific binding agent provided herein (e.g., a bispecific antibody) comprises a VL comprising the amino acid sequence of SEQ ID NO:65. In some embodiments, the multispecific binding agent provided herein (e.g., a bispecific antibody) comprises a VH comprising the amino acid sequence of SEQ ID NO:64 and a VL comprising the amino acid sequence of SEQ ID NO:65.

In some embodiments, the multispecific binding agent provided herein (e.g., a bispecific antibody) comprises a VH comprising the amino acid sequence of SEQ ID NO:64. In some embodiments, the multispecific binding agent provided herein (e.g., a bispecific antibody) comprises a VL comprising the amino acid sequence of SEQ ID NO:73. In some embodiments, the multispecific binding agent provided herein (e.g., a bispecific antibody) comprises a VH comprising the amino acid sequence of SEQ ID NO:64 and a VL comprising the amino acid sequence of SEQ ID NO:73.

In certain embodiments, the multispecific binding agent (e.g., a bispecific antibody) provided herein comprises amino acid sequences with certain percent identity (such as at least about 80%, or at least about 81%, or at least about 82%, or at least about 83%, or at least about 84%, or at least about 85%, or at least about 86%, or at least about 87%, or at least about 88%, or at least about 89%, or as at least about 90%, or at least about 91%, or at least about 92%, or at least about 93%, or at least about 94%, or at least about 95%, or at least about 96%, or at least about 97%, or at least about 98%, or at least about 99%, or higher) relative to any antibody or fragment thereof provided herein, for example, a CDR, VH or VL in Tables 1-4, or a full-length antibody chain as disclosed herein. In some embodiments, the multispecific binding agent (e.g., a bispecific antibody) provided herein comprises CDRs of any antibody or fragment thereof provided herein, for example in Tables 1-4. In further embodiments, the bispecific antibody provided herein comprises amino acid sequences with certain percent identity (such as at least about 80%, or at least about 81%, or at least about 82%, or at least about 83%, or at least about 84%, or at least about 85%, or at least about 86%, or at least about 87%, or at least about 88%, or at least about 89%, or as at least about 90%, or at least about 91%, or at least about 92%, or at least about 93%, or at least about 94%, or at least about 95%, or at least about 96%, or at least about 97%, or at least about 98%, or at least about 99%, or higher) relative to any antibody or fragment thereof provided herein, for example, a VH or VL in Tables 1-4, or a full-length antibody chain as disclosed herein.

The determination of percent identity between two sequences (e.g., amino acid sequences or nucleic acid sequences) can be accomplished using a mathematical algorithm. A non-limiting example of a mathematical algorithm utilized for the comparison of two sequences is the algorithm of Karlin and Altschul, Proc. Natl. Acad. Sci. U.S.A. 87:2264 2268 (1990), modified as in Karlin and Altschul, Proc. Natl. Acad. Sci. U.S.A. 90:5873 5877 (1993). Such an algorithm is incorporated into the NBLAST and XBLAST programs of Altschul et al., J. Mol. Biol. 215:403 (1990). BLAST nucleotide searches can be performed with the NBLAST nucleotide program parameters set, e.g., for score=100, word length=12 to obtain nucleotide sequences homologous to a nucleic acid molecule described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res. 25:3389 3402 (1997). In some embodiments, the percent identity between two sequences is calculated by dividing the number of residue(s) varied (excluding or including conservative amino acid substitution(s) or degenerate nucleotide substitution(s)) between the two sequences in the alignment with the residue number of any one of the following: (i) full length of the shorter sequence, (ii) full length of the longer sequence, (iii) mean length of the two sequences, (iv) total length of the non-gap portion of the alignment, (v) length of the alignment excluding overhangs, or (vi) length of the alignment including overhangs. Overhangs as used herein with respect to a sequence alignment refer to either or both ends of the alignment where residues of one sequence are considered as aligning to no residues (e.g., gap) in the other sequence. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, CABIOS 4:11-17 (1998). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

In some embodiments, the multispecific binding agent (e.g., a bispecific antibody) provided herein contains substitutions (e.g., conservative substitutions), insertions, or deletions relative to the reference sequence, but the multispecific binding agent comprising that sequence retains the ability to bind to NKG2A. In some embodiments, a total of 1 to 10 amino acids have been substituted, inserted and/or deleted in a reference amino acid sequence. In some embodiments, substitutions, insertions, or deletions occur in regions outside the CDRs (e.g., in the FRs and/or constant regions).

In some embodiments, the position of one or more CDRs along the VH (e.g., CDR1, CDR2, or CDR3) and/or VL (e.g., CDR1, CDR2, or CDR3) region of an NKG2A binding domain described herein may vary by one, two, three, four, five, or six amino acid positions so long as binding to NKG2A (e.g., human NKG2A) is maintained (e.g., substantially maintained, for example, at least 50%, at least 60%, at least 70%, at least 80%, at least 90%, or at least 95%). For example, in some embodiments, the position defining a CDR of any of Table 1, 2, 3, or 4 may vary by shifting the N-terminal and/or C-terminal boundary of the CDR by one, two, three, four, five, or six amino acids, relative to the current CDR position, so long as binding to NKG2A (e.g., human NKG2A) is maintained (e.g., substantially maintained, for example, at least 50%, at least 60%, at least 70%, at least 80%, at least 90%, or at least 95%). Additionally or alternatively, in some embodiments, the length of one or more CDRs along the VH (e.g., CDR1, CDR2, or CDR3) and/or VL (e.g., CDR1, CDR2, or CDR3) region of an NKG2A binding domain described herein may vary (e.g., be shorter or longer) by one, two, three, four, five, or more amino acids, so long as binding to NKG2A (e.g., human NKG2A) is maintained (e.g., substantially maintained, for example, at least 50%, at least 60%, at least 70%, at least 80%, at least 90%, or at least 95%). For example, in some embodiments, a VH and/or VL CDR1, CDR2, and/or CDR3 described herein may be one, two, three, four, five or more amino acids shorter than one or more of the CDRs described by SEQ ID NOS: 1-24, 27-44, 47-63, or 66-72, so long as binding to NKG2A (e.g., human NKG2A) is maintained (e.g., substantially maintained, for example, at least 50%, at least 60%, at least 70%, at least 80%, at least 90%, or at least 95%). In other embodiments, a VH and/or VL CDR1, CDR2, and/or CDR3 described herein may be one, two, three, four, five or more amino acids longer than one or more of the CDRs described by SEQ ID NOS: 1-24, 27-44, 47-63, or 66-72, so long as binding to NKG2A (e.g., human NKG2A) is maintained (e.g., substantially maintained, for example, at least 50%, at least 60%, at least 70%, at least 80%, at least 90%, or at least 95%). In some embodiments, the amino terminus of a VH and/or VL CDR1, CDR2, and/or CDR3 described herein may be extended or shortened by one, two, three, four, five or more amino acids compared to one or more of the CDRs described by SEQ ID NOS: 1-24, 27-44, 47-63, or 66-72, so long as binding to NKG2A (e.g., human NKG2A) is maintained (e.g., substantially maintained, for example, at least 50%, at least 60%, at least 70%, at least 80%, at least 90%, or at least 95%). Additionally or alternatively, in some embodiments, the carboxy terminus of a VH and/or VL CDR1, CDR2, and/or CDR3 described herein may be extended or shortened by one, two, three, four, five or more amino acids compared to one or more of the CDRs described by SEQ ID NOS: 1-24, 27-44, 47-63, or 66-72, so long as binding to NKG2A (e.g., human NKG2A) is maintained (e.g., substantially maintained, for example, at least 50%, at least 60%, at least 70%, at least 80%, at least 90%, or at least 95%). Any method known in the art can be used to ascertain whether binding to NKG2A (e.g., human NKG2A) is maintained, for example, the binding assays and conditions described in the “Examples” section described herein.

In other embodiments, the multispecific binding agents (e.g., bispecific antibodies) further comprise conservative sequence modifications (e.g., in an NKG2A binding domain). Conservative sequence modifications include conservative amino acid substitutions that include ones in which the amino acid residue is replaced with an amino acid residue having a similar side chain. Families of amino acid residues having similar side chains have been defined in the art. Thus, in some embodiments, a predicted nonessential amino acid residue in an NKG2A is replaced with another amino acid residue from the same side chain family. Methods of identifying amino acid conservative substitutions which do not eliminate antigen binding and nucleotides encoding thereof are well-known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)). In some embodiments, the conservative sequence modifications described herein modify the amino acid sequences of the multispecific binding agents (e.g., bispecific antibodies), including human NKG2A binding agents, by 50%, or 55%, or 60%, or 65%, or 70%, or 75%, or 80%, or 85%, or 90%, or 95%, or 98%, or 99%. In some embodiments, the amino acid sequence modifications refer to at most 1, 2, 3, 4, 5, or 6 amino acid substitutions to the CDRs, such as those described in any one of Tables 1-4. Thus, for example, each such CDR may contain up to 5 conservative amino acid substitutions, for example up to (not more than) 4 conservative amino acid substitutions, for example up to (not more than) 3 conservative amino acid substitutions, for example up to (not more than) 2 conservative amino acid substitutions, or no more than 1 conservative amino acid substitution. In some embodiments, the NKG2A binding domain contains one or more, including six, CDRs having at least 75%, 80%, 85%, 90%, 95%, 96%, 97%, 98%, or 99% identity to the CDRs of A3, A2, A42, or A11 (see, e.g., Tables 1, 2, 3, or 4).

In some embodiments, an NKG2A binding domain contains a VH and a VL comprising CDRs identical to those of A3, A2, A42, or A11 (see, e.g., Tables 1, 2, 3, or 4). In some embodiments, the amino acid sequence modifications do not include any modification within an SDR. In some embodiments, the amino acid sequence modifications do not include any modification within a CDR (such as CDR1, CDR2, CDR3, or any combination thereof). Additionally or alternatively, the amino acid sequence modifications are in the framework, constant region, and/or fragment crystallizable region (Fc).

In some embodiments, the multispecific binding agent provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:25, and/or a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:26, and the binding of the multispecific binding agent to NKG2A (e.g., human NKG2A) is maintained (e.g., substantially maintained, for example, at least 50%, at least 60%, at least 70%, at least 80%, at least 90%, or at least 95%).

In some embodiments, the multispecific binding agent provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:45, and/or a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:46, and the binding of the multispecific binding agent to NKG2A (e.g., human NKG2A) is maintained (e.g., substantially maintained, for example, at least 50%, at least 60%, at least 70%, at least 80%, at least 90%, or at least 95%).

In some embodiments, the multispecific binding agent provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:64, and/or a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:65, and the binding of the multispecific binding agent to NKG2A (e.g., human NKG2A) is maintained (e.g., substantially maintained, for example, at least 50%, at least 60%, at least 70%, at least 80%, at least 90%, or at least 95%).

In some embodiments, the multispecific binding agent provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:64, and/or a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:73, and the binding of the multispecific binding agent to NKG2A (e.g., human NKG2A) is maintained (e.g., substantially maintained, for example, at least 50%, at least 60%, at least 70%, at least 80%, at least 90%, or at least 95%).

In some embodiments, functional epitopes can be mapped, e.g., by combinatorial alanine scanning or hydrogen/deuterium exchange mass spectrometry (HDX-MS), to identify amino acids in the NKG2A protein (or a complex comprising NKG2A and CD94 or extracellular domain of each thereof) that are necessary for interaction with a multispecific binding agent, an NKG2A binding domain thereof, and/or an anti-NKG2A antibody provided herein (e.g., in the following paragraphs). In some embodiments, conformational and crystal structure of a multispecific binding agent, an NKG2A binding domain thereof, and/or an anti-NKG2A antibody (e.g., provided in the following paragraphs) bound to NKG2A may be employed to identify the epitopes. In some embodiments, the present disclosure provides a multispecific antibody comprising an NKG2A binding domain that specifically binds to the same epitope as any of the multispecific binding agents as disclosed herein, NKG2A binding domains thereof, and/or the anti-NKG2A antibodies or fragments thereof provided herein (e.g., provided in the following paragraphs).

For example, in some embodiments, the NKG2A binding domain provided herein binds to the same epitope as an anti-NKG2A antibody comprising a VH CDR1, a VH CDR2, and a VH CDR3 as set forth in a VH comprising the amino acid sequence of SEQ ID NO:25 and a VL CDR1, a VL CDR2, and a VL CDR3 as set forth in a VL comprising the amino acid sequence of SEQ ID NO:26. In some embodiments, the NKG2A binding domain provided herein binds to the same epitope as an anti-NKG2A antibody comprising a VH comprising the amino acid sequence of SEQ ID NO:25, and a VL comprising the amino acid sequence of SEQ ID NO:26.

In some embodiments, the NKG2A binding domain provided herein binds to the same epitope as an anti-NKG2A antibody comprising a VH CDR1, a VH CDR2, and a VH CDR3 as set forth in a VH comprising the amino acid sequence of SEQ ID NO:45 and a VL CDR1, a VL CDR2, and a VL CDR3 as set forth in a VL comprising the amino acid sequence of SEQ ID NO:46. In some embodiments, the NKG2A binding domain provided herein binds to the same epitope as an anti-NKG2A antibody comprising a VH comprising the amino acid sequence of SEQ ID NO:45, and a VL comprising the amino acid sequence of SEQ ID NO:46.

In some embodiments, the NKG2A binding domain provided herein binds to the same epitope as an anti-NKG2A antibody comprising a VH CDR1, a VH CDR2, and a VH CDR3 as set forth in a VH comprising the amino acid sequence of SEQ ID NO:64 and a VL CDR1, a VL CDR2, and a VL CDR3 as set forth in a VL comprising the amino acid sequence of SEQ ID NO:65. In some embodiments, the NKG2A binding domain provided herein binds to the same epitope as an anti-NKG2A antibody comprising a VH comprising the amino acid sequence of SEQ ID NO:64, and a VL comprising the amino acid sequence of SEQ ID NO:65.

In some embodiments, the NKG2A binding domain provided herein binds to the same epitope as an anti-NKG2A antibody comprising a VH CDR1, a VH CDR2, and a VH CDR3 as set forth in a VH comprising the amino acid sequence of SEQ ID NO:64 and a VL CDR1, a VL CDR2, and a VL CDR3 as set forth in a VL comprising the amino acid sequence of SEQ ID NO:73. In some embodiments, the NKG2A binding domain provided herein binds to the same epitope as an anti-NKG2A antibody comprising a VH comprising the amino acid sequence of SEQ ID NO:64, and a VL comprising the amino acid sequence of SEQ ID NO:73.

In some embodiments, the NKG2A binding domain provided herein specifically binds to one of the following NKG2A polypeptide fragments: an NKG2A polypeptide fragment comprising the amino acid sequence of TWEESL (SEQ ID NO:134), an NKG2A polypeptide fragment comprising the amino acid sequence of SIISPSSWIGV (SEQ ID NO: 135), an NKG2A polypeptide fragment comprising the amino acid sequence of FRNSSHHPW (SEQ ID NO:136), an NKG2A polypeptide fragment comprising the amino acid sequence of IKDSDNAEL (SEQ ID NO:137), an NKG2A polypeptide fragment comprising the amino acid sequence of LQVNR (SEQ ID NO:138), and an NKG2A polypeptide fragment comprising the amino acid sequence of AQCGSSI (SEQ ID NO:139). In some embodiments, the NKG2A binding domain provided herein specifically binds to two of the following NKG2A polypeptide fragments: an NKG2A polypeptide fragment comprising the amino acid sequence of TWEESL (SEQ ID NO:134), an NKG2A polypeptide fragment comprising the amino acid sequence of SIISPSSWIGV (SEQ ID NO:135), an NKG2A polypeptide fragment comprising the amino acid sequence of FRNSSHHPW (SEQ ID NO: 136), an NKG2A polypeptide fragment comprising the amino acid sequence of IKDSDNAEL (SEQ ID NO:137), an NKG2A polypeptide fragment comprising the amino acid sequence of LQVNR (SEQ ID NO:138), and an NKG2A polypeptide fragment comprising the amino acid sequence of AQCGSSI (SEQ ID NO:139). In some embodiments, the NKG2A binding domain provided herein specifically binds to three of the following NKG2A polypeptide fragments: an NKG2A polypeptide fragment comprising the amino acid sequence of TWEESL (SEQ ID NO:134), an NKG2A polypeptide fragment comprising the amino acid sequence of SIISPSSWIGV (SEQ ID NO:135), an NKG2A polypeptide fragment comprising the amino acid sequence of FRNSSHHPW (SEQ ID NO:136), an NKG2A polypeptide fragment comprising the amino acid sequence of IKDSDNAEL (SEQ ID NO: 137), an NKG2A polypeptide fragment comprising the amino acid sequence of LQVNR (SEQ ID NO:138), and an NKG2A polypeptide fragment comprising the amino acid sequence of AQCGSSI (SEQ ID NO:139). In some embodiments, the NKG2A binding domain provided herein specifically binds to four of the following NKG2A polypeptide fragments: an NKG2A polypeptide fragment comprising the amino acid sequence of TWEESL (SEQ ID NO:134), an NKG2A polypeptide fragment comprising the amino acid sequence of SIISPSSWIGV (SEQ ID NO:135), an NKG2A polypeptide fragment comprising the amino acid sequence of FRNSSHHPW (SEQ ID NO:136), an NKG2A polypeptide fragment comprising the amino acid sequence of IKDSDNAEL (SEQ ID NO:137), an NKG2A polypeptide fragment comprising the amino acid sequence of LQVNR (SEQ ID NO: 138), and an NKG2A polypeptide fragment comprising the amino acid sequence of AQCGSSI (SEQ ID NO:139). In some embodiments, the NKG2A binding domain provided herein specifically binds to five of the following NKG2A polypeptide fragments: an NKG2A polypeptide fragment comprising the amino acid sequence of TWEESL (SEQ ID NO:134), an NKG2A polypeptide fragment comprising the amino acid sequence of SIISPSSWIGV (SEQ ID NO:135), an NKG2A polypeptide fragment comprising the amino acid sequence of FRNSSHHPW (SEQ ID NO:136), an NKG2A polypeptide fragment comprising the amino acid sequence of IKDSDNAEL (SEQ ID NO:137), an NKG2A polypeptide fragment comprising the amino acid sequence of LQVNR (SEQ ID NO: 138), and an NKG2A polypeptide fragment comprising the amino acid sequence of AQCGSSI (SEQ ID NO:139). In some embodiments, the NKG2A binding domain provided herein specifically binds to all of the following NKG2A polypeptide fragments: an NKG2A polypeptide fragment comprising the amino acid sequence of TWEESL (SEQ ID NO:134), an NKG2A polypeptide fragment comprising the amino acid sequence of SIISPSSWIGV (SEQ ID NO:135), an NKG2A polypeptide fragment comprising the amino acid sequence of FRNSSHHPW (SEQ ID NO: 136), an NKG2A polypeptide fragment comprising the amino acid sequence of IKDSDNAEL (SEQ ID NO:137), an NKG2A polypeptide fragment comprising the amino acid sequence of LQVNR (SEQ ID NO:138), and an NKG2A polypeptide fragment comprising the amino acid sequence of AQCGSSI (SEQ ID NO:139).

In some embodiments, the NKG2A binding domain provided herein specifically binds to a conformational epitope formed by a group of amino acid residues comprising at least one amino acid residue from one of the following amino acid sequences: TWEESL (SEQ ID NO:134), SIISPSSWIGV (SEQ ID NO:135), FRNSSHHPW (SEQ ID NO:136), IKDSDNAEL (SEQ ID NO:137), LQVNR (SEQ ID NO:138), and AQCGSSI (SEQ ID NO: 139). In some embodiments, the NKG2A binding domain provided herein specifically binds to a conformational epitope formed by a group of amino acid residues comprising at least one amino acid residue from each one of two of the following amino acid sequences: TWEESL (SEQ ID NO:134), SIISPSSWIGV (SEQ ID NO:135), FRNSSHHPW (SEQ ID NO: 136), IKDSDNAEL (SEQ ID NO:137), LQVNR (SEQ ID NO:138), and AQCGSSI (SEQ ID NO:139). In some embodiments, the NKG2A binding domain provided herein specifically binds to a conformational epitope formed by a group of amino acid residues comprising at least one amino acid residue from each one of three of the following amino acid sequences: TWEESL (SEQ ID NO:134), SIISPSSWIGV (SEQ ID NO:135), FRNSSHHPW (SEQ ID NO:136), IKDSDNAEL (SEQ ID NO:137), LQVNR (SEQ ID NO: 138), and AQCGSSI (SEQ ID NO:139). In some embodiments, the NKG2A binding domain provided herein specifically binds to a conformational epitope formed by a group of amino acid residues comprising at least one amino acid residue from each one of four of the following amino acid sequences: TWEESL (SEQ ID NO:134), SIISPSSWIGV (SEQ ID NO: 135), FRNSSHHPW (SEQ ID NO:136), IKDSDNAEL (SEQ ID NO:137), LQVNR (SEQ ID NO:138), and AQCGSSI (SEQ ID NO:139). In some embodiments, the NKG2A binding domain provided herein specifically binds to a conformational epitope formed by a group of amino acid residues comprising at least one amino acid residue from each one of five of the following amino acid sequences: TWEESL (SEQ ID NO:134), SIISPSSWIGV (SEQ ID NO:135), FRNSSHHPW (SEQ ID NO:136), IKDSDNAEL (SEQ ID NO:137), LQVNR (SEQ ID NO:138), and AQCGSSI (SEQ ID NO:139). In some embodiments, the NKG2A binding domain provided herein specifically binds to a conformational epitope formed by a group of amino acid residues comprising at least one amino acid residue from each one of all of the following amino acid sequences: TWEESL (SEQ ID NO:134), SIISPSSWIGV (SEQ ID NO:135), FRNSSHHPW (SEQ ID NO:136), IKDSDNAEL (SEQ ID NO: 137), LQVNR (SEQ ID NO:138), and AQCGSSI (SEQ ID NO:139). In some embodiments, the group of amino acid residue forming the conformational epitope comprises one amino acid residue from an amino acid sequence mentioned above in this paragraph. In some embodiments, the group of amino acid residue forming the conformational epitope comprises two amino acid residues from an amino acid sequence mentioned above in this paragraph. In some embodiments, the group of amino acid residue forming the conformational epitope comprises three amino acid residues from an amino acid sequence mentioned above in this paragraph. In some embodiments, the group of amino acid residue forming the conformational epitope comprises four amino acid residues from an amino acid sequence mentioned above in this paragraph. In some embodiments, the group of amino acid residue forming the conformational epitope comprises five amino acid residues from an amino acid sequence mentioned above in this paragraph. In some embodiments, the group of amino acid residue forming the conformational epitope comprises more than five amino acid residues from an amino acid sequence mentioned above in this paragraph.

In some embodiments, the NKG2A binding domain provided herein specifically binds to a conformational epitope formed by a group of amino acid residues comprising at least one amino acid residue from one of the following amino acid sequences located on the surface of NKG2A: TWEESL (SEQ ID NO:134), SIISPSSWIGV (SEQ ID NO:135), FRNSSHHPW (SEQ ID NO:136), IKDSDNAEL (SEQ ID NO:137), LQVNR (SEQ ID NO: 138), and AQCGSSI (SEQ ID NO:139). In some embodiments, the NKG2A binding domain provided herein specifically binds to a conformational epitope formed by a group of amino acid residues comprising at least one amino acid residue from each one of two of the following amino acid sequences located on the surface of NKG2A: TWEESL (SEQ ID NO: 134), SIISPSSWIGV (SEQ ID NO:135), FRNSSHHPW (SEQ ID NO:136), IKDSDNAEL (SEQ ID NO:137), LQVNR (SEQ ID NO:138), and AQCGSSI (SEQ ID NO: 139). In some embodiments, the NKG2A binding domain provided herein specifically binds to a conformational epitope formed by a group of amino acid residues comprising at least one amino acid residue from each one of three of the following amino acid sequences located on the surface of NKG2A: TWEESL (SEQ ID NO:134), SIISPSSWIGV (SEQ ID NO: 135), FRNSSHHPW (SEQ ID NO:136), IKDSDNAEL (SEQ ID NO:137), LQVNR (SEQ ID NO:138), and AQCGSSI (SEQ ID NO:139). In some embodiments, the NKG2A binding domain provided herein specifically binds to a conformational epitope formed by a group of amino acid residues comprising at least one amino acid residue from each one of four of the following amino acid sequences located on the surface of NKG2A: TWEESL (SEQ ID NO:134), SIISPSSWIGV (SEQ ID NO:135), FRNSSHHPW (SEQ ID NO:136), IKDSDNAEL (SEQ ID NO:137), LQVNR (SEQ ID NO:138), and AQCGSSI (SEQ ID NO: 139). In some embodiments, the NKG2A binding domain provided herein specifically binds to a conformational epitope formed by a group of amino acid residues comprising at least one amino acid residue from each one of five of the following amino acid sequences located on the surface of NKG2A: TWEESL (SEQ ID NO:134), SIISPSSWIGV (SEQ ID NO: 135), FRNSSHHPW (SEQ ID NO:136), IKDSDNAEL (SEQ ID NO:137), LQVNR (SEQ ID NO:138), and AQCGSSI (SEQ ID NO:139). In some embodiments, the NKG2A binding domain provided herein specifically binds to a conformational epitope formed by a group of amino acid residues comprising at least one amino acid residue from each one of all of the following amino acid sequences located on the surface of NKG2A: TWEESL (SEQ ID NO: 134), SIISPSSWIGV (SEQ ID NO:135), FRNSSHHPW (SEQ ID NO:136), IKDSDNAEL (SEQ ID NO:137), LQVNR (SEQ ID NO:138), and AQCGSSI (SEQ ID NO: 139). In some embodiments, the group of amino acid residue forming the conformational epitope comprises one amino acid residue from an amino acid sequence mentioned above in this paragraph. In some embodiments, the group of amino acid residue forming the conformational epitope comprises two amino acid residues from an amino acid sequence mentioned above in this paragraph. In some embodiments, the group of amino acid residue forming the conformational epitope comprises three amino acid residues from an amino acid sequence mentioned above in this paragraph. In some embodiments, the group of amino acid residue forming the conformational epitope comprises four amino acid residues from an amino acid sequence mentioned above in this paragraph. In some embodiments, the group of amino acid residue forming the conformational epitope comprises five amino acid residues from an amino acid sequence mentioned above in this paragraph. In some embodiments, the group of amino acid residue forming the conformational epitope comprises more than five amino acid residues from an amino acid sequence mentioned above in this paragraph.

In some embodiments, the multispecific binding agent provided herein (e.g., a bispecific antibody) specifically binds to NKG2A competitively with any one of the anti-NKG2A antibodies or fragments thereof described herein.

In some embodiments, the multispecific binding agent provided herein (e.g., a bispecific antibody) specifically binds to NKG2A competitively with an anti-NKG2A antibody comprising a VH CDR1, a VH CDR2, and a VH CDR3 as set forth in a VH comprising the amino acid sequence of SEQ ID NO:25 and a VL CDR1, a VL CDR2, and a VL CDR3 as set forth in a VL comprising the amino acid sequence of SEQ ID NO:26. In some embodiments, the multispecific binding agent provided herein (e.g., a bispecific antibody) specifically binds to NKG2A competitively with an anti-NKG2A antibody comprising a VH comprising the amino acid sequence of SEQ ID NO:25, and a VL comprising the amino acid sequence of SEQ ID NO:26.

In some embodiments, the multispecific binding agent provided herein (e.g., a bispecific antibody) specifically binds to NKG2A competitively with an anti-NKG2A antibody comprising a VH CDR1, a VH CDR2, and a VH CDR3 as set forth in a VH comprising the amino acid sequence of SEQ ID NO:45 and a VL CDR1, a VL CDR2, and a VL CDR3 as set forth in a VL comprising the amino acid sequence of SEQ ID NO:46. In some embodiments, the multispecific binding agent provided herein (e.g., a bispecific antibody) specifically binds to NKG2A competitively with an anti-NKG2A antibody comprising a VH comprising the amino acid sequence of SEQ ID NO:45, and a VL comprising the amino acid sequence of SEQ ID NO:46.

In some embodiments, the multispecific binding agent provided herein (e.g., a bispecific antibody) specifically binds to NKG2A competitively with an anti-NKG2A antibody comprising a VH CDR1, a VH CDR2, and a VH CDR3 as set forth in a VH comprising the amino acid sequence of SEQ ID NO:64 and a VL CDR1, a VL CDR2, and a VL CDR3 as set forth in a VL comprising the amino acid sequence of SEQ ID NO:65. In some embodiments, the multispecific binding agent provided herein (e.g., a bispecific antibody) specifically binds to NKG2A competitively with an anti-NKG2A antibody comprising a VH comprising the amino acid sequence of SEQ ID NO:64, and a VL comprising the amino acid sequence of SEQ ID NO:65.

In some embodiments, the multispecific binding agent provided herein (e.g., a bispecific antibody) specifically binds to NKG2A competitively with an anti-NKG2A antibody comprising a VH CDR1, a VH CDR2, and a VH CDR3 as set forth in a VH comprising the amino acid sequence of SEQ ID NO:64 and a VL CDR1, a VL CDR2, and a VL CDR3 as set forth in a VL comprising the amino acid sequence of SEQ ID NO:73. In some embodiments, the multispecific binding agent provided herein (e.g., a bispecific antibody) specifically binds to NKG2A competitively with an anti-NKG2A antibody comprising a VH comprising the amino acid sequence of SEQ ID NO:64, and a VL comprising the amino acid sequence of SEQ ID NO:73.

In some embodiments, the antibodies based on which the present NKG2A binding arm is derived are superior developability based on a known assay in the art, for example, various chromatographic methods, including size exclusion chromatography (SEC), hydrophobic interaction chromatography (HIC), and standup monolayer adsorption chromatography (SMAC). In some embodiments, the antibodies based on which the present NKG2A binding arm is derived are superior developability based on measurement of monomer percentage, solubility, and/or antibody aggregation or precipitation.

### 5.2.2. PD-L1 Binding Domains

Also provided herein is a binding agent that binds to PD-L1. In some embodiments, the binding agent comprises one or more PD-L1 binding domains. In some embodiments, the multispecific binding agents provided herein further comprise one or more PD-L1 binding domains. In some embodiments, described herein are multispecific binding agents (e.g., bispecific antibodies) that bind to PD-L1. Asused herein, PD-L1 refers to a PD-L1 polypeptide, a PD-L1 polypeptide fragment, a PD-L1 peptide or a PD-L1 epitope. In some embodiments, the PD-L1 binding domains in the present binding agents, such as multispecific binding agents, are derived from human or humanized antibodies (e.g., comprising human framework regions) that bind PD-L1, including a PD-L1 polypeptide, a PD-L1 polypeptide fragment, a PD-L1 peptide or a PD-L1 epitope. In some embodiments, the binding agent (e.g., an antibody, a multispecific binding agent, or a bispecific antibody) can bind to PD-L1 expressed on the surface of a mammalian (e.g., human) cell, including a PD-L1 expressing immune cell (e.g., a T cell) and/or a PD-L1 expressing cancer or tumor cell. In some embodiments, the binding agent (e.g., an antibody, a multispecific binding agent, or a bispecific antibody) provided herein binds a PD-L1 extracellular epitope exposed on a cell such as an immune cell, and/or a cancer or tumor cell. In some embodiments, described herein is a binding agent (e.g., an antibody, a multispecific binding agent, or a bispecific antibody) that binds to PD-L1, such as human PD-L1 or portions thereof. In some embodiments, PD-L1 is a human PD-L1. In some embodiments, the binding agent (such as an antibody, a multispecific binding agent, or a bispecific antibody) provided herein is a human PD-L1 binding agent (e.g., an antibody that binds to human PD-L1). In some embodiments, the binding agent provided herein binds to both human PD-L1 and cyno PD-L1. In other embodiments, the binding agent provided herein binds to human PD-L1 but not cyno PD-L1.

In some embodiments, the binding agent (e.g., an antibody, a multispecific binding agent, or a bispecific antibody) provided herein binds to PD-L1 (e.g., human PD-L1) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸ M to 10⁻¹³ M, e.g., from 10⁻⁹ M to 10⁻¹³ M). A variety of methods of measuring binding affinity are known in the art, any of which can be used for purposes of the present disclosure, including those described herein, e.g., in Section 5.2.1 above.

In some embodiments, the binding agents (e.g., an antibody, a multispecific binding agent, or a bispecific antibody) described herein comprise a VH region, VL region, VH CDR1, VH CDR2, VH CDR3, VL CDR1, VL CDR2, and/or VL CDR3 of any anti-PD-L1 antibody, such as an amino acid sequence of a VH region, VL region, VH CDR1, VH CDR2, VH CDR3, VL CDR1, VL CDR2, and/or VL CDR3 depicted in Table 5. Accordingly, in some embodiments, a binding agent (e.g., an antibody, a multispecific binding agent, or a bispecific antibody) described herein comprises any one, any two, and/or all three heavy chain CDRs and/or any one, any two, and/or all three light chain CDRs from the antibody designated P12 as shown in Table 5. In some embodiments, a binding agent (e.g., an antibody, a multispecific binding agent, or a bispecific antibody) described herein comprises any one, any two, and/or all three heavy chain CDRs and any one, any two, and/or all three light chain CDRs from the antibody designated P12 as shown in Table 5.

In some embodiments, a binding agent (e.g., an antibody, a multispecific binding agent, or a bispecific antibody) comprises a VH region, which comprises a VH CDR1, a VH CDR2, and/or a VH CDR3, and/or a VL region, which comprises a VL CDR1, a VL CDR2, and/or a VL CDR3, of any one of the binding agents described herein (see, e.g., Table 5). Accordingly, in some embodiments, a binding agent (e.g., an antibody, a multispecific binding agent, or a bispecific antibody) described herein comprises any one, any two, and/or all three heavy chain CDRs and/or any one, any two, and/or all three light chain CDRs from Table 5.

In some embodiments, the binding agent provided herein (e.g., an antibody, a multispecific binding agent, or a bispecific antibody) comprises (i) a VH CDR1, a VH CDR2, and a VH CDR3 as set forth in a VH comprising the amino acid sequence of SEQ ID NO:98 and/or (ii) a VL CDR1, a VL CDR2, and a VL CDR3 as set forth in a VL comprising the amino acid sequence of SEQ ID NO:99. In some embodiments, the binding agent provided herein (e.g., an antibody, a multispecific binding agent, or a bispecific antibody) comprises a VH CDR1, a VH CDR2, and a VH CDR3 as set forth in a VH comprising the amino acid sequence of SEQ ID NO:98 and/or a VL CDR1, a VL CDR2, and a VL CDR3 as set forth in a VL comprising the amino acid sequence of SEQ ID NO:99. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDR sequences are determined according to a combination of any two or more of the above-mentioned numbering systems, for example, a combination of Kabat and Chothia. Various exemplary CDR numbering systems are described and illustrated above in Section 5.1.

In some embodiments, the binding agent provided herein (e.g., an antibody, a multispecific binding agent, or a bispecific antibody) comprises (a) a VH region comprising: (1) a VH CDR1 having an amino acid sequence selected from the group consisting of SEQ ID NOs: 1, 7, 12, 13, and 18; (2) a VH CDR2 having an amino acid sequence selected from the group consisting of SEQ ID NOs: 86, 89, 91, 94, and 97; and (3) a VH CDR3 having an amino acid sequence selected from the group consisting of SEQ ID NOs: 87, 90. 92, and 95; and (b) a VL region comprising: (1) a VL CDR1 having an amino acid sequence selected from the group consisting of SEQ ID NOs: 4, 10, 16, and 21; (2) VL CDR2 having an amino acid sequence selected from the group consisting of SEQ ID NOs: 5, 11, and 22; and (3) a VL CDR3 having an amino acid sequence selected from the group consisting of SEQ ID NOs: 88, 93, and 96.

In some embodiments, the binding agent provided herein (e.g., an antibody, a multispecific binding agent, or a bispecific antibody) comprises a VH region comprising a VH CDR1 comprising the amino acid sequence of SEQ ID NO:1, a VH CDR2 comprising the amino acid sequence of SEQ ID NO:86, a VH CDR3 comprising the amino acid sequence of SEQ ID NO:87; and a VL region comprising a VL CDR1 comprising the amino acid sequence of SEQ ID NO:4, a VL CDR2 comprising the amino acid sequence of SEQ ID NO: 5, and a VL CDR3 comprising the amino acid sequence of SEQ ID NO:88.

In some embodiments, the binding agent provided herein (e.g., an antibody, a multispecific binding agent, or a bispecific antibody) comprises a VH region comprising a VH CDR1 comprising the amino acid sequence of SEQ ID NO:7, a VH CDR2 comprising the amino acid sequence of SEQ ID NO:89, a VH CDR3 comprising the amino acid sequence of SEQ ID NO:90; and a VL region comprising a VL CDR1 comprising the amino acid sequence of SEQ ID NO:10, a VL CDR2 comprising the amino acid sequence of SEQ ID NO: 11, and a VL CDR3 comprising the amino acid sequence of SEQ ID NO:88.

In some embodiments, the binding agent provided herein (e.g., an antibody, a multispecific binding agent, or a bispecific antibody) comprises a VH region comprising a VH CDR1 comprising the amino acid sequence of SEQ ID NO:12, a VH CDR2 comprising the amino acid sequence of SEQ ID NO:86, a VH CDR3 comprising the amino acid sequence of SEQ ID NO:87; and a VL region comprising a VL CDR1 comprising the amino acid sequence of SEQ ID NO:4, a VL CDR2 comprising the amino acid sequence of SEQ ID NO: 5, and a VL CDR3 comprising the amino acid sequence of SEQ ID NO:88.

In some embodiments, the binding agent provided herein (e.g., an antibody, a multispecific binding agent, or a bispecific antibody) comprises a VH region comprising a VH CDR1 comprising the amino acid sequence of SEQ ID NO:13, a VH CDR2 comprising the amino acid sequence of SEQ ID NO:91, a VH CDR3 comprising the amino acid sequence of SEQ ID NO:92; and a VL region comprising a VL CDR1 comprising the amino acid sequence of SEQ ID NO:16, a VL CDR2 comprising the amino acid sequence of SEQ ID NO: 11, and a VL CDR3 comprising the amino acid sequence of SEQ ID NO:93.

In some embodiments, the binding agent provided herein (e.g., an antibody, a multispecific binding agent, or a bispecific antibody) comprises a VH region comprising a VH CDR1 comprising the amino acid sequence of SEQ ID NO:18, a VH CDR2 comprising the amino acid sequence of SEQ ID NO:94, a VH CDR3 comprising the amino acid sequence of SEQ ID NO:95; and a VL region comprising a VL CDR1 comprising the amino acid sequence of SEQ ID NO:21, a VL CDR2 comprising the amino acid sequence of SEQ ID NO: 22, and a VL CDR3 comprising the amino acid sequence of SEQ ID NO:96.

In some embodiments, the binding agent provided herein (e.g., an antibody, a multispecific binding agent, or a bispecific antibody) comprises a VH region comprising a VH CDR1 comprising the amino acid sequence of SEQ ID NO:1, a VH CDR2 comprising the amino acid sequence of SEQ ID NO:97, a VH CDR3 comprising the amino acid sequence of SEQ ID NO:87; and a VL region comprising a VL CDR1 comprising the amino acid sequence of SEQ ID NO:4, a VL CDR2 comprising the amino acid sequence of SEQ ID NO: 5, and a VL CDR3 comprising the amino acid sequence of SEQ ID NO:88.

In some embodiments, the PD-L1 binding domain in the binding agent (e.g., an antibody, a multispecific binding agent, or a bispecific antibody) provided herein further comprises one or more framework regions of SEQ ID NOs: 98 and 99. In some embodiments, the PD-L1 binding domain further comprises a framework 1 (FR1), a framework 2 (FR2), a framework 3 (FR3) and/or a framework 4 (FR4) sequence as set forth in any one of SEQ ID NOs: 98 and 99. In some embodiments, the PD-L1 binding domain provided herein is derived from a humanized antibody. Framework regions described herein are determined based upon the boundaries of the CDR numbering system as described in Section 5.2.1 above.

In some embodiments, the PD-L1 binding domains described herein comprise a VH region or VH domain. Additionally or alternatively, in some embodiments, the PD-L1 binding domains described herein comprise a VL region or VL domain. In some embodiments, the PD-L1 binding domains described herein have a combination of (i) a VH domain or VH region; and (ii) a VL domain or VL region.

In some embodiments, the binding agent provided herein (e.g., an antibody, a multispecific binding agent, or a bispecific antibody) comprises a VH comprising the amino acid sequence of SEQ ID NO:98. In some embodiments, the binding agent provided herein (e.g., an antibody, a multispecific binding agent, or a bispecific antibody) comprises a VL comprising the amino acid sequence of SEQ ID NO:99. In some embodiments, the binding agent provided herein (e.g., an antibody, a multispecific binding agent, or a bispecific antibody) comprises a VH comprising the amino acid sequence of SEQ ID NO:98 and a VL comprising the amino acid sequence of SEQ ID NO:99.

In certain embodiments, the binding agent (e.g., an antibody, a multispecific binding agent, or a bispecific antibody) provided herein comprises amino acid sequences with certain percent identity (such as at least about 80%, or at least about 81%, or at least about 82%, or at least about 83%, or at least about 84%, or at least about 85%, or at least about 86%, or at least about 87%, or at least about 88%, or at least about 89%, or as at least about 90%, or at least about 91%, or at least about 92%, or at least about 93%, or at least about 94%, or at least about 95%, or at least about 96%, or at least about 97%, or at least about 98%, or at least about 99%, or higher) relative to any antibody or fragment thereof provided herein, for example, a CDR, VH or VL in Table 5, or a full-length antibody chain as disclosed herein. In some embodiments, the binding agent (e.g., an antibody, a multispecific binding agent, or a bispecific antibody) provided herein comprises CDRs of any antibody or fragment thereof provided herein, for example in Table 5. In further embodiments, the binding agent (e.g., an antibody, a multispecific binding agent, or a bispecific antibody) provided herein comprises amino acid sequences with certain percent identity (such as at least about 80%, or at least about 81%, or at least about 82%, or at least about 83%, or at least about 84%, or at least about 85%, or at least about 86%, or at least about 87%, or at least about 88%, or at least about 89%, or as at least about 90%, or at least about 91%, or at least about 92%, or at least about 93%, or at least about 94%, or at least about 95%, or at least about 96%, or at least about 97%, or at least about 98%, or at least about 99%, or higher) relative to any antibody or fragment thereof provided herein, for example, a VH or VL in Table 5, or a full-length antibody chain as disclosed herein. The determination of percent identity between two sequences (e.g., amino acid sequences or nucleic acid sequences) can be accomplished using a method as described in Section 5.2.1 above.

In some embodiments, the binding agent (e.g., an antibody, a multispecific binding agent, or a bispecific antibody) provided herein contains substitutions (e.g., conservative substitutions), insertions, or deletions relative to the reference sequence, but the binding agent (e.g., an antibody, a multispecific binding agent, or a bispecific antibody) comprising that sequence retains the ability to bind to PD-L1. In some embodiments, a total of 1 to 10 amino acids have been substituted, inserted and/or deleted in a reference amino acid sequence. In some embodiments, substitutions, insertions, or deletions occur in regions outside the CDRs (e.g., in the FRs, constant regions, and/or Fc regions).

In some embodiments, the position of one or more CDRs along the VH (e.g., CDR1, CDR2, or CDR3) and/or VL (e.g., CDR1, CDR2, or CDR3) region of a PD-L1 binding domain described herein may vary by one, two, three, four, five, or six amino acid positions so long as binding to PD-L1 (e.g., human PD-L1) is maintained (e.g., substantially maintained, for example, at least 50%, at least 60%, at least 70%, at least 80%, at least 90%, or at least 95%). For example, in some embodiments, the position defining a CDR of Table 5 may vary by shifting the N-terminal and/or C-terminal boundary of the CDR by one, two, three, four, five, or six amino acids, relative to the current CDR position, so long as binding to PD-L1 (e.g., human PD-L1) is maintained (e.g., substantially maintained, for example, at least 50%, at least 60%, at least 70%, at least 80%, at least 90%, or at least 95%). Additionally or alternatively, in some embodiments, the length of one or more CDRs along the VH (e.g., CDR1, CDR2, or CDR3) and/or VL (e.g., CDR1, CDR2, or CDR3) region of a PD-L1 binding domain described herein may vary (e.g., be shorter or longer) by one, two, three, four, five, or more amino acids, so long as binding to PD-L1 (e.g., human PD-L1) is maintained (e.g., substantially maintained, for example, at least 50%, at least 60%, at least 70%, at least 80%, at least 90%, or at least 95%). For example, in some embodiments, a VH and/or VL CDR1, CDR2, and/or CDR3 described herein may be one, two, three, four, five or more amino acids shorter than one or more of the CDRs described by SEQ ID NOS: 1, 4-5, 7, 10-13, 16, 18, 21, 22, and 86-97, so long as binding to PD-L1 (e.g., human PD-L1) is maintained (e.g., substantially maintained, for example, at least 50%, at least 60%, at least 70%, at least 80%, at least 90%, or at least 95%). In other embodiments, a VH and/or VL CDR1, CDR2, and/or CDR3 described herein may be one, two, three, four, five or more amino acids longer than one or more of the CDRs described by SEQ ID NOS: 1, 4-5, 7, 10-13, 16, 18, 21, 22, and 86-97, so long as binding to PD-L1 (e.g., human PD-L1) is maintained (e.g., substantially maintained, for example, at least 50%, at least 60%, at least 70%, at least 80%, at least 90%, or at least 95%). In some embodiments, the amino terminus of a VH and/or VL CDR1, CDR2, and/or CDR3 described herein may be extended or shortened by one, two, three, four, five or more amino acids compared to one or more of the CDRs described by SEQ ID NOS: 1, 4-5, 7, 10-13, 16, 18, 21, 22, and 86-97, so long as binding to PD-L1 (e.g., human PD-L1) is maintained (e.g., substantially maintained, for example, at least 50%, at least 60%, at least 70%, at least 80%, at least 90%, or at least 95%). Additionally or alternatively, in some embodiments, the carboxy terminus of a VH and/or VL CDR1, CDR2, and/or CDR3 described herein may be extended or shortened by one, two, three, four, five or more amino acids compared to one or more of the CDRs described by SEQ ID NOS: 1, 4-5, 7, 10-13, 16, 18, 21, 22, and 86-97, so long as binding to PD-L1 (e.g., human PD-L1) is maintained (e.g., substantially maintained, for example, at least 50%, at least 60%, at least 70%, at least 80%, at least 90%, or at least 95%). Any method known in the art can be used to ascertain whether binding to PD-L1 (e.g., human PD-L1) is maintained, for example, the binding assays and conditions described in the “Examples” section described herein.

In other embodiments, the binding agents (e.g., an antibody, a multispecific binding agent, or a bispecific antibody) further comprise conservative sequence modifications (e.g., in a PD-L1 binding domain). Conservative sequence modifications are described in more detail in Section 5.2.1 above. In some embodiments, the conservative sequence modifications described herein modify the amino acid sequences of the binding agents (e.g., an antibody, a multispecific binding agent, or a bispecific antibody), including human PD-L1 binding agents, by 50%, or 55%, or 60%, or 65%, or 70%, or 75%, or 80%, or 85%, or 90%, or 95%, or 98%, or 99%. In some embodiments, the amino acid sequence modifications refer to at most 1, 2, 3, 4, 5, or 6 amino acid substitutions to the CDRs, such as those described in Table 5. Thus, for example, each such CDR may contain up to 5 conservative amino acid substitutions, for example up to (not more than) 4 conservative amino acid substitutions, for example up to (not more than) 3 conservative amino acid substitutions, for example up to (not more than) 2 conservative amino acid substitutions, or no more than 1 conservative amino acid substitution. In some embodiments, the PD-L1 binding domain contains one or more, including six, CDRs having at least 75%, 80%, 85%, 90%, 95%, 96%, 97%, 98%, or 99% identity to the CDRs of P12 (see, e.g., Table 5).

In some embodiments, a PD-L1 binding domain contains a VH and a VL comprising CDRs identical to those of P12 (see, e.g., Table 5). In some embodiments, the amino acid sequence modifications do not include any modification within an SDR. In some embodiments, the amino acid sequence modifications do not include any modification within a CDR (such as CDR1, CDR2, CDR3, or any combination thereof). In further embodiments, the amino acid sequence modifications are in the framework, constant region, and/or Fc region.

In some embodiments, the binding agent (e.g., an antibody, a multispecific binding agent, or a bispecific antibody) provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:98, and/or a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO:99, and the binding of the binding agent to PD-L1 (e.g., human PD-L1) is maintained (e.g., substantially maintained, for example, at least 50%, at least 60%, at least 70%, at least 80%, at least 90%, or at least 95%).

In some embodiments, functional epitopes can be mapped, e.g., by combinatorial alanine scanning or hydrogen/deuterium exchange mass spectrometry (HDX-MS), to identify amino acids in the PD-L1 protein that are necessary for interaction with a multispecific binding agent, a PD-L1 binding domain thereof, and/or an anti-PD-L1 antibody provided herein (e.g., in the following paragraphs). In some embodiments, conformational and crystal structure of a multispecific binding agent, a PD-L1 binding domain thereof, and/or an anti-PD-L1 antibody (e.g., provided in the following paragraphs) bound to PD-L1 may be employed to identify the epitopes. In some embodiments, the present disclosure provides a binding agent (e.g., an antibody, a multispecific binding agent, or a bispecific antibody) comprising a PD-L1 binding domain that specifically binds to the same epitope as any of the multispecific binding agents as disclosed herein, PD-L1 binding domains thereof, and/or the anti-PD-L1 antibodies or fragments thereof provided herein (e.g., provided in the following paragraphs).

For example, in some embodiments, the PD-L1 binding domain provided herein binds to the same epitope as an anti-PD-L1 antibody comprising a VH CDR1, a VH CDR2, and a VH CDR3 as set forth in a VH comprising the amino acid sequence of SEQ ID NO:98 and a VL CDR1, a VL CDR2, and a VL CDR3 as set forth in a VL comprising the amino acid sequence of SEQ ID NO:99. In some embodiments, the PD-L1 binding domain provided herein binds to the same epitope as an anti-PD-L1 antibody comprising a VH comprising the amino acid sequence of SEQ ID NO:98, and a VL comprising the amino acid sequence of SEQ ID NO:99.

In some embodiments, the binding agent provided herein (e.g., an antibody, a multispecific binding agent, or a bispecific antibody) specifically binds to PD-L1 competitively with any one of the anti-PD-L1 antibodies or fragments thereof described herein.

In some embodiments, the binding agent provided herein (e.g., an antibody, a multispecific binding agent, or a bispecific antibody) specifically binds to PD-L1 competitively with an anti-PD-L1 antibody comprising a VH CDR1, a VH CDR2, and a VH CDR3 as set forth in a VH comprising the amino acid sequence of SEQ ID NO:98 and a VL CDR1, a VL CDR2, and a VL CDR3 as set forth in a VL comprising the amino acid sequence of SEQ ID NO:99. In some embodiments, the binding agent provided herein (e.g., an antibody, a multispecific binding agent, or a bispecific antibody) specifically binds to PD-L1 competitively with an anti-PD-L1 antibody comprising a VH comprising the amino acid sequence of SEQ ID NO:98, and a VL comprising the amino acid sequence of SEQ ID NO: 99.

In yet another aspect, provided herein is a binding agent (e.g., an antibody or fragment thereof) that binds to PD-L1 comprising one or more CDR(s) described above. In some embodiments, provided herein is a binding agent (e.g., an antibody or fragment thereof) that binds to PD-L1 comprising the VH and/or VL described above. In some embodiments, the binding agent comprises a VH CDR1, a VH CDR2, and a VH CDR3 as set forth in a VH comprising the amino acid sequence of SEQ ID NO:98 and a VL CDR1, a VL CDR2, and a VL CDR3 as set forth in a VL comprising the amino acid sequence of SEQ ID NO:99. In some embodiments, the VH region comprises: (1) a VH CDR1 having an amino acid sequence selected from the group consisting of SEQ ID NOs: 1, 7, 12, 13, and 18; (2) a VH CDR2 having an amino acid sequence selected from the group consisting of SEQ ID NOs: 86, 89, 91, 94, and 97; and (3) a VH CDR3 having an amino acid sequence selected from the group consisting of SEQ ID NOs: 87, 90. 92, and 95; and the VL region comprises: (1) a VL CDR1 having an amino acid sequence selected from the group consisting of SEQ ID NOs: 4, 10, 16, and 21; (2) a VL CDR2 having an amino acid sequence selected from the group consisting of SEQ ID NOs: 5, 11, and 22; and (3) a VL CDR3 having an amino acid sequence selected from the group consisting of SEQ ID NOs: 88, 93, and 96.

In some embodiments, the binding agent comprises a VH region comprising a VH CDR1 comprising the amino acid sequence of SEQ ID NO:1, a VH CDR2 comprising the amino acid sequence of SEQ ID NO:86, a VH CDR3 comprising the amino acid sequence of SEQ ID NO:87; and a VL region comprising a VL CDR1 comprising the amino acid sequence of SEQ ID NO:4, a VL CDR2 comprising the amino acid sequence of SEQ ID NO: 5, and a VL CDR3 comprising the amino acid sequence of SEQ ID NO:88.

In some embodiments, the binding agent comprises a VH region comprising a VH CDR1 comprising the amino acid sequence of SEQ ID NO:7, a VH CDR2 comprising the amino acid sequence of SEQ ID NO:89, a VH CDR3 comprising the amino acid sequence of SEQ ID NO:90; and a VL region comprising a VL CDR1 comprising the amino acid sequence of SEQ ID NO:10, a VL CDR2 comprising the amino acid sequence of SEQ ID NO: 11, and a VL CDR3 comprising the amino acid sequence of SEQ ID NO:88.

In some embodiments, the binding agent comprises a VH region comprising a VH CDR1 comprising the amino acid sequence of SEQ ID NO:12, a VH CDR2 comprising the amino acid sequence of SEQ ID NO:86, a VH CDR3 comprising the amino acid sequence of SEQ ID NO:87; and a VL region comprising a VL CDR1 comprising the amino acid sequence of SEQ ID NO:4, a VL CDR2 comprising the amino acid sequence of SEQ ID NO: 5, and a VL CDR3 comprising the amino acid sequence of SEQ ID NO:88.

In some embodiments, the binding agent comprises a VH region comprising a VH CDR1 comprising the amino acid sequence of SEQ ID NO:13, a VH CDR2 comprising the amino acid sequence of SEQ ID NO:91, a VH CDR3 comprising the amino acid sequence of SEQ ID NO:92; and a VL region comprising a VL CDR1 comprising the amino acid sequence of SEQ ID NO:16, a VL CDR2 comprising the amino acid sequence of SEQ ID NO: 11, and a VL CDR3 comprising the amino acid sequence of SEQ ID NO:93.

In some embodiments, the binding agent comprises a VH region comprising a VH CDR1 comprising the amino acid sequence of SEQ ID NO:18, a VH CDR2 comprising the amino acid sequence of SEQ ID NO:94, a VH CDR3 comprising the amino acid sequence of SEQ ID NO:95; and a VL region comprising a VL CDR1 comprising the amino acid sequence of SEQ ID NO:21, a VL CDR2 comprising the amino acid sequence of SEQ ID NO: 22, and a VL CDR3 comprising the amino acid sequence of SEQ ID NO:96.

In some embodiments, the binding agent comprises a VH region comprising a VH CDR1 comprising the amino acid sequence of SEQ ID NO:1, a VH CDR2 comprising the amino acid sequence of SEQ ID NO:97, a VH CDR3 comprising the amino acid sequence of SEQ ID NO:87; and a VL region comprising a VL CDR1 comprising the amino acid sequence of SEQ ID NO:4, a VL CDR2 comprising the amino acid sequence of SEQ ID NO: 5, and a VL CDR3 comprising the amino acid sequence of SEQ ID NO:88.

In some embodiments, the binding agent provided herein comprises a VH region comprising the amino acid sequence of SEQ ID NO:98 and a VL region comprising the amino acid sequence of SEQ ID NO:99.

### 5.2.3. Mutlispecific Antibodies

Provided herein is a multispecific binding agent that binds to NKG2A and a non-NKG2A antigen (such as PD-L1). In some embodiments, the multispecific binding agent comprises one or more NKG2A binding domains and one or more non-NKG2A antigen binding domains (such as one or more PD-L1 binding domains). In certain embodiments, the present multispecific binding agent is a multispecific antibody comprising one or more NKG2A binding domains each independently selected from the NKG2A binding domains described in Section 5.2.1 above and one or more PD-L1 binding domains each independently selected from the PD-L1 binding domains described in Section 5.2.2 above.

In some specific embodiments, provided herein is a multispecific binding agent comprising one or more NKG2A binding domains each independently selected from NKG2A binding domains comprising: (i) a VH CDR1, a VH CDR2, and a VH CDR3 as set forth in a VH comprising the amino acid sequence of SEQ ID NO:25 and a VL CDR1, a VL CDR2, and a VL CDR3 as set forth in a VL comprising the amino acid sequence of SEQ ID NO:26; (ii) a VH CDR1, a VH CDR2, and a VH CDR3 as set forth in a VH comprising the amino acid sequence of SEQ ID NO:45 and a VL CDR1, a VL CDR2, and a VL CDR3 as set forth in a VL comprising the amino acid sequence of SEQ ID NO:46; (iii) a VH CDR1, a VH CDR2, and a VH CDR3 as set forth in a VH comprising the amino acid sequence of SEQ ID NO: 64 and a VL CDR1, a VL CDR2, and a VL CDR3 as set forth in a VL comprising the amino acid sequence of SEQ ID NO:65; or (iv) a VH CDR1, a VH CDR2, and a VH CDR3 as set forth in a VH comprising the amino acid sequence of SEQ ID NO:64 and a VL CDR1, a VL CDR2, and a VL CDR3 as set forth in a VL comprising the amino acid sequence of SEQ ID NO:73; and one or more PD-L1 binding domains comprising a VH CDR1, a VH CDR2, and a VH CDR3 as set forth in a VH comprising the amino acid sequence of SEQ ID NO: 98 and a VL CDR1, a VL CDR2, and a VL CDR3 as set forth in a VL comprising the amino acid sequence of SEQ ID NO:99.

In some embodiments, provided herein is a multispecific binding agent comprising one or more NKG2A binding domains, each comprising a VH CDR1, a VH CDR2, and a VH CDR3 as set forth in a VH comprising the amino acid sequence of SEQ ID NO:25 and a VL CDR1, a VL CDR2, and a VL CDR3 as set forth in a VL comprising the amino acid sequence of SEQ ID NO:26; and one or more PD-L1 binding domains, each comprising a VH CDR1, a VH CDR2, and a VH CDR3 as set forth in a VH comprising the amino acid sequence of SEQ ID NO:98 and a VL CDR1, a VL CDR2, and a VL CDR3 as set forth in a VL comprising the amino acid sequence of SEQ ID NO:99. In some embodiments, the multispecific binding agent provided herein comprises one NKG2A binding domain (e.g., with a pair of VH and VL regions that bind to NKG2A) and one PD-L1 binding domain (e.g., with a pair of VH and VL regions that bind to PD-L1). In some embodiments, the multispecific binding agent provided herein comprises two NKG2A binding domains (e.g., with two identical pairs of VH and VL regions that bind to NKG2A) and one PD-L1 binding domain (e.g., with a pair of VH and VL regions that bind to PD-L1). In some embodiments, the multispecific binding agent provided herein comprises one NKG2A binding domain (e.g., with a pair of VH and VL regions that bind to NKG2A) and two PD-L1 binding domains (e.g., with two identical pairs of VH and VL regions that bind to PD-L1).

In some embodiments, provided herein is a multispecific binding agent comprising one or more NKG2A binding domains, each comprising a VH CDR1, a VH CDR2, and a VH CDR3 as set forth in a VH comprising the amino acid sequence of SEQ ID NO:45 and a VL CDR1, a VL CDR2, and a VL CDR3 as set forth in a VL comprising the amino acid sequence of SEQ ID NO:46; and one or more PD-L1 binding domains, each comprising a VH CDR1, a VH CDR2, and a VH CDR3 as set forth in a VH comprising the amino acid sequence of SEQ ID NO:98 and a VL CDR1, a VL CDR2, and a VL CDR3 as set forth in a VL comprising the amino acid sequence of SEQ ID NO:99. In some embodiments, the multispecific binding agent provided herein comprises one NKG2A binding domain (e.g., with a pair of VH and VL regions that bind to NKG2A) and one PD-L1 binding domain (e.g., with a pair of VH and VL regions that bind to PD-L1). In some embodiments, the multispecific binding agent provided herein comprises two NKG2A binding domains (e.g., with two identical pairs of VH and VL regions that bind to NKG2A) and one PD-L1 binding domain (e.g., with a pair of VH and VL regions that bind to PD-L1). In some embodiments, the multispecific binding agent provided herein comprises one NKG2A binding domain (e.g., with a pair of VH and VL regions that bind to NKG2A) and two PD-L1 binding domains (e.g., with two identical pairs of VH and VL regions that bind to PD-L1).

In some specific embodiments, provided herein is a multispecific binding agent comprising one or more NKG2A binding domains, each comprising a VH CDR1, a VH CDR2, and a VH CDR3 as set forth in a VH comprising the amino acid sequence of SEQ ID NO: 64 and a VL CDR1, a VL CDR2, and a VL CDR3 as set forth in a VL comprising the amino acid sequence of SEQ ID NO:65; and one or more PD-L1 binding domains, each comprising a VH CDR1, a VH CDR2, and a VH CDR3 as set forth in a VH comprising the amino acid sequence of SEQ ID NO:98 and a VL CDR1, a VL CDR2, and a VL CDR3 as set forth in a VL comprising the amino acid sequence of SEQ ID NO:99. In some embodiments, the multispecific binding agent provided herein comprises one NKG2A binding domain (e.g., with a pair of VH and VL regions that bind to NKG2A) and one PD-L1 binding domain (e.g., with a pair of VH and VL regions that bind to PD-L1). In some embodiments, the multispecific binding agent provided herein comprises two NKG2A binding domains (e.g., with two identical pairs of VH and VL regions that bind to NKG2A) and one PD-L1 binding domain (e.g., with a pair of VH and VL regions that bind to PD-L1). In some embodiments, the multispecific binding agent provided herein comprises one NKG2A binding domain (e.g., with a pair of VH and VL regions that bind to NKG2A) and two PD-L1 binding domains (e.g., with two identical pairs of VH and VL regions that bind to PD-L1).

In some specific embodiments, provided herein is a multispecific binding agent comprising one or more NKG2A binding domains, each comprising a VH CDR1, a VH CDR2, and a VH CDR3 as set forth in a VH comprising the amino acid sequence of SEQ ID NO: 64 and a VL CDR1, a VL CDR2, and a VL CDR3 as set forth in a VL comprising the amino acid sequence of SEQ ID NO:73; and one or more PD-L1 binding domains, each comprising a VH CDR1, a VH CDR2, and a VH CDR3 as set forth in a VH comprising the amino acid sequence of SEQ ID NO:98 and a VL CDR1, a VL CDR2, and a VL CDR3 as set forth in a VL comprising the amino acid sequence of SEQ ID NO:99. In some embodiments, the multispecific binding agent provided herein comprises one NKG2A binding domain (e.g., with a pair of VH and VL regions that bind to NKG2A) and one PD-L1 binding domain (e.g., with a pair of VH and VL regions that bind to PD-L1). In some embodiments, the multispecific binding agent provided herein comprises two NKG2A binding domains (e.g., with two identical pairs of VH and VL regions that bind to NKG2A) and one PD-L1 binding domain (e.g., with a pair of VH and VL regions that bind to PD-L1). In some embodiments, the multispecific binding agent provided herein comprises one NKG2A binding domain (e.g., with a pair of VH and VL regions that bind to NKG2A) and two PD-L1 binding domains (e.g., with two identical pairs of VH and VL regions that bind to PD-L1).

In some embodiments, the multispecific binding agent provided herein comprises two NKG2A binding domains (e.g., with two identical or different pairs of VH and VL regions that bind to NKG2A) and one PD-L1 binding domain (e.g., with a pair of VH and VL regions that bind to PD-L1). In some embodiments, the multispecific binding agent provided herein comprises one NKG2A binding domain (e.g., with a pair of VH and VL regions that bind to NKG2A) and two PD-L1 binding domains (e.g., with two identical or different pairs of VH and VL regions that bind to PD-L1). In some embodiments, the multispecific binding agent provided herein comprises two NKG2A binding domains (e.g., with two identical or different pair of VH and VL regions that bind to NKG2A) and two PD-L1 binding domains (e.g., with two identical or different pairs of VH and VL regions that bind to PD-L1). In some embodiments, the multispecific binding agent further comprises one or more heavy chain constant regions (such as CH1, CH2, and/or CH3) and/or one or more light chain constant regions (such as CL).

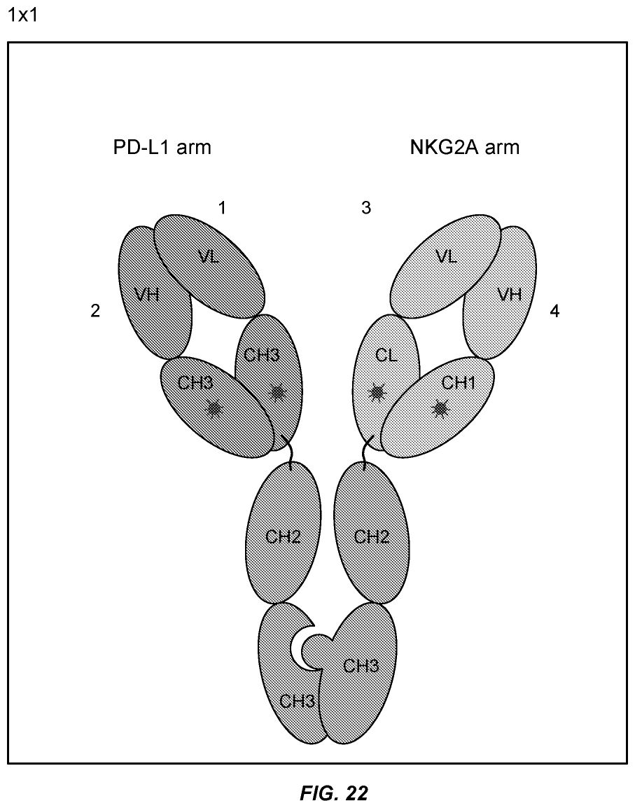

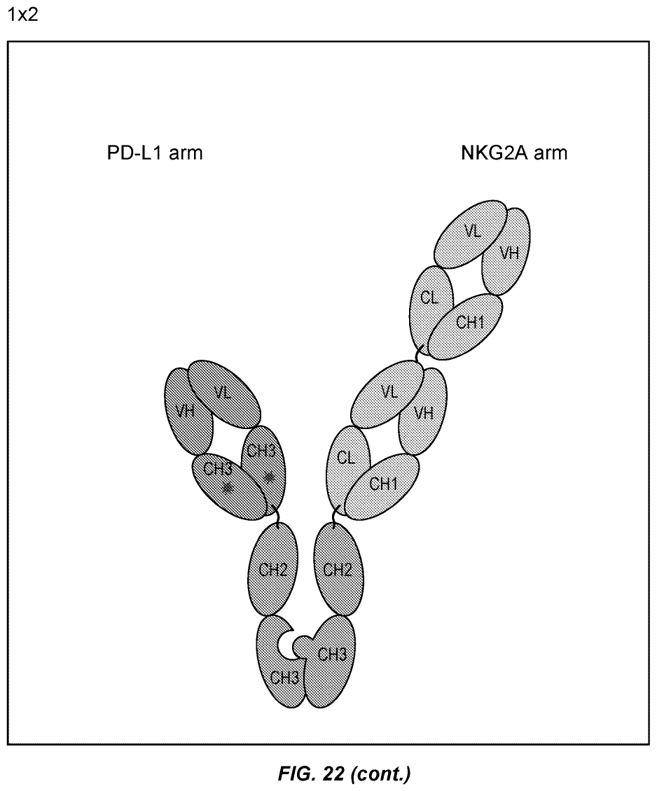

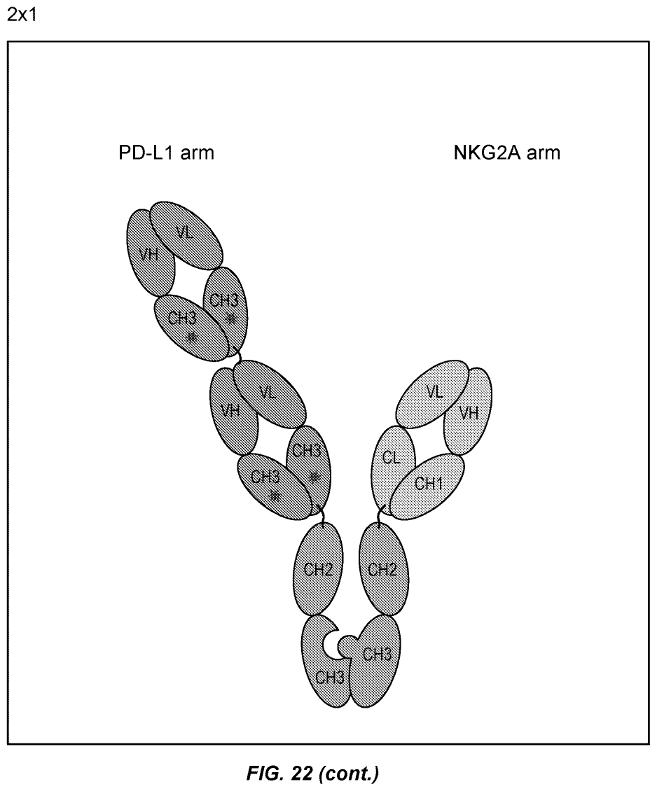

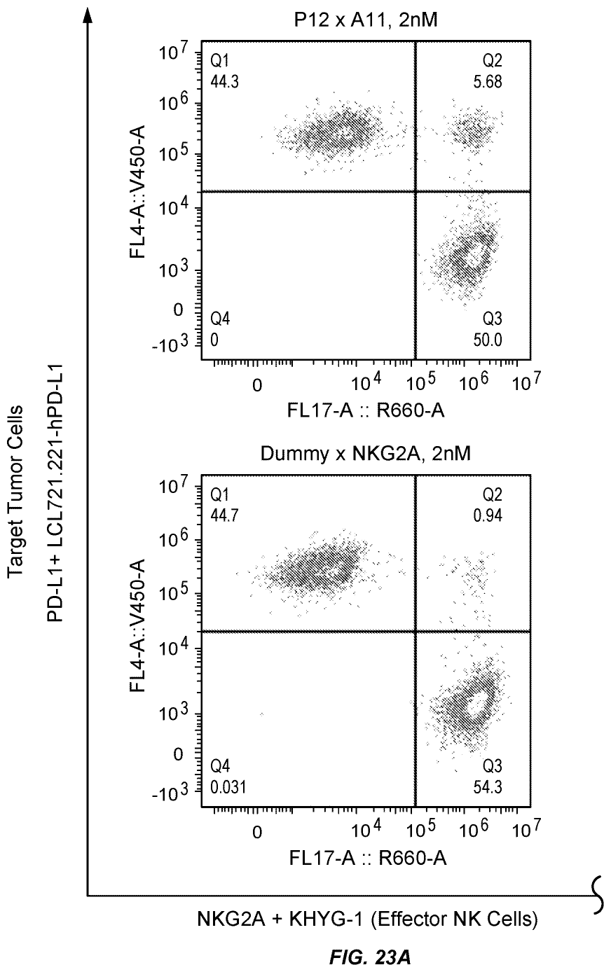

In some embodiments, the multispecific binding agent provided herein has an antibody format shown in any of the three panels in FIG. 22. In some embodiments, the multispecific binding agent provided herein has an antibody format shown in any of the three panels in FIG. 22, but its PD-L1 arm is switched with its NKG2A arm. For example, in the 1×1 format, Chain 1 and Chain 2 as labeled form a binding site for NKG2A (accordingly referred to as an NKG2A arm, instead of a PD-L1 arm as marked in the “1×1” panel of FIG. 22), while Chain 3 and Chain 4 form a binding site for PD-L1 (accordingly referred to as a PD-L1 arm, instead of an NKG2A arm as marked in the “1×1” panel of FIG. 22). Additionally or alternatively, in some embodiments, the multispecific binding agent provided herein has an antibody format shown in any of the three panels in FIG. 22, but its knob mutation(s) in one CH3 at the C terminus of CH2 is switched with its hole mutation(s) in the other CH3 at the C terminus of CH2. For example, in the 1×1 format, the knob mutation(s) can be in the CH3 which is at the C terminus of CH2 of Chain 1, while the hole mutation(s) can be in the CH3 which is at the C terminus of CH2 of Chain 3.

In some embodiments, mutations are introduced to one or more of the heavy chain constant regions (such as CH1, CH2, and/or CH3) and/or one or more light chain constant regions (such as CL), in order to achieve one or more of the following: (i) destabilizing a homodimer formed by a polypeptide of the multispecific antibody; (ii) stabilizing a multispecific antibody (also referred to herein as a heterodimer) as described herein; (iii) facilitating a proper assembly of a multispecific antibody as described herein; (iv) favoring heterodimerization over homodimerization of the constituent polypeptide chains; (v) improving the yield of a multispecific antibody as described herein; and (vi) improving the purity of a multispecific antibody as described herein. A variety of mutations have been developed that drive preferential heterodimerization, such as knob-in-hole (KIH or KiH) mutations (see, e.g., U.S. Pat. Nos. 5,731,168; 5,807,706; 5,821,333; and 8,216,805), disulfide-stabilized KIH mutations (see, e.g., U.S. Pat. Nos. 7,951,917; 8,642,745; and 9,409,989), and others (see, e.g., WO 2022/125986). Each of the patents and/or patent publications cited herein is incorporated herein by reference in its entirety.

Although specific exemplary multispecific antibody formats are described in the Example section below and in any of the three panels in FIG. 22, it is contemplated that any multispecific antibody formats known in the art can be used and are included in the present disclosure.

Multispecific binding agents (e.g., bispecific antibodies) described herein may be bispecific, trispecific or of greater multispecificity. Such agents may include multispecific antibodies. In some embodiments, multispecific antibodies, such as bispecific antibodies, are monoclonal antibodies that have binding specificities for at least two different targets (e.g., antigens) or two different epitopes on the same target (e.g., a bispecific antibody directed to NKG2A with a first binding domain for a first epitope of an NKG2A, and a second binding domain for a second epitope of NKG2A). In some embodiments, the multispecific (e.g., bispecific) antibodies can be constructed based on the sequences of the antibodies described herein, e.g., the CDR sequences listed in Tables 1-5. In some embodiments, the multispecific antibodies described herein are bispecific antibodies. In some embodiments, the multispecific antibody described herein (e.g., a bispecific antibody) has binding specificities for two targets, for example, NKG2A and PD-L1. In some embodiments, bispecific antibodies are mouse, chimeric, human or humanized antibodies.

In some embodiments, one of the binding specificities of the multispecific antibody provided herein is for NKG2A, a second binding specificity of the multispecific antibody provided herein is PD-L1, and yet one or more additional binding specificities are for any other target(s) (e.g., antigen). In some embodiments, a multispecific antibody can comprise more than one target (e.g., antigen) binding domain, in which different binding domains are specific for different targets. In some embodiments, the additional target is an immune checkpoint regulator (e.g., a negative checkpoint regulator). In some embodiments, the additional target is expressed on an immune cell. In some embodiments, the additional target is expressed on a tumor or cancer cell.

In some embodiments, multispecific (e.g., bispecific) antibody molecules can bind more than one (e.g., two or more) epitopes on the same target (e.g., antigen). In some embodiments, a multispecific (e.g., bispecific) binder as disclosed herein can bind to one or more epitopes on a first target (such as NKG2A) and one or more epitopes on a second target (such as PD-L1).

Methods for making multispecific antibodies are known in the art, such as, by co-expression of two immunoglobulin heavy chain-light chain pairs, where the two heavy chains have different specificities (see, e.g., Milstein and Cuello, 1983, Nature 305:537-40). For further details of generating multispecific antibodies (e.g., bispecific antibodies), see, for example, Bispecific Antibodies (Kontermann ed., 2011).

Exemplary structures of multispecific antibodies are known in the art and are further described in Weidle et al., 2013, Cancer Genomics & Proteomics 10:1-18; Brinkman et al., 2017, MABS, 9:2, 182-212; Godar et al., 2018, Expert Opinion on Therapeutic Patents, 28:3, 251-276; and Spiess et al., 2015, Mol. Immunol. 67 95-106.

For example, bispecific antibody molecules can be classified into different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates. As a non-limiting example, BsIgG formats can include crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, KA-body, and/or orthogonal Fab.

In some embodiments, BsIgG comprises heavy chains that are engineered for heterodimerization. For example, heavy chains can be engineered for heterodimerization using a “knobs-into-holes” strategy, a SEED platform, a common heavy chain (e.g., in KA-bodies), and use of heterodimeric Fc regions. Strategies are known in the art to avoid heavy chain pairing of homodimers in BsIgG, including knobs-into-holes, duobody, azymetric, charge pair, HA-TF, SEEDbody, and differential protein A affinity.

Another bispecific antibody format is IgG appended with an additional antigen-binding moiety. For example, monospecific IgG can be engineered to have bispecificity by appending an additional antigen-binding unit onto the monospecific IgG, e.g., at the N- or C-terminus of either the heavy or light chain. Exemplary additional antigen-binding units include single domain antibodies (e.g., variable heavy chain or variable light chain), engineered protein scaffolds, and paired antibody variable domains (e.g., single chain variable fragments or variable fragments). Non-limiting examples of appended IgG formats include dual variable domain IgG(DVD-Ig), IgG(H)-scFv, scFv-(H)IgG, IgG(L)-scFv, scFv-(L) IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, zybody, and DVI-IgG(four-in-one). See Spiess et al. Mol. Immunol. 67(2015):95-106. In some embodiments, an exemplary antibody format is a B-Body format for monospecific or multispecific (e.g., bispecific antibodies) as described in e.g. WO 2018/075692, U.S. Pat. Publ. No. 2018/0118811, and U.S. Pat. Publ. No. 2021/0155692.

Bispecific (Bs) antibody (BsAb) fragments are a format of bispecific antibody molecules that lack some or all of the antibody constant domains. For example, some BsAb lack an Fc region. In embodiments, bispecific antibody fragments include heavy and light chain regions that are connected by a peptide linker that permits efficient expression of the BsAb in a single host cell. Non-limiting examples of bispecific antibody fragments include, but are not limited to, nanobody, nanobody-HAS, BiTE, Diabody, DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)₂, F(ab′)₂-scFv2, scFv-KIH, Fab-scFv-Fc, tetravalent HCAb, scDiabody-Fc, Diabody-Fc, tandem scFv-Fc, and intrabody.

Bispecific fusion proteins include antibody fragments linked to other proteins. For example, bispecific fusion proteins can be linked to other proteins to add additional specificity and/or functionality. In some embodiments, the dock-and-lock (DNL) method can be used to generate bispecific antibody molecules with higher valency. For example, bispecific antibody fusions to albumin binding proteins or human serum albumin can be constructed to extend the serum half-life of antibody fragments. In some embodiments, chemical conjugation, e.g., chemical conjugation of antibodies and/or antibody fragments, can be used to create BsAb molecules. An exemplary bispecific antibody conjugate includes the CovX-body format, in which a low molecular weight drug is conjugated site-specifically to a single reactive lysine in each Fab arm or an antibody or fragment thereof. In some embodiments, the conjugation improves the serum half-life.

Methods of production of multispecific antibodies, including bispecific antibodies, are known in the art. For example, multispecific antibodies, including bispecific antibodies, can be produced by separate expression of the component antibodies in different host cells and subsequent purification/assembly or by expression of the component antibodies in a single host cell. Purification of multispecific (e.g., bispecific) antibody molecules can be performed by various methods known in the art, including affinity chromatography.

In some embodiments, multispecific binding agents (e.g., bispecific antibodies) disclosed herein can be provided in any antibody format disclosed herein or known in the art. As a non-limiting example, in some embodiments, the multispecific binding agents (e.g., bispecific antibodies) can be selected from Fabs-in-tandem-lg (FIT-1g); DVD-1g; hybrid hybridoma (quadroma or tetradoma); anticalin platform (Pieris); diabodies; single chain diabodies; tandem single chain Fv fragments; TandAbs, Trispecific Abs (Affimed); Darts dual affinity retargeting (Macrogenics); Bispecific Xmabs (Xencor); Bispecific T cell engagers (Bites; Amgen; 55 kDa); Triplebodies; Tribody=Fab-scFv Fusion Protein multifunctional recombinant antibody derivates (CreativeBiolabs); Duobody platform (Genmab); dock and lock platform; knobs-into-holes (KIH) platform; Humanized bispecific IgG antibody (REGN1979) (Regeneron); Mab2 bispecific antibodies (F-Star); DVD-1g=dual variable domain immunoglobulin (Abbott); kappa-lambda bodies; TBTI=tetravalent bispecific tandem Ig; B-Body; and CrossMab (Roche).

In some specific embodiments, the bispecific antibody provided herein is in a format depicted in any of the three panels in FIG. 22. In some embodiments, the bispecific antibody provided herein is in the 1×1 format as depicted in the “1×1” panel of FIG. 22. In some embodiments, the bispecific antibody provided herein is in the 1×2 format as depicted in the “1×2” panel of FIG. 22. In some embodiments, the bispecific antibody provided herein is in the 2×1 format as depicted in the “2×1” panel of FIG. 22.

In some specific embodiments, the multispecific antibody provided herein comprises four polypeptides: a first polypeptide comprising from N-terminus to C-terminus: a first VL, a first CH3, a first CH2, and a second CH3; a second polypeptide comprising from N-terminus to C-terminus: a first VH and a third CH3; a third polypeptide comprising from N-terminus to C-terminus: a second VL, a CL, a second CH2, and fourth CH3; and a fourth polypeptide comprising from N-terminus to C-terminus: a second VH and a CH1. These four polypeptides form two binding domains. In some embodiments, the first polypeptide and the second polypeptide (such as, the first VL and the first VH thereof) form a binding domain that binds to PD-L1, and the third polypeptide and the fourth polypeptide (such as, the second VL and the second VH thereof) form a binding domain that binds to NKG2A. In other embodiments, the first polypeptide and the second polypeptide (such as, the first VL and the first VH thereof) form a binding domain that binds to NKG2A, and the third polypeptide and the fourth polypeptide (such as, the second VL and the second VH thereof) form a binding domain that binds to PD-L1. In some embodiments, amino acid sequences of the first CH3, the second CH3, the third CH3 and the fourth CH3, or any subgroup thereof, are different from each other. In some embodiments, the second CH3 and the fourth CH3 provide a knobs-in-holes assembly. Additionally or alternatively, amino acid sequences of the first CH2 and the second CH2 are identical to each other. In other embodiments, amino acid sequences of the first CH2 and the second CH2 are different from each other.

In some specific embodiments, the multispecific antibody provided herein comprises five polypeptides: a first polypeptide comprising from N-terminus to C-terminus: a first VL, a first CH3, a first CH2, and a second CH3; a second polypeptide comprising from N-terminus to C-terminus: a first VH and a third CH3; a third polypeptide comprising from N-terminus to C-terminus: a second VL, a first CL, a third VL, a second CL, a second CH2, and a fourth CH3; a fourth polypeptide comprising from N-terminus to C-terminus: a second VH and a first CH1; and a fifth polypeptide comprising from N-terminus to C-terminus: a third VH and a second CH1. In some embodiments, amino acid sequences of the second VL and the third VL are identical. Additionally or alternatively, amino acid sequences of the first CL and the second CL are identical. In some embodiments, amino acid sequences of the second VH and the third VH are identical. Additionally or alternatively, amino acid sequences of the first CH1 and the second CH1 are identical. In some embodiments, amino acid sequences of the first CH3, the second CH3, the third CH3 and the fourth CH3, or any subgroup thereof, are different from each other. In some embodiments, the second CH3 and the fourth CH3 provide a knobs-in-holes assembly. Additionally or alternatively, amino acid sequences of the first CH2 and the second CH2 are identical to each other. In other embodiments, amino acid sequences of the first CH2 and the second CH2 are different from each other. In some embodiments, the fourth polypeptide is the same as the fifth polypeptide. The five polypeptides form three binding domains. In some embodiments, the first polypeptide and the second polypeptide (such as, the first VL and the first VH thereof) form a binding domain that binds to PD-L1, the third polypeptide and the fourth polypeptide (such as, the second VL and the second VH thereof) form a first binding domain that binds to NKG2A, and the third polypeptide and the fifth polypeptide (such as, the third VL and the third VH thereof) form a second binding domain that binds to NKG2A. In some embodiments, the first and the second NKG2A-binding domains are the same. In other embodiments, the first and the second NKG2A-binding domains are different from each other. In some embodiments, the first polypeptide and the second polypeptide (such as, the first VL and the first VH thereof) form a binding domain that binds to NKG2A, the third polypeptide and the fourth polypeptide (such as, the second VL and the second VH thereof) form a first binding domain that binds to PD-L1, and the third polypeptide and the fifth polypeptide (such as, the third VL and the third VH thereof) form a second binding domain that binds to PD-L1. In some embodiments, the two PD-L1 binding domains are the same.

In other more specific embodiments, the multispecific antibody provided herein comprises five polypeptides: a first polypeptide comprising from N-terminus to C-terminus: a first VL, a first CH3, a second VL, a second CH3, a first CH2, and a third CH3; a second polypeptide comprising from N-terminus to C-terminus: a first VH and a fourth CH3; a third polypeptide comprising from N-terminus to C-terminus: a second VH and a fifth CH3; a fourth polypeptide comprising from N-terminus to C-terminus: a third VL, a first CL, a second CH2 and a sixth CH3; and a fifth polypeptide comprising from N-terminus to C-terminus: a third VH and a CH1. In some embodiments, amino acid sequences of the first VL and the second VL are identical. Additionally or alternatively, amino acid sequences of the first CH3 and the second CH3 are identical. In some embodiments, amino acid sequences of the first VH and the second VH are the same. Additionally or alternatively, amino acid sequences of the fourth CH3 and the fifth CH3 are the same. In some embodiments, the second polypeptide and the third polypeptide are the same. In some embodiments, amino acid sequences of the first CH3, the second CH3, the third CH3, the fourth CH3, the fifth CH3 and the sixth CH3, or any subgroup thereof, are different from each other. In some embodiments, the third CH3 and the sixth CH3 provide a knobs-in-holes assembly. Additionally or alternatively, amino acid sequences of the first CH2 and the second CH2 are identical to each other. In other embodiments, amino acid sequences of the first CH2 and the second CH2 are different from each other. The five polypeptides form three binding domains. In some embodiments, the first polypeptide and the second polypeptide (such as, the first VL and the first VH thereof) form a first binding domain that binds to PD-L1, the first polypeptide and the third polypeptide (such as, the second VL and the second VH thereof) form a second binding domain that binds to PD-L1, and the fourth polypeptide and the fifth polypeptide (such as, the third VL and the third VH thereof) form a binding domain that binds to NKG2A. In some embodiments, the two PD-L1 binding domains are the same. In other embodiments, the first polypeptide and the second polypeptide (such as, the first VL and the first VH thereof) form a first binding domain that binds to NKG2A, the first polypeptide and the third polypeptide (such as, the second VL and the second VH thereof) form a second binding domain that binds to NKG2A, and the fourth polypeptide and the fifth polypeptide (such as, the third VL and the third VH thereof) form a binding domain that binds to PD-L1. In some embodiments, the two NKG2A binding domains are the same. In some embodiments, the first and the second NKG2A binding domains are different from each other.

In some embodiments, any one or more of the CH3 sequences comprise

GQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKS
LSLSPGK
(SEQ ID NO: 102; aa 116 to aa 222
of SEQ ID NO: 83).

In some embodiments, isoallotype mutations D356E and L358M are made in a CH3 sequence as disclosed herein. In some embodiments, one or more isoallotype mutations (for example, either or both D356E and L358M) are in one or more of the CH3 sequences immediately adjacent to the C terminus of a CH2 sequence, such as either or both of the CH3 without * marks in each construct illustrated in any of the three panels of FIG. 22. Additionally or alternatively, any one or more of the CH3 sequences are engineered to reduce the risk of immunogenicity of the antibody by replacing specific amino acids of one allotype with those of another allotype and referred to herein as isoallotype mutations, as described in more detail in Stickler et al. (Genes Immun. 2011 April; 12(3): 213-221).

Additionally or alternatively, the CH3 sequences are engineered to comprise knobs-into-holes mutations. In some embodiments, in a CH3/CH3 pair (e.g., one CH3 in one polypeptide is dimerized to another CH3 in a different polypeptide when forming a binding agent comprising the polypeptides) immediately adjacent to the C terminus of a CH2 sequence (for example, the two CH3 without * marks in each construct illustrated in any of the three panels of FIG. 22), one CH3 comprises a knob mutation (for example, T366W), while the other CH3 comprises a hole mutation (for example, any one or any two or all three of T366S, L368A, and Y407V). In other embodiments, one CH3 immediately adjacent to the C terminus of a VH or a VL in a VH/VL pair (e.g., the VH and the VL form a binding domain in a binding agent comprising the VH and VL) comprises a knob mutation (for example, T366W), while the other CH3 (immediately adjacent to the C terminus of a VL or a VH of the same VH/VL pair) comprises hole mutations (for example, any one or any two or all three of T366S, L368A, and Y407V).

As it would be understood by one of skill in the art, a domain pair, such as a CH3/CH3 pair, a VH/VL pair, a VL/VH pair, a CH2/CH2 pair, a CH1/CL pair, or a CL/CH1 pair, refers to one antibody domain (such as VH, VL, CH1, CH2, CH3, or CL) in one polypeptide chain dimerized to another antibody domain (such as VL, VH, CL, CH2, CH3, or CH, respectively) in a different polypeptide chain when forming a binding agent comprising the polypeptides.

In some embodiments, in a CH3/CH3 pair (e.g., one CH3 in one polypeptide is dimerized to another CH3 in a different polypeptide when forming a binding agent comprising the polypeptides), one CH3 comprises a Y349C mutation, while the other CH3 comprises an S354C mutation. In some embodiments, one CH3 immediately adjacent to the C terminus of a VH or a VL in a VH/VL pair comprises an S354C mutation, while the other CH3 (immediately adjacent to the C terminus of a VL or a VH of the same VH/VL pair) comprises a Y349C mutation. Additionally or alternatively, the CH3 sequences are engineered so that they can form a disulfide bond in an antibody, for example in order to stabilize the knobs-into-holes mutations as described above.

Additionally or alternatively, any one or more of the CH3 sequences are engineered to comprise other mutation(s) with the proviso that the mutation(s) do not significantly reduce the affinity and/or stability of the antibody, nor significantly increase the risk of immunogenicity of the antibody. In some embodiments, any one or more of the CH3 sequences are engineered to comprise a mutation as described in WO 2022/125986.

In some embodiments, in a CH3/CH3 pair, one CH3 comprises a mutation of S354C and the other CH3 comprises a mutation of Y349C. Additionally or alternatively, in a CH3/CH3 pair, E357 of one CH3 is substituted with a hydrophobic or aromatic amino acid. In various embodiments, the hydrophobic amino acid residue is selected from the group consisting of: isoleucine (I), leucine (L), methionine (M), proline (P) and valine (V). In various embodiments, the aromatic amino acid is selected from the group consisting of: histidine (H), tryptophan (W), phenylalanine (F), and tyrosine (Y). In some embodiments, the E357 of the CH3 is substituted with W. Additionally or alternatively, in some embodiments, a CH (for example a CH3) comprises a K370R mutation dimerized to an E357-mutation containing CH3 in a binding agent. In some embodiments, one CH3 of a CH3/CH3 pair comprises a K370R mutation. Additionally or alternatively, the other CH3 of the same CH3/CH3 pair comprises an E357W mutation. In some embodiments, one CH3 of a CH3/CH3 pair comprises a K370R mutation, while the other CH3 of the same CH3/CH3 pair comprises an E357W mutation. In some embodiments, in a CH3/CH3 pair, one CH3 comprises S354C and E357W, while the other CH3 comprises Y349C and K370R. In some embodiments, each CH3 of the CH3/CH3 pair is immediately adjacent to the C terminus of a CH2 sequence. In other embodiments, one CH3 of the CH3/CH3 pair is immediately adjacent to the C terminus of a VH or a VL in a VH/VL pair, and the other CH3 of the CH3/CH3 pair is immediately adjacent to the C terminus of a VL or a VH of the same VH/VL pair. In some embodiments, one CH3 immediately adjacent to the C terminus of a VH or a VL in a VH/VL pair comprises an E357W mutation; while the other CH3 (immediately adjacent to the C terminus of a VL or a VH of the same VH/VL pair) comprises a K370R mutation. In other embodiments, in a CH3/CH3 pair immediately adjacent to the C terminus of a CH2 sequence, one CH3 comprises both S354C and E357W, while the other CH3 comprises both Y349C and K370R.

Additionally or alternatively, one or more amino acid residues in a CH3 are swapped with the corresponding one or more amino acid residues in a CH1. In some embodiments, as used herein, a first amino acid residue in a first peptide corresponding to a second amino acid residue in a second peptide refers to the first amino acid residue aligned to the second amino acid residue in a sequence alignment between the first peptide and the second peptide. Alignment methods are available to one of skilled in the art, such as BLAST as disclosed herein and/or Clustal Omega. In some embodiments, the CH3 is immediately adjacent to C terminus of a VH or VL. Additionally or alternatively, the one or more amino acid residues are in an N-terminus fragment of a CH1, for example selected from the 1^{st} to the 10^{th} (including each range or integer therebetween, such as the 1^{st} to the 5^{th}, the 1^{st} to the 3^{rd}, the 1^{st} or the 3^{rd}) amino acids of a CH1. In some embodiments, a CH3 comprises the first amino acid residue swapped with the first amino acid residue of a CH1, which is also referred to herein as an N-terminal amino acid residue swapped with CH1. In some embodiments, the first amino acid residue of a CH3, which is a G, is substituted with the first amino acid residue of a CH1, which is an A. Such substitution is also designated as G341A herein for the ease of reference. In some embodiments, a CH3 immediately adjacent to C terminus of a VH or VL comprises G341A. Without wishing to be bound by the theory, a CH3 immediately adjacent to C terminus of a VH or VL and engineered to comprise a CH1 N-terminal fragment can improve the assembly and/or the purity of the binding agent as disclosed herein. In some embodiments, in a CH3/CH3 pair, each CH3 of which is immediately adjacent to the C terminus of a VH or a VL, one CH3 comprises the mutations of S354C and E357W, and the other CH3 comprises the mutations of Y349C and K370R. In some embodiments, in a CH3/CH3 pair, each CH3 of which is immediately adjacent to the C terminus of a VH or a VL, one CH3 comprises the mutations of G341A, S354C and E357W, and the other CH3 comprises the mutations of Y349C and K370R. In some embodiments, in a CH3/CH3 pair, each CH3 of which is immediately adjacent to the C terminus of a VH or a VL, one CH3 comprises the mutations of G341A, S354C and E357W, and the other CH3 comprises the mutations of G341A, Y349C and K370R. In some embodiments, in a CH3/CH3 pair, each CH3 of which is immediately adjacent to the C terminus of a VH or a VL, one CH3 comprises the mutations of S354C and E357W, and the other CH3 comprises the mutations of G341A, Y349C and K370R.

In some embodiments, the multispecific antibody as described herein comprises one or more CH3 mutations as disclosed in WO 2022/125986, which is incorporated herein by reference in its entirety. In some embodiments, the multispecific antibody as described herein comprises one or more CH3 domains as disclosed in WO 2022/125986.

Accordingly, any one or more of the CH3 sequences comprise any one of the following:

GQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKS
LSLSPGK
(SEQ ID NO: 102; aa 116 to aa 222 of
SEQ ID NO: 83);
GQPREPQVCTLPPSRDELTKNQVSLTCLVRGFYPSDIAVEWESNGQPENN
YKTTPPVLDSDGSFFLYSKLTVDKSRWQQGNVFSCSVMHEALHNHYTQKS
LSLSPGK
(SEQ ID NO: 103; Y349C and K370R);
AQPREPQVCTLPPSRDELTKNQVSLTCLVRGFYPSDIAVEWESNGQPENN
YKTTPPVLDSDGSFFLYSKLTVDKSRWQQGNVFSCSVMHEALHNHYTQKS
LSLSPGK
(SEQ ID NO: 104; N-terminal amino acid residue
swapped with CH1, Y349C and K370R);
GQPREPQVYTLPPCRDWLTKNQVSLTCLVKGFYPSDIAVEWESNGQPENN
YKTTPPVLDSDGSFFLYSKLTVDKSRWQQGNVFSCSVMHEALHNHYTQKS
LSLSPGK
(SEQ ID NO: 105; S354C and E357W);
AQPREPQVYTLPPCRDWLTKNQVSLTCLVKGFYPSDIAVEWESNGQPENN
YKTTPPVLDSDGSFFLYSKLTVDKSRWQQGNVFSCSVMHEALHNHYTQKS
LSLSPGK
(SEQ ID NO: 106; N-terminal amino acid residue
swapped with CH1, S354C and E357W);
GQPREPQVYTLPPSREEMTKNQVSLSCAVKGFYPSDIAVEWESNGQPENN
YKTTPPVLDSDGSFFLVSKLTVDKSRWQQGNVFSCSVMHEALHNHYTQKS
LSLSPGK
(SEQ ID NO: 107; D356E, L358M, T366S, L368A
and Y407V);
or
GQPREPQVYTLPPSRDELTKNQVSLWCLVKGFYPSDIAVEWESNGQPENN
YKTTPPVLDSDGSFFLYSKLTVDKSRWQQGNVFSCSVMHEALHNHYTQKS
LSLSPGK
(SEQ ID NO: 108; T366W).

In some embodiments, a binding agent as disclosed herein comprises a CH3 sequence as disclosed herein (such as any one of SEQ ID Nos: 70-74) but the CH3 lacks the C-terminal amino acid residue of lysine (K). In further embodiments, the CH3 is at the C terminus of any one or more polypeptides of the binding agent. In some embodiments, a C-terminus lysine was cleaved off in one or more polypeptides of the binding agent, such as any one or any two or any three of the following: the first polypeptide of a binding agent as disclosed herein, the second polypeptide of a binding agent as disclosed herein, and the third polypeptide of a binding agent as disclosed herein.

In some embodiments, one CH3 immediately adjacent to the C terminus of a VH or a VL in a VH/VL pair comprises GQPREPQVCTLPPSRDELTKNQVSLTCLVRGFYPSDIAVEWESNGQPENNYKTTPPV LDSDGSFFLYSKLTVDKSRWQQGNVFSCSVMHEALHNHYTQKSLSLSPGK (SEQ ID NO: 103; Y349C and K370R), while the other CH3 (immediately adjacent to the C terminus of a VL or a VH of the same VH/VL pair) comprises AQPREPQVYTLPPCRDWLTKNQVSLTCLVKGFYPSDIAVEWESNGQPENNYKTTPPV LDSDGSFFLYSKLTVDKSRWQQGNVFSCSVMHEALHNHYTQKSLSLSPGK (SEQ ID NO: 106; N-terminal amino acid residue swapped with CH1, S354C and E357W).

In some embodiments, in a CH3/CH3 pair immediately adjacent to the C terminus of a CH2 sequence (for example, the two CH3 without * marks in each construct illustrated in any of the three panels of FIG. 22), one CH3 comprises GQPREPQVYTLPPSREEMTKNQVSLSCAVKGFYPSDIAVEWESNGQPENNYKTTPPV LDSDGSFFLVSKLTVDKSRWQQGNVFSCSVMHEALHNHYTQKSLSLSPGK (SEQ ID NO: 107; D356E, L358M, T366S, L368A and Y407V), while the other CH3 comprises

(SEQ ID NO: 108; T366W)
GQPREPQVYTLPPSRDELTKNQVSLWCLVKGFYPSDIAVEWESNGQPENN
YKTTPPVLDSDGSFFLYSKLTVDKSRWQQGNVFSCSVMHEALHNHYTQKS
LSLSPGK.

As it will be understood by one of skill in the art and if not specified otherwise, EU numbering is referred to herein when describing a mutation in an antibody or a fragment thereof, such as an Fc or a CH3. See, more details at www.imgt.org/IMGTScientificChart/Numbering/Hu_IGHGnber.html #refs, which is hereby incorporated by reference in its entirety and identifies the residue according to its location in an endogenous constant region sequence regardless of the residue's physical location within a chain of the antibody constructs described herein. For example, in a CH3 consisting of aa 116 to aa 222 of SEQ ID NO:83, the 1^{st} aa of the CH3 (i.e., the 116^{th} aa of SEQ ID NO:83) is numbered as 341 and is referred to herein as G341; the 9^{th} aa of the CH3 is referred to herein as Y349; the 14^{th} aa of the CH3 is referred to herein as S354; the 16^{th} aa of the CH3 is referred to herein as D356; the 17^{th} aa of the CH3 is referred to herein as E357; the 18^{th} aa of the CH3 is referred to herein as L358; the 26^{th} aa of the CH3 is referred to herein as T366; the 28^{th} aa of the CH3 is referred to herein as L368; the 30^{th} aa of the CH3 is referred to herein as K370; and the 67 of the CH3 is referred to herein as Y407. Accordingly, the mutated aa residue can be added after the EU numbering in order to specify a mutation, such as S354C, E357W, Y349C, K370R, D356E, L358M, T366W, T366S, L368A, and Y407V.

In some embodiments, the binding agent as disclosed herein comprises a variant Fc region, such as a silent Fc as disclosed herein. In some embodiments, the binding agent lacks one or more effector functions, such as ADCC, ADCP, or CDC. In some embodiments, a variant Fc region comprises a CH2 as disclosed herein, such as a CH2 comprising a mutation reducing an effector function as disclosed herein.

In some embodiments, any one or more of the CH2 sequences comprise APELLGGPSVFLFPPKPKDTLMISRTPEVTCVVVDVSHEDPEVKFNWYVDGVEVHNA KTKPREEQYNSTYRVVSVLTVLHQDWLNGKEYKCKVSNKALPAPIEKTISKAK (SEQ ID NO: 109; aa 6 to aa 115 of SEQ ID NO:83, L234, L235, and P329). Additionally or alternatively, any one or more of the CH2 sequences lacks a mutation reducing an effector function of the binding agent. In other embodiments, any one or more of the CH2 sequences comprise a mutation reducing (including significantly reducing and abolishing) an effector function (such as ADCC, ADCP, or CDC) of the multispecific binding agent. In further embodiments, any one or more of the CH2 sequences comprise any one or any two or all three of the following mutations: L234A, L235A, and P329K (accordingly to the EU numbering). In some embodiments, any one or more of the CH2 sequences comprise a mutation to any one, or any two, or all three of the following amino acid residue: L234, L235, and P329 (accordingly to the EU numbering). In some embodiments, any one or more of the CH2 sequences comprise APEAAGGPSVFLFPPKPKDTLMISRTPEVTCVVVDVSHEDPEVKFNWYVDGVEVHN AKTKPREEQYNSTYRVVSVLTVLHQDWLNGKEYKCKVSNKALKAPIEKTISKAK (SEQ ID NO:110; aa 6 to aa 115 of SEQ ID NO:84, L234A, L235A, and P329K). In some embodiments, the multispecific antibody as described herein comprises one or more CH2 domains as disclosed in WO 2022/125986.

In some embodiments, one or more light chain constant domain (CL) comprises RTVAAPSVFIFPPSDEQLKSGTASVVCLLNNFYPREAKVQWKVDNALQSGNSQESVT EQDSKDSTYSLSSTLTLSKADYEKHKVYACEVTHQGLSSPVTKSFNRGEC (SEQ ID NO: 111) or RTVAAPSVFIFPPSDSQLKSGTASVVCLLNNFYPREAKVQWKVDNALQSGNSQESVT EQDSKDSTYSLSSTLTLSKADYEKHKVYACEVTHQGLSSPVTKSFNRGEC (SEQ ID NO: 82). In some embodiments, the multispecific antibody as described herein comprises one or more CL mutations as disclosed in WO 2022/125986. In some embodiments, the multispecific antibody as described herein comprises one or more CL domains as disclosed in WO 2022/125986.

In some embodiments, one or more CH1 sequences comprises

(SEQ ID NO: 112; aa 1 to aa 103 of SEQ ID NO: 85)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSC,
(SEQ ID NO: 113)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKRVEPKSC,
or
(SEQ ID NO: 114)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDRKVEPKSC.

In some embodiments, the multispecific antibody as described herein comprises one or more CH1 mutations as disclosed in WO 2022/125986. In some embodiments, the multispecific antibody as described herein comprises one or more CH1 domains as disclosed in WO 2022/125986.

In some embodiments, a binding agent as disclosed herein further comprises a hinge domain. For example, a hinge domain is immediately adjacent to the N terminus of a CH2 domain, for example, between a CH3 and a CH2, or between a light chain consistent domain (CL) and a CH2. Additionally or alternatively, a hinge domain is immediately adjacent to the C terminus of a CL and the N terminus of a light chain variable domain (VL). In further embodiments, the hinge domain comprises DKTHTCPPCP (SEQ ID NO:115). In some embodiments, the multispecific antibody as described herein comprises one or more domain junctions as disclosed in WO 2022/125986.

In some embodiments, a binding agent as disclosed herein further comprises a linker. For example, a linker can be presented between two variable regions in a single polypeptide (e.g., in a binding agent having a 2×1 format or a 1×2 format). In one example, a binding agent as disclosed herein comprises a polypeptide comprising the following, optionally from its N-terminus to its—C terminus: VL-CL-linker-VL-CL—an optional hinge-CH2-CH3. In another example, a binding agent as disclosed herein comprises a polypeptide comprising the following, optionally from its N-terminus to its—C terminus: VL-CH3-linker-VL-CH3—an optional hinge-CH2-CH3. In some embodiments, the linker comprises (SSSG)ₙ (SEQ ID NO:116) or (SSG)ₙ, wherein n is any positive integer, such as 1, 2, 3, 4, 5, 6, 7, 8, 9, 10 or more. Additionally or alternatively, the linker is about 1 amino acid long to about 30 amino acids long. In some embodiments, the linker comprises TASSGGSSSG (SEQ ID NO: 117).

In some embodiments, the multispecific binding agent as described herein has a construct as disclosed in WO 2022/125986.

In some embodiments, a multispecific binding agent as disclosed herein comprises a first polypeptide comprising SEQ ID NO:118. In some embodiments, a multispecific binding agent as disclosed herein comprises a first polypeptide comprising SEQ ID NO:119. In some embodiments, a multispecific binding agent as disclosed herein comprises a first polypeptide comprising SEQ ID NO:127. In some embodiments, a multispecific binding agent as disclosed herein comprises a first polypeptide comprising SEQ ID NO:128.

In some embodiments, a multispecific binding agent as disclosed herein comprises a third polypeptide comprising SEQ ID NO:121. In some embodiments, a multispecific binding agent as disclosed herein comprises a third polypeptide comprising SEQ ID NO:123. In some embodiments, a multispecific binding agent as disclosed herein comprises a third polypeptide comprising SEQ ID NO:124. In some embodiments, a multispecific binding agent as disclosed herein comprises a third polypeptide comprising SEQ ID NO:125. In some embodiments, a multispecific binding agent as disclosed herein comprises a third polypeptide comprising SEQ ID NO:129. In some embodiments, a multispecific binding agent as disclosed herein comprises a third polypeptide comprising SEQ ID NO: 130. In some embodiments, a multispecific binding agent as disclosed herein comprises a third polypeptide comprising SEQ ID NO:131. In some embodiments, a multispecific binding agent as disclosed herein comprises a third polypeptide comprising SEQ ID NO:132.

In some embodiments, a multispecific binding agent as disclosed herein comprises a second polypeptide comprising SEQ ID NO:120. In further embodiments, a multispecific binding agent as disclosed herein comprises two of the second polypeptides, each comprising SEQ ID NO:120.

In some embodiments, a multispecific binding agent as disclosed herein comprises a fourth polypeptide comprising SEQ ID NO:122 or SEQ ID NO:126. In further embodiments, a multispecific binding agent as disclosed herein comprises two of the fourth polypeptides, each comprising SEQ ID NO:122 or SEQ ID NO:126.

In some embodiments, a multispecific binding agent as disclosed herein comprises: a first polypeptide comprising SEQ ID NO:118, a second polypeptide comprising SEQ ID NO: 120, a third polypeptide comprising SEQ ID NO:121, and a fourth polypeptide comprising SEQ ID NO: 122.

In some embodiments, a multispecific binding agent as disclosed herein comprises: a first polypeptide comprising SEQ ID NO:118, a second polypeptide comprising SEQ ID NO: 120, a third polypeptide comprising SEQ ID NO:123, and one or more (such as two) fourth polypeptides, each of the fourth polypeptide comprises SEQ ID NO: 122.

In some embodiments, a multispecific binding agent as disclosed herein comprises: a first polypeptide comprising SEQ ID NO:119, one or more (such as two) second polypeptides, each of the second polypeptide comprises SEQ ID NO: 120, a third polypeptide comprising SEQ ID NO:121, and a fourth polypeptide comprising SEQ ID NO:122.

In some embodiments, a multispecific binding agent as disclosed herein comprises: a first polypeptide comprising SEQ ID NO: 118, a second polypeptide comprising SEQ ID NO: 120, a third polypeptide comprising SEQ ID NO:125, and a fourth polypeptide comprising SEQ ID NO:126.

In some embodiments, a multispecific binding agent as disclosed herein comprises: a first polypeptide comprising SEQ ID NO:118, a second polypeptide comprising SEQ ID NO: 120, a third polypeptide comprising SEQ ID NO:124, and one or more (such as two) fourth polypeptides, each of the fourth polypeptide comprises SEQ ID NO:126.

In some embodiments, a multispecific binding agent as disclosed herein comprises: a first polypeptide comprising SEQ ID NO:119, one or more (such as two) second polypeptides, each of the second polypeptide comprises SEQ ID NO:120, a third polypeptide comprising SEQ ID NO: 125, and a fourth polypeptide comprising SEQ ID NO: 126.

In some embodiments, a multispecific binding agent as disclosed herein comprises: a first polypeptide comprising SEQ ID NO: 127, a second polypeptide comprising SEQ ID NO: 120, a third polypeptide comprising SEQ ID NO: 129, and a fourth polypeptide comprising SEQ ID NO:122.

In some embodiments, a multispecific binding agent as disclosed herein comprises: a first polypeptide comprising SEQ ID NO: 127, a second polypeptide comprising SEQ ID NO: 120, a third polypeptide comprising SEQ ID NO:130, and one or more (such as two) fourth polypeptides, each of the fourth polypeptide comprises SEQ ID NO: 122.

In some embodiments, a multispecific binding agent as disclosed herein comprises: a first polypeptide comprising SEQ ID NO: 128, one or more (such as two) second polypeptides, each of the second polypeptide comprises SEQ ID NO: 120, a third polypeptide comprising SEQ ID NO:129, and a fourth polypeptide comprising SEQ ID NO: 122.

In some embodiments, a multispecific binding agent as disclosed herein comprises: a first polypeptide comprising SEQ ID NO: 127, a second polypeptide comprising SEQ ID NO: 120, a third polypeptide comprising SEQ ID NO:132, and a fourth polypeptide comprising SEQ ID NO:126.

In some embodiments, a multispecific binding agent as disclosed herein comprises: a first polypeptide comprising SEQ ID NO:127, a second polypeptide comprising SEQ ID NO: 120, a third polypeptide comprising SEQ ID NO:131, and one or more (such as two) fourth polypeptides, each of the fourth polypeptide comprises SEQ ID NO:126.

In some embodiments, a multispecific binding agent as disclosed herein comprises: a first polypeptide comprising SEQ ID NO:128, one or more (such as two) second polypeptides, each of the second polypeptide comprises SEQ ID NO: 120, a third polypeptide comprising SEQ ID NO:132, and a fourth polypeptide comprising SEQ ID NO:126.

In some embodiments, any one, any two, any three, or all four polypeptides of a multispecific binding agent as disclosed herein (such as any polypeptides comprising any of SEQ ID NOs: 118-132) are modified by one or more insertions, one or more deletions, or one or more substitutions in the amino acid sequence. In further embodiments, these insertions, deletions, or substitutions are not within a CDR. In some embodiments, the modified polypeptide is at least 80%, or at least 85%, or at least 90%, or at least 91%, or at least 92%, or at least 93%, or at least 94%, or at least 95%, or at least 96%, or at least 97%, or at least 98%, or at least 99% identical to the polypeptide from which it is modified.

In some aspect, provided herein is a multispecific binding agent comprising four polypeptide chains as disclosed herein: a first polypeptide, a second polypeptide, a third polypeptide and a fourth polypeptide. In some embodiments, the first polypeptide comprises (i) an amino acid sequence as disclosed herein or (ii) the amino acid sequence as disclosed herein but lacking the C-terminal lysine (K). Additionally or alternatively, the second polypeptide comprises (i) an amino acid sequence as disclosed herein, or (ii) the amino acid sequence as disclosed herein but lacking the C-terminal lysine (K). Additionally or alternatively, the third polypeptide chain comprises (i) an amino acid sequence as disclosed herein, or (ii) the amino acid sequence as disclosed herein but lacking the C-terminal lysine (K). Additionally or alternatively, the fourth polypeptide chain comprises an amino acid sequence as disclosed herein.

In some embodiments, the multispecific binding agent as disclosed herein comprises a first polypeptide chain as disclosed herein, a second polypeptide chain as disclosed herein, a third polypeptide chain as disclosed herein and a fourth polypeptide chain as disclosed herein. In further embodiments, the C-terminal lysine of the first polypeptide chain of the multispecific binding agent is removed (such as, cleaved off). Additionally or alternatively, the C-terminal lysine of the second polypeptide chain of the multispecific binding agent is removed (such as, cleaved off). Additionally or alternatively, the C-terminal lysine of the third polypeptide chain of the multispecific binding agent is removed (such as, cleaved off). Additionally or alternatively, the C-terminal lysine of the first polypeptide chain and C-terminal lysine of the second polypeptide chain of the multispecific binding agent are removed (such as, cleaved off). Additionally or alternatively, the C-terminal lysine of the first polypeptide chain and C-terminal lysine of the third polypeptide chain of the multispecific binding agent are removed (such as, cleaved off). Additionally or alternatively, the C-terminal lysine of the second polypeptide chain and C-terminal lysine of the third polypeptide chain of the multispecific binding agent are removed (such as, cleaved off). Additionally or alternatively, each C-terminal lysine of the first, second and third polypeptide chain is removed (such as, cleaved off) from the multispecific binding agent. As used herein, a multispecific binding agent having any one or any two or all three of its C-terminal lysine amino acid residues removed is referred to as a C-terminal variant of the multispecific binding agent. Accordingly, also provided herein is a composition comprising the multispecific binding agent and one or more C-terminal variants thereof, or a composition comprising one or more C-terminal variants of the multispecific binding agent. As it would be understood by one of skill in the art, a composition, a method, a use or any other embodiments relating to the multispecific binding agent as disclosed herein also extend to a composition, a method, a use or an embodiment of (i) a C-terminal variant of the multispecific binding agent or (ii) a composition comprising any one or more of: the the multispecific binding agent and/or a C-terminal variant thereof.

In some embodiments, the multispecific antibody provided herein further comprises one or more heavy chain constant domains (e.g., CH1, Hinge, CH2, and CH3). As described above, a typical heavy chain has at the N-terminus, a variable domain (VH) followed by three constant domains (CH)—CH1, CH2, and CH3. The hinge region is a stretch of heavy chains between the Fab and Fc portions. Each of CH1, Hinge, CH2, and CH3 can be derived from any species, naturally occurring or artificial, and may contain variations. An exemplary IgG heavy chain comprises the following CH1, Hinge, CH2, and CH3 amino acid sequence:

(SEQ ID NO: 81)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPGK.

Another exemplary IgG heavy chain comprises the following CH1, Hinge, CH2, and CH3 amino acid sequence:

(SEQ ID NO: 85)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPEAAGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALKAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPGK.

In some embodiments, the multispecific antibody provided herein further comprises one or more light chain constant domain (CL). An exemplary light chain comprises the following CL amino acid sequence:

(SEQ ID NO: 82)
RTVAAPSVFIFPPSDSQLKSGTASVVCLLNNFYPREAKVQWKVDNALQS
GNSQESVTEQDSKDSTYSLSSTLTLSKADYEKHKVYACEVTHQGLSSPV
TKSFNRGEC.

In some embodiments, the binding agent provided herein inhibits the HLA-E/NKG2A (such as HLA-E/NKG2A/CD94) signaling. Additionally or alternatively, the binding agent provided herein inhibits the PD-1/PD-L1 checkpoint signaling. In some embodiments, the binding agent provided herein promotes NK cell mediated cytotoxicity. In some embodiments, the binding agent provided herein promotes NK cell's degranulation. In some embodiments, the binding agent provided herein activates CD8⁺ T cells.

Other exemplary binding molecules are described in more detail in the following sections. In some embodiments, the multispecific binding agents according to any of the above embodiments may incorporate any of the features, singly or in combination, as described in Sections 5.2.4 to 5.2.7 below.

TABLE 1
Antibody Clone A3

| | | Exemplary | IMGT | Kabat | Chothia | Contact | AbM |
|---|---|---|---|---|---|---|---|
| VH | VH | GFTFSSYYIH | GFTFSSYY | SYYIH | GFTFSSY | SSYYIH | GFTFSSYYIH |
| CDR | CDR1 | (SEQ ID | (SEQ ID | (SEQ ID | (SEQ ID | (SEQ ID | (SEQ ID |
| Seq. | | NO: 1) | NO: 7) | NO: 12) | NO: 13) | NO: 18) | NO: 1) |
| | VH | SISPYDGY | ISPYDGYT | SISPYDGYT | PYDG | WVASISPYDGYT | SISPYDGYTD |
| | CDR2 | TDYADSVKG | (SEQ ID | DYADSVKG | (SEQ ID | D | (SEQ ID |
| | | (SEQ ID | NO: 8) | (SEQ ID | NO: 14) | (SEQ ID | NO: 24) |
| | | NO: 2) | | NO: 2) | | NO: 19) | |
| | VH | SSDFSPWTS | ARSSDFSPW | SSDFSPWTS | SDFSPWTSS | ARSSDFSPWT | SSDFSPWTSSGG |
| | CDR3 | SGGMDY | TSSGGMDY | SGGMDY | GGMD | SSGGMD | MDY |
| | | (SEQ ID | (SEQ ID | (SEQ ID | (SEQ ID | (SEQ ID | (SEQ ID |
| | | NO: 3) | NO: 9) | NO: 3) | NO: 15) | NO: 20) | NO: 3) |
| VL | VL | RASQSVSSAVA | QSVSSA | RASQSVSSAVA | SQSVSSA | SSAVAWY | RASQSVSSAVA |
| CDR | CDR1 | (SEQ ID | (SEQ ID | (SEQ ID | (SEQ ID | (SEQ ID | (SEQ ID |
| Seq. | | NO: 4) | NO: 10) | NO: 4) | NO: 16) | NO: 21) | NO: 4) |
| | VL | SASSLYS | SAS | SASSLYS | SAS | LLIYSASSLY | SASSLYS |
| | CDR2 | (SEQ ID | (SEQ ID | (SEQ ID | (SEQ ID | (SEQ ID | (SEQ ID |
| | | NO: 5) | NO: 11) | NO: 5) | NO: 11) | NO: 22) | NO: 5) |
| | VL | QQYGSSLRT | QQYGSSLRT | QQYGSSLRT | YGSSLR | QQYGSSLR | QQYGSSLRT |
| | CDR3 | (SEQ ID | (SEQ ID | (SEQ ID | (SEQ ID | (SEQ ID | (SEQ ID |
| | | NO: 6) | NO: 6) | NO: 6) | NO: 17) | NO: 23) | NO: 6) |

VH Sequence:
EVQLVESGGGLVQPGGSLRLSCAASGFTFSSYYIHWVRQAPGKGLEWVASISPYDGYTDYADSVKGRFTISADTSKNTAYLQMNSLRAED
TAVYYCARSSDFSPWTSSGGMDYWGQGTLVTVSS (SEQ ID NO: 25)
VL Sequence:
DIQMTQSPSSLSASVGDRVTITCRASQSVSSAVAWYQQKPGKAPKLLIYSASSLYSGVPSRFSGSRSGTDFTLTISSLQPEDFATYYCQQ
YGSSLRTFGQGTKVEIK (SEQ ID NO: 26)

TABLE 2
Antibody Clone A2

| | | Exemplary | IMGT | Kabat | Chothia | Contact | AbM |
|---|---|---|---|---|---|---|---|
| VH | VH | GFTFSSYYIH | GFTFSSYY | SYYIH | GFTFSSY | SSYYIH | GFTFSSYYIH |
| CDR | CDR1 | (SEQ ID | (SEQ ID | (SEQ ID | (SEQ ID | (SEQ ID | (SEQ ID |
| Seq. | | NO: 1) | NO: 7) | NO: 12) | NO: 13) | NO: 18) | NO: 1) |
| | VH | SYVPEDGY | YVPEDGYT | SYVPEDGY | PEDG | WVASYVPEDGY | SYVPEDGYTD |
| | CDR2 | TDYADSVKG | (SEQ ID | TDYADSVKG | | TD | NO: 44) |
| | | (SEQ ID | NO: 32) | (SEQ ID | (SEQ ID | (SEQ ID | (SEQ ID |
| | | NO: 27) | | NO: 27) | NO: 35) | NO: 39) | |
| | VH | SSDFIPW | ARSSDFIP | SSDFIPWT | SDFIPWT | ARSSDFIPWTSSG | SSDFIPWT |
| | CDR3 | TSSGGMDY | WTSSGGMDY | SSGGMDY | SSGGMD | GMD | SSGGMDY |
| | | (SEQ ID | (SEQ ID | (SEQ ID | (SEQ ID | (SEQ ID | (SEQ ID |
| | | NO: 28) | NO: 33) | NO: 28) | NO: 36) | NO: 40) | NO: 28) |
| VL | VL | RASQSVSRAVA | QSVSRA | RASQSVSRAVA | SQSVSRA | SRAVAWY | RASQSVSRAVA |
| CDR | CDR1 | (SEQ ID | (SEQ ID | (SEQ ID | (SEQ ID | (SEQ ID | (SEQ ID |
| Seq. | | NO: 29) | NO: 34) | NO: 29) | NO: 37) | NO: 41) | NO: 29) |
| | VL | SASSLHS | SAS | SASSLHS | SAS | LLIYSASSLH | SASSLHS |
| | CDR2 | NO: 30) | NO: 11) | NO: 30) | NO: 11) | NO: 42) | NO: 30) |
| | VL | QQYGSSART | QQYGSSART | QQYGSSART | YGSSAR | QQYGSSAR | QQYGSSART |
| | CDR3 | (SEQ ID | (SEQ ID | (SEQ ID | (SEQ ID | (SEQ ID | (SEQ ID |
| | | NO: 31) | NO: 31) | NO: 31) | NO: 38) | NO: 43) | NO: 31) |

VH Sequence:
EVQLVESGGGLVQPGGSLRLSCAASGFTFSSYYIHWVRQAPGKGLEWVASYVPEDGYTDYADSVKGRFTISADTSKNTAYLQMNSLRA
EDTAVYYCARSSDFIPWTSSGGMDYWGQGTLVTVSS (SEQ ID NO: 45)
VL Sequence:
DIQMTQSPSSLSASVGDRVTITCRASQSVSRAVAWYQQKPGKAPKLLIYSASSLHSGVPSRFSGSRSGTDFTLTISSLQPEDFATYYC
QQYGSSARTFGQGTKVEIK (SEQ ID NO: 46)

TABLE 3
Antibody Clone A42

| | | Exemplary | IMGT | Kabat | Chothia | Contact | AbM |
|---|---|---|---|---|---|---|---|
| VH | VH | GFTFSWYYIH | GFTFSWYY | WYYIH | GFTFSWY | SWYYIH | GFTFSWYYIH |
| CDR | CDR1 | (SEQ ID | (SEQ ID | (SEQ ID | (SEQ ID | (SEQ ID | (SEQ ID |
| Seq. | | NO: 47) | NO: 51) | NO: 54) | NO: 55) | NO: 59) | NO: 47) |
| | VH | VIDPISSYT | IDPISSYT | VIDPISSYTYYAD | PISS | WVAVIDPISSYTY | VIDPISSYTY |
| | CDR2 | YYADSVKG | (SEQ ID | SVKG | (SEQ ID | (SEQ ID | (SEQ ID |
| | | (SEQ ID | NO: 52) | (SEQ ID | NO: 56) | NO: 60) | NO: 63) |
| | | NO: 48) | | NO: 48) | | | |
| | VH | GFDRAMDY | ARGFDRAMDY | GFDRAMDY | FDRAMD | ARGFDRAMD | GFDRAMDY |
| | CDR3 | (SEQ ID | (SEQ ID | (SEQ ID | (SEQ ID | (SEQ ID | (SEQ ID |
| | | NO: 49) | NO: 53) | NO: 49) | NO: 57) | NO: 61) | NO: 49) |
| VL | VL | RASQSVSSA | QSVSSA | RASQSVSSAVA | SQSVSSA | SSAVAWY | RASQSVSSAVA |
| CDR | CDR1 | VA (SEQ ID | (SEQ ID | (SEQ ID | (SEQ ID | (SEQ ID | (SEQ ID |
| Seq. | | NO: 4) | NO: 10) | NO: 4) | NO: 16) | NO: 21) | NO: 4) |
| | VL | SASSLYS | SAS | SASSLYS | SAS | LLIYSASSLY | SASSLYS |
| | CDR2 | (SEQ ID | (SEQ ID | (SEQ ID | (SEQ ID | (SEQ ID | (SEQ ID |
| | | NO: 5) | NO: 11) | NO: 5) | NO: 11) | NO: 22) | NO: 5) |
| | VL | QQYYSSPYT | QQYYSSPYT | QQYYSSPYT | YYSSPY | QQYYSSPY | QQYYSSPYT |
| | CDR3 | (SEQ ID | (SEQ ID | (SEQ ID | (SEQ ID | (SEQ ID | (SEQ ID |
| | | NO: 50) | NO: 50) | NO: 50) | NO: 58) | NO: 62) | NO: 50) |

VH Sequence:
EVQLVESGGGLVQPGGSLRLSCAASGFTFSWYYIHWVRQAPGKGLEWVAVIDPISSYTYYADSVKGRFTISADTSKNTAYLQM
NSLRAEDTAVYYCARGFDRAMDYWGQGTLVTVSS (SEQ ID NO: 64)
VL Sequence:
DIQMTQSPSSLSASVGDRVTITCRASQSVSSAVAWYQQKPGKAPKLLIYSASSLYSGVPSRFSGSRSGTDFTLTISSLQPEDF
ATYYCQQYYSSPYTFGQGTKVEIK (SEQ ID NO: 65)

TABLE 4
Antibody Clone A11

| | | Exemplary | IMGT | Kabat | Chothia | Contact | AbM |
|---|---|---|---|---|---|---|---|
| VH | VH | GFTFSWYYIH | GFTFSWYY | WYYIH | GFTFSWY | SWYYIH | GFTFSWYYIH |
| CDR | CDR1 | (SEQ ID | (SEQ ID | (SEQ ID | (SEQ ID | (SEQ ID | (SEQ ID |
| Seq. | | NO: 47) | NO: 51) | NO: 54) | NO: 55) | NO: 59) | NO: 47) |
| | VH | VIDPISSYT | IDPISSYT | VIDPISSYT | PISS | WVAVIDPIS | VIDPISSYTY |
| | CDR2 | YYADSVKG | (SEQ ID | YYADSVKG | (SEQ ID | SYTY (SEQ ID | (SEQ ID |
| | | (SEQ ID | NO: 52) | (SEQ ID | NO: 56) | NO: 60) | NO: 63) |
| | | NO: 48) | | NO: 48) | | | |
| | VH | GFDRAMDY | ARGFDRAMDY | GFDRAMDY | FDRAMD | ARGFDRAMD | GFDRAMDY |
| | CDR3 | (SEQ ID | (SEQ ID | (SEQ ID | (SEQ ID | (SEQ ID | (SEQ ID |
| | | NO: 49) | NO: 53) | NO: 49) | NO: 57) | NO: 61) | NO: 49) |
| VL | VL | RASKSVSSALA | KSVSSA | RASKSVSSALA | SKSVSSA | SSALAWY | RASKSVSSALA |
| CDR | CDR1 | (SEQ ID | (SEQ ID | (SEQ ID | (SEQ ID | (SEQ ID | (SEQ ID |
| Seq. | | NO: 66) | NO: 68) | NO: 66) | NO: 70) | NO: 71) | NO: 66) |
| | VL | VGSSRYS | VGS | VGSSRYS | VGS | LLIYVGSSRY | VGSSRYS |
| | CDR2 | (SEQ ID | (SEQ ID | (SEQ ID | (SEQ ID | (SEQ ID | (SEQ ID |
| | | NO: 67) | NO: 69) | NO: 67) | NO: 69) | NO: 72) | NO: 67) |
| | VL | QQYYSSPYT | QQYYSSPYT | QQYYSSPYT | YYSSPY | QQYYSSPY | QQYYSSPYT |
| | CDR3 | (SEQ ID | (SEQ ID | (SEQ ID | (SEQ ID | (SEQ ID | (SEQ ID |
| | | NO: 50) | NO: 50) | NO: 50) | NO: 58) | NO: 62) | NO: 50) |

VH Sequence:
EVQLVESGGGLVQPGGSLRLSCAASGFTFSWYYIHWVRQAPGKGLEWVAVIDPISSYTYYADSVKGRFTISADTSKNTAYLQMNSLRAED
TAVYYCARGFDRAMDYWGQGTLVTVSS (SEQ ID NO: 64)
VL Sequence:
DIQMTQSPSSLSASVGDRVTITCRASKSVSSALAWYQQKPGKAPKLLIYVGSSRYSGVPSRFSGSRSGTDFTLTISSLQPEDFATYYCQQ
YYSSPYTFGQGTKVEIK (SEQ ID NO: 73)

TABLE 5
Antibody Clone P12

| | | Exemplary | IMGT | Kabat | Chothia | Contact | AbM |
|---|---|---|---|---|---|---|---|
| VH | VH | GFTFSSYYIH | GFTFSSYY | SYYIH | GFTFSSY | SSYYIH | GFTFSSYYIH |
| CDR | CDR1 | (SEQ ID | (SEQ ID | (SEQ ID | (SEQ ID | (SEQ ID | (SEQ ID |
| Seq. | | NO: 1) | NO: 7) | NO: 12) | NO: 13) | NO: 18) | NO: 1) |
| | VH | TINSRGGF | INSRGGFT | TINSRGGF | SRGG | WVATINSR | TINSRGGFTL |
| | CDR2 | TLYADSVKG | (SEQ ID | TLYADSVKG | (SEQ ID | GGFTL | (SEQ ID |
| | | NO: 86) | NO: 89) | NO: 86) | NO: 91) | NO: 94) | NO: 97) |
| | | (SEQ ID | | (SEQ ID | | (SEQ ID | |
| | VH | GYTLTPVLDY | ARGYTLTPVL | GYTLTPVLDY | YTLTPVLD | ARGYTLTPVLD | GYTLTPVLDY |
| | CDR3 | (SEQ ID | DY (SEQ ID | (SEQ ID | (SEQ ID | (SEQ ID | (SEQ ID |
| | | NO: 87) | NO: 90) | NO: 87) | NO: 92) | NO: 95) | NO: 87) |
| VL | VL | RASQSVSSAVA | QSVSSA | RASQSVSSAVA | SQSVSSA | SSAVAWY | RASQSVSSAVA |
| CDR | CDR1 | (SEQ ID | (SEQ ID | (SEQ ID | (SEQ ID | (SEQ ID | (SEQ ID |
| Seq. | | NO: 4) | NO: 10) | NO: 4) | NO: 16) | NO: 21) | NO: 4) |
| | VL | SASSLYS | SAS | SASSLYS | SAS | LLIYSASSLY | SASSLYS |
| | CDR2 | (SEQ ID | (SEQ ID | (SEQ ID | (SEQ ID | (SEQ ID | (SEQ ID |
| | | NO: 5) | NO: 11) | NO: 5) | NO: 11) | NO: 22) | NO: 5) |
| | VL | QQFMLEPIT | QQFMLEPIT | QQFMLEPIT | FMLEPI | QQFMLEPI | QQFMLEPIT |
| | CDR3 | (SEQ ID | (SEQ ID | (SEQ ID | (SEQ ID | (SEQ ID | (SEQ ID |
| | | NO: 88) | NO: 88) | NO: 88) | NO: 93) | NO: 96) | NO: 88) |

VH Sequence:
EVQLVESGGGLVQPGGSLRLSCAASGFTFSSYYIHWVRQAPGKGLEWVATINSRGGFTLYADSVKGR
FTISADTSKNTAYLQMNSLRAEDTAVYYCARGYTLTPVLDYWGQGTLVTVSS (SEQ ID NO: 98)
VL Sequence:
DIQMTQSPSSLSASVGDRVTITCRASQSVSSAVAWYQQKPGKAPKLLIYSASSLYSGVPSRFSGSRS
GTDFTLTISSLQPEDFATYYCQQFMLEPITFGQGTKVEIK (SEQ ID NO: 99)

### 5.2.4. Antibody Fragments

Even though the term “antibody” is sometimes used in a phrase of “antibody or fragment thereof” herein, it should be understood the term “antibody” as used herein also includes various antibody fragments, such as an antigen-binding fragment or epitope-binding fragment. Thus, when the term “antibody” is used alone without being followed by “fragment thereof” or similar terms, it should be understood the term “antibody” includes an antibody fragment, such as an antigen-binding fragment or epitope-binding fragment. Antibodies provided herein include, but are not limited to, immunoglobulin molecules and immunologically active portions of immunoglobulin molecules. In some embodiments, the multispecific binding agents provided herein comprise one or more antibody fragments.

Variants and derivatives of antibodies include antibody functional fragments that retain the ability to bind to an antigen. Antibody fragments include but are not limited to those described in Section 5.1 above. Exemplary functional fragments include Fab fragments (e.g., an antibody fragment that contains the antigen-binding domain and comprises a light chain and part of a heavy chain bridged by a disulfide bond); Fab′ (e.g., an antibody fragment containing a single antigen-binding domain comprising an Fab and an additional portion of the heavy chain through the hinge region); F(ab′)₂ (e.g., two Fab′ molecules joined by interchain disulfide bonds in the hinge regions of the heavy chains; the Fab′ molecules may be directed toward the same or different epitopes); a bispecific Fab (e.g., a Fab molecule having two antigen-binding domains, each of which may be directed to a different epitope); a single chain comprising a variable region, also known as, scFv (e.g., the variable, antigen-binding determinative region of a single light and heavy chain of an antibody linked together by a chain of, e.g., 10-25 amino acids); a disulfide-linked Fv, or dsFv (e.g., the variable, antigen-binding determinative region of a single light and heavy chain of an antibody linked together by a disulfide bond); a bispecific scFv (e.g., an scFv or a dsFv molecule having two antigen-binding domains, each of which may be directed to a different epitope); a diabody (e.g., a dimerized scFv formed when the VH domain of a first scFv assembles with the VL domain of a second scFv and the VL domain of the first scFv assembles with the VH domain of the second scFv; the two antigen-binding regions of the diabody may be directed towards the same or different epitopes); a triabody (e.g., a trimerized scFv, formed in a manner similar to a diabody, but in which three antigen-binding domains are created in a single complex; the three antigen-binding domains may be directed towards the same or different epitopes); and a tetrabody (e.g., a tetramerized scFv, formed in a manner similar to a diabody, but in which four antigen-binding domains are created in a single complex; the four antigen-binding domains may be directed towards the same or different epitopes).

Various techniques have been developed for the production of antibody fragments. Traditionally, these fragments were derived via proteolytic digestion of intact antibodies (see, e.g., Morimoto et al., 1992, J. Biochem. Biophys. Methods 24:107-17; and Brennan et al., 1985, Science 229:81-83). However, these fragments can now be produced directly by recombinant host cells. For example, Fab, Fv, and scFv antibody fragments can all be expressed in and secreted from E. coli, yeast or insect cells, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., 1992, Bio/Technology 10:163-67). According to another approach, F(ab′)₂ fragments can be isolated directly from recombinant host cell culture. Fab and F(ab′)₂ fragments with increased in vivo half-life comprising salvage receptor binding epitope residues are described in, for example, U.S. Pat. No. 5,869,046. Other techniques for the production of antibody fragments will be apparent to the skilled practitioner. In certain embodiments, an antibody is a single chain Fv fragment (scFv) (see, e.g., WO 93/16185; U.S. Pat. Nos. 5,571,894 and 5,587,458). Fv and scFv have intact combining sites that are devoid of constant regions; thus, they may be suitable for reduced nonspecific binding during in vivo use. scFv fusion proteins may be constructed to yield fusion of an effector protein at either the amino or the carboxy terminus of an scFv (See, e.g., Borrebaeck ed., supra). The antibody fragment may also be a “linear antibody,” for example, as described in the references cited above. Such linear antibodies may be monospecific or multi-specific, such as bispecific.

### 5.2.5. Humanized Antibodies

The present disclosure provides humanized antibodies that bind to NKG2A and PD-L1, including human NKG2A and human PD-L1. Humanized antibodies of the present disclosure may comprise one or more CDRs from a VH and/or VL disclosed herein, such as those as shown in Tables 1-5. Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanized antibodies that bind NKG2A may be produced using techniques known to those skilled in the art (Zhang et al., Molecular Immunology, 42(12): 1445-1451, 2005; Hwang et al., Methods, 36(1): 35-42, 2005; Dall'Acqua et al., Methods, 36(1): 43-60, 2005; Clark, Immunology Today, 21(8): 397-402, 2000, and U.S. Pat. Nos. 6,180,370; 6,054,927; 5,869,619; 5,861,155; 5,712,120; and 4,816,567).

In some cases, the humanized antibodies are constructed by CDR grafting, in which the amino acid sequences of the six CDRs of a VH and a VL of the parent non-human antibody (e.g., rodent) are grafted onto a human antibody framework. For example, Padlan et al. (FASEB J. 9:133-139, 1995) determined that only about one third of the residues in the CDRs actually contact the antigen, and termed these the “specificity determining residues,” or SDRs. In the technique of SDR grafting, only the SDR residues are grafted onto the human antibody framework (see, e.g., Kashmiri et al., Methods 36:25-34, 2005).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence which is closest to that of the rodent may be selected as the human framework for the humanized antibody (Sims et al. (1993) J. Immunol. 151:2296; Chothia et al. (1987) J. Mol. Biol. 196:901). Another method uses a particular framework derived from the consensus sequence of all human antibodies of a particular subgroup of light or heavy chains. The same framework may be used for several different humanized antibodies (Carter et al. (1992) Proc. Natl. Acad. Sci. USA, 89:4285; Presta et al. (1993) J. Immunol., 151:2623). In some cases, the framework is derived from the consensus sequences of the most abundant human subclasses, V_{L}6 subgroup I (V_{L}6I) and V_{H} subgroup III (V_{H}III). In another method, human germline genes are used at the source of the framework regions.

In an alternative paradigm based on comparison of CDRs, called Superhumanization, framework homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as framework donors. Finally, the non-human CDRs are grafted onto these frameworks (see, e.g., Tan et al., J. Immunol. 169:1119-1125, 2002).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, Protein Eng. 13:819-824, 2000), Modeller (Sali and Blundell, J. Mol. Biol. 234:779-815, 1993), and Swiss PDB Viewer (Guex and Peitsch, Electrophoresis 18:2714-2713, 1997). Inspection of these displays permits analysis of the likely role of the residues in the functioning of the candidate immunoglobulin sequence, e.g., the analysis of residues that influence the ability of the candidate immunoglobulin to bind its antigen. In this way, framework residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes and the differences are scored as HSC. The target sequence is then humanized by maximizing its HSC rather than using a global identity measure to generate multiple diverse humanized variants. See, e.g., Lazar et al., Mol. Immunol. 44:1986-1998, 2007.

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome and yeast display libraries as well as by bacterial colony screening (see, e.g., Hoogenboom, Nat. Biotechnol. 23:1105-1116, 2005; Dufner et al., Trends Biotechnol. 24:523-529, 2006; Feldhaus et al., Nat. Biotechnol. 21:163-70, 2003; Schlapschy et al., Protein Eng. Des. Sel. 17:847-60, 2004).

In the framework library approach, a collection of residue variants are introduced at specific positions in the framework followed by selection of the library to select the framework that best supports the grafted CDR. The residues to be substituted may include some or all of the “Vernier” residues identified as potentially contributing to CDR structure (see, e.g., Foote and Winter, J. Mol. Biol. 224:487-499, 1992), or from the more limited set of target residues identified by Baca et al. (J. Biol. Chem. 272:10678-10684, 1997).

In framework shuffling, whole frameworks are combined with the non-human CDRs instead of creating combinatorial libraries of selected residue variants (see, e.g., Dall'Acqua et al., Methods 36:43-60, 2005). The libraries may be screened for binding in a two-step selection process, first humanizing VL, followed by VH. Alternatively, a one-step framework shuffling process may be used. Such a process has been shown to be more efficient than the two-step screening, as the resulting antibodies exhibited improved biochemical and physico-chemical properties including enhanced expression, increased affinity and thermal stability (see, e.g., Damschroder et al., Mol. Immunol. 44:3049-60, 2007).

The “humaneering” method is based on experimental identification of essential minimum specificity determinants (MSDs) and is based on sequential replacement of non-human fragments into libraries of human frameworks and assessment of binding. It begins with regions of the CDR3 of non-human VH and VL chains and progressively replaces other regions of the non-human antibody into the human frameworks, including the CDR1 and CDR2 of both VH and VL. This methodology typically results in epitope retention and identification of antibodies from multiple sub-classes with distinct human V-segment CDRs. Humaneering allows for isolation of antibodies that are 91-96% homologous to human germline gene antibodies. See, e.g., Alfenito, Cambridge Healthtech Institute's Third Annual PEGS, The Protein Engineering Summit, 2007.

The “human engineering” method involves altering a non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk”, “moderate risk”, or “high risk” residues. The classification is performed using a global risk/reward calculation that evaluates the predicted benefits of making a particular substitution (e.g., for immunogenicity in humans) against the risk that the substitution will affect the resulting antibody's folding and/or are substituted with human residues. The particular human amino acid residue to be substituted at a given position (e.g., low or moderate risk) of a non-human (e.g., mouse) antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., Protein Engineering, 7:805-814 (1994), U.S. Pat. Nos. 5,766,886, 5,770,196, 5,821,123, and 5,869,619, and WO 93/11794.

### 5.2.6. Antibody Variants

Modifications of the multispecific antibodies that bind to NKG2A and PD-L1 described herein are contemplated. For example, it may be desirable to optimize the binding affinity and/or other biological properties of the antibody, including but not limited to specificity, thermostability, expression level, effector functions, glycosylation, reduced immunogenicity, or solubility. Thus, it is contemplated that variants of the antibodies described herein can be prepared and are included in the present disclosure. In some embodiments, antibody variants are antibodies with amino acid sequence variations as compared with the original antibody, for example, substitution, deletion, or insertion of one or more amino acid(s), as described above. For example, variations may be a substitution, deletion, or insertion of one or more codons encoding the antibody or polypeptide that results in a change in the amino acid sequence (e.g., a conservative substitution) as compared with the original antibody or polypeptide. Sites of interest for substitutional mutagenesis include the CDRs, FRs and/or constant regions. For example, antibody variants can be prepared by introducing appropriate nucleotide changes into the encoding DNA, and/or by synthesis of the desired antibody or polypeptide. Those skilled in the art who appreciate that amino acid changes may alter post-translational processes of the antibody (e.g., multispecific antibody).

### Chemical Modifications

Other exemplary modifications include chemical modifications, for example, by the covalent attachment of any type of molecule to the multispecific antibody. Antibody derivatives may include antibodies that have been chemically modified, for example, by glycosylation, acetylation, pegylation, phosphorylation, amidation, derivatization by known protecting/blocking groups, proteolytic cleavage, linkage to a cellular ligand or other protein, or conjugation to one or more immunoglobulin domains (e.g., Fc or a portion of an Fc). Any of numerous chemical modifications may be carried out by known techniques, including, but not limited to, specific chemical cleavage, acetylation, formulation, metabolic synthesis of tunicamycin, etc. Additionally, the antibody may contain one or more non-classical amino acids.

In some embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

When the antibody provided herein (e.g., a bispecific antibody) is fused to an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in the binding molecules provided herein may be made in order to create variants with certain improved properties.

In other embodiments, when the antibody provided herein is fused to an Fc region, antibody variants provided herein may have a carbohydrate structure that lacks fucose attached (directly or indirectly) to said Fc region. For example, the amount of fucose in such antibody may be from 1% to 80%, from 1% to 65%, from 5% to 65%, or from 20% to 40%. The amount of fucose is determined by calculating the average amount of fucose within the sugar chain at Asn297, relative to the sum of all glycostructures attached to Asn297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., U.S. Pat. Publ. Nos. 2003/0157108 and 2004/0093621. Examples of publications related to “defucosylated” or “fucose-deficient” antibody variants include: U.S. Pat. Publ. No. 2003/0157108; WO 2000/61739; WO 2001/29246; U.S. Pat. Publ. No. 2003/0115614; U.S. Pat. Publ. No. 2002/0164328; U.S. Pat. Publ. No. 2004/0093621; U.S. Pat. Publ. No. 2004/0132140; U.S. Pat. Publ. No. 2004/0110704; U.S. Pat. Publ. No. 2004/0110282; U.S. Pat. Publ. No. 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87:614 (2004). Examples of cell lines capable of producing defucosylated antibodies include Lec13 CHO cells deficient in protein fucosylation (Ripka et al. Arch. Biochem. Biophys. 249:533-545 (1986); U.S. Pat. Publ. No. 2003/0157108; and WO 2004/056312, and knockout cell lines, such as alpha-1,6-fucosyltransferase gene, FUT8, knockout CHO cells (see, e.g., Yamane-Ohnuki et al. Biotech. Bioeng. 87:614 (2004); Kanda, Y. et al., Biotechnol. Bioeng., 94(4):680-688 (2006); and WO2003/085107).

The binding molecules comprising an antibody provided herein are further provided with bisected oligosaccharides, e.g., in which a biantennary oligosaccharide attached to the Fc region is bisected by GlcNAc. Such variants may have reduced fucosylation and/or improved ADCC function. Examples of such variants are described, e.g., in WO 2003/011878 (Jean-Mairet et al.); U.S. Pat. No. 6,602,684 (Umana et al.); and U.S. Pat. Publ. No. 2005/0123546 (Umana et al.). Variants with at least one galactose residue in the oligosaccharide attached to the Fc region are also provided. Such variants may have improved CDC function. Such variants are described, e.g., in WO 1997/30087; WO 1998/58964; and WO 1999/22764.

In molecules that comprise the present antibody and an Fc region, one or more amino acid modifications may be introduced into the Fc region, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In some embodiments, the present application contemplates variants that possess some but not all effector functions, which make it a desirable candidate for applications in which the half-life of the binding molecule in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the binding molecule lacks FcγR binding (hence likely lacking ADCC activity), but retains FcRn binding ability. Non-limiting examples of in vitro assays to assess ADCC activity of a molecule of interest are described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, CA; and CYTOTOX 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Binding molecules with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, a variant comprises an Fc region with one or more amino acid substitutions which improve ADCC, e.g., substitutions at positions 298, 333, and/or 334 of the Fc region (EU numbering of residues). In some embodiments, alterations are made in the Fc region that result in altered (e.g., either improved or diminished) C1q binding and/or Complement Dependent Cytotoxicity (CDC), e.g., as described in U.S. Pat. No. 6,194,551, WO 99/51642, and Idusogie et al. J. Immunol. 164:4178-4184 (2000).

Binding molecules with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those molecules comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821; and WO 94/29351 concerning other examples of Fc region variants.

In some embodiments, it may be desirable to create cysteine engineered antibodies, in which one or more residues of an antibody are substituted with cysteine residues. In some embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein.

Other known covalent modifications of antibodies are included within the scope of the present disclosure. Covalent modifications include reacting targeted amino acid residues of an antibody with an organic derivatizing agent that is capable of reacting with selected side chains or the N- or C-terminal residues of the antibody. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (see, e.g., Creighton, Proteins: Structure and Molecular Properties 79-86 (1983)), acetylation of the N-terminal amine, and amidation of any C-terminal carboxyl group.

The antibody that binds to NKG2A and PD-L1 of the present disclosure may also be modified to form chimeric molecules comprising the antibody fused or conjugated to another, heterologous polypeptide or amino acid sequence or a small molecule compound, for example, an immune activator (such as a cytokine), an epitope tag (see, e.g., Terpe, Appl. Microbiol. Biotechnol. 60:523-33 (2003)) or the Fc region of an IgG molecule (see, e.g., Aruffo, Antibody Fusion Proteins 221-42 (Chamow and Ashkenazi eds., 1999)).

Also provided herein are fusion proteins comprising the multispecific antibody that binds to NKG2A and PD-L1 of the disclosure and a heterologous polypeptide. In some embodiments, the heterologous polypeptide to which the antibody is genetically fused or chemically conjugated is useful for targeting the antibody to cells having cell surface-expressed NKG2A and/or PD-L1. Genetically fused or chemically conjugated antibodies are described in more detail in sections below.

### In Vitro Affinity Maturation

In some embodiments, antibody variants having an improved property such as affinity, stability, or expression level as compared to a parent antibody may be prepared by in vitro affinity maturation. Like the natural prototype, in vitro affinity maturation is based on the principles of mutation and selection. Libraries of antibodies are displayed on the surface of an organism (e.g., phage, bacteria, yeast, or mammalian cell) or in association (e.g., covalently or non-covalently) with their encoding mRNA or DNA. Affinity selection of the displayed antibodies allows isolation of organisms or complexes carrying the genetic information encoding the antibodies. Two or three rounds of mutation and selection using display methods such as phage display usually results in antibody fragments with affinities in the low nanomolar range. Affinity matured antibodies can have nanomolar or even picomolar affinities for the target antigen.

Phage display is a widespread method for display and selection of antibodies. The antibodies are displayed on the surface of Fd or M13 bacteriophages as fusions to the bacteriophage coat protein. Selection involves exposure to antigen to allow phage-displayed antibodies to bind their targets, a process referred to as “panning.” Phage bound to antigen are recovered and used to infect bacteria to produce phage for further rounds of selection. For review, see, for example, Hoogenboom, Methods. Mol. Biol. 178:1-37 (2002); and Bradbury and Marks, J. Immunol. Methods 290:29-49 (2004).

In a yeast display system (see, e.g., Boder et al., Nat. Biotech. 15:553-57 (1997); and Chao et al., Nat. Protocols 1:755-68 (2006)), the antibody may be fused to the adhesion subunit of the yeast agglutinin protein Aga2p, which attaches to the yeast cell wall through disulfide bonds to Aga1p. Display of a protein via Aga2p projects the protein away from the cell surface, minimizing potential interactions with other molecules on the yeast cell wall. Magnetic separation and flow cytometry are used to screen the library to select for antibodies with improved affinity or stability. Binding to a soluble antigen of interest is determined by labeling yeast with biotinylated antigen and a secondary reagent such as streptavidin conjugated to a fluorophore. Variations in surface expression of the antibody can be measured through immunofluorescence labeling of either the hemagglutinin or c-Myc epitope tag flanking the single-chain antibody (e.g., scFv). Expression has been shown to correlate with the stability of the displayed protein, and thus antibodies can be selected for improved stability as well as affinity (see, e.g., Shusta et al., J. Mol. Biol. 292:949-56 (1999)). An additional advantage of yeast display is that displayed proteins are folded in the endoplasmic reticulum of the eukaryotic yeast cells, taking advantage of endoplasmic reticulum chaperones and quality-control machinery. Once maturation is complete, antibody affinity can be conveniently “titrated” while displayed on the surface of the yeast, eliminating the need for expression and purification of each clone. A theoretical limitation of yeast surface display is the potentially smaller functional library size than that of other display methods; however, a recent approach uses the yeast cells' mating system to create combinatorial diversity estimated to be 10¹⁴ in size (see, e.g., U.S. Pat. Publ. No. 2003/0186374; and Blaise et al., Gene 342:211-18 (2004)).

In ribosome display, antibody-ribosome-mRNA (ARM) complexes are generated for selection in a cell-free system. The DNA library coding for a particular library of antibodies is genetically fused to a spacer sequence lacking a stop codon. This spacer sequence, when translated, is still attached to the peptidyl tRNA and occupies the ribosomal tunnel, and thus allows the protein of interest to protrude out of the ribosome and fold. The resulting complex of mRNA, ribosome, and protein can bind to surface-bound ligand, allowing simultaneous isolation of the antibody and its encoding mRNA through affinity capture with the ligand. The ribosome-bound mRNA is then reverse transcribed back into cDNA, which can then undergo mutagenesis and be used in the next round of selection (see, e.g., Fukuda et al., Nucleic Acids Res. 34:e127 (2006)). In mRNA display, a covalent bond between antibody and mRNA is established using puromycin as an adaptor molecule (Wilson et al., Proc. Natl. Acad. Sci. USA 98:3750-55 (2001)).

As these methods are performed entirely in vitro, they provide two main advantages over other selection technologies. First, the diversity of the library is not limited by the transformation efficiency of bacterial cells, but only by the number of ribosomes and different mRNA molecules present in the test tube. Second, random mutations can be introduced easily after each selection round, for example, by non-proofreading polymerases, as no library must be transformed after any diversification step. In some embodiments, mammalian display systems may be used.

Diversity may also be introduced into the CDRs of the antibody libraries in a targeted manner or via random introduction. The former approach includes sequentially targeting all the CDRs of an antibody via a high or low level of mutagenesis or targeting isolated hot spots of somatic hypermutations (see, e.g., Ho et al., J. Biol. Chem. 280:607-17 (2005)) or residues suspected of affecting affinity on experimental basis or structural reasons. Diversity may also be introduced by replacement of regions that are naturally diverse via DNA shuffling or similar techniques (see, e.g., Lu et al., J. Biol. Chem. 278:43496-507 (2003); U.S. Pat. Nos. 5,565,332 and 6,989,250). Alternative techniques target hypervariable loops extending into framework-region residues (see, e.g., Bond et al., J. Mol. Biol. 348:699-709 (2005)) employ loop deletions and insertions in CDRs or use hybridization-based diversification (see, e.g., U.S. Pat. Publ. No. 2004/0005709). Additional methods of generating diversity in CDRs are disclosed, for example, in U.S. Pat. No. 7,985,840. Further methods that can be used to generate antibody libraries and/or antibody affinity maturation are disclosed, e.g., in U.S. Pat. Nos. 8,685,897 and 8,603,930, and U.S. Publ. Nos. 2014/0170705, 2014/0094392, 2012/0028301, 2011/0183855, and 2009/0075378, each of which is incorporated herein by reference.

Screening of the libraries can be accomplished by various techniques known in the art. For example, antibodies can be immobilized onto solid supports, columns, pins, or cellulose/poly (vinylidene fluoride) membranes/other filters, expressed on host cells affixed to adsorption plates or used in cell sorting, or conjugated to biotin for capture with streptavidin-coated beads or used in any other method for panning display libraries.

For reviews of in vitro affinity maturation methods, see, e.g., Hoogenboom, Nature Biotechnology 23:1105-16 (2005); Quiroz and Sinclair, Revista Ingeneria Biomedia 4:39-51 (2010); and references therein.

An antibody internalization assay may be used to determine receptor-mediated endocytosis when binding to an antibody. In some embodiments, the efficacy of certain antibody-based therapeutics depends on antibody internalization process. In some embodiments, an antibody internalization assay examines the rate and extent of antibody internalization in order to evaluate the antibody's ability of delivering treatments to sites or cells of interest. A non-limiting exemplary assay is briefly described below. Target cells of interest are seeded at an appropriate seeding density (e.g., in a 96-well U-bottom plate), and a tested antibody is labelled with a signal reporting reagent, for example, fluorescent compounds, Horseradish peroxidase (HRP) reagent, radiolabeled compounds, or biotin. Then the tested antibody and the target cells are incubated at an appropriate molar ratio. Following the incubation, unbound antibodies are removed by wash. The cells can be left on ice or incubated at 37° C. for a period of time to facilitate internalization. The cells may then be incubated for a period of time in the presence of a stop reagent to inhibit internalization. Subsequently, the cells are washed and incubated with the signal developing reagent. The final signal can be studied using plate reader or imaging instrument and an analytical software. For example, mean fluorescence intensity (MFI) of the cells can be measured using a flow cytometer, and MFI reduction can represent antibody internalization, antibody dissociation or a combination of both. Cell imaging can be scanned and acquired to analyze the signal intensity, size and shape. Alternatively, the cells are lysed, releasing internalized antibody. This antibody is then captured in a microtiter well plate coated with specific antigen against which the antibody was raised. Bound antibody in the well is detected using an alkaline phosphatase or HRP-conjugated secondary antibody and a chromogenic substrate. Alternative detectable labels for the antibody and means for detecting internalized labeled antibody will be obvious to those skilled in the art upon this disclosure. Any methods known in the art to determine antibody internalization can be used in the present disclosure.

### 5.2.7. Other Binding Agents Comprising the Multispecific Antibodies

In some embodiments, the multispecific antibody or fragment thereof provided herein is a part of a larger binding agent. Non-limiting exemplary binding agents comprising the antibody or fragment provided herein are described below.

The present disclosure provides binding agents (e.g., an antibody, a multispecific binding agent, or a bispecific antibody) with a masking moiety and/or cleavable moiety in which one or more of the NKG2A binding domains and/or PD-L1 binding domains of the binding agent (e.g., multispecific antibody) are masked (e.g., via a masking moiety) and/or activatable (e.g., via a cleavable moiety). Technologies for masking of antibodies are well known in the art, including SAFEbody masking technology (see, e.g., U.S. Pat. Publ. No. 2019/0241886) and Probody masking technology (see, e.g., U.S. Pat. Publ. No. 2015/0079088). Such technologies can be used to generate a binding agent (e.g., an antibody, a multispecific binding agent, or a bispecific antibody) that is masked and/or activatable. Such masked and/or activatable binding agents (e.g., an antibody, a multispecific binding agent, or bispecific antibody) are also useful for the preparation of conjugates, including immunoconjugates, antibody-drug conjugates (ADCs), masked ADCs and activatable ADCs (AADCs), comprising any one of the binding agents (e.g., an antibody, a multispecific binding agent, or a bispecific antibody) of the present disclosure, including those directly or indirectly linked another agent such as a drug and/or an immune activator (such as a cytokine). For example, binding agents (e.g., an antibody, a multispecific binding agent, or a bispecific antibody) of the present disclosure may be covalently bound by a synthetic linker to one or more agents such as drugs and/or immune activators.

If desired, a binding agent (e.g., an antibody, a multispecific binding agent, or a bispecific antibody) provided herein is linked or conjugated (directly or indirectly) to a moiety with effector function, such as cytotoxic activity (e.g., a chemotherapeutic moiety or a radioisotope), immune recruitment or modulating activity. Moieties that are linked or conjugated (directly or indirectly) include drugs that are cytotoxic (e.g., toxins such as aurostatins) or non-cytotoxic, e.g., signal transduction modulators such as kinases or masking moieties that mask one or more binding domains of the binding agent provided herein, or cleavable moieties that allow for activating a binding agent by cleaving off a cleavable moiety to unmask one or more binding domains in the tumor microenvironment in the form of masked conjugates. Moieties that promote immune recruitment can include other antigen-binding agents, such as viral proteins that bind selectively to cells of the innate and/or adaptive immune system. Alternatively or in addition, a binding agent (e.g., an antibody, a multispecific binding agent, or a bispecific antibody) provided herein is optionally linked or conjugated (directly or indirectly) to a moiety that facilitates isolation from a mixture (e.g., a tag) or a moiety with reporter activity (e.g., a detection label or reporter protein). It will be appreciated that the features of a binding agent described herein extend also to a polypeptide comprising a binding agent fragment.

In some embodiments, multispecific binding agents (e.g., bispecific antibodies) described herein may be linked or conjugated (directly or indirectly) to a polypeptide, which can result in the generation of an activatable antibody. In some embodiments, the multispecific binding agent provided herein is linked or conjugated (directly or indirectly) to an additional agent. In some embodiments, the additional agent is a drug, resulting in an ADC or an AADC when the antibody of the ADC comprises a masking moiety and a cleavable moiety.

In some embodiments, binding agents (e.g., an antibody, a multispecific binding agent, or a bispecific antibody), described herein are conjugated or recombinantly linked (directly or indirectly) to a therapeutic agent (e.g., a cytotoxic agent or a cytokine) or to a diagnostic or detectable agent. The conjugated or recombinantly linked antibodies, including masked or activatable conjugates, can be useful, for example, for treating or preventing a disease, disorder or condition. The conjugated or recombinantly linked multispecific binding agents (e.g., bispecific antibodies), including masked or activatable conjugates, can be useful, for example, for monitoring or prognosing the onset, development, progression, and/or severity of a disease, disorder or condition.

Such diagnosis and detection can be accomplished, for example, by coupling a binding agent (e.g., an antibody, a multispecific binding agent, or a bispecific antibody) to detectable substances including, for example: enzymes, including, but not limited to, horseradish peroxidase, alkaline phosphatase, beta-galactosidase, or acetylcholinesterase; prosthetic groups, including, but not limited to, streptavidin/biotin or avidin/biotin; fluorescent materials, including, but not limited to, umbelliferone, fluorescein, fluorescein isothiocynate, rhodamine, dichlorotriazinylamine fluorescein, dansyl chloride, or phycoerythrin; luminescent materials, including, but not limited to, luminol; bioluminescent materials, including, but not limited to, luciferase, luciferin, or aequorin; chemiluminescent material, including, but not limited to, an acridinium based compound or a HALOTAG; radioactive materials, including, but not limited to, iodine (¹³¹I, ¹²⁵I, ¹²³I, and ¹²¹I), carbon (¹⁴C), sulfur (³⁵S), tritium (³H), indium (¹¹⁵In, ¹¹³In, ¹¹²In, and ¹¹¹In), technetium (⁹⁹Tc), thallium (²⁰¹Ti), gallium (⁶⁸Ga and ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, or ¹¹⁷Sn; positron emitting metals using various positron emission tomographies; and non-radioactive paramagnetic metal ions.

Also described herein are multispecific binding agents (e.g., bispecific antibodies) that are recombinantly linked or conjugated (covalent or non-covalent conjugations, directly or indirectly) to a heterologous protein or polypeptide or fragment thereof, for example, to a polypeptide (e.g., of about 10, about 20, about 30, about 40, about 50, about 60, about 70, about 80, about 90, or about 100 amino acids) to generate fusion proteins, as well as uses thereof. In some embodiments, the heterologous protein, polypeptide, or peptide that a binding agent (e.g., an antibody, a multispecific binding agent, or a bispecific antibody) is linked to is useful for targeting the binding agent to a particular cell (e.g., an NKG2A-expressing cell, such as, an immune cell; and/or PD-L1 expressing cell, including an immune cell, and/or a cancer or tumor cell). Other non-limiting heterologous protein, polypeptide, or peptide that a binding agent (e.g., an antibody, a multispecific binding agent, or a bispecific antibody) is linked to can be useful as an internalization signal or engaging a tumor cell with an immune cell.

Moreover, binding agents (e.g., an antibody, a multispecific binding agent, or a bispecific antibody) described herein can be linked (directly or indirectly) to marker or “tag” sequences, such as a peptide, to facilitate purification. In some embodiments, the marker or tag amino acid sequence is a hexa-histidine peptide, such as the tag provided in a pQE vector (see, e.g., QIAGEN, Inc.), among others, many of which are commercially available. For example, as described in Gentz et al., 1989, Proc. Natl. Acad. Sci. USA 86:821-24, hexa-histidine provides for convenient purification of a fusion protein. Other peptide tags useful for purification include, but are not limited to, the hemagglutinin (“HA”) tag, which corresponds to an epitope derived from the influenza hemagglutinin protein (Wilson et al., 1984, Cell 37:767-78), and the “FLAG” tag.

Methods for linking or conjugating (directly or indirectly) moieties (including polypeptides) to antibodies are well known in the art, any one of which can be used to make an antibody-drug conjugate or fusion protein described herein.

In some embodiments, a binding agent (e.g., an antibody, a multispecific binding agent, or a bispecific antibody) described herein is a fusion protein. The term “fusion protein” as used herein refers to a polypeptide that comprises an amino acid sequence of a binding agent (e.g., an antibody) and an amino acid sequence of a heterologous polypeptide or protein (e.g., a polypeptide or protein not normally a part of the antibody). In certain embodiments, the fusion protein retains the biological activity of a binding agent.

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of binding agents (e.g., an antibody, a multispecific binding agent, or a bispecific antibody) as described herein with higher affinities and lower dissociation rates (see, e.g., U.S. Pat. Nos. 5,605,793; 5,811,238; 5,830,721; 5,834,252; and U.S. Pat. No. 5,837,458; Patten et al., 1997, Curr. Opinion Biotechnol. 8:724-33; Harayama, 1998, Trends Biotechnol. 16(2):76-82; Hansson et al., 1999, J. Mol. Biol. 287:265-76; and Lorenzo and Blasco, 1998, Biotechniques 24(2):308-13). In some embodiments, the binding agents provided herein may be altered by being subjected to random mutagenesis by error-prone PCR, random nucleotide insertion, or other methods prior to recombination. A polynucleotide encoding a binding agent described herein may be recombined with one or more components, motifs, sections, parts, domains, fragments, etc. of one or more heterologous molecules.

The binding agents (e.g., an antibody, a multispecific binding agent, or a bispecific antibody) described herein may also be attached to solid supports, which are useful for immunoassays or purification of the target antigen. Such solid supports include, but are not limited to, glass, cellulose, polyacrylamide, nylon, polystyrene, polyvinyl chloride, or polypropylene.

The binding agents (e.g., an antibody, a multispecific binding agent, or a bispecific antibody) described herein can also be linked or conjugated (directly or indirectly) to a second antibody to form an antibody heteroconjugate.

The linker may be a “cleavable moiety” facilitating release of the linked or conjugated agent in a cell, but non-cleavable linkers are also contemplated herein. Linkers for use in conjugates (e.g., ADCs or AADCs) of the present disclosure include, without limitation, acid labile linkers (e.g., hydrazone linkers), disulfide-containing linkers, peptidase-sensitive linkers (e.g., peptide linkers comprising amino acids, for example, valine and/or citrulline such as citrulline-valine or phenylalanine-lysine), photolabile linkers, dimethyl linkers, thioether linkers, or hydrophilic linkers designed to evade multidrug transporter-mediated resistance.

Conjugates of an antibody and agent, including wherein the agent is a drug for the preparation of ADC or an AADC, may be made using a variety of bifunctional protein coupling agents such as N-(β-maleimidopropyloxy)succinimide ester (BMPS); N-ε-malemidocaproyl-oxysuccinimide ester (ECMS); N-γ-malemidocaproyl-oxysuccinimide ester (GMBS); 1,6-hexane-bis-vinylsulfone (HBVS); succinimidyl 4 (-N-maleimidomethyl)cyclohexane-1-carboxy-(6-amidocaproate)) (LC-SMCC); m-maleimidobenzoyl-N-hydroxysuccinimide ester (MBS); 4-(4-N-maleimidophenyl) butyric acid hydrazide (MPBH); succinimidil 3-(bromoacetamido) propionate (SBAP); succinimidyl iodoacetate (SIA); succinimidyl (4-iodoacetyl)aminobenzoate (SIAB); succinimidyl-4-(N-maleimidomethyl)cyclohexane-1-carboxylate (SMCC); succinimidyl 4-(p-maleimido-phenyl) butyrate (SMPB); succinimidyl-6-(β-maleimidopropionamido) hexanoate (SMPH); N-(ε-maleimidocaproyloxy)sulfosuccinimide ester (sulfo-ECMS); N-(γ-maleimidobutryloxy)sulfosuccinimide ester (sulfo-GMBS); N-(κ-maleimidoundecanoyloxy)sulfosuccinimide ester (sulfo-KMUS); m-maleimidobenzoyl-N-hydroxysulfosuccinimide ester (sulfo-MBS); sulfosuccinimidyl (4-iodo-acetyl)aminobenzoate (sulfo-SIAB); sulfosuccinimidyl 4-(N-maleimido-methyl)cyclohexane-1-carboxylate (sulfo-SMCC); sulfosuccinimidyl 4-(p-maleimidophenyl) butyrate (sulfo-SMPB); and succinimidyl-(4-vinylsulfone)benzoate) (SVSB).

The present disclosure further contemplates conjugates of antibodies and agents, including wherein the agent is a drug for the preparation of ADC or AADC, may be prepared using any suitable methods as disclosed in the art (see, e.g., Bioconjugate Techniques (Hermanson ed., 2d ed. 2008)).

Conventional conjugation strategies for antibodies and agents, including wherein the agent is a drug for the preparation of ADC or AADC, have been based on random conjugation chemistries involving the ε-amino group of Lys residues or the thiol group of Cys residues, which results in heterogeneous conjugates. Recently developed techniques allow site-specific conjugation to antibodies, resulting in homogeneous loading and avoiding conjugate subpopulations with altered antigen-binding or pharmacokinetics. These include engineering of “thiomabs” comprising cysteine substitutions at positions on the heavy and light chains that provide reactive thiol groups and do not disrupt immunoglobulin folding and assembly or alter antigen binding (see, e.g., Junutula et al., 2008, J. Immunol. Meth. 332:41-52; and Junutula et al., 2008, Nature Biotechnol. 26:925-32). In another method, selenocysteine is co-translationally inserted into an antibody sequence by recoding the stop codon UGA from termination to selenocysteine insertion, allowing site specific covalent conjugation at the nucleophilic selenol group of selenocysteine in the presence of the other natural amino acids (see, e.g., Hofer et al., 2008, Proc. Natl. Acad. Sci. USA 105:12451-56; and Hofer et al., 2009, Biochemistry 48(50):12047-57).

In some embodiments, the binding agent (an antibody, a multispecific binding agent, or a bispecific antibody) described herein is conjugated to an agent, for example, an immune activator (such as a cytokine) or a cytotoxic agent. In some embodiments, a binding agent (an antibody, a multispecific binding agent, or a bispecific antibody) described herein can be optionally conjugated with one or more cytotoxic agent(s) disclosed herein or known in the art in order to generate an ADC or AADC. In some embodiments, the cytotoxic agent is a chemotherapeutic agent including, but not limited to, methotrexate, adriamycin, doxorubicin, melphalan, mitomycin C, chlorambucil, daunorubicin or other intercalating agents. In some embodiments, the cytotoxic agent is an enzymatically active toxin of bacterial, fungal, plant, or animal origin, or fragments thereof, including, but not limited to, diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, the cytotoxic agent is a radioisotope to produce a radioconjugate or a radioconjugated agent. A variety of radionuclides are available for the production of radioconjugated agents including, but not limited to, ⁹⁰Y, ¹²⁵I, ¹³¹I, ¹²³I, ¹¹¹In, ¹³¹In, ¹⁰⁵Rh, ¹⁵³Sm, ⁶⁷Cu, ⁶⁷Ga, ¹⁶⁶Ho, ¹⁷⁷Lu, ¹⁸⁶Re, ¹⁸⁸Re, and ²¹²Bi. Conjugates of a polypeptide or molecule and one or more small molecule toxins, such as a calicheamicin, maytansinoids, a trichothene, and CC1065, and the derivatives of these toxins that have toxin activity, can also be used. Conjugates of a polypeptide or molecule and cytotoxic agent are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). In other embodiments, the agent is a signal transduction modulator, a pro-apoptotic agent, a mitotic inhibitor, an anti-tumor antibiotic, an immunomodulating agent, a nucleic acid for gene therapy, an alkylating agent, an anti-angiogenic agent, an anti-metabolite, a boron-containing agent, a chemoprotective agent, a hormone agent, an anti-hormone agent, a corticosteroid, a photoactive therapeutic agent, an oligonucleotide, a radionuclide agent, a radiosensitizer, a topoisomerase inhibitor, such as camptothecin or an analog thereof, and a tyrosine kinase inhibitor. In some embodiments, the mitotic inhibitor is a dolastatin, an auristatin, a maytansinoid, and a plant alkaloid. In some embodiments, the agent is a dolastatin, an auristatin, a maytansinoid, and a plant alkaloid. An example of an auristatin is monomethylaurisatin F (MMAF) or monomethyauristatin E (MMAE). Examples of maytansinoids include, but are not limited to, DM1, DM2, DM3, and DM4. In some embodiments, the anti-tumor antibiotic is selected from the group consisting of an actinomycine, an anthracycline, a calicheamicin, and a duocarmycin. An example of an actinomycine is a pyrrolobenzodiazepine (PBD). An example of an anthracycline is a PNU-anthracycline such as PNU-159682 or derivative.

In another aspect, the antibody or antigen-binding fragment thereof provided herein can be part of an engineered cell surface receptor such as a chimeric antigen receptor (CAR). Typically, a CAR comprises an extracellular domain, a transmembrane domain, and an intracellular signaling domain. In some embodiments, provided herein is a CAR comprising an extracellular domain that comprises one or more antibody or fragment thereof provided herein. In some embodiments, the extracellular domain of a CAR provided herein comprises VH and VL CDRs disclosed herein, such as those as set forth in Table 1, Table 2, Table 3, Table 4 or Table 5. The CARs of the present disclosure comprise a transmembrane domain that can be directly or indirectly fused to the extracellular antigen-binding domain. The transmembrane domain may be derived either from a natural or from a synthetic source. As used herein, a “transmembrane domain” refers to any protein structure that is thermodynamically stable in a cell membrane, preferably a eukaryotic cell membrane. Transmembrane domains compatible for use in the CARs described herein may be obtained from a naturally occurring protein. Alternatively, it can be a synthetic, non-naturally occurring protein segment, e.g., a hydrophobic protein segment that is thermodynamically stable in a cell membrane. The CARs of the present disclosure comprise an intracellular signaling domain. The intracellular signaling domain is responsible for activation of at least one of the normal effector functions of the immune effector cell expressing the CARs. The term “effector function” refers to a specialized function of a cell. Effector function of a T cell, for example, may be cytolytic activity or helper activity including the secretion of cytokines. Thus the term “cytoplasmic signaling domain” refers to the portion of a protein which transduces the effector function signal and directs the cell to perform a specialized function. While usually the entire cytoplasmic signaling domain can be employed, in many cases it is not necessary to use the entire chain. To the extent that a truncated portion of the cytoplasmic signaling domain is used, such truncated portion may be used in place of the intact chain as long as it transduces the effector function signal. The term cytoplasmic signaling domain is thus meant to include any truncated portion of the cytoplasmic signaling domain sufficient to transduce the effector function signal. In some embodiments, the intracellular signaling domain comprises a primary intracellular signaling domain of an immune effector cell. In some embodiments, the CAR comprises an intracellular signaling domain consisting essentially of a primary intracellular signaling domain of an immune effector cell. “Primary intracellular signaling domain” refers to cytoplasmic signaling sequence that acts in a stimulatory manner to induce immune effector functions. Many immune effector cells require co-stimulation, in addition to stimulation of an antigen-specific signal, to promote cell proliferation, differentiation and survival, as well as to activate effector functions of the cell. In some embodiments, the CAR comprises at least one co-stimulatory signaling domain. The term “co-stimulatory signaling domain,” as used herein, refers to at least a portion of a protein that mediates signal transduction within a cell to induce an immune response such as an effector function. The CARs of the present disclosure may comprise a hinge domain that is located between the extracellular antigen-binding domain and the transmembrane domain. A hinge domain is an amino acid segment that is generally found between two domains of a protein and may allow for flexibility of the protein and movement of one or both of the domains relative to one another. Any amino acid sequence that provides such flexibility and movement of the extracellular antigen-binding domain relative to the transmembrane domain of the effector molecule can be used. The CARs of the present disclosure may comprise a signal peptide (also known as a signal sequence) at the N-terminus of the polypeptide. In general, signal peptides are peptide sequences that target a polypeptide to the desired site in a cell. Other engineered transmembrane receptors comprising the antibody or fragment provided herein are also included in the present disclosure.

### 5.3. Nucleic Acids, Vectors and Cells

Additionally provided are a nucleic acid encoding a binding agent (e.g., a multispecific antibody or fragment thereof) or a fusion polypeptide as disclosed herein, a nucleic acid complementary thereto; a vector comprising a nucleic acid as disclosed herein; and a cell comprising any one or more of: a binding agent as disclosed herein, a nucleic acid as disclosed herein, or a vector as disclosed herein. In some embodiments, the cell expresses the binding agent provided herein. In some embodiments, the cell replicates the nucleic acid or the vector. In some embodiments, provided are the materials for generating binding agents and fragments thereof. For example, an isolated cell may produce a binding agent (e.g., multispecific antibody or antibody fragment). In this regard, a cell (e.g., an isolated cell) may produce an antibody or fragment thereof comprising a VH and a VL as disclosed herein. In some embodiments, polynucleotides described herein may comprise one or more nucleic acid sequences encoding a binding agent (e.g., antibody or antibody fragment). In some embodiments, the polynucleotide is an isolated and/or recombinant polynucleotide. In various aspects, the isolated polynucleotide comprises a nucleotide sequence that encodes a VH and/or a VL, wherein the VH and the VL comprise complementarity determining regions (CDRs) identical to CDRs as disclosed herein.

As used herein, the term “complementary” refers to specific binding between polynucleotides based on the sequences of the polynucleotides. As used herein, a first polynucleotide and a second polynucleotide are complementary if they bind to each other in a hybridization assay under stringent conditions, e.g., if they produce a given or detectable level of signal in a hybridization assay. Portions of polynucleotides are complementary to each other if they follow conventional base-pairing rules, e.g., A pairs with T (or U) and G pairs with C, although small regions (e.g., fewer than about 3 bases) of mismatch, insertion, or deleted sequence may be present. The term “stringent assay conditions” refers to conditions that are compatible to produce binding pairs of nucleic acids, e.g., probes and target mRNAs, of sufficient complementarity to provide for the desired level of specificity in the assay while being generally incompatible to the formation of binding pairs between binding members of insufficient complementarity to provide for the desired specificity. The term “stringent assay conditions” generally refers to the combination of hybridization and wash conditions.

In some embodiments, one or more vectors (e.g., expression vectors) may comprise one or more polynucleotides for expression of the one or more polynucleotides in a suitable host cell. Such vectors are useful, for example, for amplifying the polynucleotides in host cells to create useful quantities thereof, and for expressing binding agents, such as antibodies or antibody fragments, using recombinant techniques.

In some embodiments, one or more vectors are expression vectors wherein one or more polynucleotides are operatively linked to one or more polynucleotides comprising expression control sequences. Autonomously replicating recombinant expression constructs such as plasmid and viral DNA vectors incorporating one or more polynucleotides encoding antibody sequences that bind NKG2A and/or PD-L1 are specifically contemplated. Expression control DNA sequences include promoters, enhancers, and operators, and are generally selected based on the expression systems in which the expression construct is to be utilized. Promoter and enhancer sequences are generally selected for the ability to increase gene expression, while operator sequences are generally selected for the ability to regulate gene expression. Expression constructs may also include sequences encoding one or more selectable markers that permit identification of host cells bearing the construct. Expression constructs may also include sequences that facilitate, and preferably promote, homologous recombination in a host cell. In some embodiments, expression constructs of the can also include sequences necessary for replication in a host cell.

Exemplary expression control sequences include promoter/enhancer sequences, e.g., cytomegalovirus promoter/enhancer (Lehner et al., J. Clin. Microbiol., 29:2494-2502, 1991; Boshart et al., Cell, 41:521-530, 1985); Rous sarcoma virus promoter (Davis et al., Hum. Gene Ther., 4:151, 1993); Tie promoter (Korhonen et al., Blood, 86(5): 1828-1835, 1995); simian virus 40 promoter; DRA (downregulated in adenoma; Alrefai et al., Am. J. Physiol. Gastrointest. Liver Physiol., 293: G923-G934, 2007); MCT1 (monocarboxylate transporter 1; Cuff et al., Am. J. Physiol. Gastrointet. Liver Physiol., G977-G979. 2005); and Math 1 (mouse atonal homolog 1; Shroyer et al., Gastroenterology, 132:2477-2478, 2007), for expression in mammalian cells, the promoter being operatively linked upstream (e.g., 5′) of a polypeptide coding sequence. In another variation, the promoter is an epithelial-specific promoter or endothelial-specific promoter. Polynucleotides may also optionally include a suitable polyadenylation sequence (e.g., the SV40 or human growth hormone gene polyadenylation sequence) operably linked downstream (e.g., 3′) of the polypeptide coding sequence.

If desired, the one or more polynucleotides also optionally comprise nucleotide sequences encoding secretory signal peptides fused in frame with the polypeptide sequences. The secretory signal peptides direct secretion of the antibody polypeptides by the cells that express the one or more polynucleotides, and are cleaved by the cell from the secreted polypeptides. The one or more polynucleotides may further optionally comprise sequences whose only intended function is to facilitate large scale production of the vector. One can manufacture and administer polynucleotides for gene therapy using procedures that have been described in the literature for a variety of transgenes. See, e.g., Isner et al., Circulation, 91:2687-2692, 1995; and Isner et al., Human Gene Therapy, 7:989-1011, 1996.

In some embodiments, polynucleotides may further comprise additional sequences to facilitate uptake by host cells and expression of the antibody or fragment thereof (and/or any other peptide). In some embodiments, a “naked” transgene encoding an antibody or fragment thereof described herein (e.g., a transgene without a viral, liposomal, or other vector to facilitate transfection) is employed.

The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand. In some embodiments, the polynucleotide is in the form of cDNA. In some embodiments, the polynucleotide is a synthetic polynucleotide.

The present disclosure further relates to variants of the polynucleotides described herein, wherein the variant encodes, for example, fragments, analogs, and/or derivatives of the binding molecules of the disclosure. In certain embodiments, the present disclosure provides a polynucleotide comprising a polynucleotide having a nucleotide sequence at least about 75% identical, at least about 80% identical, at least about 85% identical, at least about 90% identical, at least about 95% identical, and in some embodiments, at least about 96%, 97%, 98% or 99% identical to a polynucleotide encoding the binding molecule of the disclosure. As used herein, the phrase “a polynucleotide having a nucleotide sequence at least, for example, 95% “identical” to a reference nucleotide sequence is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

Any suitable vectors may be used to introduce one or more polynucleotides that encode an antibody or fragment thereof into the host. Exemplary vectors that have been described include replication deficient retroviral vectors, including but not limited to lentivirus vectors (Kim et al., J. Virol., 72(1): 811-816, 1998; Kingsman & Johnson, Scrip Magazine, October 1998, pp. 43-46); parvoviral vectors, such as adeno-associated viral (AAV) vectors (U.S. Pat. Nos. 5,474,9351; 5,139,941; 5,622,856; 5,658,776; 5,773,289; 5,789,390; 5,834,441; 5,863,541; 5,851,521; 5,252,479; Gnatenko et al., J. Invest. Med., 45:87-98, 1997); adenoviral (AV) vectors (U.S. Pat. Nos. 5,792,453; 5,824,544; 5,707,618; 5,693,509; 5,670,488; 5,585,362; Quantin et al., Proc. Natl. Acad. Sci. USA, 89:2581-2584, 1992; Stratford Perricaudet et al., J. Clin. Invest., 90:626-630, 1992; and Rosenfeld et al., Cell, 68:143-155, 1992); an adenoviral adeno-associated viral chimeric (U.S. Pat. No. 5,856,152) or a vaccinia viral or a herpesviral vector (U.S. Pat. Nos. 5,879,934; 5,849,571; 5,830,727; 5,661,033; 5,328,688); Lipofectin mediated gene transfer (BRL); liposomal vectors (U.S. Pat. No. 5,631,237); and combinations thereof. Any of these expression vectors can be prepared using standard recombinant DNA techniques described in, e.g., Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994). Optionally, viral vectors are rendered replication-deficient by, e.g., deleting or disrupting select genes required for viral replication.

Other non-viral delivery mechanisms contemplated include calcium phosphate precipitation (Graham and Van Der Eb, Virology, 52:456-467, 1973; Chen and Okayama, Mol. Cell Biol., 7:2745-2752, 1987; Rippe et al., Mol. Cell Biol., 10:689-695, 1990) DEAE-dextran (Gopal, Mol. Cell Biol., 5:1188-1190, 1985), electroporation (Tur-Kaspa et al., Mol. Cell Biol., 6:716-718, 1986; Potter et al., Proc. Nat. Acad. Sci. USA, 81:7161-7165, 1984), direct microinjection (Harland and Weintraub, J. Cell Biol., 101:1094-1099, 1985), DNA-loaded liposomes (Nicolau and Sene, Biochim. Biophys. Acta, 721:185-190, 1982; Fraley et al., Proc. Natl. Acad. Sci. USA, 76:3348-3352, 1979; Felgner, Sci Am., 276(6): 102-6, 1997; Felgner, Hum Gene Ther., 7(15): 1791-3, 1996), cell sonication (Fechheimer et al., Proc. Natl. Acad. Sci. USA, 84:8463-8467, 1987), gene bombardment using high velocity microprojectiles (Yang et al., Proc. Natl. Acad. Sci USA, 87:9568-9572, 1990), and receptor-mediated transfection (Wu and Wu, J. Biol. Chem., 262:4429-4432, 1987; Wu and Wu, Biochemistry, 27:887-892, 1988; Wu and Wu, Adv. Drug Delivery Rev., 12:159-167, 1993).

A vector (or the antibody or fragment thereof or a nucleic acid as discussed herein) may be entrapped in a liposome. See, e.g., Ghosh and Bachhawat, In: Liver diseases, targeted diagnosis and therapy using specific receptors and ligands, Wu G, Wu C ed., New York: Marcel Dekker, pp. 87-104 (1991); Radler et al., Science, 275(5301): 810-814, (1997). Also contemplated are various commercial approaches involving “lipofection” technology. In some embodiments, the liposome may be complexed with a hemagglutinating virus (HVJ). This has been shown to facilitate fusion with the cell membrane and promote cell entry of liposome-encapsulated DNA (Kaneda et al., Science, 243:375-378, 1989). In some embodiments, the liposome is complexed or employed in conjunction with nuclear nonhistone chromosomal proteins (HMG-1) (Kato et al., J. Biol. Chem., 266:3361-3364, 1991). In some embodiments, the liposome is complexed or employed in conjunction with both HVJ and HMG-1. Such expression constructs have been successfully employed in transfer and expression of nucleic acid in vitro and in vivo. In some embodiments, a binding agent (e.g., an antibody) is included in the liposome to target the liposome to cells (such as an immune cell) expressing NKG2A and/or PD-L1 on their surface.

A cell may comprise one or more polynucleotides or one or more vectors, e.g., the cell is transformed or transfected with one or more polynucleotides encoding a binding agent (e.g., an antibody) or the one or more vectors comprising the one or more polynucleotides. In some embodiments, cells express a binding agent (e.g., an antibody) containing one or more, including six CDRs having at least 75%, 80%, 85%, 90%, 95%, 96%, 97%, 98%, or 99% identity to the CDRs of A2, A3, A11, A42 and/or P12 (see, e.g., Tables 1, 2, 3, 4 and/or 5). In some embodiments, the cell expresses a binding agent (e.g., an antibody) containing the VH and the VL comprising CDRs identical to those of A2, A3, A11, A42 and/or P12 (see, e.g., Tables 1, 2, 3, 4 and/or 5). The cells may be prokaryotic cells, such as Escherichia coli (see, e.g., Pluckthun et al., Methods Enzymol., 178:497-515, 1989), or eukaryotic cells, such as an animal cell (e.g., a myeloma cell, Chinese Hamster Ovary (CHO) cell, or hybridoma cell), yeast (e.g., Saccharomyces cerevisiae), an insect cell, or a plant cell (e.g., a tobacco, corn, soybean, or rice cell). Use of mammalian host cells may provide for translational modifications (e.g., glycosylation, truncation, lipidation, and phosphorylation) that may be desirable to confer optimal biological activity on recombinant expression products. Similarly, polypeptides (e.g., multispecific binding agents (e.g., bispecific antibodies) may be glycosylated or non-glycosylated and/or have been covalently modified to include one or more water soluble polymer attachments such as polyethylene glycol, polyoxyethylene glycol, or polypropylene glycol.

Methods for introducing DNA or RNA into host cells are well known and include transformation, transfection, electroporation, nuclear injection, or fusion with carriers such as liposomes, micelles, ghost cells, and protoplasts. Such host cells are useful for amplifying polynucleotides and also for expressing polypeptides encoded by the polynucleotides. In this regard, a process for the production of a binding agent (e.g., a multispecific binding agent or a bispecific antibody) may comprise culturing a host cell and isolating the binding agent. Transferring a naked DNA expression construct into cells can be accomplished using particle bombardment, which depends on the ability to accelerate DNA coated microprojectiles to a high velocity allowing them to pierce cell membranes and enter cells without killing them (Klein et al., Nature, 327:70-73, 1987). Several devices for accelerating small particles have been developed. One such device relies on a high voltage discharge to generate an electrical current, which in turn provides the motive force (Yang et al., Proc. Natl. Acad. Sci USA, 87:9568-9572, 1990). The microprojectiles used have consisted of biologically inert substances such as tungsten or gold beads. A host cell may be isolated and/or purified. A host cell also may be a cell transformed in vivo to cause transient or permanent expression of the polypeptide in vivo. A host cell may also be an isolated cell transformed ex vivo and introduced post-transformation, e.g., to produce the polypeptide in vivo for therapeutic purposes. The definition of host cell explicitly excludes a transgenic human being.

### 5.4. Methods of Making

Multispecific antibodies that bind to NKG2A and PD-L1 may be obtained by any suitable method. Each NKG2A and PD-L1 binding domain can be derived from an anti-NKG2A antibody or anti-PD-L1 antibody, which may be obtained by suitable methods, such as (but not limited to) immunization with whole cells comprising NKG2A or PD-L1 and collection of antibodies, recombinant, or screening libraries of antibodies or antibody fragments using NKG2A or PD-L1 extracellular domain epitopes. Monoclonal antibodies may be generated using a variety of known techniques (see, for example, Coligan et al. (eds.), Current Protocols in Immunology, 1:2.5.12.6.7 (John Wiley & Sons 1991); Monoclonal Antibodies, Hybridomas: A New Dimension in Biological Analyses, Plenum Press, Kennett, McKearn, and Bechtol (eds.) (1980); Antibodies: A Laboratory Manual, Harlow and Lane (eds.), Cold Spring Harbor Laboratory Press (1988); and Picksley et al., “Production of monoclonal antibodies against proteins expressed in E. coli,” in DNA Cloning 2: Expression Systems, 2nd Edition, Glover et al. (eds.), page 93 (Oxford University Press 1995)). One exemplary technique for generating monoclonal antibodies comprises immunizing an animal with a human NKG2A antigen or a human PD-L1 antigen and generating a hybridoma from spleen cells taken from the animal. A hybridoma may produce a monoclonal antibody or antibody fragment that binds NKG2A or PD-L1.

In a further embodiment, monoclonal antibodies or antibody fragments can be isolated from antibody phage libraries generated using the techniques described in, for example, Antibody Phage Display: Methods and Protocols, P. M. O'Brien and R. Aitken, eds, Humana Press, Totawa N.J., 2002. In principle, synthetic antibody clones are selected by screening phage libraries containing phage that display various fragments of antibody variable region (Fv) fused to phage coat protein. Such phage libraries are screened for against the desired antigen. Clones expressing Fv fragments capable of binding to the desired antigen are adsorbed to the antigen and thus separated from the non-binding clones in the library. The binding clones are then eluted from the antigen, and can be further enriched by additional cycles of antigen adsorption/elution.

Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described, for example, in Winter et al., Ann. Rev. Immunol., 12:433-455 (1994).

Repertoires of VH and VL genes can be separately cloned by polymerase chain reaction (PCR) and recombined randomly in phage libraries, which can then be searched for antigen-binding clones as described in Winter et al., supra. Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization as described by Griffiths et al., EMBO J, 12:725-734 (1993). Finally, naive libraries can also be made synthetically by cloning the unrearranged V-gene segments from stem cells, and using PCR primers containing random sequence to encode the highly variable CDR3 regions and to accomplish rearrangement in vitro as described, for example, by Hoogenboom and Winter, J. Mol. Biol., 227:381-388 (1992).

Screening of the libraries can be accomplished by various techniques known in the art. For example, NKG2A (e.g., an NKG2A polypeptide, fragment or epitope) or PD-L1 (e.g., a PD-L1 polypeptide, fragment or epitope) can be used to coat the wells of adsorption plates, expressed on host cells affixed to adsorption plates or used in cell sorting, or conjugated to biotin for capture with streptavidin-coated beads, or used in any other method for panning display libraries. The selection of antibodies with slow dissociation kinetics (e.g., good binding affinities) can be promoted by use of long washes and monovalent phage display as described in Bass et al., Proteins, 8:309-314 (1990) and in WO 92/09690, and a low coating density of antigen as described in Marks et al., Biotechnol., 10:779-783 (1992).

Multispecific binding agents (e.g., bispecific antibodies) can be obtained by designing a suitable antigen screening procedure to select for the phage clone of interest followed by construction of a full length binding agent (e.g., an antibody) clone using VH and/or VL sequences (e.g., the Fv sequences), or various CDR sequences from VH and VL sequences, from the phage clone of interest and suitable constant region (e.g., Fc) sequences described in Kabat et al., Sequences of Proteins of Immunological Interest, Fifth Edition, NIH Publication 91-3242, Bethesda MD (1991), vols. 1-3.

Likewise, human antibodies that bind NKG2A or PD-L1 may be generated by any of a number of techniques including, but not limited to, Epstein Barr Virus (EBV) transformation of human peripheral blood cells (e.g., containing B lymphocytes), in vitro immunization of human B cells, fusion of spleen cells from immunized transgenic mice carrying inserted human immunoglobulin genes, isolation from human immunoglobulin V region phage libraries, or other procedures as known in the art and based on the disclosure herein. Methods for obtaining human antibodies from transgenic animals are further described, for example, in Bruggemann et al., Curr. Opin. Biotechnol., 8:455 58, 1997; Jakobovits et al., Ann. N. Y. Acad. Sci., 764:525 35, 1995; Green et al., Nature Genet., 7:13-21, 1994; Lonberg et al., Nature, 368:856-859, 1994; Taylor et al., Int. Immun. 6:579-591, 1994; and U.S. Pat. No. 5,877,397.

For example, human antibodies that bind NKG2A or PD-L1 may be obtained from transgenic animals that have been engineered to produce specific human antibodies in response to antigenic challenge. For example, WO 98/24893 discloses transgenic animals having a human Ig locus, wherein the animals do not produce functional endogenous immunoglobulins due to the inactivation of endogenous heavy and light chain loci. Transgenic non-primate mammalian hosts capable of mounting an immune response to an immunogen, wherein the antibodies have primate constant and/or variable regions, and wherein the endogenous immunoglobulin encoding loci are substituted or inactivated, also have been described. WO 96/30498 discloses the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy chains and express an exogenous immunoglobulin locus comprising one or more xenogeneic constant regions. Using a transgenic animal, such as a transgenic animal described herein, an immune response can be produced to a selected antigenic molecule, and antibody producing cells can be removed from the animal and used to produce hybridomas that secrete human-derived monoclonal antibodies. Immunization protocols, adjuvants, and the like are known in the art, and are used in immunization of, for example, a transgenic mouse as described in WO 96/33735. The monoclonal antibodies can be tested for the ability to inhibit or neutralize the biological activity or physiological effect of the corresponding protein.

In some embodiments, a binding agent described herein comprises a non-antibody protein scaffold. Non-limiting examples of such a non-antibody protein scaffold include a fibronectin scaffold, an anticalin, an adnectin, an affibody, a DARPin, a fynomer, an affitin, an affilin, an avimer, a cysteine-rich knottin peptide, or an engineered Kunitz-type inhibitor. Methods for generating such non-antibody protein scaffolds are well known in the art, any one of which can be used to generate an NKG2A and/or PD-L1 binding agent comprising a non-antibody protein scaffold (see, e.g., Simeon and Chen, Protein Cell, 9(1):3-14 (2018); Yang et al., Annu Rev Anal Chem (Palo Alto Calif). 10(1):293-320 (2017)).

A variety of methods for producing antibodies from polynucleotides are generally well-known. For example, basic molecular biology procedures are described by Maniatis et al., Molecular Cloning, A Laboratory Manual, 2nd ed., Cold Spring Harbor Laboratory, New York, 1989 (see also Maniatis et al., 3rd ed., Cold Spring Harbor Laboratory, New York, 2001). Additionally, numerous publications describe techniques suitable for the preparation of antibodies by manipulation of DNA, creation of expression vectors, and transformation and culture of appropriate cells (see, e.g., Mountain and Adair, Chapter 1 in Biotechnology and Genetic Engineering Reviews, Tombs ed., Intercept, Andover, UK, 1992; and Current Protocols in Molecular Biology, Ausubel ed., Wiley Interscience, New York, 1999).

A binding agent (e.g., an antibody) is produced using any suitable method, e.g., isolated from an immunized animal, recombinantly or synthetically generated, or genetically-engineered, including as described above. Antibody fragments derived from an antibody are obtained by, e.g., proteolytic hydrolysis of an antibody. For example, papain or pepsin digestion of whole antibodies yields a 5S fragment termed F(ab′)₂ or two monovalent Fab fragments and an Fc fragment, respectively. F(ab)₂ can be further cleaved using a thiol reducing agent to produce 3.5S Fab monovalent fragments. Methods of generating antibody fragments are further described in, for example, Edelman et al., Methods in Enzymology, 1:422 Academic Press (1967); Nisonoff et al., Arch. Biochem. Biophys., 89:230-244, 1960; Porter, Biochem. J., 73:119-127, 1959; U.S. Pat. No. 4,331,647; and by Andrews, S. M. and Titus, J. A. in Current Protocols in Immunology (Coligan et al., eds), John Wiley & Sons, New York (2003), pages 2.8.1 2.8.10 and 2.10A.12.10A.5.

A binding agent (e.g., an antibody) can be genetically engineered. For example, a binding agent (e.g., an antibody) comprises, for example, a variable region domain generated by recombinant DNA engineering techniques. In this regard, a variable region is optionally modified by insertions, deletions, or changes in the amino acid sequence of the antibody to produce an antibody of interest, including as described above. Polynucleotides encoding CDRs of interest are prepared, for example, by using polymerase chain reaction to synthesize variable regions using mRNA of antibody producing cells as a template (see, for example, Courtenay Luck, “Genetic Manipulation of Monoclonal Antibodies,” in Monoclonal Antibodies: Production, Engineering and Clinical Application, Ritter et al. (eds.), page 166 (Cambridge University Press 1995); Ward et al., “Genetic Manipulation and Expression of Antibodies,” in Monoclonal Antibodies: Principles and Applications, Birch et al., (eds.), page 137 (Wiley Liss, Inc. 1995); and Larrick et al., Methods: A Companion to Methods in Enzymology, 2:106-110, 1991). Current antibody manipulation techniques allow construction of engineered variable region domains containing at least one CDR and, optionally, one or more framework amino acids from a first antibody and the remainder of the variable region domain from a second antibody. Such techniques are used, e.g., to humanize an antibody or to improve its affinity for a binding target. Exemplary methods for generating humanized antibodies are described in sections above too.

### 5.5. Pharmaceutical Compositions

In one aspect, the present disclosure further provides a composition, such as a pharmaceutical composition, comprising at least one of the following: a binding agent provided herein (e.g., a multispecific binding agent, or one antibody or antigen-binding fragment thereof of the present disclosure), a nucleic acid provided herein, a vector provided herein, or a cell provided herein. In some embodiments, a pharmaceutical composition comprises a therapeutically effective amount of a binding agent provided herein (e.g., a multispecific binding agent or a bispecific antibody or antigen-binding fragment thereof provided herein) and a pharmaceutically acceptable excipient. In some embodiments, a pharmaceutical composition comprises a therapeutically effective amount of a nucleic acid provided herein (such as a nucleic acid encoding a bispecific antibody or antigen-binding fragment thereof provided herein) and a pharmaceutically acceptable excipient. In some embodiments, a pharmaceutical composition comprises a therapeutically effective amount of a vector provided herein (such as a vector comprising a nucleic acid as disclosed herein and expressing a binding agent as disclosed herein) and a pharmaceutically acceptable excipient. In some embodiments, a pharmaceutical composition comprises a therapeutically effective amount of a cell provided herein (such as a cell comprising a nucleic acid encoding a bispecific antibody or antigen-binding fragment thereof provided herein and/or expressing a bispecific antibody or antigen-binding fragment thereof provided herein) and a pharmaceutically acceptable excipient.

In some embodiments, pharmaceutical compositions provided herein are prepared for storage by mixing the binding agents, nucleic acids, vectors, or cells provided herein having the desired degree of purity with optional physiologically acceptable excipients (see, e.g., Remington, Remington's Pharmaceutical Sciences (18th ed. 1980)) in the form of aqueous solutions or lyophilized or other dried forms.

The binding agents, nucleic acids, vectors, or cells of the present disclosure may be formulated in any suitable form for delivery to a target cell/tissue, e.g., as microcapsules or macroemulsions (Remington, supra; Park et al., 2005, Molecules 10:146-61; Malik et al., 2007, Curr. Drug. Deliv. 4:141-51), as sustained release formulations (Putney and Burke, 1998, Nature Biotechnol. 16:153-57), or in liposomes (Maclean et al., 1997, Int. J. Oncol. 11:325-32; Kontermann, 2006, Curr. Opin. Mol. Ther. 8:39-45).

The binding agents, nucleic acids, vectors, or cells provided herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles, and nanocapsules) or in macroemulsions. Such techniques are disclosed, for example, in Remington, supra.

Various compositions and delivery systems are known and can be used with the binding agents, nucleic acids, vectors or cells as described herein, including, but not limited to, encapsulation in liposomes, microparticles, microcapsules, recombinant cells capable of expressing the antibody or antigen-binding fragment thereof, receptor-mediated endocytosis (see, e.g., Wu and Wu, 1987, J. Biol. Chem. 262:4429-32), construction of a nucleic acid as part of a retroviral or other vector, etc. In another embodiment, a composition can be provided as a controlled release or sustained release system. In one embodiment, a pump may be used to achieve controlled or sustained release (see, e.g., Langer, supra; Sefton, 1987, Crit. Ref. Biomed. Eng. 14:201-40; Buchwald et al., 1980, Surgery 88:507-16; and Saudek et al., 1989, N. Engl. J. Med. 321:569-74). In another embodiment, polymeric materials can be used to achieve controlled or sustained release of a prophylactic or therapeutic agent (e.g., an antibody or antigen-binding fragment thereof as described herein) or a composition provided herein (see, e.g., Medical Applications of Controlled Release (Langer and Wise eds., 1974); Controlled Drug Bioavailability, Drug Product Design and Performance (Smolen and Ball eds., 1984); Ranger and Peppas, 1983, J. Macromol. Sci. Rev. Macromol. Chem. 23:61-126; Levy et al., 1985, Science 228:190-92; During et al., 1989, Ann. Neurol. 25:351-56; Howard et al., 1989, J. Neurosurg. 71:105-12; U.S. Pat. Nos. 5,679,377; 5,916,597; 5,912,015; 5,989,463; and 5,128,326; WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable.

In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, 1990, Science 249:1527-33. Any technique known to one of skill in the art can be used to produce sustained release formulations comprising one or more antibody or antigen-binding fragment thereof as described herein (see, e.g., U.S. Pat. No. 4,526,938, WO 91/05548 and WO 96/20698, Ning et al., 1996, Radiotherapy & Oncology 39:179-89; Song et al., 1995, PDA J. of Pharma. Sci. & Tech. 50:372-97; Cleek et al., 1997, Pro. Int'l. Symp. Control. Rel. Bioact. Mater. 24:853-54; and Lam et al., 1997, Proc. Int'l. Symp. Control Rel. Bioact. Mater. 24:759-60).

### 5.6. Methods of Uses

In another aspect, provided herein are methods for using the binding agents such as the multispecific binding agents or the composition provided herein. In a further aspect, provided herein are multispecific binding agents or compositions as disclosed herein for use as a medicament. In yet a further aspect, provided herein are multispecific binding agents or compositions as disclosed herein for use in the treatment of a disease or condition, such as those disclosed herein. In one aspect, provided herein are multispecific binding agents or compositions as disclosed herein for use in a method as disclosed herein.

In some embodiments, the multispecific binding agents bind to NKG2A. Additionally or alternatively, the multispecific binding agents bind to a complex comprising NKG2A and CD94, or a complex comprising the extracellular domains of NKG2A and CD94. In various embodiments, the NKG2A is a human NKG2A. In further embodiments, the NKG2A also refers to a cyno NKG2A. In other embodiments, the multispecific binding agents do not bind to cyno NKG2A or a complex comprising a cyno NKG2A. Additionally or alternatively, the multispecific binding agent does not bind to a complex comprising NKG2C and CD94, or a complex comprising the extraceullular domains of NKG2C and CD94.

In some embodiments, provided herein is a method of inhibiting interaction between NKG2A (for example, expressed on and/or in a first cell) and HLA-E (for example, expressed on and/or in a second cell), comprising contacting NKG2A (for example, the first cell expressing NKG2A) with a multispecific binding agent (e.g., a multispecific antibody or fragment thereof) provided herein. In some embodiments, provided herein is a use of the multispecific binding agent provided herein for inhibiting interaction between NKG2A (for example, expressed on and/or in a first cell) and HLA-E (for example, expressed on and/or in a second cell), wherein the use comprises contacting NKG2A (for example, the first cell expressing NKG2A) with a multispecific binding agent (e.g., the antibody or fragment thereof) provided herein.

In a further aspect, provided herein is a method of inhibiting interaction between a complex comprising NKG2A and CD94 or an extracellular domain of each thereof (for example, expressed on and/or in a first cell) and HLA-E (for example, expressed on and/or in a second cell), comprising contacting the complex comprising NKG2A and CD94 (for example, the first cell expressing NKG2A and CD94) with a multispecific binding agent (e.g., a multispecific antibody or fragment thereof) provided herein. In some embodiments, provided herein is a use of the multispecific binding agent provided herein for inhibiting interaction between a complex comprising NKG2A and CD94 or an extracellular domain of each thereof (for example, expressed on and/or in a first cell) and HLA-E (for example, expressed on and/or in a second cell), wherein the use comprises contacting the complex comprising NKG2A and CD94 (for example, the first cell expressing NKG2A and CD94) with a multispecific binding agent (e.g., the antibody or fragment thereof) provided herein.

In some embodiments, the method or use as disclosed herein does not inhibit interaction between NKG2C (or a complex comprising NKG2C and CD94 or extracellular domain of each thereof) and HLA-E. In other embodiments, the method or use as disclosed herein inhibits the interaction between NKG2C (or a complex comprising NKG2C and CD94 or an extracellular domain of each thereof) and HLA-E, but the inhibition is significantly less (for example, at least about 10% less, or at least about 20% less, or at least about 30% less, or at least about 40% less, or at least about 50% less, or at least about 60% less, or at least about 70% less, or at least about 80% less, or at least about 90% less, or at least about 95% less, or at least about 99% less) than that of a benchmark anti-NKG2A antibody.

As used herein, the term “inhibit” means decrease or reduce. For example, in some embodiments, the binding agent provided herein inhibits the interaction between NKG2A (or a complex comprising NKG2A and CD94 or extracellular domain of each thereof) and HLA-E by 10%-99%. In other embodiments, the binding agent provided herein inhibits the interaction between NKG2A (or a complex comprising NKG2A and CD94 or extracellular domain of each thereof) and HLA-E 100% (e.g., completely abolish the interaction as measured by an assay). In some embodiments, the binding agent provided herein inhibits the interaction between NKG2A (or a complex comprising NKG2A and CD94 or extracellular domain of each thereof) and HLA-E by at least 10%. In some embodiments, the binding agent provided herein inhibits the interaction between NKG2A (or a complex comprising NKG2A and CD94 or extracellular domain of each thereof) and HLA-E by at least 20%. In some embodiments, the binding agent provided herein inhibits the interaction between NKG2A (or a complex comprising NKG2A and CD94 or extracellular domain of each thereof) and HLA-E by at least 30%. In some embodiments, the binding agent provided herein inhibits the interaction between NKG2A (or a complex comprising NKG2A and CD94 or extracellular domain of each thereof) and HLA-E by at least 40%. In some embodiments, the binding agent provided herein inhibits the interaction between NKG2A (or a complex comprising NKG2A and CD94 or extracellular domain of each thereof) and HLA-E by at least 50%. In some embodiments, the binding agent provided herein inhibits the interaction between NKG2A (or a complex comprising NKG2A and CD94) and HLA-E by at least 60%. In some embodiments, the binding agent provided herein inhibits the interaction between NKG2A (or a complex comprising NKG2A and CD94 or extracellular domain of each thereof) and HLA-E by at least 70%. In some embodiments, the binding agent provided herein inhibits the interaction between NKG2A (or a complex comprising NKG2A and CD94 or extracellular domain of each thereof) and HLA-E by at least 80%. In some embodiments, the binding agent provided herein inhibits the interaction between NKG2A (or a complex comprising NKG2A and CD94 or extracellular domain of each thereof) and HLA-E by at least 90%. In some embodiments, the binding agent provided herein inhibits the interaction between NKG2A (or a complex comprising NKG2A and CD94 or extracellular domain of each thereof) and HLA-E by about 10%-90%. In some embodiments, the binding agent provided herein inhibits the interaction between NKG2A (or a complex comprising NKG2A and CD94 or extracellular domain of each thereof) and HLA-E by about 20%-80%. In some embodiments, the binding agent provided herein inhibits the interaction between NKG2A (or a complex comprising NKG2A and CD94 or extracellular domain of each thereof) and HLA-E by about 30%-70%. In some embodiments, the binding agent provided herein inhibits the interaction between NKG2A (or a complex comprising NKG2A and CD94 or extracellular domain of each thereof) and HLA-E by about 40%-60%. In some embodiments, the NKG2A (or a complex comprising NKG2A and CD94 or extracellular domain of each thereof) and the HLA-E are expressed on different cells. In some embodiments, the NKG2A cell is expressed on a first cell, such as an immune cell. In some embodiments, the immune cell is an NK cell. In some embodiments, the immune cell is a T cell. In some embodiments, the T cell is a cytotoxic T cell such as a CD8⁺ T cell.

An immune cell is a cell in immune system and can be a cell of lymphoid lineage. Non-limiting examples of cells of lymphoid lineage include neutrophils, eosinophils, basophils, mast cells, monocytes, macrophages, dendritic cells, natural killer (NK) cells, and lymphocytes (B cells and T cells). T cells are a type of lymphocytes and can be characterized by expressing T cell receptors (TCRs). T cells play a central role in the adaptive immune response. T cell subtypes have a variety of important functions in controlling and shaping the immune response. For example, cytotoxic T cells (also called cytotoxic T lymphocyte and killer T cell) are T lymphocytes that kill certain cells, e.g., cancer cells, cells that are infected by intracellular pathogens (such as viruses or bacteria), or cells that are damaged in other ways. Most cytotoxic T cells express T-cell receptors (TCRs) that can recognize a specific antigen. CD8⁺ T cells are a subpopulation of MHC class I-restricted T cell and are mediators of adaptive immunity, which are important for killing cancerous or virally infected cells. NK cells are a type of cytotoxic lymphocytes critical to the innate immune system that belong to the family of innate lymphoid cells (ILC). In some embodiments, NK cells can be identified by the presence of CD56 and the absence of CD3 (CD56+, CD3−). NK cells have the ability to recognize and kill stressed cells in the absence of antibodies and MHC, allowing for a much faster immune reaction.

In some embodiments, the HLA-E is expressed on a second cell, such as a cancer or tumor cell.

In some embodiments, provided herein is a method of inhibiting interaction between PD-1 (for example, expressed on and/or in a first cell) and PD-L1 (for example, expressed on and/or in a second cell), comprising contacting PD-L1 (and/or the second cell expressing the PD-L1) with a binding agent (e.g., a multispecific binding agent, a multispecific antibody or fragment thereof) provided herein. In some embodiments, provided herein is a use of the binding agent (e.g., a multispecific binding agent, a multispecific antibody or fragment thereof) provided herein for inhibiting interaction between PD-1 (for example, expressed on and/or in a first cell) and PD-L1 (for example, expressed on and/or in a second cell), wherein the use comprises contacting PD-L1 (and/or the second cell expressing the PD-L1) with a binding agent (e.g., a multispecific binding agent, the multispecific antibody or fragment thereof) provided herein.

As used herein, the term “inhibit” means decrease or reduce. For example, in some embodiments, the binding agent provided herein inhibits the interaction between PD-L1 and PD-1 by 10%-99%. In other embodiments, the binding agent provided herein inhibits the interaction between PD-L1 and PD-1 by 100% (e.g., completely abolish the interaction as measured by an assay). In some embodiments, the binding agent provided herein inhibits the interaction between PD-L1 and PD-1 by at least 10%. In some embodiments, the binding agent provided herein inhibits the interaction between PD-L1 and PD-1 by at least 20%. In some embodiments, the binding agent provided herein inhibits the interaction between PD-L1 and PD-1 by at least 30%. In some embodiments, the binding agent provided herein inhibits the interaction between PD-L1 and PD-1 by at least 40%. In some embodiments, the binding agent provided herein inhibits the interaction between PD-L1 and PD-1 by at least 50%. In some embodiments, the binding agent provided herein inhibits the interaction between PD-L1 and PD-1 by at least 60%. In some embodiments, the binding agent provided herein inhibits the interaction between PD-L1 and PD-1 by at least 70%. In some embodiments, the binding agent provided herein inhibits the interaction between PD-L1 and PD-1 by at least 80%. In some embodiments, the binding agent provided herein inhibits the interaction between PD-L1 and PD-1 by at least 90%. In some embodiments, the binding agent provided herein inhibits the interaction between PD-L1 and PD-1 by about 10%-90%. In some embodiments, the binding agent provided herein inhibits the interaction between PD-L1 and PD-1 by about 20%-80%. In some embodiments, the binding agent provided herein inhibits the interaction between PD-L1 and PD-1 by about 30%-70%. In some embodiments, the binding agent provided herein inhibits the interaction between PD-L1 and PD-1 by about 40%-60%. In some embodiments, the PD-1 cell is expressed on a cell, such as an immune cell. In some embodiments, the immune cell is a T cell. In some embodiments, the T cell is a cytotoxic T cell such as a CD8⁺ T cell. In some embodiments, the immune cell is an NK cell. In some embodiments, PD-1 and PD-L1 are expressed on different cells. In some embodiments, the first cell expressing PD-1 is an immune cell, such as an NK cell or a T cell (for example a cytotoxic T cell and/or a CD8⁺ T cell). Additionally or alternatively, the second cell expressing PD-L1 is a cancer or tumor cell.

In some embodiments, provided herein is a method of inhibiting interaction between NKG2A (or a complex comprising NKG2A and CD94 or an extracellular domain of each thereof) (for example, expressed on and/or in a first cell) and HLA-E (for example, expressed on and/or in a second cell) and inhibiting interaction between PD-1 (for example, expressed on and/or in a third cell) and PD-L1 (for example, expressed on and/or in a fourth cell), comprising contacting NKG2A (or a complex comprising NKG2A and CD94 or extracellular domain of each thereof, or the first cell expressing NKG2A or the complex) and PD-L1 (and/or the fourth cell expressing the PD-L1) with a binding agent (e.g., a multispecific binding agent, a multispecific antibody or fragment thereof) provided herein. In some embodiments, provided herein is a use of the binding agent (e.g., a multispecific binding agent, a multispecific antibody or fragment thereof) provided herein for inhibiting interaction between NKG2A (or a complex comprising NKG2A and CD94 or an extracellular domain of each thereof) (for example, expressed on and/or in a first cell) and HLA-E (for example, expressed on and/or in a second cell) and inhibiting interaction between PD-1 (for example, expressed on and/or in a third cell) and PD-L1 (for example, expressed on and/or in a fourth cell), wherein the use comprises contacting NKG2A (or a complex comprising NKG2A and CD94 or extracellular domain of each thereof, or the first cell expressing NKG2A or the complex) and PD-L1 (and/or the fourth cell expressing the PD-L1) with a binding agent (e.g., a multispecific binding agent, a multispecific antibody or fragment thereof) provided herein. In some embodiments, NKG2A (or the complex comprising NKG2A and CD94) and PD-1 are expressed on and/or in the same cell (e.g., the first cell and the third cell are the same cell), such as an immune cell (e.g., an NK cell or a T cell). Additionally or alternatively, HLA-E and PD-L1 are expressed on and/or in the same cell (e.g., the second cell and the fourth cell are the same cell), such as a cancer or tumor cell.

In some embodiments, provided herein is a method of preventing (e.g., reducing or decreasing, including but not limited to abolishing) or inhibiting suppression of an immune cell, such as a tumor/cancer associated immune suppression. The method comprises (i) inhibiting interaction between NKG2A (or a complex comprising NKG2A and CD94 or extracellular domain of each thereof) (for example, expressed on and/or in a first cell) and HLA-E (for example, expressed on and/or in a second cell) and (ii) inhibiting interaction between PD-1 (for example, expressed on and/or in a third cell) and PD-L1 (for example, expressed on and/or in a fourth cell). Also provided herein is a use of a binding agent as disclosed herein in preventing (e.g., reducing or decreasing, including but not limited to abolishing) or inhibiting suppression of an immune cell, such as a tumor/cancer associated immune suppression. The use comprises (i) inhibiting interaction between NKG2A (or a complex comprising NKG2A and CD94 or extracellular domain of each thereof) (for example, expressed on and/or in a first cell) and HLA-E (for example, expressed on and/or in a second cell) and (ii) inhibiting interaction between PD-1 (for example, expressed on and/or in a third cell) and PD-L1 (for example, expressed on and/or in a fourth cell). In some embodiments, the method or the use comprises contacting NKG2A (or a complex comprising NKG2A and CD94 or extracellular domain of each thereof, or the first cell expressing NKG2A or the complex) and PD-L1 (and/or the fourth cell expressing the PD-L1) with a binding agent (e.g., a multispecific binding agent, a multispecific antibody or fragment thereof) provided herein. In some embodiments, NKG2A (or the complex comprising NKG2A and CD94) and PD-1 are expressed on and/or in the same cell (e.g., the first cell and the third cell are the same cell), such as an immune cell (e.g., an NK cell or a T cell). Additionally or alternatively, HLA-E and PD-L1 are expressed on and/or in the same cell (e.g., the second cell and the fourth cell are the same cell), such as a cancer or tumor cell.

Provided herein among others are binding agents comprising (i) a first means for inhibiting interaction between PD-1 (for example, expressed on and/or in a third cell, or alternatively in a subject in need thereof) and PD-L1 (for example, expressed on and/or in a fourth cell, or alternatively in the subject), and (ii) a second means for inhibiting interaction between NKG2A (or a complex comprising NKG2A and CD94, or an extracellular domain of each thereof) (for example, expressed on and/or in a first cell, or alternatively in a subject in need thereof) and HLA-E (for example, expressed on and/or in a second cell, or alternatively in the subject). In some embodiments, NKG2A (or the complex comprising NKG2A and CD94) and PD-1 are expressed on and/or in the same cell (e.g., the first cell and the third cell are the same cell), such as an immune cell (e.g., an NK cell or a T cell). Additionally or alternatively, HLA-E and PD-L1 are expressed on and/or in the same cell (e.g., the second cell and the fourth cell are the same cell), such as a cancer or tumor cell. In some embodiments, the binding agents are bispecific antibodies comprising a binding domain that binds to PD-L1 and a binding domain that binds to NKG2A. Nucleic acid encoding the present binding agents, pharmaceutical compositions comprising the present binding agents or nucleic acids, and uses of the binding agents and pharmaceutical compositions are also provided herein.

In some embodiments, the NKG2A is a human NKG2A. Additionally or alternatively, the second means inhibits the interaction between a cyno NKG2A (or a complex comprising the cyno NKG2A and a CD94 (e.g., cyno or human), or an extracellular domain of each thereof) and an HLA-E (e.g., cyno or human). In some embodiments, the second means does not significantly inhibit interactions between NKG2C (or a complex comprising NKG2C and CD94 or extracellular domain of each thereof) and HLA-E. In some embodiments, the NKG2C is a human NKG2C. Additionally or alternatively, the second means does not significantly inhibit the interaction between a cyno NKG2C (or a complex comprising the cyno NKG2C and a CD94 (e.g., cyno or human), or an extracellular domain of each thereof) and an HLA-E (e.g., cyno or human).

Additionally provided herein is a method of treating a disease, disorder, or condition (such as those disclosed herein) in a subject in need thereof. In some embodiments, the method comprises administering to the subject an effective amount of (i) a first means for inhibiting interaction between PD-1 and PD-L1, and (ii) a second means for inhibiting interaction between NKG2A (or a complex comprising NKG2A and CD94 or an extracellular domain of each thereof) and HLA-E. Also provided herein is a use of the multispecific binding agent provided herein for treating a disease, disorder, or condition (such as those disclosed herein) in a subject in need thereof. In some embodiments, the use comprises administering to the subject an effective amount of (i) a first means for inhibiting interaction between PD-1 and PD-L1, and (ii) a second means for inhibiting interaction between NKG2A (or a complex comprising NKG2A and CD94 or an extracellular domain of each thereof) and HLA-E. In further embodiments, the method and use comprise administering to the subject an effective amount of a multispecific binding agent as disclosed herein.

In some embodiments, the NKG2A is a human NKG2A. Additionally or alternatively, the second means inhibits the interaction between a cyno NKG2A (or a complex comprising the cyno NKG2A and a CD94 (e.g., cyno or human), or an extracellular domain of each thereof) and an HLA-E (e.g., cyno or human). In some embodiments, the second means does not significantly inhibit interactions between NKG2C (or a complex comprising NKG2C and CD94 or extracellular domain of each thereof) and HLA-E. In some embodiments, the NKG2C is a human NKG2C. Additionally or alternatively, the second means does not significantly inhibit the interaction between a cyno NKG2C (or a complex comprising the cyno NKG2C and a CD94 (e.g., cyno or human), or an extracellular domain of each thereof) and an HLA-E (e.g., cyno or human).

In some embodiments, a method as disclosed herein is an in vitro method or an ex vivo method. In other embodiments, a method as disclosed herein is an in vivo method. In some embodiments, a use as disclosed herein is an in vitro use or an ex vivo use. In other embodiments, a use as disclosed herein is an in vivo use. In some embodiments, an in vivo method or an in vivo use as disclosed herein comprises administering a binding agent as disclosed herein to a subject having a cell expressing either or both of HLA-E and PD-L1, such as a tumor cell expressing either or both of HLA-E and PD-L1.

In other embodiments, provided herein is a method of preventing or inhibiting suppression of an immune cell, e.g., a suppression mediated by the interaction between NKG2A (or a complex comprising NKG2A and CD94 or extracellular domain of each thereof) expressed on the immune cell with HLA-E (e.g., expressed on a cancer or tumor cell), and/or a suppression mediated by the interaction between PD-1 (e.g., expressed on the immune cell) and PD-L1 (e.g., expressed on a cancer or tumor cell). In yet other embodiments, provided herein is a method of activating a response mediated by an immune cell, e.g., an anti-tumor response. In some embodiments, the immune cell suppression is a tumor/cancer associated immune cell suppression, such as in a tumor microenvironment. In some embodiments, the method comprises contacting the immune cell with a binding agent (e.g., a multispecific antibody or fragment thereof) provided herein. In some embodiments, provided herein is a use of the binding agent provided herein for preventing suppression of an immune cell or activating a response mediated by an immune cell. In some embodiments, the immune cell is an NK cell. In some embodiments, the immune cell is a T cell. In some embodiments, the T cell is a cytotoxic T cell such as a CD8⁺ T cell. In some embodiments, the immune cell expresses NKG2A. Additionally or alternatively, the immune cell expresses PD-1. In some embodiments, the immune cell mediates an anti-cancer/tumor response. In further embodiments, the cancer or tumor cell expresses PD-L1. Additionally or alternatively, the cancer or tumor cell expresses HLA-E.

In some embodiments, binding agents (e.g., multispecific binding agents, such as bispecific antibodies) described herein are useful in compositions and in methods of treating a disease or disorder. Thus, in some embodiments, provided herein is a method for treating a disease or disorder in a subject comprising administering the binding agent or the pharmaceutical composition provided herein to the subject. In other embodiments, provided herein is a use of the binding agent or pharmaceutical composition provided herein for treating a disease or disorder in a subject. In other embodiments, provided herein is a binding agent or pharmaceutical composition provided herein for use in the manufacture of a medicament for the treatment of a disease or disorder.

In some embodiments, the treatment provided herein includes alleviating one or more symptoms associated with a disease or disorder (e.g., a cancer or a tumor or an autoimmune or inflammatory disease or disorder).

Thus, in some embodiments, provided herein is a method for alleviating one or more symptoms associated with a cancer or a tumor in a subject comprising administering to the subject a binding agent (e.g., a multispecific binding agent, such as a bispecific antibody) described herein or a pharmaceutical composition described herein. In some embodiments, provided herein is a binding agent (e.g., a multispecific binding agent, such as a bispecific antibody) described herein or a pharmaceutical composition described herein for use in alleviating one or more symptoms associated with a cancer or a tumor in a subject. In some embodiments, provided herein is a use of a binding agent (e.g., a multispecific binding agent, such as a bispecific antibody) described herein or a pharmaceutical composition described herein in the manufacture of a medicament for alleviating one or more symptoms associated with a cancer or a tumor in a subject.

In some embodiments, the treatment provided herein includes decreasing tumor size in a subject with a tumor. Thus, in some embodiments, described herein is a method for decreasing tumor size in a subject with a tumor comprising administering to the subject a binding agent (e.g., a multispecific binding agent, such as a bispecific antibody) described herein or a pharmaceutical composition described herein. In some embodiments, provided herein is a binding agent (e.g., a multispecific binding agent, such as a bispecific antibody) described herein or a pharmaceutical composition described herein for use in decreasing tumor size in a subject with a tumor. In some embodiments, provided herein is a use of a binding agent (e.g., a multispecific binding agent, such as a bispecific antibody) described herein or a pharmaceutical composition described herein in the manufacture of a medicament for decreasing tumor size in a subject with a tumor.

In some embodiments, described herein is a method for enhancing tumor cell removal in a subject with a tumor comprising administering to the subject a binding agent (e.g., a multispecific binding agent, such as a bispecific antibody) described herein or a pharmaceutical composition described herein. In some embodiments, provided herein is a binding agent (e.g., a multispecific binding agent, such as a bispecific antibody) described herein or a pharmaceutical composition described herein for use in enhancing tumor cell removal in a subject with a tumor. In some embodiments, provided herein is a use of a binding agent (e.g., a multispecific binding agent, such as a bispecific antibody) described herein or a pharmaceutical composition described herein in the manufacture of a medicament for enhancing tumor cell removal in a subject with a tumor.

“Enhancing” tumor cell removal includes, yet does not require a 100% enhancement of removal. Any enhancement in the rate of removal is contemplated. Similarly, “modulating” tumor growth refers to reducing the size of the tumor, slowing tumor growth, or inhibiting an increase in the size of an existing tumor. Complete abolition of a tumor is included but not required; any decrease in tumor size or slowing of tumor growth constitutes a beneficial biological effect in a subject. In this regard, tumor cell removal may be enhanced by, for example, at least about 5%, at least about 10% or at least about 20% compared to levels of removal observed in the absence of the method (e.g., in a biologically-matched control subject or specimen that is not exposed to the agent of the method). The effect is detected by, for example, a reduction in tumor size, a decrease or maintenance of the levels of tumor markers, or reduction or maintenance of a tumor cell population. In some embodiments, removal of tumor cells is enhanced by, for example, at least about 30%, at least about 40%, at least about 50%, at least about 60%, at least about 70%, at least about 80%, at least about 90%, or more (about 100%) compared to the removal of tumor cells in the absence of a binding agent (e.g., a multispecific binding agent, such as a bispecific antibody) or a pharmaceutical composition of the method.

A method of modulating (e.g., inhibiting, reducing, preventing) tumor growth in a subject also is provided. For example, the method comprises administering to the subject a composition comprising a binding agent (e.g., a multispecific binding agent, such as a bispecific antibody) or another composition as disclosed herein in an amount effective to modulate tumor growth in the subject.

In some embodiments, provided herein is a method of forming a first complex comprising an NKG2A expressing cell, a binding agent, and an PD-L1 expressing cell. The method comprises contacting a first cell expressing NKG2A or a second complex comprising NKG2A and CD94 (also referred to herein as an NKG2A expressing cell), and a second cell expressing PD-L1 (also referred to herein as a PD-L1 expressing cell) with a binding agent (e.g., a multispecific binding agent, a multispecific antibody or fragment thereof) provided herein. In some embodiments, provided herein is a use of a binding agent provided herein for forming a first complex comprising an NKG2A expressing cell, the binding agent, and an PD-L1 expressing cell. The use comprises contacting a first cell expressing NKG2A or a second complex comprising NKG2A and CD94 (also referred to herein as an NKG2A expressing cell), and a second cell expressing PD-L1 (also referred to herein as a PD-L1 expressing cell) with a binding agent (e.g., a multispecific binding agent, a multispecific antibody or fragment thereof) provided herein. In some embodiments, the method or use is in vitro. In some embodiments, the method or use is ex vivo. In some embodiments, the method or use is in vivo. In some embodiments, the NKG2A expressing cell is an immune cell. In further embodiments, the NKG2A expressing cell is a T cell. Additionally or alternatively, the NKG2A expressing cell is an NK cell. In some embodiments, the PD-L1 expressing cell is a cancer or a tumor cell. In some embodiments, the NKG2A expressing cell is an NK cell and the PD-L1 expressing cell is a cancer or a tumor cell.

In some embodiments, provided herein is a method comprising contacting NKG2A (or a complex comprising NKG2A and CD94, or a first cell expressing NKG2A or the complex) and PD-L1 (or a second cell expressing the PD-L1) with a binding agent (e.g., a multispecific binding agent, a multispecific antibody or fragment thereof) provided herein. In some embodiments, provided herein is a use of a binding agent provided herein comprising contacting NKG2A (or a complex comprising NKG2A and CD94, or a first cell expressing NKG2A or the complex) and PD-L1 (or a second cell expressing the PD-L1) with the binding agent (e.g., a multispecific binding agent, a multispecific antibody or fragment thereof) provided herein. In some embodiments, the method or use is in vitro. In some embodiments, the method or use is ex vivo. In some embodiments, the method or use is in vivo. In some embodiments, the NKG2A expressing cell is an immune cell. In further embodiments, the NKG2A expressing cell is a T cell. Additionally or alternatively, the NKG2A expressing cell is an NK cell. In some embodiments, the PD-L1 expressing cell is a cancer or a tumor cell. In some embodiments, the NKG2A expressing cell is an NK cell and the PD-L1 expressing cell is a cancer or a tumor cell.

Additionally or alternatively, a method or a use as described herein thereby forms a complex comprising an NKG2A expressing cell, the binding agent, and an PD-L1 expressing cell. In some embodiments, the NKG2A expressing cell is an immune cell. In further embodiments, the NKG2A expressing cell is a T cell. Additionally or alternatively, the NKG2A expressing cell is an NK cell. In some embodiments, the PD-L1 expressing cell is a cancer or a tumor cell. In some embodiments, the NKG2A expressing cell is an NK cell and the PD-L1 expressing cell is a cancer or a tumor cell.

Additionally or alternatively, a method or a use as described herein further comprises forming a complex comprising an NKG2A expressing cell, the binding agent, and an PD-L1 expressing cell. In some embodiments, the NKG2A expressing cell is an immune cell. In further embodiments, the NKG2A expressing cell is a T cell. Additionally or alternatively, the NKG2A expressing cell is an NK cell. In some embodiments, the PD-L1 expressing cell is a cancer or a tumor cell. In some embodiments, the NKG2A expressing cell is an NK cell and the PD-L1 expressing cell is a cancer or a tumor cell.

Additionally or alternatively, a method or a use as described herein brings an NKG2A expressing cell and a PD-L1 expressing cell in proximity, thereby forming a complex comprising the NKG2A expressing cell, the binding agent and the PD-L1 expressing cell. In some embodiments, the NKG2A expressing cell is an immune cell. In further embodiments, the NKG2A expressing cell is a T cell. Additionally or alternatively, the NKG2A expressing cell is an NK cell. In some embodiments, the PD-L1 expressing cell is a cancer or a tumor cell. In some embodiments, the NKG2A expressing cell is an NK cell and the PD-L1 expressing cell is a cancer or a tumor cell.

Additionally or alternatively, a method or a use as described herein brings an NKG2A expressing cell and a PD-L1 expressing cell in proximity, thereby prevents (e.g., reducing or decreasing, including but not limited to abolishing) or inhibits suppression of the NKG2A expressing cell, such as a tumor/cancer associated immune suppression. In some embodiments, the NKG2A expressing cell is an immune cell. In further embodiments, the NKG2A expressing cell is a T cell. Additionally or alternatively, the NKG2A expressing cell is an NK cell. In some embodiments, the PD-L1 expressing cell is a cancer or a tumor cell. In some embodiments, the NKG2A expressing cell is an NK cell and the PD-L1 expressing cell is a cancer or a tumor cell.

Additionally or alternatively, a method or a use as described herein brings an NKG2A expressing cell and a PD-L1 expressing cell in proximity, thereby activates a response mediated by the NKG2A expressing cell and upon the PD-L1 expressing cell, e.g., an anti-tumor response. In some embodiments, the NKG2A expressing cell is an immune cell. In further embodiments, the NKG2A expressing cell is a T cell. Additionally or alternatively, the NKG2A expressing cell is an NK cell. In some embodiments, the PD-L1 expressing cell is a cancer or a tumor cell. In some embodiments, the NKG2A expressing cell is an NK cell and the PD-L1 expressing cell is a cancer or a tumor cell.

Additionally or alternatively, a method or a use as described herein brings an NKG2A expressing cell and a PD-L1 expressing cell in proximity, thereby modulates (e.g., inhibiting, reducing, preventing) tumor growth. In some embodiments, the NKG2A expressing cell is an immune cell. In further embodiments, the NKG2A expressing cell is a T cell. Additionally or alternatively, the NKG2A expressing cell is an NK cell. In some embodiments, the PD-L1 expressing cell is a cancer or a tumor cell. In some embodiments, the NKG2A expressing cell is an NK cell and the PD-L1 expressing cell is a cancer or a tumor cell.

Additionally or alternatively, a method or a use as described herein brings an NKG2A expressing cell and a PD-L1 expressing cell in proximity, thereby enhances tumor cell removal in a subject with a tumor. In some embodiments, the NKG2A expressing cell is an immune cell. In further embodiments, the NKG2A expressing cell is a T cell. Additionally or alternatively, the NKG2A expressing cell is an NK cell. In some embodiments, the PD-L1 expressing cell is a cancer or a tumor cell. In some embodiments, the NKG2A expressing cell is an NK cell and the PD-L1 expressing cell is a cancer or a tumor cell.

In some embodiments, the disease or disorder is a cancer or a tumor. In further embodiments, the cancer or tumor expresses HLA-E. Additionally or alternatively, the cancer or tumor expresses PD-L1. In some embodiments, the subject is a human subject.

As used herein, “tumor” refers to any neoplastic cell growth or proliferation, whether malignant or benign, and to all pre-cancerous and cancerous cells and tissues. The terms “cancer” and “cancerous” refer to or describe the physiological condition in mammals that is typically characterized by unregulated cell growth. Examples of cancers include, but are not limited to: breast cancer, colon cancer, renal cancer, lung cancer, squamous cell myeloid leukemia, hemangiomas, melanomas, astrocytomas, and glioblastomas as well as other cellular-proliferative disease states, including but not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hanlartoma, inesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinorna, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma}, lymphoma, leukemia, renal cell carcinoma), bladder and urethra (squamous cell carcinoma, transitional cell carcinoma, adenocarcinoma), prostate (adenocarcinoma, sarcoma, small cell carcinoma of the prostate), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis defornians), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma) serous cystadenocarcinoma, mucinous cystadenocarcinoma, unclassified carcinoma, granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma; as well as cancers of the thyroid including medullary thyroid cancer.

In some embodiments, the tumor is a solid tumor. In some embodiments, the tumor or cancer is not a solid tumor. In further embodiments, the cancer is a leukemia cancer. In some embodiments, the tumor or cancer is a relapsed tumor or cancer. In some embodiments, the tumor or cancer is a metastatic tumor or cancer. In some embodiments, the tumor or cancer is a primary tumor or cancer. In some embodiments, the tumor or cancer reaches a remission, but can relapse. In some embodiments, the tumor or cancer is unresectable. Additionally or alternatively, the tumor or cancer is resistant to a chemotherapy or other anti-cancer therapy. In further embodiments, the cancer or tumor expresses HLA-E. Additionally or alternatively, the cancer or tumor expresses PD-L1.

Additionally, binding agents (e.g., multispecific binding agents, such as bispecific antibodies) may be used to alleviate or reduce side effects associated with cancer, for example, bone deterioration, vertebral collapse, and paralysis. In one aspect, the subject suffers from or is at risk of suffering from bone metastases and a binding agent (e.g., a multispecific binding agent such as a bispecific antibody) is administered in an amount to reduce deterioration of surrounding bone. Accordingly, in some aspects, a binding agent provided herein prevents bone deterioration due to bone metastases, wherein tumor cell proliferation is or is not reduced. In some aspects, a binding agent (e.g., a multispecific binding agent such as a bispecific antibody) both prevents bone deterioration due to bone metastases and reduces tumor cell proliferation. In general, the effect on tumor cell proliferation (e.g., inhibition of proliferation or no effect on proliferation) depends on the microenvironment of a particular metastasis. For example, proliferation of metastases located in microenvironments with substantial amounts of type 1 collagen may be inhibited. In contrast, proliferation of metastases located in microenvironments lacking substantial amounts of type 1 collagen may not be inhibited, yet bone deterioration in the vicinity of the metastasis is reduced or prevented.

In other embodiments, provided herein is a method for alleviating one or more symptoms associated with an autoimmune or inflammatory disease or disorder in a subject comprising administering to the subject a multispecific binding agent (e.g., a bispecific antibody) described herein or a pharmaceutical composition described herein. In some embodiments, provided herein is a multispecific binding agent (e.g., a bispecific antibody) described herein or a pharmaceutical composition described herein for use in alleviating one or more symptoms associated with an autoimmune or inflammatory disease or disorder in a subject. In some embodiments, provided herein is a use of a multispecific binding agent (e.g., a bispecific antibody) described herein or a pharmaceutical composition described herein in the manufacture of a medicament for alleviating one or more symptoms associated with an autoimmune or inflammatory disease or disorder in a subject.

Autoimmune disease refers to a disease in which the body's immune system attacks healthy cells. In further embodiments, an autoimmune disease refers to a disease in which the body's adaptive immune system attacks healthy cells. Inflammatory disease refers to a condition related to abnormal inflammation, for example, when inflammation response is misdirected, typically due to that the immune system attacks healthy tissues (resulting in inflammation). In some embodiments, an inflammatory disease is triggered by an infection by a pathogen, such as a virus or a bacterium. Additionally or alternatively, an inflammatory disease refers to a condition related to an abnormal innate immune system attaching healthy cells.

Also provided is a method of treating a disease or disorder (e.g., a cancer or an autoimmune or inflammatory disease or disorder) by administering a binding agent (e.g., a multispecific binding agent such as a bispecific antibody) provided herein, or a pharmaceutical composition as disclosed herein, to a subject in need thereof, alone or in combination with another agent.

The subject of a method described herein can be administered one or more additional therapeutic agents in combination with a binding agent (e.g., a multispecific binding agent such as a bispecific antibody) described herein or fragment thereof or a pharmaceutical composition described herein. An additional agent can be an agent that targets a tumor or a cancer cell. An additional agent can also be an agent that targets an immune cell (e.g., an NK cell or a T cell). In some embodiments, the binding agent or pharmaceutical composition provided herein increases a therapeutic effect of the additional agent by about 10%-90% or about 2-100 folds. In some embodiments, the binding agent or pharmaceutical composition provided herein increases a therapeutic effect of the additional agent by at least 10%. In some embodiments, the binding agent or pharmaceutical composition provided herein increases a therapeutic effect of the additional agent by at least 20%. In some embodiments, the binding agent or pharmaceutical composition provided herein increases a therapeutic effect of the additional agent by at least 30%. In some embodiments, the binding agent or pharmaceutical composition provided herein increases a therapeutic effect of the additional agent by at least 40%. In some embodiments, the binding agent or pharmaceutical composition provided herein increases a therapeutic effect of the additional agent by at least 50%. In some embodiments, the binding agent or pharmaceutical composition provided herein increases a therapeutic effect of the additional agent by at least 60%. In some embodiments, the binding agent or pharmaceutical composition provided herein increases a therapeutic effect of the additional agent by at least 70%. In some embodiments, the binding agent or pharmaceutical composition provided herein increases a therapeutic effect of the additional agent by at least 80%. In some embodiments, the binding agent or pharmaceutical composition provided herein increases a therapeutic effect of the additional agent by at least 90%. In some embodiments, the binding agent or pharmaceutical composition provided herein increases a therapeutic effect of the additional agent by at least 2 fold. In some embodiments, the binding agent or pharmaceutical composition provided herein increases a therapeutic effect of the additional agent by at least 5 fold. In some embodiments, the binding agent or pharmaceutical composition provided herein increases a therapeutic effect of the additional agent by at least 10 fold. In some embodiments, the binding agent or pharmaceutical composition provided herein increases a therapeutic effect of the additional agent by at least 20 fold. In some embodiments, the binding agent or pharmaceutical composition provided herein increases a therapeutic effect of the additional agent by more than 50 fold.

A particular administration regimen of a binding agent (e.g., a multispecific binding agent such as a bispecific antibody) or pharmaceutical composition as disclosed herein for a particular subject will depend, in part, upon the agent used, the amount of agent administered, the route of administration, and the cause and extent of any side effects. The amount of the binding agent (e.g., a bispecific antibody) administered to a subject (e.g., a mammal, such as a human) should be sufficient to effect the desired response over a reasonable time frame. According, in some embodiments, the amount of a binding agent (e.g., a multispecific binding agent such as a bispecific antibody) or pharmaceutical composition described herein administered to a subject is an effective amount.

Suitable routes of administering a binding agent (e.g., an antibody), such as a multispecific binding agent (e.g., a bispecific antibody) or a composition described herein, are well known in the art, such as intravenous injection (such as intravenous infusion), intratumoral injection, or injection adjacent to a tumor or cancer. Although more than one route can be used to administer an agent (e.g., an antibody), a particular route can provide a more immediate and more effective reaction than another route.

### Gene and Cellular Therapies

In some embodiments, the composition for use according to the present disclosure comprises one or more nucleic acids encoding a binding agent provided herein (e.g., an antibody or fragment thereof) or complementary nucleic acids thereto. In a specific embodiment, the nucleic acids are administered to a subject for use in a method provided herein, for example, to prevent, manage, treat and/or ameliorate a disease or disorder (e.g., a cancer, for example a cancer expressing HLA-E and/or PD-L1) by way of gene and/or cell therapy. Such therapy encompasses that performed by the administration to a subject of an expressed or expressible nucleic acid. In an embodiment, the nucleic acids produce their encoded antibody, and the antibody mediates a prophylactic or therapeutic effect.

Any of the methods for recombinant gene expression (or gene therapy) available in the art can be used.

For general review of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May 1993, TIBTECH 11(5):155-215. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a specific embodiment, a composition comprises nucleic acids encoding a binding agent or a fusion protein provided herein, the nucleic acids being part of an expression vector that expresses the binding agent or fusion proteins or heavy or light chains thereof in a suitable host. In particular, such nucleic acids have promoters, such as heterologous promoters, operably linked to the coding region, the promoter being inducible or constitutive, and, optionally, tissue-specific and/or tumor/cancer-specific. In another particular embodiment, nucleic acid molecules are used in which the coding sequences and any other desired sequences are flanked by regions that promote homologous recombination at a desired site in the genome, thus providing for intrachromosomal expression of the antibody or fusion protein encoding nucleic acids (Koller and Smithies, 1989, Proc. Natl. Acad. Sci. USA 86:8932-8935; Zijlstra et al., 1989, Nature 342:435-438).

Delivery of the nucleic acids into a subject can be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy.

In a specific embodiment, the nucleic acid sequences are directly administered in vivo, where the sequences are expressed to produce the encoded product. This can be accomplished by any of numerous methods known in the art, e.g., by constructing them as part of an appropriate nucleic acid expression vector and administering the vector so that the sequences become intracellular, e.g., by infection using defective or attenuated retroviral or other viral vectors (see U.S. Pat. No. 4,980,286), or by direct injection of naked DNA, or by use of microparticle bombardment (e.g., a gene gun; Biolistic, Dupont), or coating with lipids or cell surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429-4432) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., WO 92/06180; WO 92/22635; WO 92/20316; WO 93/14188, WO 93/20221). Alternatively, the nucleic acid can be introduced intracellularly and incorporated within host cell DNA for expression, by homologous recombination (Koller and Smithies, 1989, Proc. Natl. Acad. Sci. USA 86:8932-8935; and Zijlstra et al., 1989, Nature 342:435-438).

In a specific embodiment, viral vectors that contains nucleic acid sequences encoding an antibody are used. For example, a retroviral vector can be used (see Miller et al., 1993, Meth. Enzymol. 217:581-599). These retroviral vectors contain the components necessary for the correct packaging of the viral genome and integration into the host cell DNA. The nucleic acid sequences encoding the antibody to be used in gene therapy can be cloned into one or more vectors, which facilitates delivery of the gene into a subject. More detail about retroviral vectors can be found in Boesen et al., 1994, Biotherapy 6:291-302, which describes the use of a retroviral vector to deliver the MDR1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in the recombinant production of antibodies. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang et al., 1995, Gene Therapy 2:775-783. In a specific embodiment, adenovirus vectors are used.

Adeno-associated virus (AAV) can also be utilized (Walsh et al., 1993, Proc. Soc. Exp. Biol. Med. 204:289-300; and U.S. Pat. No. 5,436,146). In a specific embodiment, AAV vectors are used to express a bispecific antibody provided herein. In certain embodiments, the AAV comprises a nucleic acid encoding a VH domain. In other embodiments, the AAV comprises a nucleic acid encoding a VL domain. In certain embodiments, the AAV comprises a nucleic acid encoding a VH domain and a VL domain. In some embodiments of the methods provided herein, a subject is administered an AAV comprising a nucleic acid encoding a VH domain and an AAV comprising a nucleic acid encoding a VL domain. In other embodiments, a subject is administered an AAV comprising a nucleic acid encoding a VH domain and a VL domain. In certain embodiments, the VH and VL domains are over-expressed.

In some embodiments, oncolytic viruses may be used in the recombinant production of antibodies provided herein. An oncolytic virus may preferentially infect and kill cancer cells. As the infected cancer cells are destroyed by oncolysis, they may release new infectious virus particles or virions to help destroy the remaining tumor. In a specific embodiment, an oncolytic virus is a virus that when injected into a tumor results in tumor regression. In another specific embodiment, an oncolytic virus is a virus that selectively replicates in and kills cancer cells, and spreads within the tumor. In another specific embodiment, an oncolytic virus is a virus that selectively replicates in and kills cancer cells, and spreads within the tumor without causing any significant damage to normal tissue. In some embodiments, an in vitro or ex vivo assay known to one skilled in the art is used to determine the selectively of a virus to replicate in cancer cells versus non-cancerous cells (e.g., healthy cells). In one embodiment, a virus selectively replicates in cancer cells if a statistically significant increase in the number of virus particles is detected in cancer cells in an in vitro assay or ex vivo assay relative to the number of virus particles detected in non-cancerous cells (e.g., healthy cells) in the same assay after incubation with the virus. In another embodiment, a virus selectively kills cancer cells if a statistically significant amount of the cancer cells are killed in an in vitro or ex vivo assay relative to the amount of non-cancerous cells (e.g., healthy cells) killed in the same assay. In one embodiment, an oncolytic virus naturally preferentially replicates in cancer cells and is non-pathogenic in humans. An oncolytic virus may be non-pathogenic in humans due to elevated sensitivity to innate antiviral signal or dependence on oncogenic signaling pathways. In some embodiments, an oncolytic virus is a parovirus (e.g., an autonomous parvovirus), a myxoma virus, an avian paramyxovirus (e.g., Newcastle disease virus), a reovirus, or Seneca valley virus. In one embodiment, an oncolytic virus is wild-type parvovirus H1 (ParvOryx). In another embodiment, an oncolytic virus is Vesicular stomatitis virus. In another embodiment, an oncolytic virus is an avian paramyxovirus. In some embodiments, an oncolytic virus is a genetically engineered influenza virus, measles virus, poliovirus, vaccinia virus, poxvirus, picornavirus, alphavirus, retrovirus, rhabdovirus, reovirus, adenovirus, herpes simplex virus, or vesicular stomatitis virus. In some embodiments, such viruses are attenuated.

Another approach to gene and cell therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcellmediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Clin. Pharma. Ther. 29:69-92 (1985)) and can be used in accordance with the methods provided herein, provided that the necessary developmental and physiological functions of the recipient cells are not disrupted. The technique should provide for the stable transfer of the nucleic acid to the cell, so that the nucleic acid is expressible by the cell, such as heritable and expressible by its cell progeny.

The resulting recombinant cells can be delivered to a subject by various methods known in the art. Recombinant blood cells (e.g., hematopoietic stem or progenitor cells) can be administered intravenously. The amount of cells envisioned for use depends on the desired effect, patient state, etc., and can be determined by one skilled in the art.

Cells into which a nucleic acid can be introduced for purposes of gene and/or cell therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a specific embodiment, the cell used for cell and/or gene therapy is autologous to the subject. In other embodiments, the cell used for cell and/or gene therapy is allogeneic to the subject, such as an NK cell.

In an embodiment in which recombinant cells are used in gene and/or therapy, nucleic acid sequences encoding an antibody are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the methods provided herein (see e.g., WO 94/08598; Stemple and Anderson, 1992, Cell 7 1:973-985; Rheinwald, 1980, Meth. Cell Bio. 21A: 229; and Pittelkow and Scott, 1986, Mayo Clinic Proc. 61:771).

In a specific embodiment, the nucleic acid to be introduced for purposes of gene and/or therapy comprises an inducible promoter operably linked to the coding region, such that expression of the nucleic acid is controllable by controlling the presence or absence of the appropriate inducer of transcription.

### Diagnostic Uses and Methods of Detection

Labeled binding molecules such as labeled antibodies and derivatives and analogs thereof, which immunospecifically bind to an antigen can be used for diagnostic purposes to detect, diagnose, or monitor a disease.

Antibodies provided herein can be used to assay an antigen level in a biological sample using classical immunohistological methods as described herein or as known to those of skill in the art (e.g., see Jalkanen et al., 1985, J. Cell. Biol. 101:976-985; and Jalkanen et al., 1987, J. Cell. Biol. 105:3087-3096). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹Tc. The labeled antibody will then accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982)).

Depending on several variables, including the type of label used and the mode of administration, the time interval following the administration for permitting the labeled antibody to concentrate at sites in the subject and for unbound labeled antibody to be cleared to background level is 6 to 48 hours or 6 to 24 hours or 6 to 12 hours. In another embodiment the time interval following administration is 5 to 20 days or 5 to 10 days.

Presence of the labeled molecule can be detected in the subject using methods known in the art for in vivo scanning. These methods depend upon the type of label used. Skilled artisans will be able to determine the appropriate method for detecting a particular label. Methods and devices that may be used in the diagnostic methods provided herein include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patient using positron emission-tomography. In yet another embodiment, the molecule is labeled with a paramagnetic label and is detected in a patient using magnetic resonance imaging (MRI).

### 5.7. Kits

Also provided herein are kits comprising a binding agent (e.g., a multispecific agent such as a bispecific antibody) provided herein, or a composition (e.g., a pharmaceutical composition) provided herein, packaged into suitable packaging material. A kit optionally includes a label or packaging insert including a description of the components or instructions for use in vitro, in vivo, or ex vivo, of the components therein.

The term “packaging material” refers to a physical structure housing the components of the kit. The packaging material can maintain the components sterilely, and can be made of material commonly used for such purposes (e.g., paper, corrugated fiber, glass, plastic, foil, ampoules, vials, tubes, etc.).

Kits provided herein can include labels or inserts. Labels or inserts include “printed matter,” e.g., paper or cardboard, separate or affixed to a component, a kit or packing material (e.g., a box), or attached to, for example, an ampoule, tube, or vial containing a kit component. Labels or inserts can additionally include a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media, or memory type cards. Labels or inserts can include information identifying manufacturer information, lot numbers, manufacturer location, and date.

Kits provided herein can additionally include other components. Each component of the kit can be enclosed within an individual container, and all of the various containers can be within a single package. Kits can also be designed for cold storage. A kit can further be designed to contain antibodies provided herein, or cells that contain nucleic acids encoding the antibodies provided herein. The cells in the kit can be maintained under appropriate storage conditions until ready to use.

Also provided herein are panels of antibodies that immunospecifically bind to an NKG2A antigen and/or PD-L1 antigen. In specific embodiments, provided herein are panels of antibodies having different association rate constants, different dissociation rate constants, different affinities for NKG2A antigen and/or PD-L1 antigen, and/or different specificities for an NKG2A antigen and/or PD-L1 antigen. In certain embodiments, provided herein are panels of about 10, preferably about 25, about 50, about 75, about 100, about 125, about 150, about 175, about 200, about 250, about 300, about 350, about 400, about 450, about 500, about 550, about 600, about 650, about 700, about 750, about 800, about 850, about 900, about 950, or about 1000 antibodies or more. Panels of antibodies can be used, for example, in 96 well or 384 well plates, such as for assays such as ELISAs.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described herein.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

In addition, reference to a range of 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250 includes 1, 2, 3, 4, 5, 6, 7, 8, 9, 10, 11, 12, 13, 14, 15, 16, 17, 18, 19, 20, etc. In a further example, reference to a range of 25-250, 250-500, 500-1,000, 1,000-2,500, 2,500-5,000, 5,000-25,000, 25,000-50,000 includes any numerical value or range within or encompassing such values, e.g., 25, 26, 27, 28, 29 . . . 250, 251, 252, 253, 254 . . . 500, 501, 502, 503, 504 . . . , etc.

As also used herein a series of ranges are disclosed throughout this document. The use of a series of ranges includes combinations of the upper and lower ranges to provide another range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, includes ranges such as 5-20, 5-30, 5-40, 5-50, 5-75, 5-100, 5-150, and 10-30, 10-40, 10-50, 10-75, 10-100, 10-150, and 20-40, 20-50, 20-75, 20-100, 20-150, and so forth.

It is understood that modifications which do not substantially affect the activity of the various embodiments described herein are also provided within the definition of the subject matter described herein. Accordingly, the following examples are intended to illustrate but not limit the present disclosure.

### 6. EMBODIMENTS

The present disclosure encompasses the following non-limiting embodiments:
1. A multispecific antibody or fragment thereof comprising a first binding domain that binds to NKG2A and a second binding domain that binds to PD-L1, wherein the first binding domain comprises any one or more of (i)-(iv):
(i) a VH CDR1, a VH CDR2, and a VH CDR3 as set forth in a VH comprising the amino acid sequence of SEQ ID NO:25 and a VL CDR1, a VL CDR2, and a VL CDR3 as set forth in a VL comprising the amino acid sequence of SEQ ID NO:26;
(ii) a VH CDR1, a VH CDR2, and a VH CDR3 as set forth in a VH comprising the amino acid sequence of SEQ ID NO:45 and a VL CDR1, a VL CDR2, and a VL CDR3 as set forth in a VL comprising the amino acid sequence of SEQ ID NO:46;
(iii) a VH CDR1, a VH CDR2, and a VH CDR3 as set forth in a VH comprising the amino acid sequence of SEQ ID NO:64 and a VL CDR1, a VL CDR2, and a VL CDR3 as set forth in a VL comprising the amino acid sequence of SEQ ID NO:65; or
(iv) a VH CDR1, a VH CDR2, and a VH CDR3 as set forth in a VH comprising the amino acid sequence of SEQ ID NO:64 and a VL CDR1, a VL CDR2, and a VL CDR3 as set forth in a VL comprising the amino acid sequence of SEQ ID NO:73.
2. The multispecific antibody or fragment thereof of embodiment 1, wherein the first binding domain comprises
(a) a VH region comprising:
(1) a VH CDR1 having an amino acid sequence selected from the group consisting of SEQ ID NOs: 1, 7, 12, 13, and 18;
(2) a VH CDR2 having an amino acid sequence selected from the group consisting of SEQ ID NOs: 2, 8, 14, 19, and 24; and
(3) a VH CDR3 having an amino acid sequence selected from the group consisting of SEQ ID NOs: 3, 9, 15, and 20; and
(b) a VL region comprising:
(1) a VL CDR1 having an amino acid sequence selected from the group consisting of SEQ ID NOs: 4, 10, 16, and 21;
(2) a VL CDR2 having an amino acid sequence selected from the group consisting of SEQ ID NOs: 5, 11, and 22; and
(3) a VL CDR3 having an amino acid sequence selected from the group consisting of SEQ ID NOs: 6, 17, and 23.
3. The multispecific antibody or fragment thereof of embodiment 1, wherein the first binding domain comprises any one or more of (i)-(vi):
(i) a VH region comprising a VH CDR1 comprising the amino acid sequence of SEQ ID NO: 1, a VH CDR2 comprising the amino acid sequence of SEQ ID NO:2, a VH CDR3 comprising the amino acid sequence of SEQ ID NO:3; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6;
(ii) a VH region comprising a VH CDR1 comprising the amino acid sequence of SEQ ID NO: 7, a VH CDR2 comprising the amino acid sequence of SEQ ID NO:8, a VH CDR3 comprising the amino acid sequence of SEQ ID NO:9; and a VL region comprising a VL CDR1 comprising the amino acid sequence of SEQ ID NO:10, a VL CDR2 comprising the amino acid sequence of SEQ ID NO:11, and a VL CDR3 comprising the amino acid sequence of SEQ ID NO:6;
(iii) a VH region comprising a VH CDR1 comprising the amino acid sequence of SEQ ID NO: 12, a VH CDR2 comprising the amino acid sequence of SEQ ID NO:2, a VH CDR3 comprising the amino acid sequence of SEQ ID NO:3; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6;
(iv) a VH region comprising a VH CDR1 comprising the amino acid sequence of SEQ ID NO: 13, a VH CDR2 comprising the amino acid sequence of SEQ ID NO:14, a VH CDR3 comprising the amino acid sequence of SEQ ID NO:15; and a VL region comprising a VL CDR1 comprising the amino acid sequence of SEQ ID NO:16, a VL CDR2 comprising the amino acid sequence of SEQ ID NO:11, and a VL CDR3 comprising the amino acid sequence of SEQ ID NO:17;
(v) a VH region comprising a VH CDR1 comprising the amino acid sequence of SEQ ID NO: 18, a VH CDR2 comprising the amino acid sequence of SEQ ID NO:19, a VH CDR3 comprising the amino acid sequence of SEQ ID NO:20; and a VL region comprising a VL CDR1 comprising the amino acid sequence of SEQ ID NO:21, a VL CDR2 comprising the amino acid sequence of SEQ ID NO:22, and a VL CDR3 comprising the amino acid sequence of SEQ ID NO:23; or
(vi) a VH region comprising a VH CDR1 comprising the amino acid sequence of SEQ ID NO: 1, a VH CDR2 comprising the amino acid sequence of SEQ ID NO:24, a VH CDR3 comprising the amino acid sequence of SEQ ID NO:3; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6.
4. The multispecific antibody or fragment thereof of embodiment 1, wherein the first binding domain comprises
(a) a VH region comprising:
(1) a VH CDR1 having an amino acid sequence selected from the group consisting of SEQ ID NOs: 1, 7, 12, 13, and 18;
(2) a VH CDR2 having an amino acid sequence selected from the group consisting of SEQ ID NOs: 27, 32, 35, 39, and 44; and
(3) a VH CDR3 having an amino acid sequence selected from the group consisting of SEQ ID NOs: 28, 33, 36, and 40; and
(b) a VL region comprising:
(1) a VL CDR1 having an amino acid sequence selected from the group consisting of SEQ ID NOs: 29, 34, 37, and 41;
(2) a VL CDR2 having an amino acid sequence selected from the group consisting of SEQ ID NOs: 30, 11, and 42; and
(3) a VL CDR3 having an amino acid sequence selected from the group consisting of SEQ ID NOs: 31, 38, and 43.
5. The multispecific antibody or fragment thereof of embodiment 1, wherein the first binding domain comprises any one or more of (i)-(vi):
(i) a VH region comprising a VH CDR1 comprising the amino acid sequence of SEQ ID NO: 1, a VH CDR2 comprising the amino acid sequence of SEQ ID NO:27, a VH CDR3 comprising the amino acid sequence of SEQ ID NO:28; and a VL region comprising a VL CDR1 comprising the amino acid sequence of SEQ ID NO:29, a VL CDR2 comprising the amino acid sequence of SEQ ID NO:30, and a VL CDR3 comprising the amino acid sequence of SEQ ID NO:31;
(ii) a VH region comprising a VH CDR1 comprising the amino acid sequence of SEQ ID NO: 7, a VH CDR2 comprising the amino acid sequence of SEQ ID NO:32, a VH CDR3 comprising the amino acid sequence of SEQ ID NO:33; and a VL region comprising a VL CDR1 comprising the amino acid sequence of SEQ ID NO:34, a VL CDR2 comprising the amino acid sequence of SEQ ID NO:11, and a VL CDR3 comprising the amino acid sequence of SEQ ID NO:31;
(iii) a VH region comprising a VH CDR1 comprising the amino acid sequence of SEQ ID NO: 12, a VH CDR2 comprising the amino acid sequence of SEQ ID NO:27, a VH CDR3 comprising the amino acid sequence of SEQ ID NO:28; and a VL region comprising a VL CDR1 comprising the amino acid sequence of SEQ ID NO:29, a VL CDR2 comprising the amino acid sequence of SEQ ID NO:30, and a VL CDR3 comprising the amino acid sequence of SEQ ID NO:31;
(iv) a VH region comprising a VH CDR1 comprising the amino acid sequence of SEQ ID NO: 13, a VH CDR2 comprising the amino acid sequence of SEQ ID NO:35, a VH CDR3 comprising the amino acid sequence of SEQ ID NO:36; and a VL region comprising a VL CDR1 comprising the amino acid sequence of SEQ ID NO:37, a VL CDR2 comprising the amino acid sequence of SEQ ID NO:11, and a VL CDR3 comprising the amino acid sequence of SEQ ID NO:38;
(v) a VH region comprising a VH CDR1 comprising the amino acid sequence of SEQ ID NO: 18, a VH CDR2 comprising the amino acid sequence of SEQ ID NO:39, a VH CDR3 comprising the amino acid sequence of SEQ ID NO:40; and a VL region comprising a VL CDR1 comprising the amino acid sequence of SEQ ID NO:41, a VL CDR2 comprising the amino acid sequence of SEQ ID NO:42, and a VL CDR3 comprising the amino acid sequence of SEQ ID NO:43; or
(vi) a VH region comprising a VH CDR1 comprising the amino acid sequence of SEQ ID NO: 1, a VH CDR2 comprising the amino acid sequence of SEQ ID NO:44, a VH CDR3 comprising the amino acid sequence of SEQ ID NO:28; and a VL region comprising a VL CDR1 comprising the amino acid sequence of SEQ ID NO:29, a VL CDR2 comprising the amino acid sequence of SEQ ID NO:30, and a VL CDR3 comprising the amino acid sequence of SEQ ID NO:31.
6. The multispecific antibody or fragment thereof of embodiment 1, wherein the first binding domain comprises
(a) a VH region comprising:
(1) a VH CDR1 having an amino acid sequence selected from the group consisting of SEQ ID NOs: 47, 51, 54, 55, and 59;
(2) a VH CDR2 having an amino acid sequence selected from the group consisting of SEQ ID NOs: 48, 52, 56, 60, and 63; and
(3) a VH CDR3 having an amino acid sequence selected from the group consisting of SEQ ID NOs: 49, 53, 57, and 61; and
(b) a VL region comprising:
(1) a VL CDR1 having an amino acid sequence selected from the group consisting of SEQ ID NOs: 4, 10, 16, and 21;
(2) a VL CDR2 having an amino acid sequence selected from the group consisting of SEQ ID NOs: 5, 11, and 22; and
(3) a VL CDR3 having an amino acid sequence selected from the group consisting of SEQ ID NOs: 50, 58, and 62.
7. The multispecific antibody or fragment thereof of embodiment 1, wherein the first binding domain comprises any one or more of (i)-(vi):
(i) a VH region comprising a VH CDR1 comprising the amino acid sequence of SEQ ID NO: 47, a VH CDR2 comprising the amino acid sequence of SEQ ID NO:48, a VH CDR3 comprising the amino acid sequence of SEQ ID NO:49; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:50;
(ii) a VH region comprising a VH CDR1 comprising the amino acid sequence of SEQ ID NO: 51, a VH CDR2 comprising the amino acid sequence of SEQ ID NO:52, a VH CDR3 comprising the amino acid sequence of SEQ ID NO:53; and a VL region comprising a VL CDR1 comprising the amino acid sequence of SEQ ID NO:10, a VL CDR2 comprising the amino acid sequence of SEQ ID NO:11, and a VL CDR3 comprising the amino acid sequence of SEQ ID NO:50;
(iii) a VH region comprising a VH CDR1 comprising the amino acid sequence of SEQ ID NO: 54, a VH CDR2 comprising the amino acid sequence of SEQ ID NO:48, a VH CDR3 comprising the amino acid sequence of SEQ ID NO:49; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:50;
(iv) a VH region comprising a VH CDR1 comprising the amino acid sequence of SEQ ID NO: 55, a VH CDR2 comprising the amino acid sequence of SEQ ID NO:56, a VH CDR3 comprising the amino acid sequence of SEQ ID NO:57; and a VL region comprising a VL CDR1 comprising the amino acid sequence of SEQ ID NO:16, a VL CDR2 comprising the amino acid sequence of SEQ ID NO:11, and a VL CDR3 comprising the amino acid sequence of SEQ ID NO:58;
(v) a VH region comprising a VH CDR1 comprising the amino acid sequence of SEQ ID NO: 59, a VH CDR2 comprising the amino acid sequence of SEQ ID NO:60, a VH CDR3 comprising the amino acid sequence of SEQ ID NO:61; and a VL region comprising a VL CDR1 comprising the amino acid sequence of SEQ ID NO:21, a VL CDR2 comprising the amino acid sequence of SEQ ID NO:22, and a VL CDR3 comprising the amino acid sequence of SEQ ID NO:62; or
(vi) a VH region comprising a VH CDR1 comprising the amino acid sequence of SEQ ID NO: 47, a VH CDR2 comprising the amino acid sequence of SEQ ID NO:63, a VH CDR3 comprising the amino acid sequence of SEQ ID NO:49; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:50.
8. The multispecific antibody or fragment thereof of embodiment 1, wherein the first binding domain comprises
(a) a VH region comprising:
(1) a VH CDR1 having an amino acid sequence selected from the group consisting of SEQ ID NOs: 47, 51, 54, 55, and 59;
(2) a VH CDR2 having an amino acid sequence selected from the group consisting of SEQ ID NOs: 48, 52, 56, 60, and 63; and
(3) a VH CDR3 having an amino acid sequence selected from the group consisting of SEQ ID NOs: 49, 53, 57, and 61; and
(b) a VL region comprising:
(1) a VL CDR1 having an amino acid sequence selected from the group consisting of SEQ ID NOs: 66, 68, 70, and 71;
(2) a VL CDR2 having an amino acid sequence selected from the group consisting of SEQ ID NOs: 67, 69, and 72; and
(3) a VL CDR3 having an amino acid sequence selected from the group consisting of SEQ ID NOs: 50, 58, and 62.
9. The multispecific antibody or fragment thereof of embodiment 1, wherein the first binding domain comprises any one or more of (i)-(vi):
(i) a VH region comprising a VH CDR1 comprising the amino acid sequence of SEQ ID NO: 47, a VH CDR2 comprising the amino acid sequence of SEQ ID NO:48, a VH CDR3 comprising the amino acid sequence of SEQ ID NO:49; and a VL region comprising a VL CDR1 comprising the amino acid sequence of SEQ ID NO:66, a VL CDR2 comprising the amino acid sequence of SEQ ID NO:67, and a VL CDR3 comprising the amino acid sequence of SEQ ID NO:50;
(ii) a VH region comprising a VH CDR1 comprising the amino acid sequence of SEQ ID NO: 51, a VH CDR2 comprising the amino acid sequence of SEQ ID NO:52, a VH CDR3 comprising the amino acid sequence of SEQ ID NO:53; and a VL region comprising a VL CDR1 comprising the amino acid sequence of SEQ ID NO:68, a VL CDR2 comprising the amino acid sequence of SEQ ID NO:69, and a VL CDR3 comprising the amino acid sequence of SEQ ID NO:50;
(iii) a VH region comprising a VH CDR1 comprising the amino acid sequence of SEQ ID NO: 54, a VH CDR2 comprising the amino acid sequence of SEQ ID NO:48, a VH CDR3 comprising the amino acid sequence of SEQ ID NO:49; and a VL region comprising a VL CDR1 comprising the amino acid sequence of SEQ ID NO:66, a VL CDR2 comprising the amino acid sequence of SEQ ID NO:67, and a VL CDR3 comprising the amino acid sequence of SEQ ID NO:50;
(iv) a VH region comprising a VH CDR1 comprising the amino acid sequence of SEQ ID NO: 55, a VH CDR2 comprising the amino acid sequence of SEQ ID NO:56, a VH CDR3 comprising the amino acid sequence of SEQ ID NO:57; and a VL region comprising a VL CDR1 comprising the amino acid sequence of SEQ ID NO:70, a VL CDR2 comprising the amino acid sequence of SEQ ID NO:69, and a VL CDR3 comprising the amino acid sequence of SEQ ID NO:58;
(v) a VH region comprising a VH CDR1 comprising the amino acid sequence of SEQ ID NO: 59, a VH CDR2 comprising the amino acid sequence of SEQ ID NO:60, a VH CDR3 comprising the amino acid sequence of SEQ ID NO:61; and a VL region comprising a VL CDR1 comprising the amino acid sequence of SEQ ID NO:71, a VL CDR2 comprising the amino acid sequence of SEQ ID NO:72, and a VL CDR3 comprising the amino acid sequence of SEQ ID NO:62; or
(vi) a VH region comprising a VH CDR1 comprising the amino acid sequence of SEQ ID NO: 47, a VH CDR2 comprising the amino acid sequence of SEQ ID NO:63, a VH CDR3 comprising the amino acid sequence of SEQ ID NO:49; and a VL region comprising a VL CDR1 comprising the amino acid sequence of SEQ ID NO:66, a VL CDR2 comprising the amino acid sequence of SEQ ID NO:67, and a VL CDR3 comprising the amino acid sequence of SEQ ID NO:50.
10. The multispecific antibody or fragment thereof of any one of embodiments 1 to 9, wherein the first binding domain further comprises a framework 1 (FR1), a framework 2 (FR2), a framework 3 (FR3) and/or a framework 4 (FR4) sequence, optionally as set forth in any one of SEQ ID NOs: 25, 26, 45, 46, 64, 65, and 73.
11. The multispecific antibody or fragment thereof of any one of embodiments 1 to 10, wherein the first binding domain further comprises human framework sequences.
12. The multispecific antibody or fragment thereof of any one of embodiments 1 to 11, wherein the first binding domain comprises:
(i) a VH comprising the amino acid sequence of SEQ ID NO:25 and a VL comprising the amino acid sequence of SEQ ID NO:26;
(ii) a VH comprising the amino acid sequence of SEQ ID NO:45 and a VL comprising the amino acid sequence of SEQ ID NO:46;
(iii) a VH comprising the amino acid sequence of SEQ ID NO:64 and a VL comprising the amino acid sequence of SEQ ID NO:65; or
(iv) a VH comprising the amino acid sequence of SEQ ID NO:64 and a VL comprising the amino acid sequence of SEQ ID NO:73.
13. The multispecific antibody or fragment thereof of any one of embodiments 1-12, wherein the second binding domain comprises a VH CDR1, a VH CDR2, and a VH CDR3 as set forth in a VH comprising the amino acid sequence of SEQ ID NO:98 and a VL CDR1, a VL CDR2, and a VL CDR3 as set forth in a VL comprising the amino acid sequence of SEQ ID NO:99.
14. A multispecific antibody or fragment thereof comprising a first binding domain that binds to NKG2A and a second binding domain that binds to PD-L1, wherein the second binding domain comprises a VH CDR1, a VH CDR2, and a VH CDR3 as set forth in a VH comprising the amino acid sequence of SEQ ID NO:98 and a VL CDR1, a VL CDR2, and a VL CDR3 as set forth in a VL comprising the amino acid sequence of SEQ ID NO:99.
15 The multispecific antibody or fragment thereof of embodiment 13 or 14, wherein the second binding domain comprises
(a) a VH region comprising:
(1) a VH CDR1 having an amino acid sequence selected from the group consisting of SEQ ID NOs: 1, 7, 12, 13, and 18;
(2) a VH CDR2 having an amino acid sequence selected from the group consisting of
SEQ ID NOs: 86, 89, 91, 94, and 97; and
(3) a VH CDR3 having an amino acid sequence selected from the group consisting of SEQ ID NOs: 87, 90. 92, and 95; and
(b) a VL region comprising:
(1) a VL CDR1 having an amino acid sequence selected from the group consisting of SEQ ID NOs: 4, 10, 16, and 21;
(2) a VL CDR2 having an amino acid sequence selected from the group consisting of SEQ ID NOs: 5, 11, and 22; and
(3) a VL CDR3 having an amino acid sequence selected from the group consisting of SEQ ID NOs: 88, 93, and 96.
16. The multispecific antibody or fragment thereof of embodiment 13 or 14, wherein the second binding domain comprises any one or more of (i)-(vi):
(i) a VH region comprising a VH CDR1 comprising the amino acid sequence of SEQ ID NO: 1, a VH CDR2 comprising the amino acid sequence of SEQ ID NO:86, a VH CDR3 comprising the amino acid sequence of SEQ ID NO:87; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:88;
(ii) a VH region comprising a VH CDR1 comprising the amino acid sequence of SEQ ID NO: 7, a VH CDR2 comprising the amino acid sequence of SEQ ID NO:89, a VH CDR3 comprising the amino acid sequence of SEQ ID NO:90; and a VL region comprising a VL CDR1 comprising the amino acid sequence of SEQ ID NO:10, a VL CDR2 comprising the amino acid sequence of SEQ ID NO:11, and a VL CDR3 comprising the amino acid sequence of SEQ ID NO:88;
(iii) a VH region comprising a VH CDR1 comprising the amino acid sequence of SEQ ID NO: 12, a VH CDR2 comprising the amino acid sequence of SEQ ID NO:86, a VH CDR3 comprising the amino acid sequence of SEQ ID NO:87; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:88;
(iv) a VH region comprising a VH CDR1 comprising the amino acid sequence of SEQ ID NO: 13, a VH CDR2 comprising the amino acid sequence of SEQ ID NO:91, a VH CDR3 comprising the amino acid sequence of SEQ ID NO:92; and a VL region comprising a VL CDR1 comprising the amino acid sequence of SEQ ID NO:16, a VL CDR2 comprising the amino acid sequence of SEQ ID NO:11, and a VL CDR3 comprising the amino acid sequence of SEQ ID NO:93;
(v) a VH region comprising a VH CDR1 comprising the amino acid sequence of SEQ ID NO: 18, a VH CDR2 comprising the amino acid sequence of SEQ ID NO:94, a VH CDR3 comprising the amino acid sequence of SEQ ID NO:95; and a VL region comprising a VL CDR1 comprising the amino acid sequence of SEQ ID NO:21, a VL CDR2 comprising the amino acid sequence of SEQ ID NO:22, and a VL CDR3 comprising the amino acid sequence of SEQ ID NO:96; or
(vi) a VH region comprising a VH CDR1 comprising the amino acid sequence of SEQ ID NO: 1, a VH CDR2 comprising the amino acid sequence of SEQ ID NO:97, a VH CDR3 comprising the amino acid sequence of SEQ ID NO:87; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:88.
17. The multispecific antibody or fragment thereof of embodiment 13 or 14, wherein the second binding domain comprises VH region comprising the amino acid sequence of SEQ ID NO:98 and a VL region comprising the amino acid sequence of SEQ ID NO: 99.
18. The multispecific antibody or fragment thereof of any one of embodiments 1-17, wherein the multispecific antibody or fragment thereof comprises:
(i) a first polypeptide comprising from N-terminus to C-terminus: a first VL, a first CH3, a first CH2, and a second CH3;
(ii) a second polypeptide comprising from N-terminus to C-terminus: a first VH and a third CH3;
(iii) a third polypeptide comprising from N-terminus to C-terminus: a second VL, a CL, a second CH2, and a fourth CH3;
(iv) a fourth polypeptide comprising from N-terminus to C-terminus: a second VH and a CH1;
wherein the first polypeptide and the second polypeptide form the second binding domain that binds to PD-L1, and the third polypeptide and the fourth polypeptide form the first binding domain that binds to NKG2A.
19. The multispecific antibody or fragment thereof of any one of embodiments 1-18, wherein the multispecific antibody or fragment thereof further comprises a third binding domain.
20. The multispecific antibody or fragment thereof of embodiment 19, wherein the third binding domain binds to NKG2A.
21. The multispecific antibody or fragment thereof of embodiment 19, wherein the third binding domain is same as the first binding domain.
22. The multispecific antibody or fragment thereof of embodiment 20, wherein the multispecific antibody or fragment thereof comprises:
(i) a first polypeptide comprising from N-terminus to C-terminus: a first VL, a first CH3, a first CH2, and a second CH3;
(ii) a second polypeptide comprising from N-terminus to C-terminus: a first VH and a third CH3;
(iii) a third polypeptide comprising from N-terminus to C-terminus: a second VL, a first CL, a third VL, a second CL, a second CH2, and a fourth CH3, wherein the second VL and the third VL are the same, and the first CL and the second CL are the same;
(iv) a fourth polypeptide comprising from N-terminus to C-terminus: a second VH and a first CH1;
(v) a fifth polypeptide comprising from N-terminus to C-terminus: a third VH and a second CH1;
wherein the second VH and the third VH are the same, and the first CH1 and the second CH1 are the same; and
wherein the first polypeptide and the second polypeptide form the second binding domain that binds to PD-L1, the third polypeptide and the fourth polypeptide form the first binding domain that binds to NKG2A, and the third polypeptide and the fifth polypeptide form the third binding domain that binds to NKG2A.
23. The multispecific antibody or fragment thereof of embodiment 19, wherein the third binding domain binds to PD-L1.
24. The multispecific antibody or fragment thereof of embodiment 23, wherein the third binding domain is same as the second binding domain.
25. The multispecific antibody or fragment thereof of embodiment 23, wherein the multispecific antibody or fragment thereof comprises:
(i) a first polypeptide comprising from N-terminus to C-terminus: a first VL, a first CH3, a second VL, a second CH3, a first CH2, and a third CH3, wherein the first VL and the second VL are the same, and the first CH3 and the second CH3 are the same;
(ii) a second polypeptide comprising from N-terminus to C-terminus: a first VH and a fourth CH3;
(iii) a third polypeptide comprising from N-terminus to C-terminus: a second VH and a fifth CH3;
(iv) a fourth polypeptide comprising from N-terminus to C-terminus: a third VL, a first
CL, a second CH2 and a sixth CH3;
(v) a fifth polypeptide comprising from N-terminus to C-terminus: a third VH and a CH1; wherein the first VH and the second VH are the same, and the fourth CH3 and the fifth CH3 are the same; and wherein the first polypeptide and the second polypeptide form the second binding domain that binds to PD-L1, the first polypeptide and the third polypeptide form the third binding domain that binds to PD-L1, and the fourth polypeptide and the fifth polypeptide form the first binding domain that binds to NKG2A.
26. The multispecific antibody or fragment thereof of any one of embodiments 1-25, wherein the first binding domain specifically binds to one, two, three, four, five, or all of the following NKG2A polypeptide fragments: an NKG2A polypeptide fragment comprising the amino acid sequence of TWEESL (SEQ ID NO:134), an NKG2A polypeptide fragment comprising the amino acid sequence of SIISPSSWIGV (SEQ ID NO: 135), an NKG2A polypeptide fragment comprising the amino acid sequence of FRNSSHHPW (SEQ ID NO: 136), an NKG2A polypeptide fragment comprising the amino acid sequence of IKDSDNAEL (SEQ ID NO:137), an NKG2A polypeptide fragment comprising the amino acid sequence of LQVNR (SEQ ID NO:138), and an NKG2A polypeptide fragment comprising the amino acid sequence of AQCGSSI (SEQ ID NO:139).
27. The multispecific antibody or fragment thereof of any one of embodiments 1-26, wherein the first binding domain specifically binds to a conformational epitope formed by a group of amino acid residues comprising at least one amino acid residue (1) from one of the following amino acid sequences: TWEESL (SEQ ID NO:134), SIISPSSWIGV (SEQ ID NO: 135), FRNSSHHPW (SEQ ID NO:136), IKDSDNAEL (SEQ ID NO:137), LQVNR (SEQ ID NO:138), and AQCGSSI (SEQ ID NO:139), or (2) from each one of two, three, four, five or all of the following amino acid sequences: TWEESL (SEQ ID NO:134), SIISPSSWIGV (SEQ ID NO:135), FRNSSHHPW (SEQ ID NO:136), IKDSDNAEL (SEQ ID NO: 137), LQVNR (SEQ ID NO:138), and AQCGSSI (SEQ ID NO:139).
28. The multispecific antibody or fragment thereof of embodiment 27, wherein the conformational epitope is formed by a group of amino acid residues comprising at least one amino acid residue (1) from one of the following amino acid sequences located on the surface of NKG2A: TWEESL (SEQ ID NO:134), SIISPSSWIGV (SEQ ID NO:135), FRNSSHHPW (SEQ ID NO:136), IKDSDNAEL (SEQ ID NO:137), LQVNR (SEQ ID NO:138), and AQCGSSI (SEQ ID NO:139), or (2) from each one of two, three, four, five or all of the following amino acid sequences located on the surface of NKG2A: TWEESL (SEQ ID NO: 134), SIISPSSWIGV (SEQ ID NO:135), FRNSSHHPW (SEQ ID NO:136), IKDSDNAEL (SEQ ID NO:137), LQVNR (SEQ ID NO:138), and AQCGSSI (SEQ ID NO: 139).
29. A multispecific antibody or fragment thereof comprising a first binding domain that binds to NKG2A and a second binding domain that binds to PD-L1, wherein the first binding domain specifically binds to one, two, three, four, five, or all of the following NKG2A polypeptide fragments: an NKG2A polypeptide fragment comprising the amino acid sequence of TWEESL (SEQ ID NO:134), an NKG2A polypeptide fragment comprising the amino acid sequence of SIISPSSWIGV (SEQ ID NO:135), an NKG2A polypeptide fragment comprising the amino acid sequence of FRNSSHHPW (SEQ ID NO:136), an NKG2A polypeptide fragment comprising the amino acid sequence of IKDSDNAEL (SEQ ID NO: 137), an NKG2A polypeptide fragment comprising the amino acid sequence of LQVNR (SEQ ID NO:138), and an NKG2A polypeptide fragment comprising the amino acid sequence of AQCGSSI (SEQ ID NO:139).
30. A multispecific antibody or fragment thereof comprising a first binding domain that binds to NKG2A and a second binding domain that binds to PD-L1, wherein the first binding domain specifically binds to a conformational epitope formed by a group of amino acid residues comprising at least one amino acid residue (1) from one of the following amino acid sequences: TWEESL (SEQ ID NO:134), SIISPSSWIGV (SEQ ID NO:135), FRNSSHHPW (SEQ ID NO:136), IKDSDNAEL (SEQ ID NO:137), LQVNR (SEQ ID NO: 138), and AQCGSSI (SEQ ID NO:139), or (2) from each one of two, three, four, five or all of the following amino acid sequences: TWEESL (SEQ ID NO:134), SIISPSSWIGV (SEQ ID NO:135), FRNSSHHPW (SEQ ID NO:136), IKDSDNAEL (SEQ ID NO:137), LQVNR (SEQ ID NO:138), and AQCGSSI (SEQ ID NO:139).
31. A multispecific antibody or fragment thereof comprising a first binding domain that binds to NKG2A and a second binding domain that binds to PD-L1, wherein the first binding domain specifically binds to a conformational epitope formed by a group of amino acid residues comprising at least one amino acid residue (1) from one of the following amino acid sequences located on the surface of NKG2A: TWEESL (SEQ ID NO:134), SIISPSSWIGV (SEQ ID NO:135), FRNSSHHPW (SEQ ID NO:136), IKDSDNAEL (SEQ ID NO: 137), LQVNR (SEQ ID NO:138), and AQCGSSI (SEQ ID NO:139), or (2) from each one of two, three, four, five or all of the following amino acid sequences located on the surface of NKG2A: TWEESL (SEQ ID NO:134), SIISPSSWIGV (SEQ ID NO:135), FRNSSHHPW (SEQ ID NO:136), IKDSDNAEL (SEQ ID NO:137), LQVNR (SEQ ID NO: 138), and AQCGSSI (SEQ ID NO:139).
32. A polynucleotide encoding the multispecific antibody or fragment thereof of any one of embodiments 1-31.
33. One or more vectors comprising one or more polynucleotides of embodiment 32 or a complementary polynucleotide thereto.
34. A cell comprising any one or more of: the multispecific antibody or fragment thereof of any one of embodiments 1-31, the polynucleotide of embodiment 32, or the one or more vectors of embodiment 33.
35. A pharmaceutical composition that comprises a pharmaceutically acceptable excipient and any one or more of: the multispecific antibody or fragment thereof of any one of embodiments 1-31, the polynucleotide of embodiment 32, the one or more vectors of embodiment 33, or the cell of embodiment 34.
36. A method of inhibiting interaction between HLA-E and NKG2A and/or inhibiting interaction between PD-1 and PD-L1, comprising contacting the NKG2A and/or PD-L1 with the multispecific antibody or fragment thereof of any one of embodiments 1-31 or the pharmaceutical composition of embodiment 35.
37. The method of embodiment 36, wherein the NKG2A cell is expressed on an immune cell.
38. The method of embodiment 37, wherein the immune cell is an NK cell or a T cell, optionally wherein the T cell is a CD8⁺ T cell.
39. The method of any one of embodiments 36-38, wherein the HLA-E is expressed on a cancer cell.
40. A method of inhibiting interaction between PD-1 and PD-L1, comprising contacting the PD-L1 with the multispecific antibody or fragment thereof of any one of embodiments 1-31 or the pharmaceutical composition of embodiment 35.
41. The method of any one of embodiments 36-40, wherein the PD-L1 is on an immune cell or a cancer cell.
42. The method of any one of embodiments 36-41, wherein the PD-1 is expressed on an immune cell, optionally an NK cell or a T cell, further optionally a CD8⁺ T cell.
43. A method of preventing suppression of an immune cell or activating a response mediated by an immune cell, comprising contacting the immune cell with the multispecific antibody or fragment thereof of any one of embodiments 1-31 or the pharmaceutical composition of embodiment 35.
44. The method of embodiment 43, wherein the immune cell is an NK cell or a T cell.
45. The method of embodiment 44, wherein the T cell is a CD8⁺ T cell.
46. The method of any one of embodiments 43-45, wherein the immune cell expresses NKG2A and/or PD-1.
47. The method of any one of clams 43-46, wherein the response mediated by the immune cell is an anti-tumor response, optionally wherein the tumor cell expresses HLA-E and/or PD-L1.
48. A method for treating a disease or disorder in a subject comprising
administering to the subject the multispecific antibody or fragment thereof of any one of embodiments 1-31, or the pharmaceutical composition of embodiment 35.
49. The method of embodiment 48, wherein the disease or disorder is a cancer, optionally the cancer expresses HLA-E and/or PD-L1.
50. The method of embodiment 48, wherein the disease or disorder is an autoimmune and inflammatory disease.
51. The method of any one of embodiments 48-50, wherein the subject is a human subject.
52. An antibody or fragment thereof that binds to PD-L1, comprising a VH CDR1, a VH CDR2, and a VH CDR3 as set forth in a VH comprising the amino acid sequence of SEQ ID NO:98 and a VL CDR1, a VL CDR2, and a VL CDR3 as set forth in a VL comprising the amino acid sequence of SEQ ID NO:99.
53. The antibody or fragment thereof of embodiment 52, comprising
(a) a VH region comprising:
(1) a VH CDR1 having an amino acid sequence selected from the group consisting of SEQ ID NOs: 1, 7, 12, 13, and 18;
(2) a VH CDR2 having an amino acid sequence selected from the group consisting of SEQ ID NOs: 86, 89, 91, 94, and 97; and
(3) a VH CDR3 having an amino acid sequence selected from the group consisting of SEQ ID NOs: 87, 90. 92, and 95; and
(b) a VL region comprising:
(1) a VL CDR1 having an amino acid sequence selected from the group consisting of SEQ ID NOs: 4, 10, 16, and 21;
(2) a VL CDR2 having an amino acid sequence selected from the group consisting of SEQ ID NOs: 5, 11, and 22; and
(3) a VL CDR3 having an amino acid sequence selected from the group consisting of SEQ ID NOs: 88, 93, and 96.
54. The antibody or fragment thereof of embodiment 52 or 53, comprising any one or more of (i)-(vi):
(i) a VH region comprising a VH CDR1 comprising the amino acid sequence of SEQ ID NO: 1, a VH CDR2 comprising the amino acid sequence of SEQ ID NO:86, a VH CDR3 comprising the amino acid sequence of SEQ ID NO:87; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:88;
(ii) a VH region comprising a VH CDR1 comprising the amino acid sequence of SEQ ID NO: 7, a VH CDR2 comprising the amino acid sequence of SEQ ID NO:89, a VH CDR3 comprising the amino acid sequence of SEQ ID NO:90; and a VL region comprising a VL CDR1 comprising the amino acid sequence of SEQ ID NO:10, a VL CDR2 comprising the amino acid sequence of SEQ ID NO:11, and a VL CDR3 comprising the amino acid sequence of SEQ ID NO:88;
(iii) a VH region comprising a VH CDR1 comprising the amino acid sequence of SEQ ID NO: 12, a VH CDR2 comprising the amino acid sequence of SEQ ID NO:86, a VH CDR3 comprising the amino acid sequence of SEQ ID NO:87; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:88;
(iv) a VH region comprising a VH CDR1 comprising the amino acid sequence of SEQ ID NO: 13, a VH CDR2 comprising the amino acid sequence of SEQ ID NO:91, a VH CDR3 comprising the amino acid sequence of SEQ ID NO:92; and a VL region comprising a VL CDR1 comprising the amino acid sequence of SEQ ID NO:16, a VL CDR2 comprising the amino acid sequence of SEQ ID NO:11, and a VL CDR3 comprising the amino acid sequence of SEQ ID NO:93;
(v) a VH region comprising a VH CDR1 comprising the amino acid sequence of SEQ ID NO: 18, a VH CDR2 comprising the amino acid sequence of SEQ ID NO:94, a VH CDR3 comprising the amino acid sequence of SEQ ID NO:95; and a VL region comprising a VL CDR1 comprising the amino acid sequence of SEQ ID NO:21, a VL CDR2 comprising the amino acid sequence of SEQ ID NO:22, and a VL CDR3 comprising the amino acid sequence of SEQ ID NO:96; or
(vi) a VH region comprising a VH CDR1 comprising the amino acid sequence of SEQ ID NO: 1, a VH CDR2 comprising the amino acid sequence of SEQ ID NO:97, a VH CDR3 comprising the amino acid sequence of SEQ ID NO:87; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:88.
55. The antibody or fragment thereof of any one of embodiments 52-54, wherein the VH region comprises the amino acid sequence of SEQ ID NO:98 and the VL region comprises the amino acid sequence of SEQ ID NO:99.
56. A polynucleotide encoding the antibody or fragment thereof of any one of embodiments 52-55.
57. One or more vectors comprising one or more polynucleotides of embodiment 56 or a complementary polynucleotide thereto.
58. A cell comprising any one or more of: the antibody or fragment thereof of any one of embodiments 52-54, the polynucleotide of embodiment 56, or the one or more vectors of embodiment 57.
59. A pharmaceutical composition that comprises a pharmaceutically acceptable excipient and any one or more of: the antibody or fragment thereof of any one of embodiments 52-54, the polynucleotide of embodiment 56, the one or more vectors of embodiment 51, or the cell of embodiment 58.
60. A method of inhibiting interaction between PD-1 and PD-L1, comprising contacting the PD-L1 with the antibody or fragment thereof of any one of embodiments 52-54 or the pharmaceutical composition of embodiment 59.
61. A method of preventing suppression of an immune cell or activating a response mediated by an immune cell, comprising contacting the immune cell with the antibody or fragment thereof of any one of embodiments 52-54 or the pharmaceutical composition of embodiment 59.
62. A method for treating a disease or disorder in a subject comprising administering to the subject the antibody or fragment thereof of any one of embodiments 52-54 or the pharmaceutical composition of embodiment 59.

### 7. EXAMPLES

Phage display was performed to screen for binders that bind to a heterodimer complex comprising the extracellular domains of NKG2A/CD94 (Example 1). The obtained binders were then evaluated for (i) binding to a hererodimer complex comprising the extracellular domains of NKG2C/CD94 or not (Example 2); (ii) binding to the NKG2A/CD94 heterodimer expressed on cells or not (Example 3); (iii) inhibition of HLA-E (tetramer) and NKG2A binding (Example 4); and (iv) developability (Example 5). Those advantageously having (a) binding to NKG2A/CD94 but not NKG2C/CD94 and (b) having colloidal stability and low propensity to aggregate and precipitate were selected, including A3, A2, A42 and A11 (Example 6).

Bispecific antibodies derived from the selected binders were then constructed (Example 7). Their binding affinities (Example 8) and developability (Example 13) were then evaluated. Further assays were utilized to evaluate the bispecific antibodies, including HLA-E/NKG2A inhibitory assays (Example 9), PD-1/PD-L1 signaling inhibitory assays (Example 10), NK92 mediated cytotoxicity assays (Example 11), NK cell degranulation and cell killing assays (Example 12), and flu-stimulated CD8⁺ T cell assays (Example 12). Results showed advantageous effects of those bispecific antibodies, such as inhibition of the HLA-E/NKG2A (such as HLA-E/NKG2A/CD94) signaling, inhibition of the PD-1/PD-L1 checkpoint signaling, promoting NK cell mediated cytotoxicity, promoting NK cell's degranulation, and activating CD8⁺ T cells.

### Example 1. Antibody Generation

Antibodies to NKG2A were generated by phage display. To obtain binders for human NKG2A, antibody discovery was conducted by phage display of human Fab. The extracellular domain of human NKG2A/CD94 heterodimer (SEQ ID NOS: 74 and 76) was expressed and purified using standard procedure. Briefly, the extracellular domain of human NKG2A was expressed and purified from HEK293 cells as heterodimer with CD94 as Fc fusions. The extracellular domain of human NKG2A, encompassing amino acid residues Pro 94 and Leu 233 (SEQ ID NO:74) of gene accession No: P26715-1 was synthesized from IDT and PCR-amplified using appropriate primers. The PCR product was cloned into a mammalian expression construct such that the NKG2A was expressed as an N-terminal Fc (knob) fusion. In addition to the Fc tag, the fusion protein also harbored an N-terminal Avi Tag (for biotinylating) and 6×His tag for purification. The gene encoding CD94 (Accession No. Q13241-1; the extracellular domain of human CD94 is as shown in SEQ ID NO:76), Lys33-Ile159 was synthesized from IDT and cloned into mammalian expression vector as an N-terminal Fc (hole) fusion. Plasmids encoding NKG2A-Fc fusion and CD94-Fc fusion were co-transfected into HEK293T cells using standard procedures. NKG2A/CD94 heterodimer was purified from cell supernatant using Protein A resins. Purified protein complex was biotinylated site-specifically using BirA-biotin protein ligase (Avidity: BirA 500 BirA biotin ligase standard reaction kit) using manufacturer's protocol. Additionally, the purified protein complex was subjected to random biotinylation using EZ-Link NHS-PEG12-Biotin reagent (ThermoScientific Cat. No. 21312) using standard protocol. Briefly, the purified heterodimer was treated with 10-fold molar excess of EZ-Link NHS-PEG12-Biotin in PBS (200 μl) at room temperature for 30 min. The reaction was quenched by adding Tris-HCl (1 mM final). Excess biotin reagent was removed by dialysis against 500 ml PBS buffer for 48 hours with two buffer exchanges, after 2 hours and 24 hours. Phage clones were screened for the ability to bind to biotinylated human NKG2A by phage ELISA using standard protocols. Briefly, Fab-formatted phage libraries were constructed using expression vectors capable of replication and expression in phage (also referred to as a phagemid). Both the heavy chain and the light chain were encoded in the same expression vector, where the heavy chain was fused to a truncated variant of the phage coat protein pIII. The light chain and heavy chain-pIII fusion were expressed as separate polypeptides and assembled in the bacterial periplasm, where the redox potential enables disulfide bond formation, to form the antigen-binding domain (Fab) of the candidate antibody.

The library was created using sequences derived from a human heavy chain variable domain and a human light chain variable domain. Light chain variable domains within the screened library were generated with diversity was introduced into the VL CDR3 (L3) and where the light chain VL CDR1 (L1) and CDR2 (L2) remained the human germline sequence. For the screened library, all three CDRs of the VH domain were diversified to match the positional amino acid frequency by CDR length found in the human antibody repertoire. The phage display heavy chain (SEQ ID NO:79) and light chain (SEQ ID NO:80) scaffolds used in the library are listed below, where a “X” represents CDR amino acids that were varied to create the library, and bold italic represents the CDR sequences that were constant.

The sequence for SEQ ID NO:79 was

EVQLVESGGGLVQPGGSLRLSCAASGFTFSXXXXXWVRQAPGKGLE
WVAXXXXXXXXXXXXXXXXXRFTISADTSKNTAYLQMNSLRAEDTA
VYYCARXXXXXXXXXXXXXXWGQGTLVTVSSASTKGPSVFPLAPSS
KSTSGGTAALGCLVKDYFPEPVTVSWNSGALTSGVHTFPAVLQSSG
LYSLSSVVTVPSSSLGTQTYICNVNHKPSNTKVDKKVEPKSC.

The sequence for SEQ ID NO:80 was

DIQMTQSPSSLSASVGDRVTITCRASQSVSSAVAWYQQKPGKAPKLL
IYSASSLYSGVPSRFSGSRSGTDFTLTISSLQPEDFATYYCXXXXXX
XXXFGQGTKVEIKRTVAAPSVFIFPPSDSQLKSGTASVVCLLNNFYP
REAKVQWKVDNALQSGNSQESVTEQDSKDSTYSLSSTLTLSKADYEK
HKVYACEVTHQGLSSPVTKSFNRGEC.

Diversity was created through mutagenesis using degenerate DNA oligonucleotide primers to introduce diversity into VL CDR3 and VH CDR1 (H1), CDR2 (H2) and CDR3 (H3) to mimic the diversity found in the natural antibody repertoire, as described in more detail in Kunkel, TA (PNAS Jan. 1, 1985. 82 (2) 488-492), herein incorporated by reference in its entirety. Briefly, uracil-incorporated single-stranded circular DNAs were prepared from isolated phage using standard procedures and Kunkel mutagenesis carried out to introduce diversity to the four CDRs. Chemically-synthesized DNA was then electroporated into TG1 cells, followed by recovery. Recovered cells were sub-cultured and infected with M13K07 helper phage to produce the phage library.

Phage panning was performed using standard procedures. Briefly, the first round of phage panning was performed with target immobilized on streptavidin magnetic beads which were subjected to approximately 1×10¹² phages from the prepared library in a volume of 1 mL in PBST-2% BSA. After a one-hour incubation, the bead-bound phage were separated from the supernatant using a magnetic stand. Beads were washed three times to remove non-specifically bound phage and were then added to ER2738 cells (5 mL) at OD₆₀₀ of approximately 0.6. After 20 minutes incubation at room temperature, infected cells were sub-cultured in 25 mL 2×YT+Ampicillin and M13K07 helper phage (final concentration of approximately 1×10¹⁰ pfu/ml) and allowed to grow overnight at 37° C. with vigorous shaking. The next day, phage were prepared using standard procedures by PEG precipitation. Pre-clearance of phage specific to SAV-coated beads was performed prior to panning. The second round of panning was performed using the KingFisher magnetic bead handler with bead-immobilized NKG2A target antigen using standard procedures (round 3:100 nM NKG2A, round 4:50 nM NKG2A). In total, 3-4 rounds of phage panning were performed to enrich in phage displaying Fabs specific for the target antigen. Target-specific enrichment was confirmed using polyclonal ELISA and individual clones were isolated and further verified by performing monoclonal phage ELISA. DNA sequencing was used to determine the sequence of the CDRs of isolated Fab clones containing a candidate antibody. A total of 73 clones were screened and selected from the phage library.

The genes encoding heavy chain and light chain variable domains of the candidate antibodies were cloned separately into mammalian expression vectors for expression as full length IgGs in mammalian cells.

For the full length IgGs, the heavy chain constant regions (e.g., CH1=regular text; Hinge=italicized text; CH2=bold text; and CH3=underline text) included the following amino acid sequence:

(SEQ ID NO: 81)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPGK.

For the full length IgGs, the light chain constant region (e.g., CL) included the following amino acid sequence:

(SEQ ID NO: 82)
RTVAAPSVFIFPPSDSQLKSGTASVVCLLNNFYPREAKVQWKVDNA
LQSGNSQESVTEQDSKDSTYSLSSTLTLSKADYEKHKVYACEVTHQ
GLSSPVTKSFNRGEC.

The IgG antibodies were purified from culture supernatant using Protein A resin.

### Example 2. Binding Assays

A total of 58 binders formatted as IgG antibodies from phage display as described in Example 1 were further tested for binding. For example, to determine qualitative binding, bio-layer interferometry (BLI) was used to confirm the specific interaction of the antigens to the candidate antibodies obtained in Example 1.

The antibodies were evaluated for binding to (1) human NKG2A, (2) human NKG2C, and (3) cyno NKG2A. Bivalent interaction of binders to biotinylated human NKG2A (see Example 1), biotinylated human NKG2C/CD94 heterodimer, or biotinylated cyno NKG2A/CD94 heterodimer immobilized on a streptavidin biosensor was monitored using Octet (Pall ForteBio) instrument. For these assays, human NKG2C/CD94 and cyno NKG2A/CD94 were prepared using the procedure as described in Example 1. Similarly, the extracellular domain of human NKG2C was expressed and purified from HEK293 cells as heterodimer with CD94 as Fc fusions. The extracellular domain of NKG2C used for selection campaigns encompassed amino acid sequence Ile94-Leu231 (Accession No. P26717-1; SEQ ID NO: 75). The gene encoding CD94 (Accession No. Q13241-1, SEQ ID NO:76), Lys33-Ile159 was synthesized from IDT and cloned into mammalian expression vector as an N-terminal Fc (hole) fusion. Plasmids encoding NKG2C-Fc fusion and CD94-Fc fusion were co-transfected into HEK293T cells using standard procedures. NKG2C/CD94 heterodimer was purified from cell supernatant using Protein A resins. Similar approach was used to obtain Cynomolgus NKG2A protein. Specifically, extracellular domain of Cynomolgus NKG2A contained amino acid Pro 94-Leu 233. Variations included Cys 114 to Arg point mutation (Accession No. Q68VD2-1; SEQ ID NO:77). Extracellular domain of Cynomolgus CD94 contained amino acid residues Lys 33-Ile 179 (Accession No. Q68VD4-1; SEQ ID NO: 78).

Results of the qualitative bivalent Octet binding assays showed that all 58 antibodies tested bound to human NKG2A, 26 of the 58 antibodies bound to NKG2C, and 4 of the antibodies bound to cyno NKG2A.

TABLE 6
Extracellular Domains

| Description | Amino Acid Sequences |
|---|---|
| Extracellular | PSTLIQRHNNSSLNTRTQKARHCGHCPEEWITYSNSCYYIGKERRT |
| domain of human | WEESLLACTSKNSSLLSIDNEEEMKFLSIISPSSWIGVFRNSSHHPW |
| NKG2A | VTMNGLAFKHEIKDSDNAELNCAVLQVNRLKSAQCGSSIIYHCKH |
| | KL (SEQ ID NO: 74) |
| Extracellular | IPFLEQNNSSPNTRTQKARHCGHCPEEWITYSNSCYYIGKERRTWE |
| domain of human | ESLLACTSKNSSLLSIDNEEEMKFLASILPSSWIGVFRNSSHHPWVT |
| NKG2C | INGLAFKHKIKDSDNAELNCAVLQVNRLKSAQCGSSMIYHCKHKL |
| | (SEQ ID NO: 75) |
| Extracellular | KNSFTKLSIEPAFTPGPNIELQKDSDCCSCQEKWVGYRCNCYFISSE |
| domain of human | QKTWNESRHLCASQKSSLLQLQNTDELDFMSSSQQFYWIGLSYSE |
| CD94 | EHTAWLWENGSALSQYLFPSFETFNTKNCIAYNPNGNALDESCED |
| | KNRYICKQQLI (SEQ ID NO: 76) |
| Extracellular | PSTLTQKHNNSSLNTRTQKARHCGHCPEEWITYSNSCYYIGKEKR |
| domain of | TWAESLLACTLKNSSLLSIDNEEEMKFLTAISPSTWTGVFRDSSQH |
| cynomolgus | PWVTINGLTFKHEIKDSDNAEHNCAMLHARGLKSDRCGSSKIYHC |
| NKG2A | KHKL (SEQ ID NO: 77) |
| Extracellular | KNSFTKLSVEPAYTPGPNIELQKDSDCCSCHEKWVGYRCNCYFISS |
| domain of | EEKTWNESRHFCASQKSSLLQLQNRDELDFMSSSQHFYWIGLSYS |
| cynomolgus | EEHTAWLWENGSALSQYLFPSFETFKPKNCIAYNSKGNALDESCE |
| CD94 | TKNRYICKQQLI (SEQ ID NO: 78) |

### Example 3. Cell Binding Assays

Antibodies that were selected for binding to NKG2A, for example, such as those described in Examples 1 and 2, were evaluated for binding to cells that express NKG2A. For example, the 58 antibodies tested in binding assays in Example 2 were also evaluated using flow cytometry for binding to KHYG-1 cells expressing endogenous NKG2A (Accegen, ABC-TC0506), which have a surface NKG2A copy number of approximately 10-20K.

Cells were harvested at 70-90% confluence on the day of the assay. Cells were collected by centrifugation at 200×g for 5 minutes and media was removed. Cells were resuspended at 2×10⁶ cells per mL in cold PBS. An 8-point antibody dilution series (2× concentration) was prepared in PBS to cover the expected binding affinities of the antibodies being tested. 50 μL per well of the antibody dilution was plated in a 96 well V-bottom plate (Costar 3897). 50 μL per well of cell suspension was added. Plates were placed at 4° C. for 45-60 minutes.

Cells were collected by centrifugation at 400×g for 7 minutes and primary antibody was removed. 50 μL per well of AF488 goat anti human IgG Fab (Jackson Immuno Research 109-547-003) at 1:100 dilution was added. Plates were placed at 4° C. for 30 minutes.

Cells were collected by centrifugation at 400×g for 7 minutes and secondary antibody was removed. Cells were resuspended in 50 μL per well of PBS and analyzed by flow cytometry. Binding curves were calculated using the mean fluorescence intensity (MFI) of the FITC fluorescence signal on the cells.

From the cell binding assays, affinity of NKG2A antibody binding to KHYG-1 cells was grouped into “strong,” “moderate,” and “weak or not detectable” binding by EC₅₀ value. Antibodies with an EC₅₀ value lower than 10 nM showed strong binding, antibodies with an EC₅₀ value of 10 to 200 nM showed moderated binding, and antibodies with an EC₅₀ value of higher than 200 nM showed weak or non-detectable binding. Results from the 58 antibodies tested showed that 16 demonstrated strong cell binding, 27 demonstrated moderate cell binding, and 15 demonstrated weak or not-detectable cell binding. These results indicated that the NKG2A antibodies exhibited a wide range of cell binding activity represented by EC₅₀ values.

### Example 4. Functional Assays

Antibodies that were selected for binding to NKG2A, for example, such as those described in Examples 1 and 2, were evaluated in HLA-E/NKG2A inhibitory assays.

For example, the 58 antibodies tested in Examples 2 and 3 were also evaluated for inhibition of HLA-E (tetramer) and NKG2A binding in KHYG-1 cells expressing endogenous NKG2A. For these assays, 15 μL per well of binder dilution at 2× concentration was plated in a 96-well plate (Costar 3897). Then 15 μL per well of cell suspension at 5×10⁶ cells per mL was added. Plates were placed at 4° C. for 30 minutes. Following the incubation, 5 μL per well of Fc block (BD Pharmingen Cat #564220) was added at a final concentration of 500 ng per well. Plates were placed at 4° C. for 10 minutes. Following the incubation, 4 μL per well of diluted HLA-E was added. Plates were placed at 4° C. for 1 hour. Following the incubation, 100 μL per well of PBS was added and plate was centrifuged at 400×g for 7 minutes. Supernatant was removed by flicking the plate. Cells were resuspended in 50 μL per well of PBS and analyzed by flow cytometry (Intellicyt).

Results of HLA-E/NKG2A inhibitory assays showed that 15 of the 58 antibodies tested demonstrated strong competitive inhibition of HLA-E/NKG2A binding, 34 demonstrated moderate competitive inhibition of HLA-E/NKG2A binding, and 9 demonstrated weak or not-detectable competitive inhibition of HLA-E/NKG2A binding. These results indicated that the NKG2A antibodies exhibited a wide range of binding inhibition activity.

### Example 5. Developability Assays

Antibodies that were selected for binding to NKG2A, for example, such as those described in Examples 1 and 2, were evaluated in various developability methods. For example, the 58 antibodies tested in Examples 2-4 were also evaluated using various chromatographic methods, including size exclusion chromatography (SEC), hydrophobic interaction chromatography (HIC), and standup monolayer adsorption chromatography (SMAC). These methods were employed to assess developability factors, such as monomer percentage, solubility, and antibody aggregation or precipitation.

Size exclusion chromatography (SEC) analysis was performed using a 7.8 mm ID×30 cm TSKgel G3000SWXL column (Tosoh Bioscience LLC, PN 08541) on an Agilent 1100 HPLC. The NKG2A binder as described herein was normalized to 1 mg/mL concentration in Dulbecco's PBS (pH 7.4, without Ca²⁺/Mg²⁺) and clarified via centrifugation to pellet particulates while still retaining soluble aggregates. The mobile phase buffer was Dulbecco's PBS (pH 7.4, without Ca²⁺/Mg²⁺). 10 μL sample was loaded and isocratically eluted at 1.0 mL/min over 20 minutes. Absorbance was monitored at 280 nm. Chromatographic peaks were integrated to determine % homogeneity and retention time. The column stationary phase along with choice of mobile phase supports hydrophobic interaction in addition to molecular sizing (hydrophobic interaction much milder compared to SMAC). Data analysis was performed using Agilent ChemStation B.04.03.

For the 58 antibodies tested, SEC results showed that 44 of the 58 had strong developability based on SEC, 15 had moderate developability based on SEC, and 1 had weak or not-detectable developability based on SEC.

Hydrophobic interaction chromatography (HIC) analysis was performed using a 4.6 mm ID×3.5 cm TSKgel Butyl-NPR column (Tosoh Bioscience LLC, PN 14947) on an Agilent 1100 HPLC. The NKG2A binders as described herein was normalized to 2 mg/mL concentration in dPBS (pH 7.4) and then diluted with an equal volume of mobile phase buffer B to a final protein concentration of 1 mg/mL. The column was equilibrated with 100% mobile phase Buffer B (2 M ammonium sulfate/20 mM sodium phosphate, pH 7.0) at a flow rate of 1 mL/min. 10 μL sample was loaded and eluted using a gradient from 100% mobile phase buffer B to 100% mobile phase buffer A (20 mM sodium phosphate, pH 7.0) at 1.0 mL/min over 15 min, held at 100% A for 3 min to wash the column, and returned 100% B for 2 min for equilibration. Absorbance was monitored at 280 nm. Sample retention time was calculated and compared to a set of standard controls to identify binders with increased retention time (increased hydrophobicity).

Antibody hydrophobicity can impact antibody aggregation, solubility and viscosity. Antibody HIC elution profiles (e.g., sharpness of elution peak and uniform retention times) suggest propensities to aggregate and/or precipitate. For the 58 antibodies tested, results showed that 50 of the 58 had strong developability based on HIC, 7 had moderate developability based on HIC, and 1 had weak or not-detectable developability based on HIC.

Standup monolayer adsorption chromatography (SMAC) analysis was performed using a 4.6 mm ID×300 mm Zenix SEC 300 column (Sepax Technologies, PN 213300P-4630) on an Agilent 1100 HPLC. The NKG2A binder as described herein was normalized to 1 mg/mL concentration in dPBS (pH 7.4) and clarified via centrifugation to pellet particulates. The mobile phase buffer was dPBS (pH 7.4, without calcium and magnesium). 10 μL sample was loaded and isocratically eluted at 0.25 mL/min over 32 min. Absorbance was monitored at 280 nm. Sample retention time was calculated and compared to a set of standard controls to identify binders with increased retention time (increased propensity to form aggregates).

For the 58 antibodies tested, results showed that 35 of the 58 had strong developability based on SMAC, 15 had moderate developability based on SMAC, and 8 had weak or not-detectable developability based on SMAC.

### Example 6. Antibody Selection

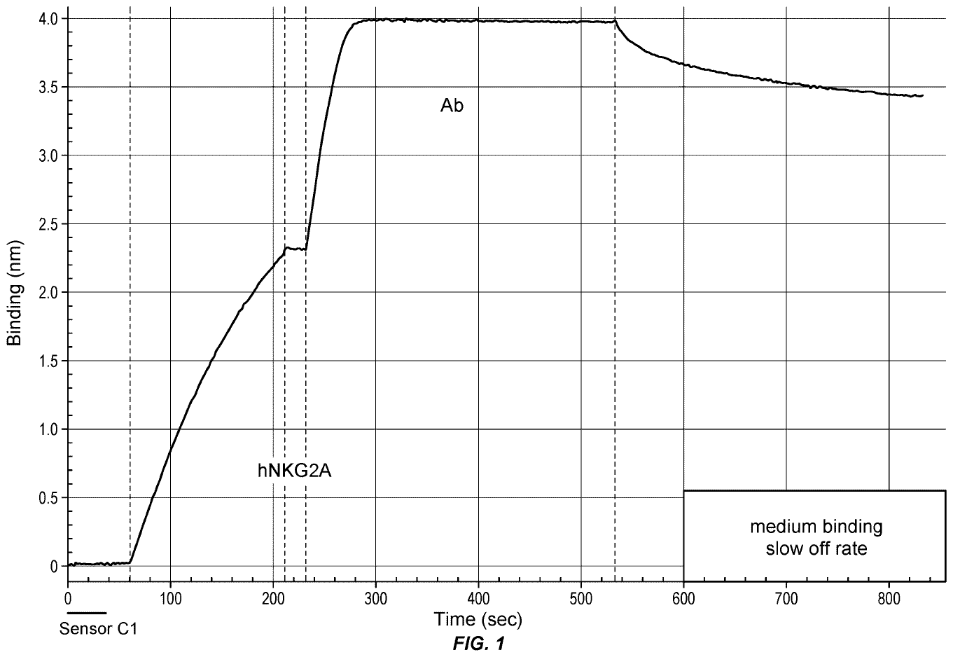

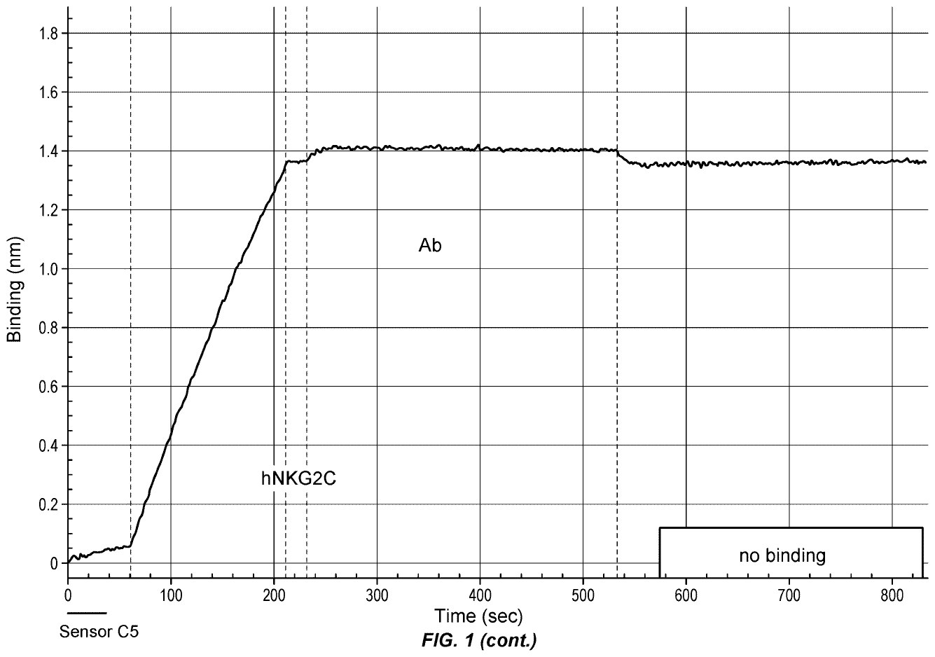

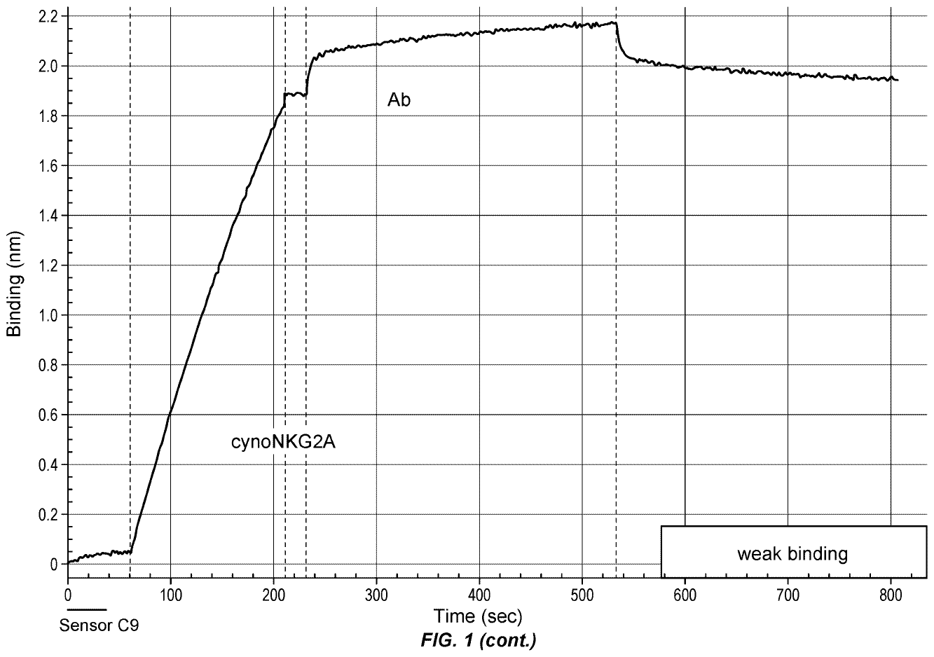

From the 73 antibodies screened from the phage library as described in Example 1, 67 antibodies bound to NKG2A on octet and 15 antibodies failed to bind cells expressing NKG2A. 58 antibodies that were still able to bind to NKG2A after formatting into IgG were further tested as described in Examples 2-5. Two antibodies were selected based on a multiplicity of activities, including their specificity for human NKG2A but not NKG2C binding, their binding to cyno NKG2A, their cell surface binding signal, and their moderate or strong developability as assessed by a variety of methods such as SEC, HIC, and SMAC. These two selected antibodies were designated A3 and A42. An affinity matured variant of each of these antibodies was also selected and designated A2 and A11, respectively. The VH, VL, and CDR sequences of these antibodies are shown in Tables 1-4. Exemplary results for these antibodies that were tested as described in Examples 2-5, are shown in FIGS. 1-12C.

Exemplary bivalent Octet binding for A3 is depicted in FIG. 1. Results indicate that A3 demonstrated moderate binding and slow off rate to human NKG2A. No binding to human NKG2C or cyno NKG2A was observed for A3.

Exemplary cell binding for A3 with NKG2A-expressing cells is represented by an EC₅₀ value. Results show that A3 demonstrated strong cell binding with an EC₅₀ value of 3.032E-10 M.

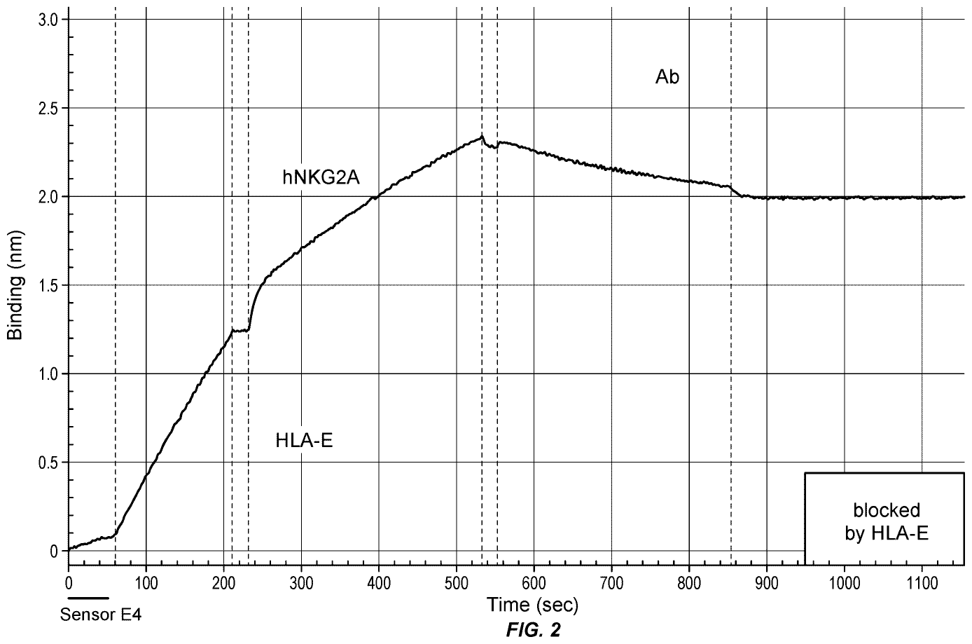

Exemplary HLA-E/NKG2A inhibition for A3 is depicted in FIG. 2. Results indicate that HLA-E and A3 compete for the same binding site on human NKG2A.

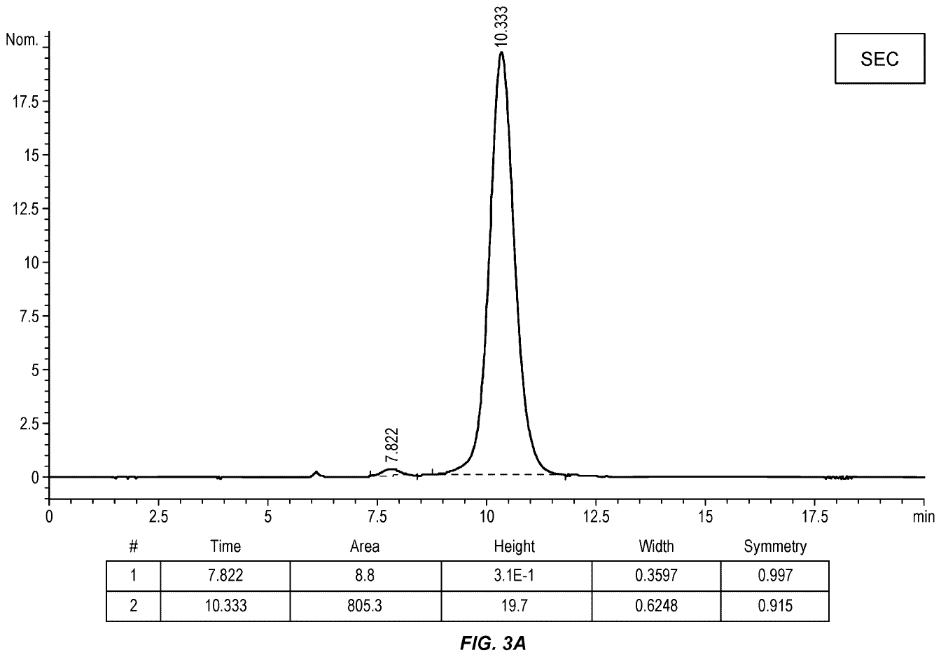

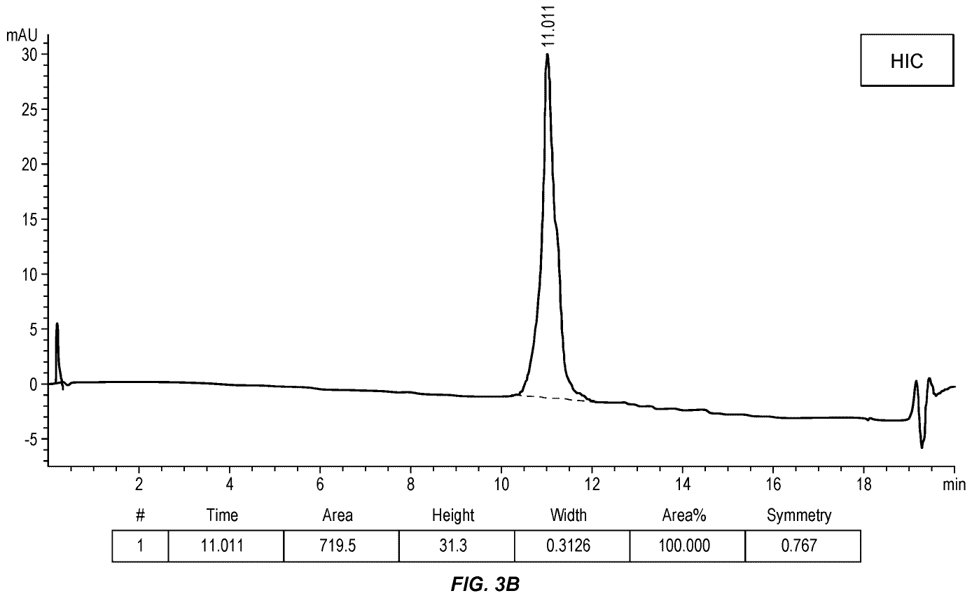

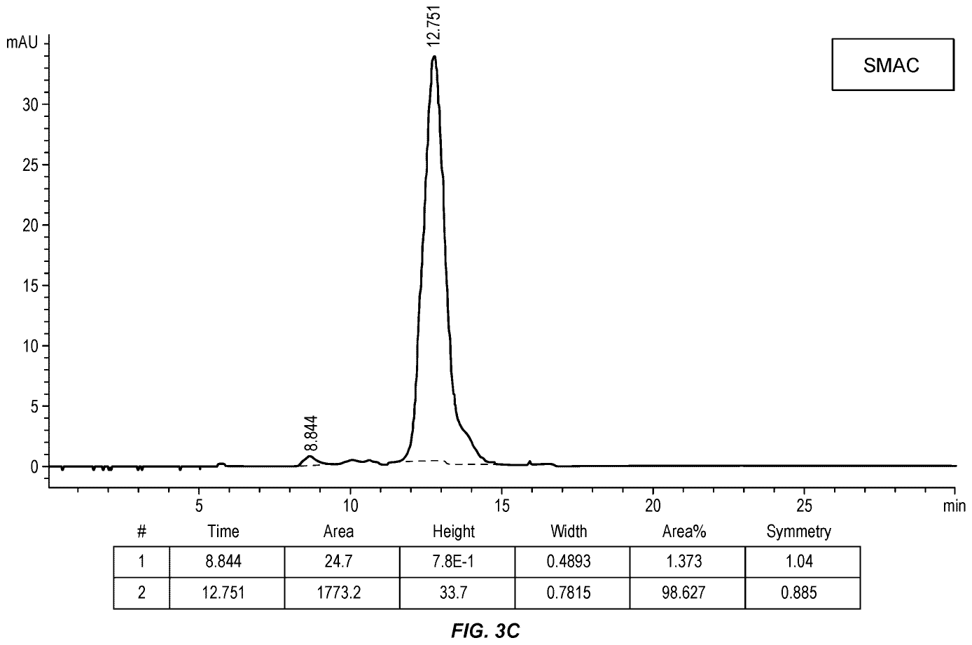

Exemplary SEC results for A3 are shown in FIG. 3A. Results indicate that A3 had low antibody aggregation. Exemplary HIC results for A3 are shown in FIG. 3B. Antibody hydrophobicity can impact antibody aggregation, solubility and viscosity. The HIC elution profiles (e.g., sharpness of elution peak and uniform retention times) of A3 suggested low propensities to aggregate and/or precipitate. Exemplary SMAC results for A3 are shown in FIG. 3C. Results indicated colloidal stability and low propensity to aggregate. Overall, the results of the developability assays show that A3 antibody met the developability criteria.

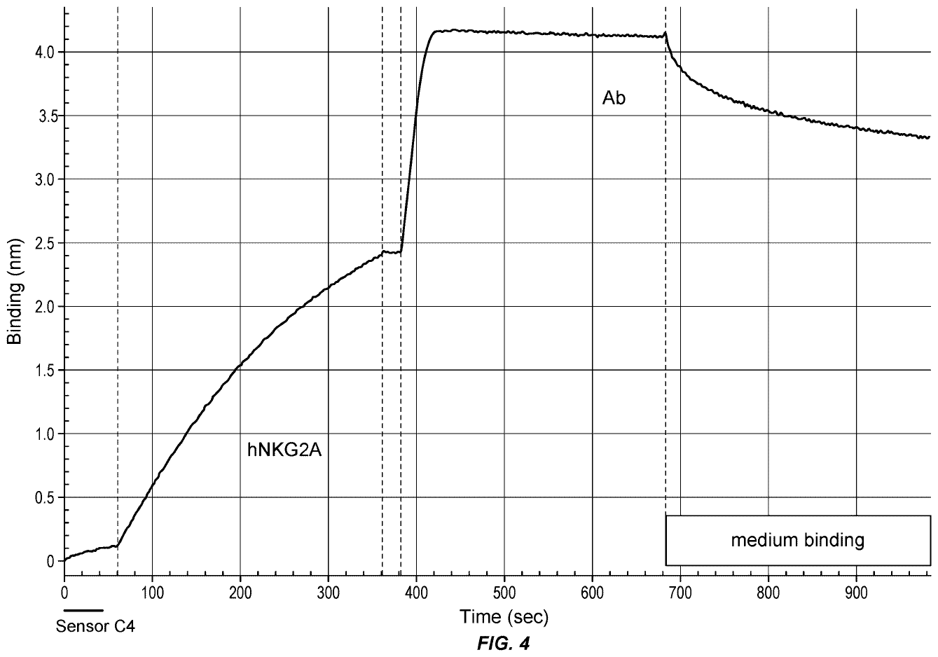

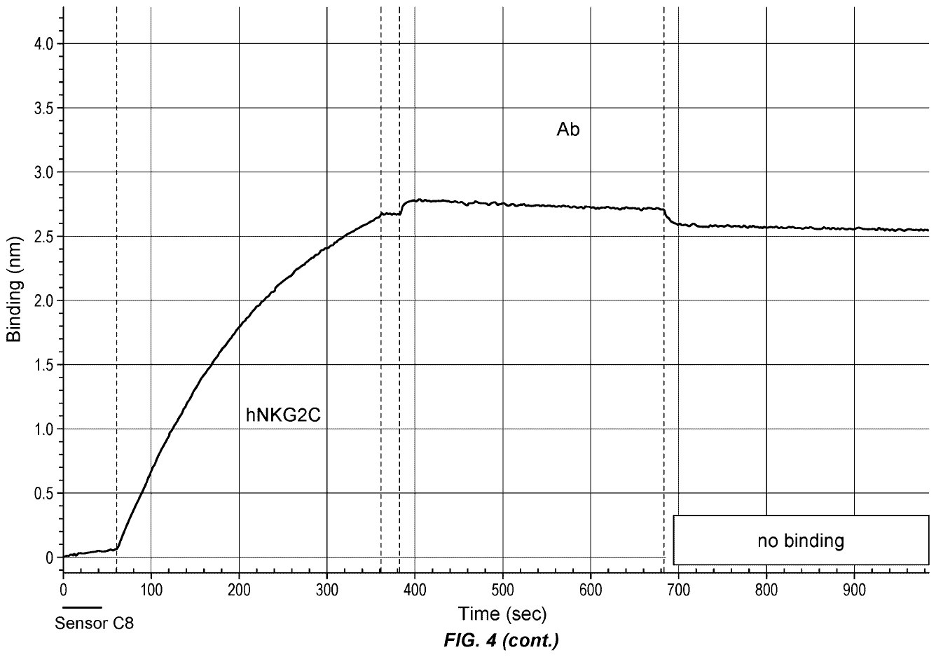

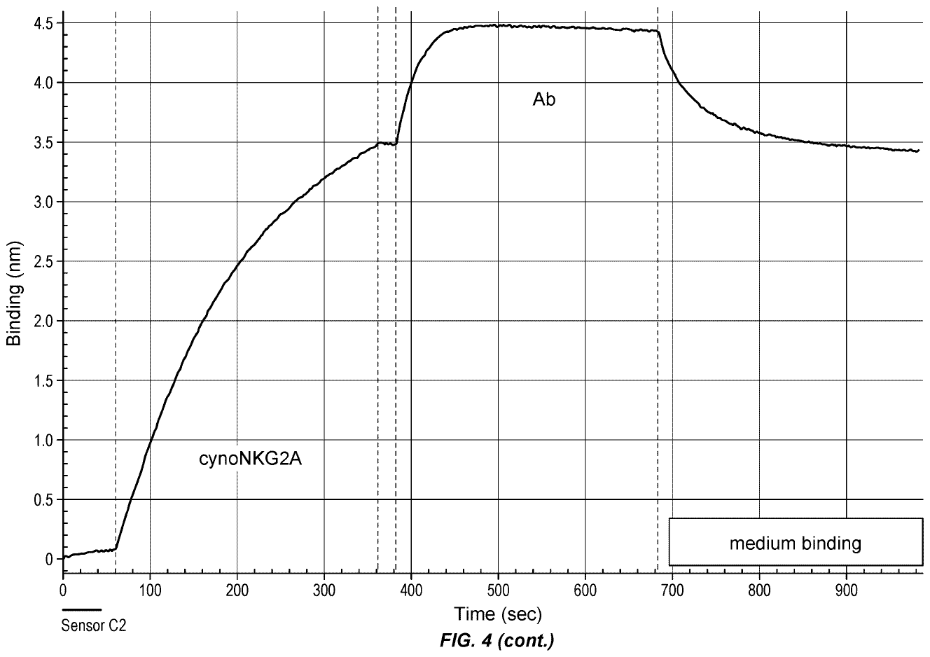

Exemplary bivalent Octet binding for A42 is depicted in FIG. 4. Results indicate that A42 demonstrated moderate binding to human NKG2A and moderate binding to cyno NKG2A. No binding to human NKG2C was observed for A42.

Exemplary cell binding result for A42 is represented by an EC₅₀ value. Results show that A42 demonstrated moderate cell binding with an EC₅₀ value of 2.67E-8 M.

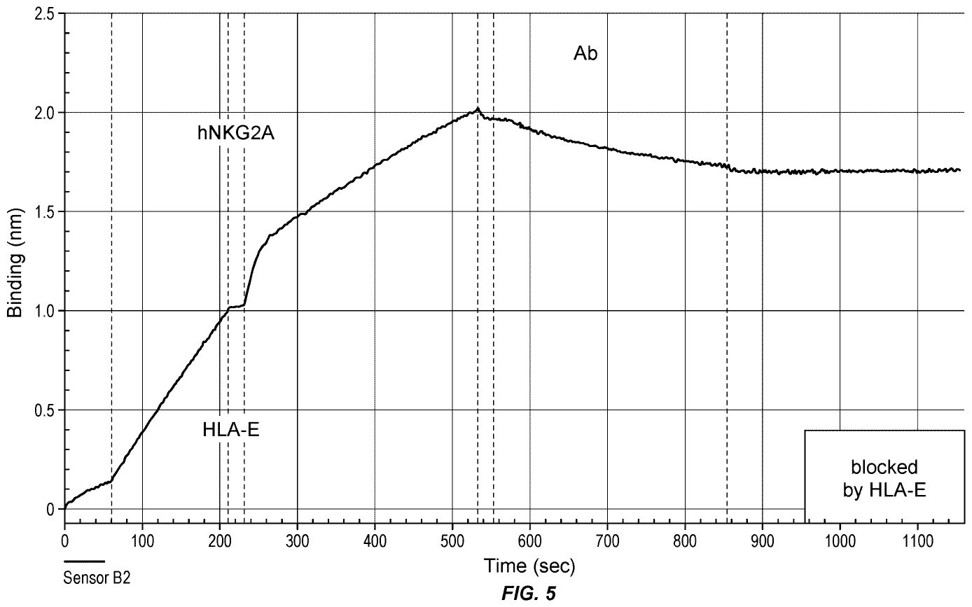

Exemplary HLA-E/NKG2A inhibition for A42 is depicted in FIG. 5. Results indicate that HLA-E and A42 compete for the same binding site on human NKG2A.

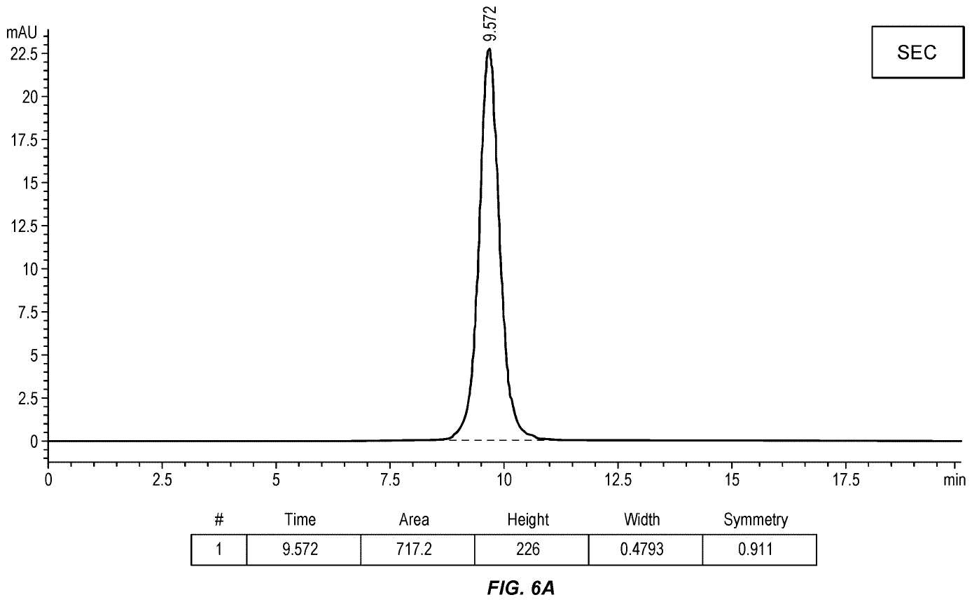

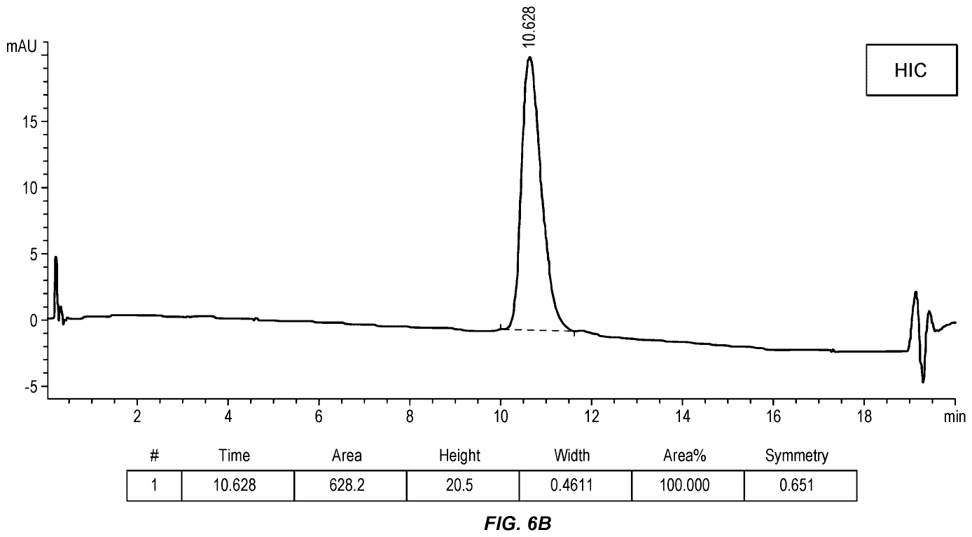

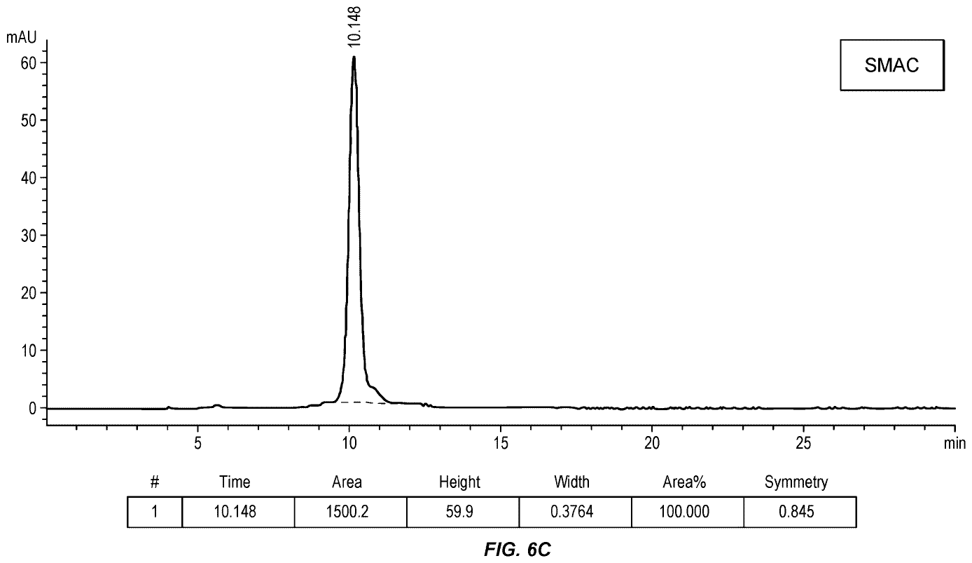

Exemplary SEC results for A42 are shown in FIG. 6A. Results indicate that A42 had low antibody aggregation. Exemplary HIC results for A42 are shown in FIG. 6B. Antibody hydrophobicity can impact antibody aggregation, solubility and viscosity. The HIC elution profiles (e.g., sharpness of elution peak and uniform retention times) of A42 suggested low propensities to aggregate and/or precipitate. Exemplary SMAC results for A42 are shown in FIG. 6C. Results indicated colloidal stability and low propensity to aggregate. Overall, the results of the developability assays show that A42 antibody met the developability criteria.

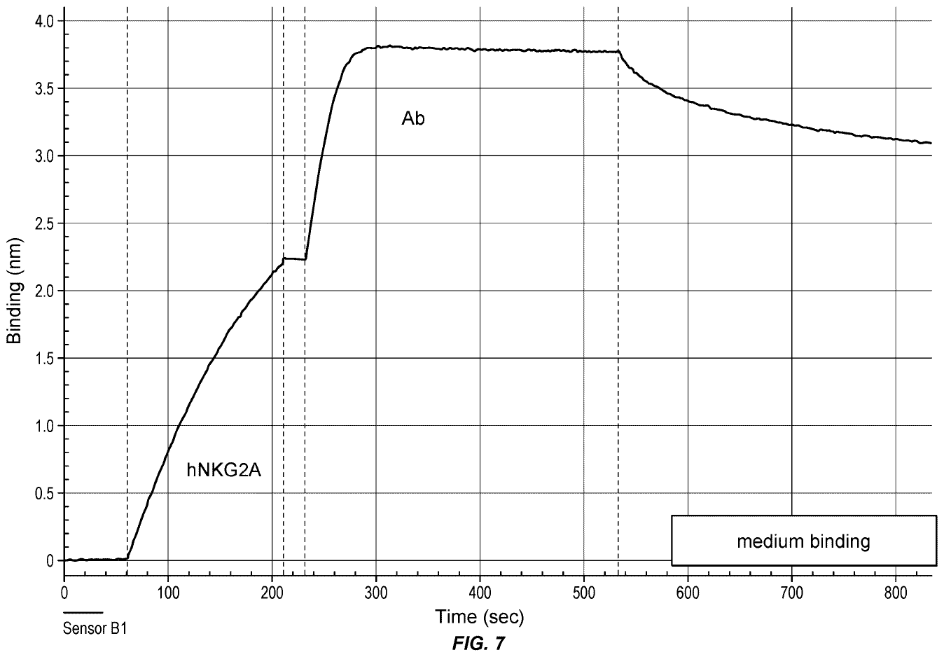

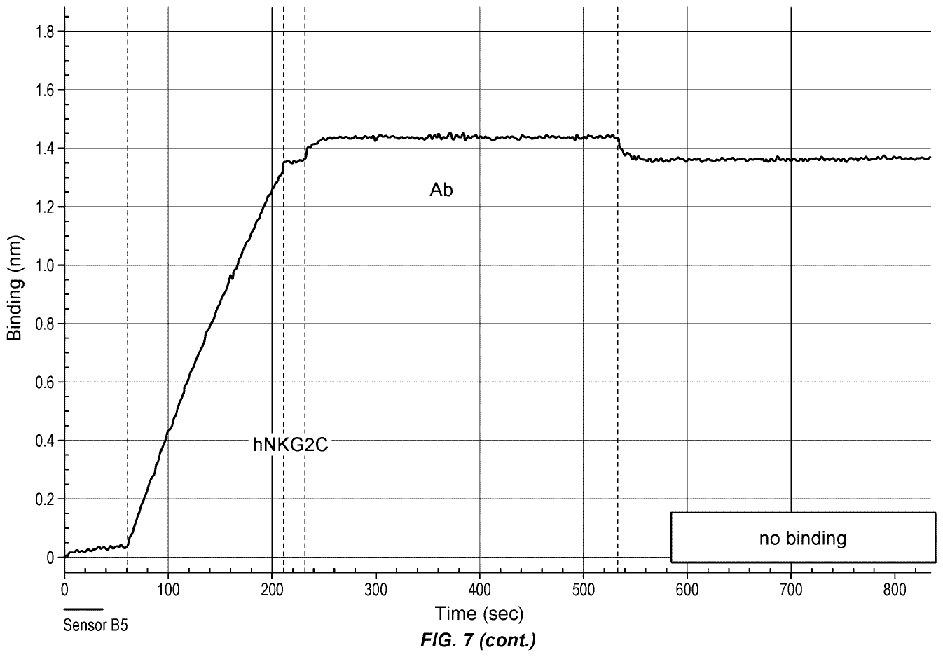

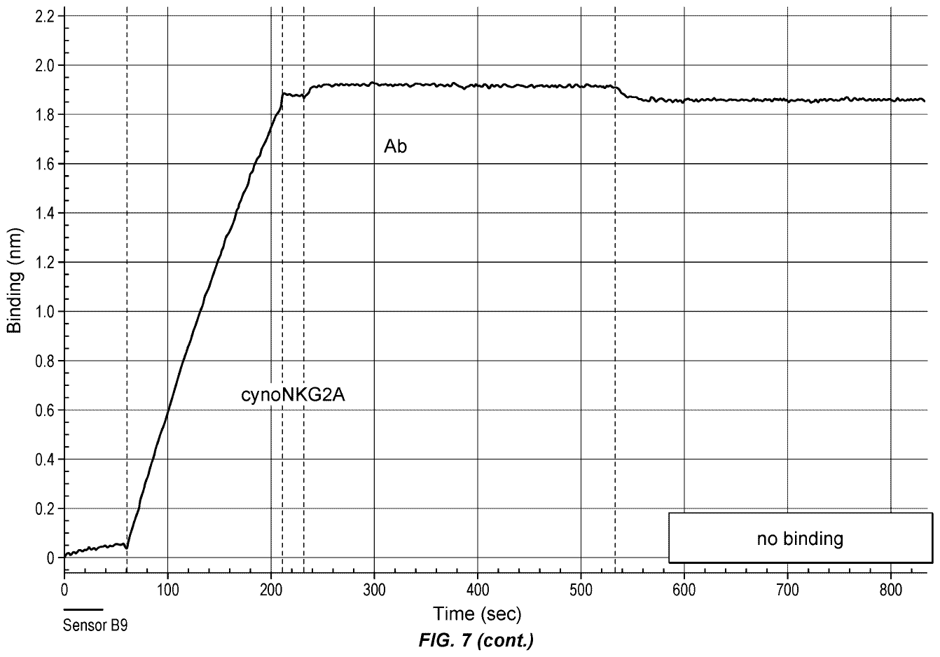

Exemplary bivalent Octet binding for an affinity matured variant of A3 designated A2 is depicted in FIG. 7. Results indicate that A2 demonstrated moderate binding to human NKG2A. No binding to human NKG2C or cyno NKG2A was observed for A2.

Exemplary cell binding result for A2 is represented by an EC₅₀ value. Results show that A2 demonstrated moderate cell binding with an EC₅₀ value of 3.75E-8 M.

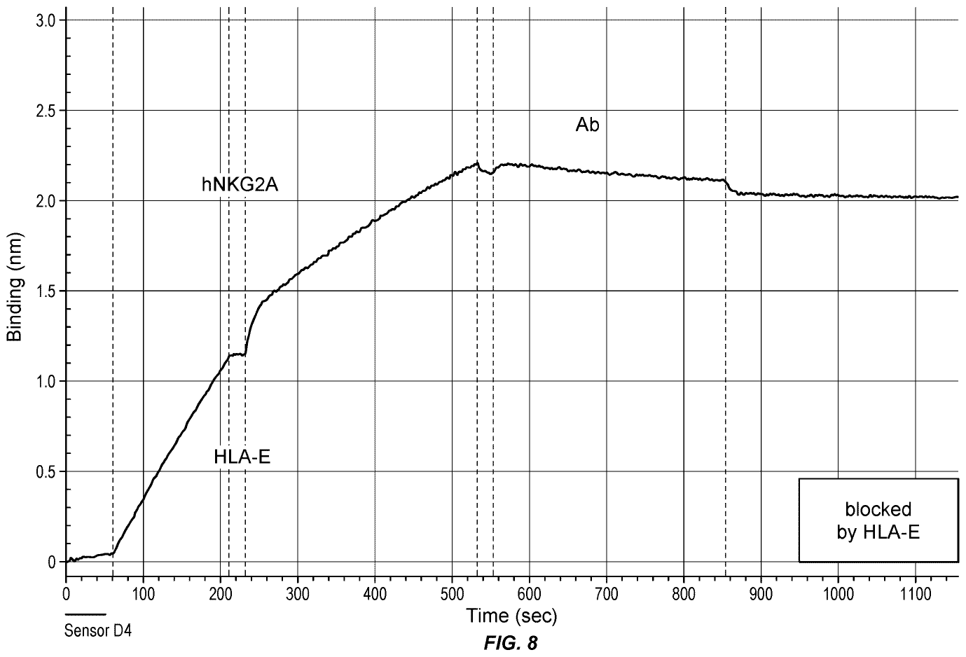

Exemplary HLA-E/NKG2A inhibition for A2 is depicted in FIG. 8. Results indicate that HLA-E and A2 compete for the same binding site on human NKG2A.

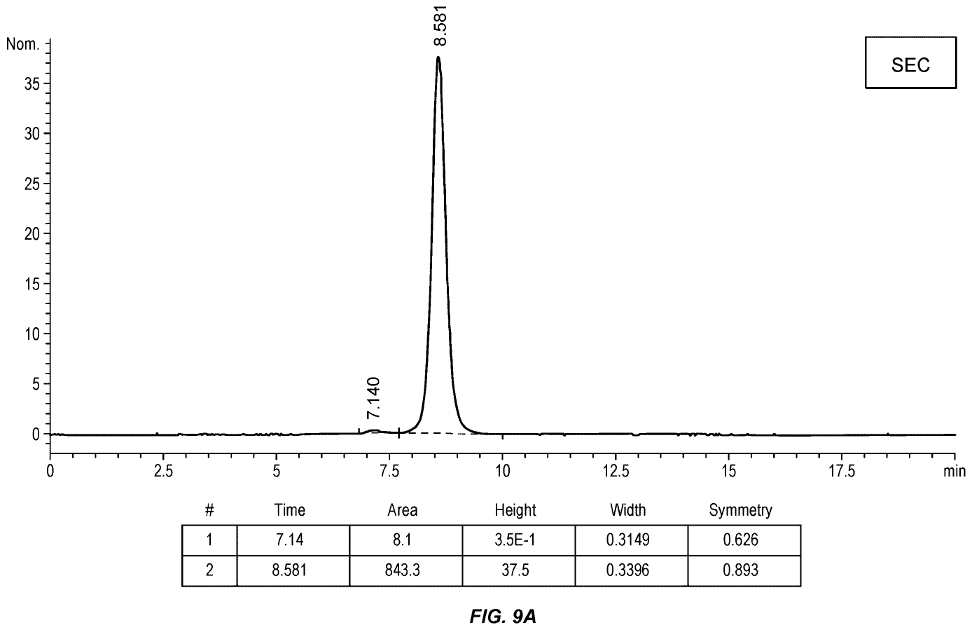

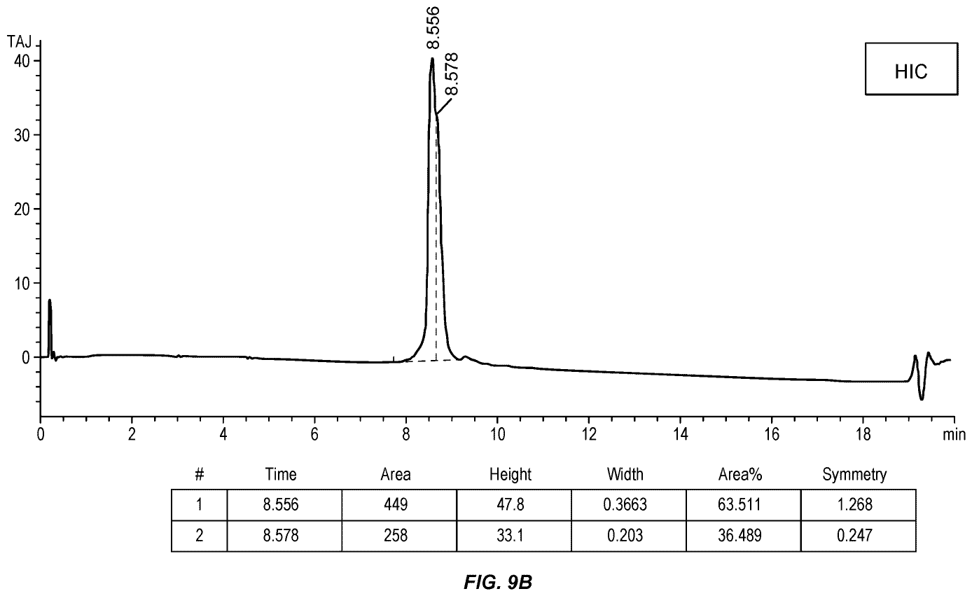

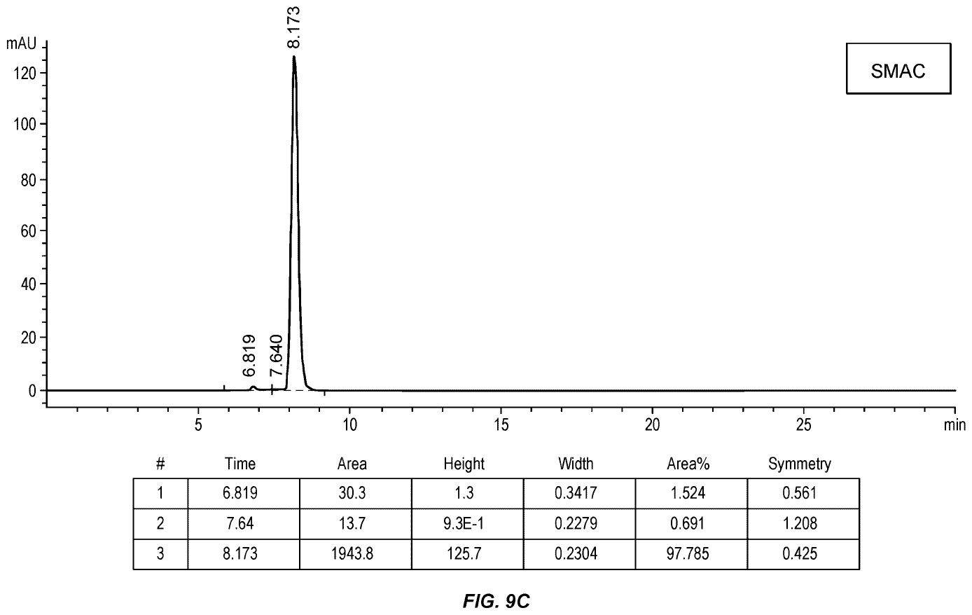

Exemplary SEC results for A2 are shown in FIG. 9A. Results indicate that A2 had low antibody aggregation. Exemplary HIC results for A2 are shown in FIG. 9B. Antibody hydrophobicity can impact antibody aggregation, solubility and viscosity. The HIC elution profiles (e.g., sharpness of elution peak and uniform retention times) of A2 suggested low propensities to aggregate and/or precipitate. Exemplary SMAC results for A2 are shown in FIG. 9C. Results indicated colloidal stability and low propensity to aggregate. Overall, the results of the developability assays show that A2 antibody met the developability criteria.

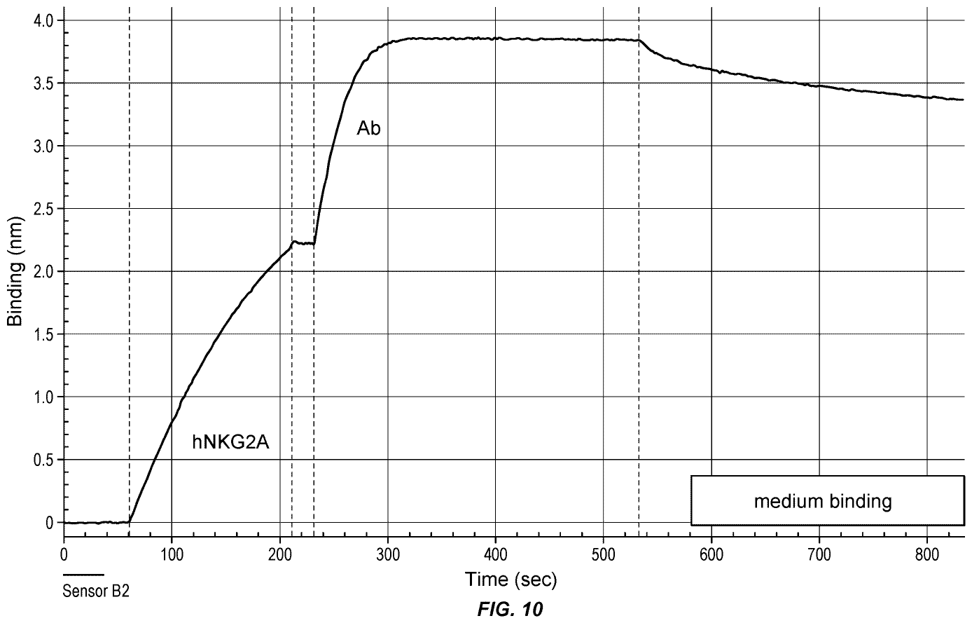

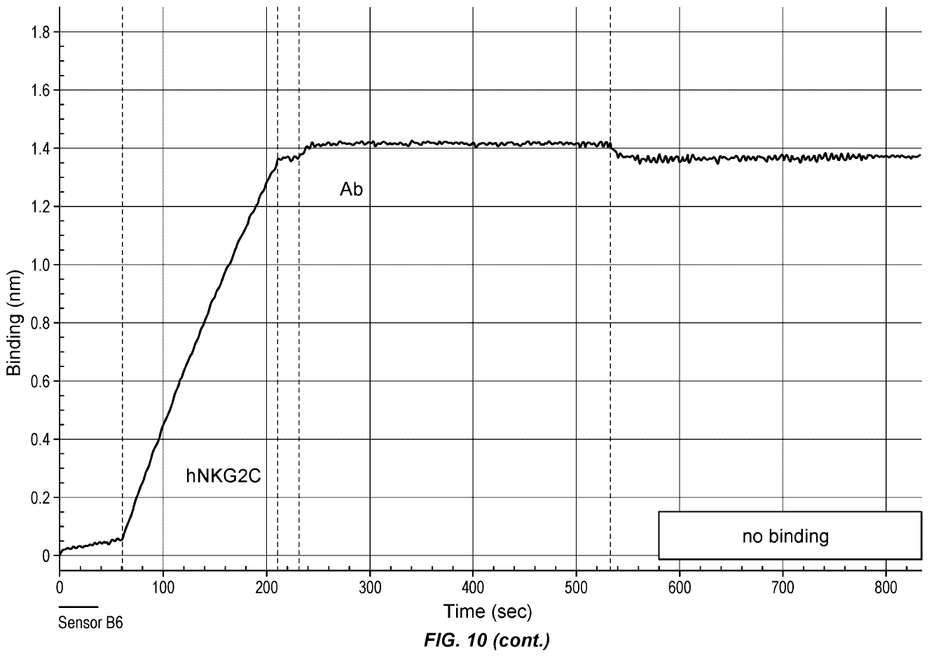

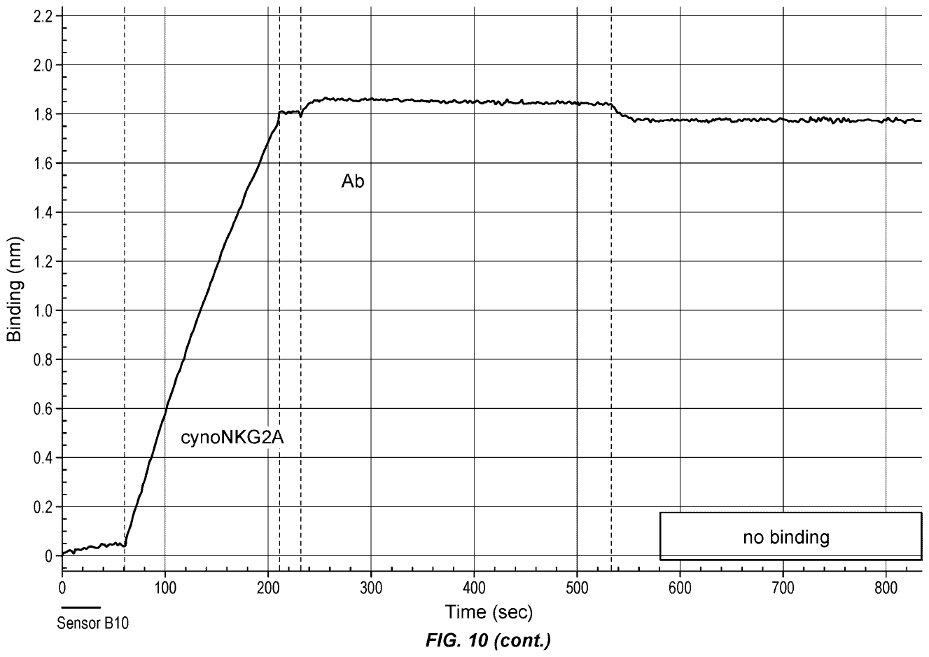

Exemplary bivalent Octet binding for an affinity matured variant of A42 designated A11 is depicted in FIG. 10. Results indicate that A11 demonstrated moderate binding to human NKG2A. No binding to human NKG2C or cyno NKG2A was observed for A11.

Exemplary cell binding result for A11 is represented by an EC₅₀ value. Results show that A11 demonstrated moderate cell binding with an EC₅₀ value of 4.35E-9 M.

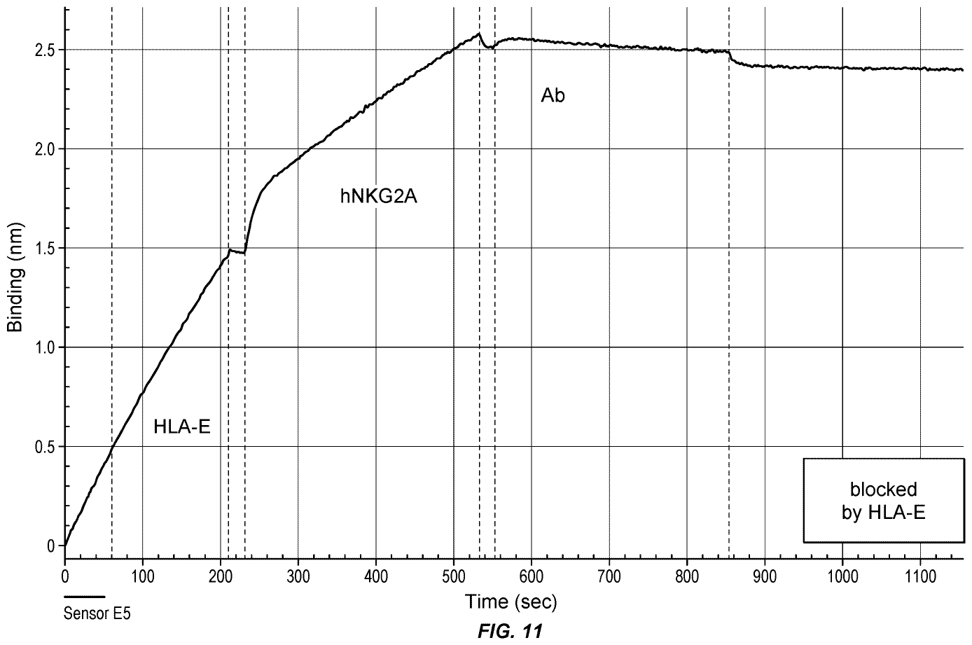

Exemplary HLA-E/NKG2A inhibition for A11 is depicted in FIG. 11. Results indicate that HLA-E and A11 compete for the same binding site on human NKG2A.

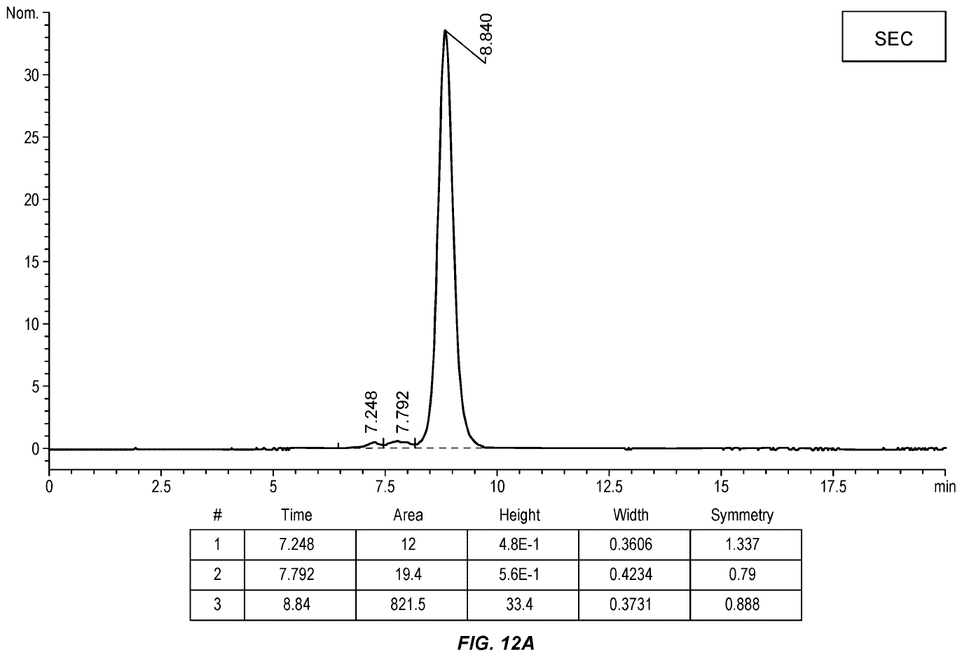

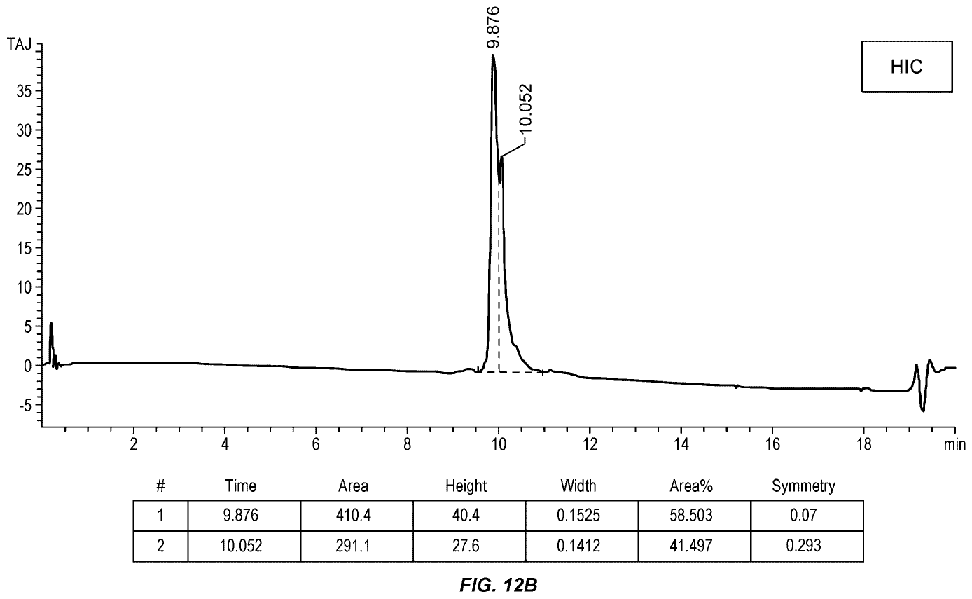

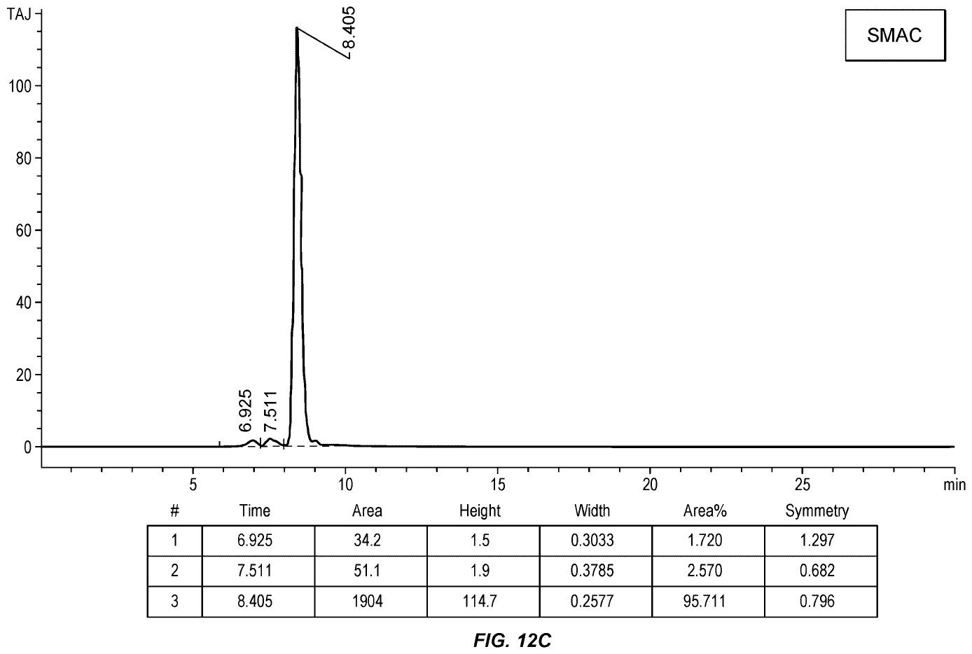

Exemplary SEC results for A11 are shown in FIG. 12A. Results indicate that A11 had low antibody aggregation. Exemplary HIC results for A11 are shown in FIG. 12B. Antibody hydrophobicity can impact antibody aggregation, solubility and viscosity. The HIC elution profiles (e.g., sharpness of elution peak and uniform retention times) of A11 suggested low propensities to aggregate and/or precipitate. Exemplary SMAC results for A11 are shown in FIG. 12C. Results indicated colloidal stability and low propensity to aggregate. Overall, the results of the developability assays show that A11 antibody met the developability criteria.

### Example 7. Bispecific Antibody Generation

Bispecific antibodies were constructed from selected NKG2A binding antibodies as described in Examples 1-6.

To generate bispecific antibodies with one or more NKG2A binding sites and one or more PD-L1 binding sites from NKG2A or PD-L1 binder discovery and affinity maturation campaigns, VL and VH domains identified were formatted into multivalent IgG1 constructs, including bispecific constructs with silent Fc regions. From these campaigns, three affinity-matured NKG2A binding arms (including A2 and A11 as described in Example 6) and four affinity matured PD-L1 binding arms were reformatted to generate 36 different combinations of bispecific antibody constructs. These exemplary constructs included 12 having one PD-L1 binding site and one NKG2A binding site (e.g., a 1×1 format), 12 having two PD-L1 binding sites and one NKG2A binding site (e.g., a 2×1 format), and 12 having one PD-L1 binding site and two NKG2A binding sites (e.g., a 1×2 format). Exemplary formats are shown in any of the three panels of FIG. 22.

Bispecific antibodies were affinity purified using anti-IgG10-CH1 resign by batch-mode gravity filtration. Clarified supernatants generated from 500 mL or 1000 mL transfection volumes were affinity purified using CAPTURESELECT™ CH1-XLAffinity Matrix bulk resin (ThermoFisher PN 194346201L). Supernatants from 1000 mL transfections were divided into 500 mL portions and all supernatants were transferred to 1 L sterile shake flasks. Resin was washed free of storage buffer using Dulbecco's PBS (pH 7.4, without Ca²⁺/Mg²⁺) by gravity filtration using disposable 10 mL columns (ThermoFisher Scientific PN 29924) and resuspended in PBS as a 50% slurry. Resin was aliquoted to shake flasks on a scale of 6 mL of 50% resin slurry per 500 mL of supernatant. Supernatant was incubated with resin for 1-2 hours at room temperature on a flask shaker operated at 170 RPM. Resin was captured on a gravity filtration column equipped with reservoir attachment (GE Healthcare Life Sciences PN 18-3216-03) and washed with 30 mL DPBS (10× settled resin bed volume). The resin bed was stringently washed with 30 mL with DPBS containing 500 mM NaCl. An additional wash was performed using 30 mL DPBS to reduce the concentration of NaCl prior to elution. Bound antibody was eluted from the gravity column using 9 mL 0.1 M sodium acetate (pH 3.5) into 2.25 mL neutralization buffer (2 M Tris-HCl, pH 7.5; Sigma PN T2944) contained in a 50 mL conical tube. Samples in neutralized elution buffer were dialyzed into DPBS overnight at 4° C. using a dialysis cassette with a 3.5 kDa molecular weight cutoff (Slide-A-Lyzer; ThermoFisher Scientific, PN 66110).

Each affinity-purified preparation was polished by strong cation exchange (SCX) using an AKTA pure fast protein liquid chromatography instrument (FPLC, GE Healthcare Life Sciences) running GE Unicorn v7.2 software. Aseptic techniques were used at all times. Prior to the day of purification, the entire flow-path (including the mobile phase reservoirs, samples loop, column, and switching valves) was cleaned using 500 mM NaOH in 20% ethanol by slowly pumping the cleaning solution through the instrument (0.1 mL/min for 2 hours). The instrument was then flushed with 20% ethanol and stored overnight. Just prior to polishing, a sample was buffer exchanged into 20 mM MES buffer [pH 6.0, MES=(2-(Nmorpholino) ethanesulfonic acid]. A buffer exchange method was employed using molecular weight cutoff (MWCO) filters with buffer exchange performed within 1-2 hours of polishing with storage at room temperature until polishing commenced. Approximately 35 mg of antibody (~4 mg/mL) in DPBS was added to a 15 mL, 30 kDa MWCO centrifugal spin filter (Amicon Ultra 30) and brought up to volume with MES buffer. Sample was centrifuged at 4k RPM for 15 minutes or until the original sample volume was achieved. MES buffer was added to bring the volume up to 15 mL and the sample was centrifuged again. This process was repeated three more times. 10 mL of low-endotoxin sample representing ~35 mg of protein at 3.5 mg/mL was loaded onto a MonoS 5/50 GL SCX column (0.5 cm×5 cm, 1 mL, 10 μm particle diameter; GE Healthcare Life Sciences, PN17516801) at 0.5 mL/min in mobile phase A (20 mM MES, pH 6.0) using 12 mL of mobile phase A. An additional 2 CV (column volume) of mobile phase A was used to remove unbound sample at a flow rate of 2.0 mL/min. 1.0 CV was 0.982 mL, as defined in GE Unicorn software for this particular column. Sample was eluted at 2.0 mL/min using a linear gradient from 0 to 30% mobile phase B (20 mM MES/1 M NaCl, pH 6.0) over 50 CV with fractions collected at 1-mL intervals. The system was washed with 7 CV of 100% B at 2.0 mL/min. Fractions from each major peak were pooled and assessed by non-reducing SDS-PAGE. The pooled sample was buffer exchanged into 1×DPBS as described in the procedure above, sterile filtered through a 0.2 μm filter, and re-tested for endotoxin using the ToxinSensor Chromogenic LAL Endotoxin Assay Kit from GenScript (PN L00350).

This process was repeated several times until unpolished input material was exhausted. A master pool was created from purified material from each injection and was subjected to full analytics screening and freeze/thaw analysis.

The purified bispecific antibodies were tested in NKG2A and/or PD-L1 binding and activity assays similar to those described in Examples 2-5. From such testing in multiple assays, a number of bispecific antibodies were selected based on an unexpected combination of structural characteristics and functional properties.

### Example 8. Bispecific Antibody Binding

### A. Affinities

Bispecific antibodies in exemplary formats as described in Example 7 were evaluated for binding. Carterra was used to confirm the specific interaction of the antigens to the selected bispecific antibody and to determine the antigen binding affinities.

To monitor the interaction with the antigen, the selected bispecific antibody was immobilized on a biosensor, and its interaction with soluble antigen was measured using a Carterra instrument. First, the selected bispecific antibody was covalently linked to a Carterra HC30M chip. To obtain accurate kinetic constants, the bispecific antibody was prepared at concentrations ranging from 1 to 10 μg/mL in 10 mM NaOAc (pH 5.0), and the coupling reaction was performed in running buffer (25 mM MES, pH 5.5) using multiple chip locations for each concentration.

In the next association step, the binding interaction of antigen to the coupled antibody was measured. Human PD-L1 with His tag (AcroBiosystems), or Human NKG2A & CD94 with mouse IgG2a Fc tag (AcroBiosystems) were buffer-exchanged into PBS buffer, then serially diluted into 900, 300, 100, 33.3, 11.1, 3.7, 1.2, 0.4 nM concentrations in 1×HBSTE. Injections at each concentration were performed in series with a regeneration step consisting of 3×25″ injections of Glycine pH 2.8, 1 M NaCl between each antigen binding event. The following injection series was performed across the chip using the Single Flow Cell apparatus: each binding event consisted of a one-minute baseline interval followed by a 15-minute antigen injection, a 45-minute dissociation with 1×HBSTE buffer (10 mM HEPES, 150 mM NaCl, 3 mM EDTA, 0.01% Tween-20, pH 7.4) as running buffer, regeneration and a 1-minute stabilizing baseline as described.

Binding data for hNKG2A and hPD-L1 were recovered by double-referencing: subtraction of the closest reference cell and a blank injection. 18 chip locations were used as input for the determination of each bispecific antibody—antigen K_{d}. K_{d} values were calculated from the kinetics of each antibody—antigen pair. Composite association rate constants were calculated from the sensorgrams using antigen concentrations that elicited a sufficiently slow association time (half-times of 30 seconds or more) to be measured precisely. The intrinsic association rate constants were then calculated from the rate dependence on analyte concentration. Intrinsic dissociation rate constants were calculated by averaging the dissociation rates observed in dissociation events at all antigen concentrations. K_{d} values were then calculated from the average association and dissociation rate constants.

Exemplary results for Carterra analysis are shown in Table 7.

TABLE 7
Binding Parameters to Human PD-L1, Cyno PD-L1, and Human NKG2A/CD94.

| | | | | | | | Human | | |
|---|---|---|---|---|---|---|---|---|---|
| | Human PD-L1 | | | Cyno PD-L1 | | | NKG2A/CD94 | | |
| | | kₒₙ | | | kₒₙ | | | kₒₙ | |
| | | (M⁻¹ | k_{off} | | (M⁻¹ | k_{off} | | (M⁻¹ | k_{off} |
| | K_{d} | s⁻¹ × | (s⁻¹ × | Kd | s⁻¹ × | (s⁻¹ × | K_{d} | s⁻¹ × | (s⁻¹ × |
| | (nM) | 10⁻⁵) | 10³) | (nM) | 10⁻⁵) | 10³) | (nM) | 10⁻⁵) | 10³) |
| P12 × A2 (1 × 1 | 0.13 (± | 5.3 (± | 0.07 (± | 0.10 (± | 5.6 (± | 0.06 (± | 0.21 (± | 3.6 (± | 0.08 (± |
| format) | 0.02) | 1.0) | 0.01) | 0.03) | 0.7) | 0.01) | 0.04) | 0.2) | 0.01) |
| P12 × A2A2 (1 × 2 | 0.15 (± | 4.7 (± | 0.07 (± | 0.12 (± | 4.8 (± | 0.05 (± | 0.15 (± | 3.6 (± | 0.05 (± |
| format) | 0.02) | 1.0) | 0.01) | 0.02) | 0.5) | 0.01) | 0.02) | 0.2) | 0.01) |
| P12 × A11 (1 × 1 | 0.17 (± | 4.7 (± | 0.08 (± | 0.11 (± | 5.4 (± | 0.06 (± | 0.38 (± | 2.4 (± | 0.09 (± |
| format) | 0.03) | 1.3) | 0.01) | 0.03) | 1.3) | 0.01) | 0.09) | 0.3) | 0.01) |
| P12 × A11A11 | 0.24 (± | 3.8 (± | 0.09 (± | 0.13 (± | 4.7 (± | 0.06 (± | 0.34 (± | 2.7 (± | 0.09 (± |
| (1 × 2 format) | 0.04) | 0.5) | 0.01) | 0.05) | 0.7) | 0.02) | 0.09) | 0.3) | 0.02) |
| Benchmark anti- | — | — | — | — | — | — | 0.06 (± | 5.1 (± | 0.03 (± |
| NKG2A | | | | | | | 0.01) | 0.5) | 0.01) |
| antibody (IgG4) | | | | | | | | | |
| Benchmark anti- | 0.17 (± | 3.2 (± | 0.05 (± | 0.47 (± | 3.4 (± | 0.16 (± | — | — | — |
| PD-L1 antibody- | 0.04) | 0.6) | 0.01) | 0.05) | 0.2) | 0.02) | | | |
| 2 | | | | | | | | | |
| Benchmark anti- | 0.13 (± | 3.3 (± | 0.04 (± | 0.33 (± | 3.9 (± | 0.13 (± | 0.05 (± | 6.2 (± | 0.03 (± |
| PD-L1 antibody | 0.03) | 0.8) | 0.00) | 0.06) | 0.3) | 0.01) | 0.01) | 0.7) | 0.01) |
| 2 × Benchmark | | | | | | | | | |
| anti-NKG2A | | | | | | | | | |
| antibody (1 × 1 | | | | | | | | | |
| format) (silent | | | | | | | | | |
| Fc, sFc) | | | | | | | | | |
| Benchmark anti- | 0.20 (± | 2.1 (± | 0.04 (± | 0.44 (± | 3.4 (± | 0.15 (± | 0.06 (± | 4.9 (± | 0.03 (± |
| PD-L1 antibody- | 0.06) | 0.9) | 0.01) | 0.10) | 0.3) | 0.03) | 0.01) | 0.6) | 0.01) |
| 2 × Benchmark | | | | | | | | | |
| anti-NKG2A | | | | | | | | | |
| antibody (1 × 2 | | | | | | | | | |
| format) (K/H | | | | | | | | | |
| swap sFc) | | | | | | | | | |

* Parameters determined from separately determining on and off-rate kinetics using antigen concentrations of 1.2, 3.7, 11.1, and 33.3 nM
* Uncertainties reflect standard deviation obtained from parallel measurements at 10 separate ROI positions.

In addition, a method using an equilibrium approach was performed. K_{d} determinations for cynomolgus NKG2A/CD94 were performed by measuring the signal response at equilibrium. The chip surface was first prepared by chemically coupling either P12×A2, P12×A11, or the benchmark antibodies to the SPR chip surface. The affinity was then determined by injecting antigen at concentrations ranging from 0.4 nM to 900 nM, and the response signal was observed to equilibrate over the course of each injection through utilization of an extended 2-hour time window for this association step. The equilibrium response signals and corresponding antigen concentrations were then input into a Langmuir binding isotherm model, and the K_{d} was determined through nonlinear regression. Averages and standard deviations were calculated from 24 replicate chip locations for each molecule. An example result is shown in Table 8 below.

TABLE 8
K_{d} of Binding to Cyno NKG2A/CD94 Calculated
by an Equilibrium Approach

| | K_{d} (±stdev)/nM |
|---|---|
| P12 × A2 | 42 ± 3 |
| P12 × A11 | 49 ± 5 |
| Benchmark anti-NKG2A antibody | 11 ± 2 |
| Benchmark anti-PD-L1 antibody − 2 | — |
| Benchmark anti-PD-L1 antibody − 2 × Benchmark | 7.4 ± 0.4 |
| anti-NKG2A antibody (1 × 1 format) | |
| Benchmark anti-PD-L1 antibody − 2 × Benchmark | 9.2 ± 1.0 |
| anti-NKG2A antibody (1 × 2 format) | |

### B. Cell Binding

Bispecific antibodies in exemplary formats as described in Example 7 were evaluated for binding to cells expressing endogenous NKG2A, for example, KHYG-1 cells, and PD-L1, for example, MC/CAR cells.

Cells were harvested at 70-90% confluence on the day of the assay. Cells were collected by centrifugation at 200×g for 5 minutes and media was removed. Cells were resuspended at 2×10⁶ cells per mL in cold PBS. An 8-point antibody dilution series (2× concentration) was prepared in PBS to cover the expected binding affinities of the antibodies being tested. 50 μL per well of the antibody dilution was plated in a 96 well V-bottom plate (Costar 3897). 50 μL per well of cell suspension was added. Plates were placed at 4° C. for 45-60 minutes.

Cells were collected by centrifugation at 400×g for 7 minutes and primary antibody was removed. 50 μL per well of AF488 goat anti human IgG Fab (Jackson Immuno Research 109-547-003) at 1:100 dilution was added. Plates were placed at 4° C. for 30 minutes.

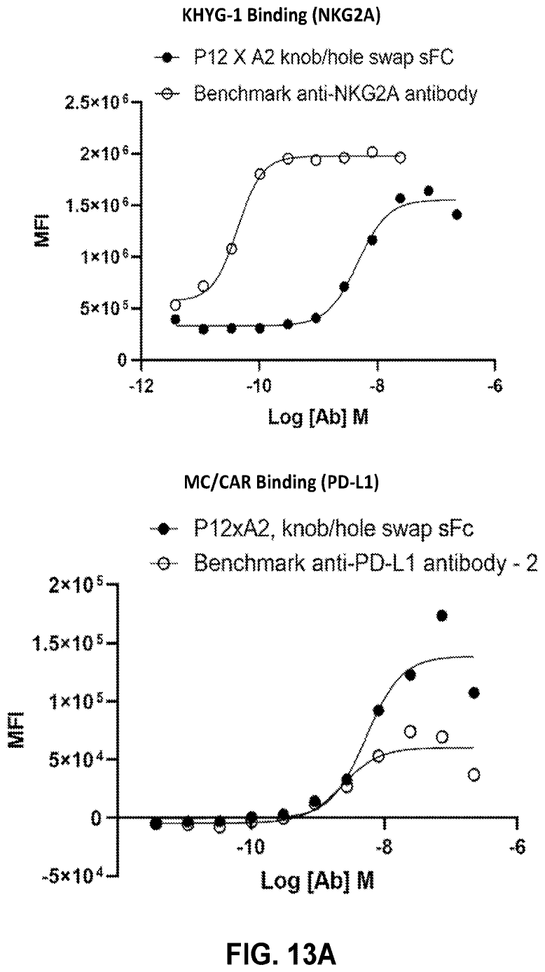

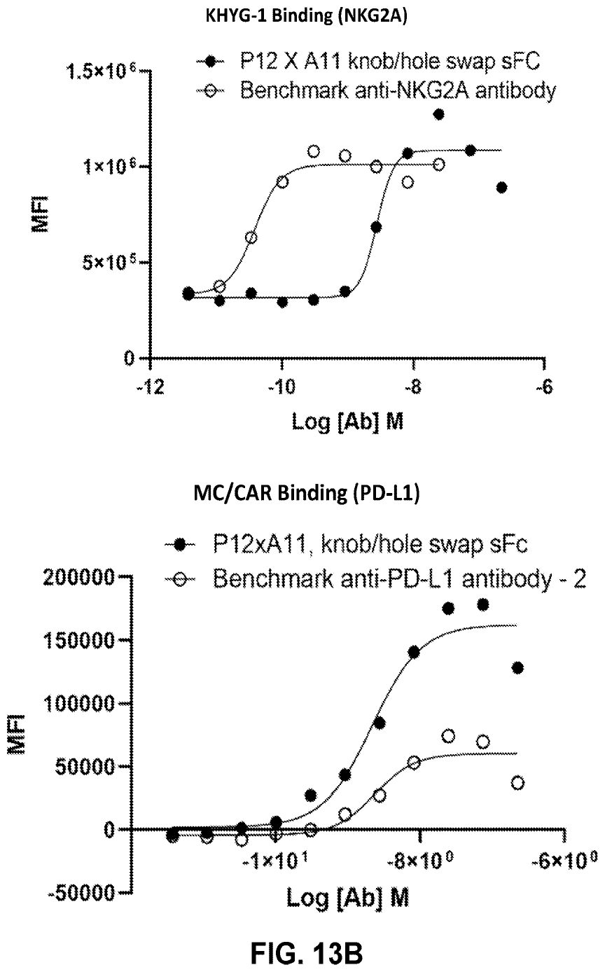

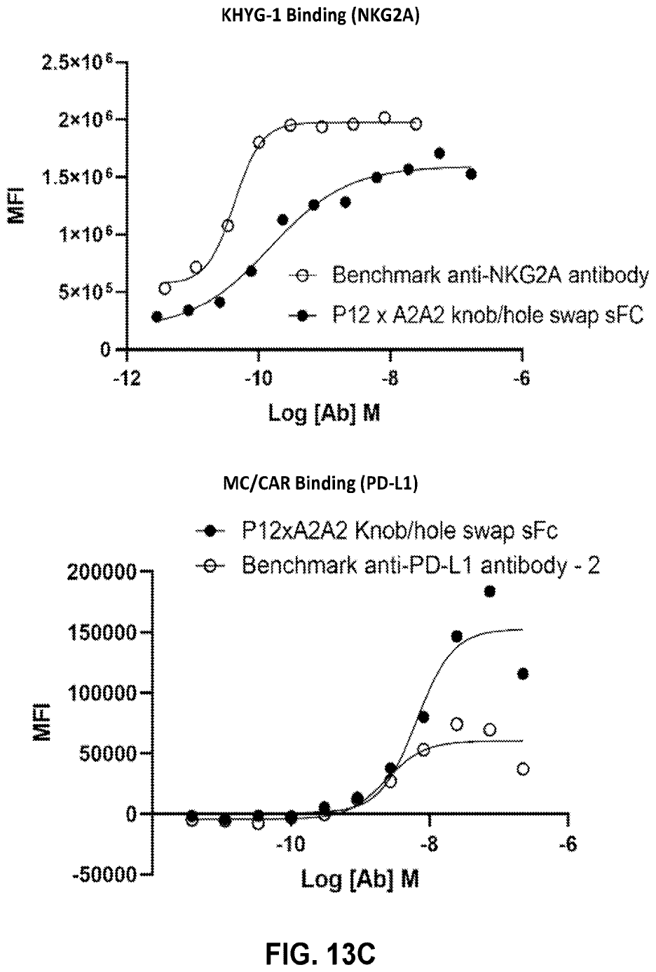

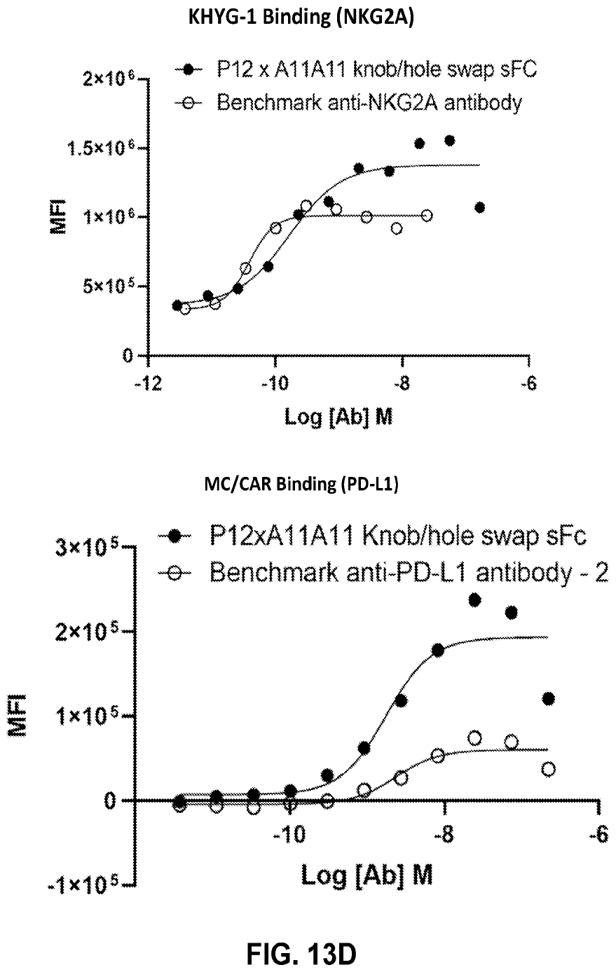

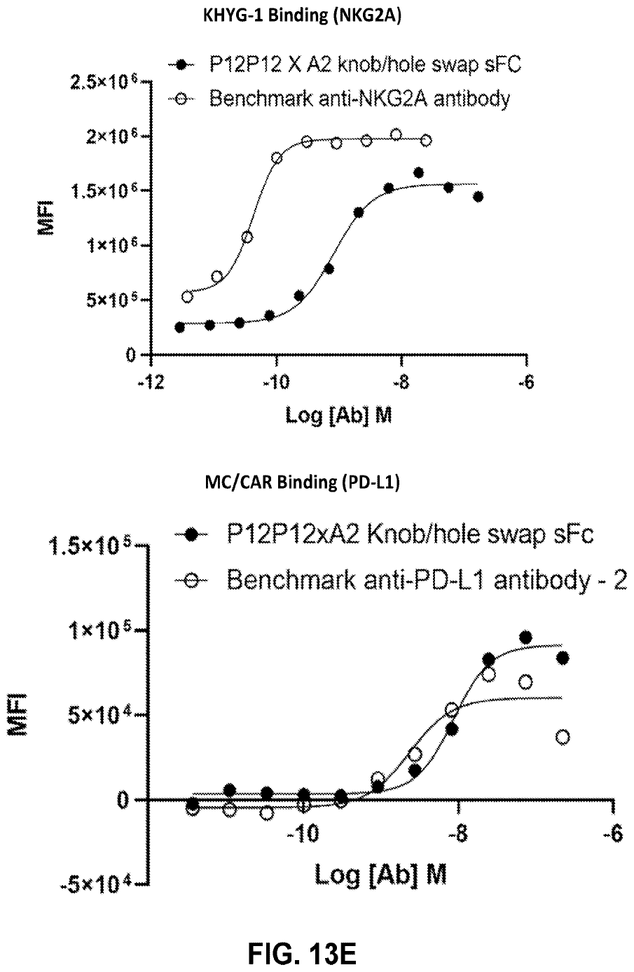

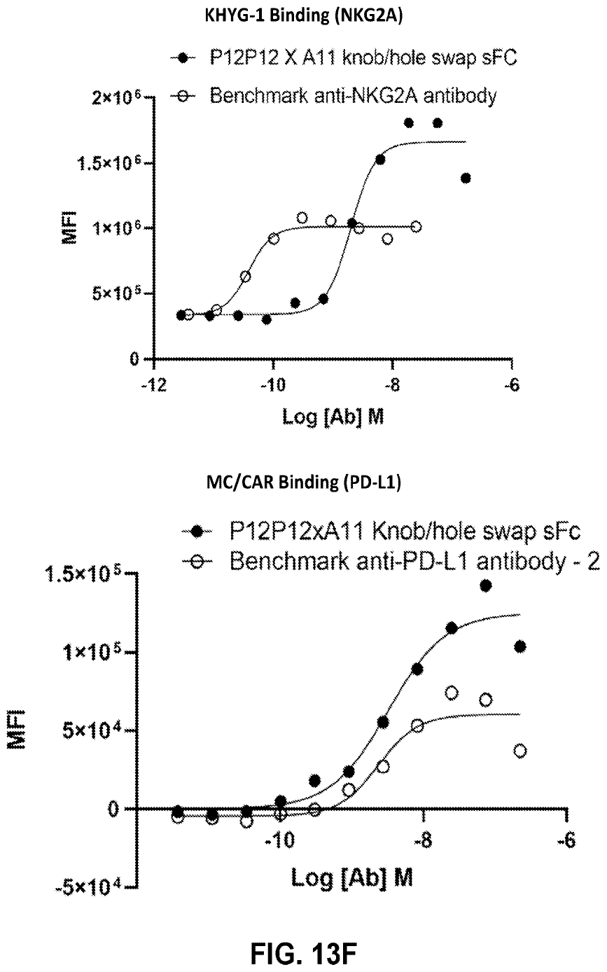

Cells were collected by centrifugation at 400×g for 7 minutes and secondary antibody was removed. Cells were resuspended in 50 μL per well of PBS and analyzed by flow cytometry. Binding curves were calculated using the mean fluorescence intensity (MFI) of the FITC fluorescence signal on the cells. Bispecific antibody binding was evaluated in KHYG-1 cells expressing endogenous NKG2A and in MC/CAR cells expressing PD-L1. Cells were harvested and assayed for binding as described above. Exemplary binding curves are set forth in FIGS. 13A-13F (benchmark anti-NKG2A antibody, CAS #1228763-95-8; and benchmark anti-PD-L1 antibody-2, CAS #1537032-82-8). Exemplary EC₅₀ value of binding are shown in Table 9.

TABLE 9
Cell binding analysis

| | EC₅₀ of NKG2A | EC₅₀ of PD-L1 |
|---|---|---|
| | KHYG-1 cell | MC/CAR cell |
| | binding (M) | binding (M) |
| P12 × A2 | 4.66E−09 | 5.59E−08 |
| P12 × A11 | 2.80E−09 | 2.17E−09 |
| P12 × A2A2 | 1.49E−10 | 6.49E−09 |
| P12 × A11A11 | 1.68E−10 | 1.76E−09 |
| P12P12 × A2 | 8.27E−10 | 8.67E−09 |
| P12P12 × A11 | 1.99E−09 | 3.23E−09 |

### Example 9. Functional Assays

Bispecific antibodies in exemplary formats as described in Example 7 were evaluated in HLA-E/NKG2A inhibitory assays.

Bispecific antibodies were evaluated for inhibition of HLA-E (tetramer) and NKG2A binding in KHYG-1 cells expressing endogenous NKG2A. 15 μL per well of binder dilution at 2× concentration was plated in a 96-well plate (Costar 3897). 15 μL per well of cell suspension at 5×10⁶ cells per mL was added. Plates were placed at 4° C. for 30 minutes. Following the incubation, 5 μL per well of Fc block (BD Pharmingen Cat #564220) was added at a final concentration of 500 ng per well. Plates were placed at 4° C. for 10 minutes. Following the incubation, 4 μL per well of diluted HLA-E was added. Plates were placed at 4° C. for 1 hour. Following the incubation, 100 μL per well of PBS was added and plate was centrifuged at 400×g for 7 minutes. Supernatant was removed by flicking the plate. Cells were resuspended in 50 μL per well of PBS and analyzed by flow cytometry (Intellicyt).

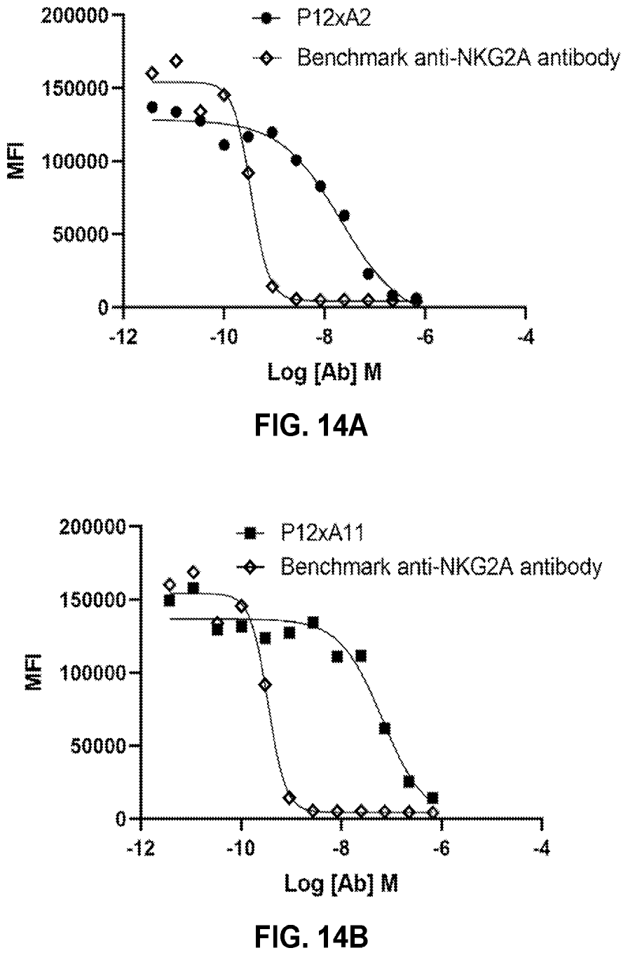

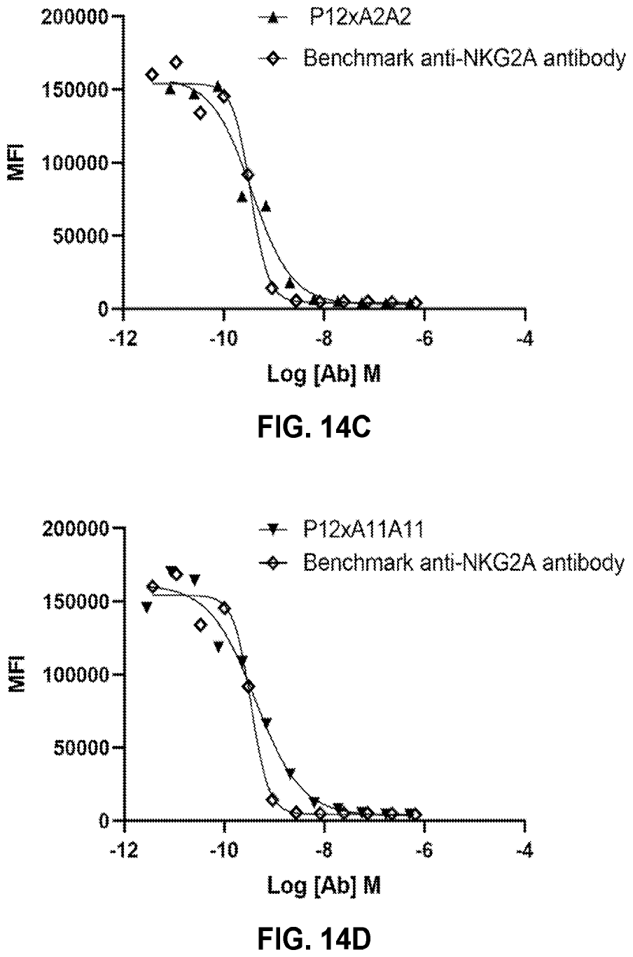

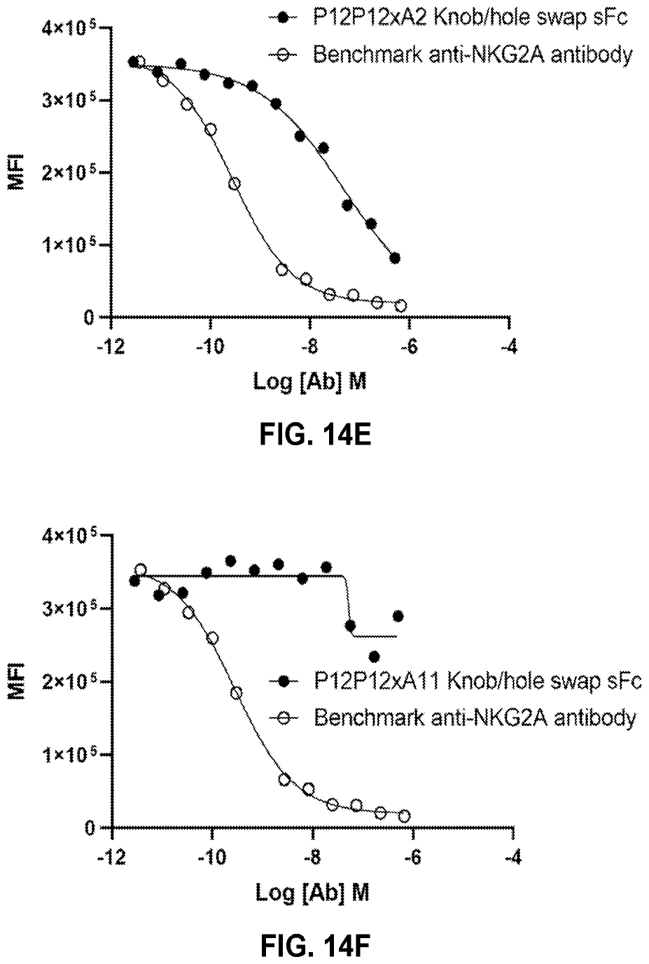

Exemplary inhibition curve of HLA-E/NKG2A inhibitory assays are set forth in FIGS. 14A-14F (benchmark anti-NKG2A antibody, CAS #1228763-95-8). The EC₅₀ values of P12×A2 inhibiting HLA-E/NKG2A binding in KHYG-1 NKG2A cells were 2.1E-08 M, and 6.52E-08 M. In the same assays, the EC₅₀ value of P12×A11 inhibition was 6.6E-08 M; the EC₅₀ value of P12×A2A2 inhibition was 3.6E-10 M M; the EC₅₀ value of P12×A11A11 inhibition was 4.2E-10 M; and the EC₅₀ value of P12P12×A2 inhibition was 4.64E-08 M. Results indicate that the selected bispecific antibodies showed a shift in fluorescence intensity compared to control antibody. Thus, the bispecific antibodies efficiently blocked the interaction of HLA-E and NKG2A binding.

### Example 10. Additional Functional Assays

Bispecific antibodies in exemplary formats as described in Example 7 were evaluated for inhibition of a PD-1/PD-L1 signaling in cell types expressing PD-L1, for example, MDA-MB-231 cells.

To test the ability of bispecific antibody to inhibit PD-1/PD-L1 signaling, bispecific antibody as described herein was assayed using a PD-1/PD-L1 Signaling Bioassay Kit (93-1104Y19-00117, Eurofins DiscoverX) using the manufacturer protocol. The assay was conducted using PD-L1+MDA-MB-231 target cells.

Briefly, 40 μL of target cells (30,000 cells per 40 μL) was added to each well of a white 96-well plate in Cell Plating Reagent 0 (CPO). Plates were incubated at 37° C. overnight. PD-L1+MDA-MB-231 cells were thawed for overnight recovery from thaw by adding 9.6 mL of pre-warmed Cell Plating Reagent (CPO) to a T25 flask, removing two cryovials of PD-L1+MDA-MB-231 cells from liquid nitrogen, and thawing the pellet by immediately adding 1 mL of pre-warmed CPO from the T25 flask to the cryovial. Cells were mixed by gently pipetting up and down several times to break up any clumps. The cell suspension was transferred to the T25 flask containing the remaining CPO and any media/suspension left in the cryovial was removed to ensure complete recovery of all the cells from the vial. Cells were incubated at 37° C. overnight.

Following the incubation, 20 μL of bispecific antibody was added to each well and plates were incubated at 37° C. for 1 hour. Then, 40 μL of PD-L1+MDA-MB-231 cells in CPO was added to each well of the 96-well plate and the plate was incubated at room temperature in the dark for 1 hour. Then, 10 μL of Bioassay Detection Reagent 1 was added to each well and plates were incubated for 15 minutes at room temperature in the dark. Next, 40 μL of Bioassay Detection Reagent 2 as added to each well of the assay plate and the plate was incubated for 3 hours at room temperature in the dark. Plates were analyzed on a ClarioStar Plate Reader (BMG Labtech).

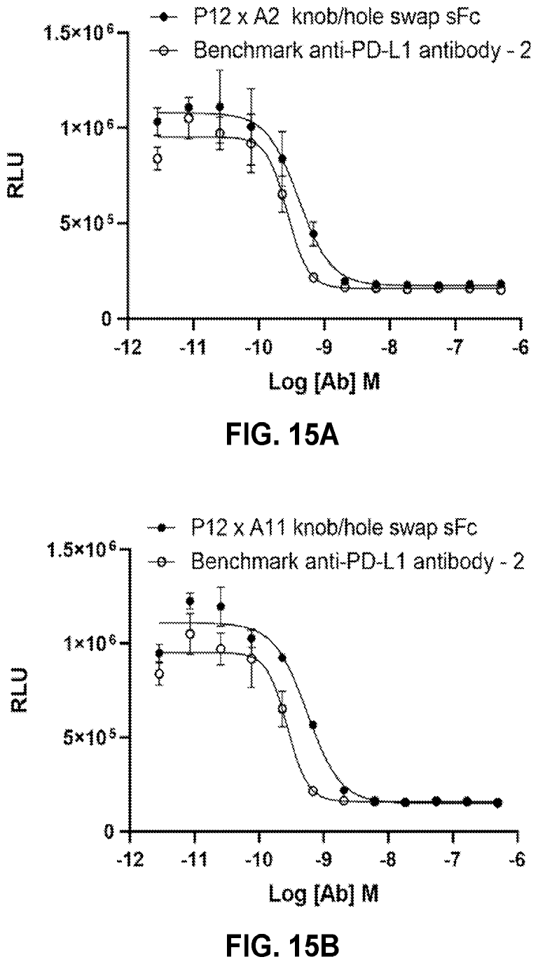

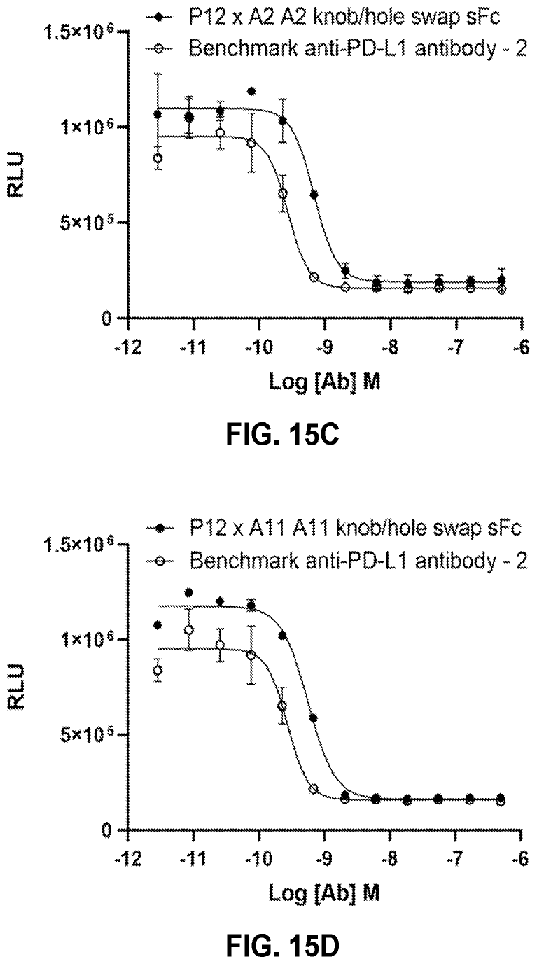

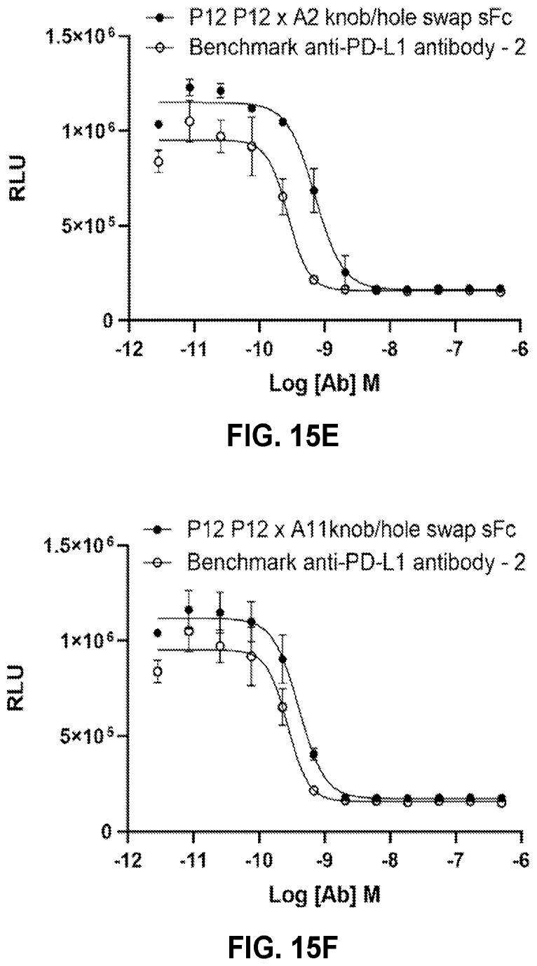

Exemplary results of the PD-1/PD-L1 checkpoint signaling assay in PD-L1+MDA-MB-231 cells are shown in FIGS. 15A-15F (benchmark anti-PD-L1 antibody-2, CAS #1537032-82-8). The IC₅₀ value of P12×A2 inhibiting PD-1/PD-L1 interaction in PD-L1+MDA-MB-231 cells was 4.08E-10 M. In the same assays, the IC₅₀ value of P12×A11 inhibition was 5.57E-10 M; the IC₅₀ value of P12×A2A2 inhibition was 6.89E-10 M; the IC₅₀ value of P12×A11A11 inhibition was 5.67E-10 M; the IC₅₀ value of P12P12×A2 inhibition was 7.14E-10 M; and the IC₅₀ value of P12P12×A11 inhibition was 4.05E-10 M. Results indicate that the selected bispecific antibodies showed a reduction in the chemiluminescent signal. Thus, the bispecific antibody efficiently blocked the interaction of PD-L1 and PD-1, inhibiting signaling.

### Example 11: Cytotoxicity Assays

Bispecific antibodies in exemplary formats as described in Example 7 were evaluated in NK92 mediated cytotoxicity assays using DELFIA EuTDA cytotoxicity Reagents AD0116.

LCL721.221 cells (Biovector NTCC) were cultured in RPMI-1640 medium (Life technology 11875-093) containing 10% FBS. NK92 cells (ATCC CRL-2407) were cultured in MEM-α medium (Life technology 12561-056) containing 0.2 mm inositol, 0.02 mm folic acid, 0.01 mm 2-mercaptoethanol, 12.5% FBS, 12.5% horse serum, and 10 ng/ml hIL-2. On Day 1, LCL721.221 cells were induced by loading 1.0 mM peptide3447 (ChemPartner) at 26° C. overnight, 5% CO₂. An aliquot of cells was incubated at 26° C. overnight without loading peptide3447. An assay medium was prepared in RPMI-1640 (without Phenol Red) (Life technology 11875-030) containing 5% Fetal Bovine Serum (FBS) and 10 ng per mL human IL-2 (Peprotech 200-02). On Day 2, NK92 cells were centrifuged and washed 2 times with assay medium. Following the wash, NK92 cells were resuspended at 5×10⁵ cells per mL in assay medium. 50 μL per well of NK92 cells were plated in a 96-well U-bottom plate (Costar 3897) at a final concentration of 2.5×10⁴ cells per well.

Serial dilutions of tested antibody were prepared in assay medium by either (a) 200 nM, 5× serial dilution for 9 points plus a zero control; or (b) 1000 nM, 5× serial dilution for 9 points plus a zero control. 50 μL per well of diluted antibody was added to the wells containing NK92 cells and incubated at 37° C., 5% CO₂ for 30 minutes.

Target cells LCL721.221 were loaded with fluorescence enhancing ligand for testing cytotoxicity. Briefly, LCL721.221 cells were centrifuged and washed 2 times with assay medium. 2 mL of cells at 1×10⁶ cells per mL were added to 1 μL of the fluorescence enhancing ligand (DELFIA BATDA Reagent) and incubated at 37° C., 5% CO₂ for 20 minutes. Following the incubation, cells were centrifuged and resuspended in PBS. The cells were further washed 3 times. In each wash, cells were resuspended and concentrated carefully to avoid carryover from one wash step to the following. After the final wash, cell pellet was resuspended to about 1×10⁵ cells per mL in assay medium containing 0.5 mM peptide3447 (2× concentrated for a final assay concentration of 0.25 mM).

Following the BATDA Reagent treatment, 100 μL per well of treated LCL721.221 cells at a final cell concentration of 1×10⁴ cells per well were added to pre-incubated antibody and NK92 cells. At the same time, controls were prepared on the plate. Briefly, 200 μL assay medium was used as background control. Wells for measuring target cell spontaneous release were prepared by mixing 100 μL assay medium and 100 μL LCL721.221 target cell suspension with or without peptide induction. Wells for measuring effector cell spontaneous release were prepared by mixing 150 μL assay medium and 50 μL NK92 effector cells. Following the incubation, the 96-well plate was centrifuged at 250×g for 5 minutes to make cells attached completely and placed at 37° C. with 5% CO₂ for 2 hours. Maximum release was determined by complete lysis of target cells, in which 5 μL of Lysis Buffer (as provided in the kit specified above and contains 0.03% digitonin and 19% dimethylsulphoxide (DMSO)) was added for 5 minutes to lyse the cells. Following the lysis, the plated was centrifuged at 250×g for 5 minutes.

To measure the cytotoxicity, 25 μL of the supernatant from all wells of the 96-well assay plate were transferred to a flat-bottom detection plate. 200 μL of DELFIA Europium Solution (as provided in the kit specified above) was added to all wells. The plate was shaken at 250 rpm for 15 minutes at room temperature. Fluorescence was recorded in a time-resolved fluorometer within 5 hours. Cytotoxicity percent was calculated by the following formula: % Cytotoxicity=(Sample−target cell spontaneous−effect cell spontaneous)/(target cell maximum−target cell spontaneous)×100%.

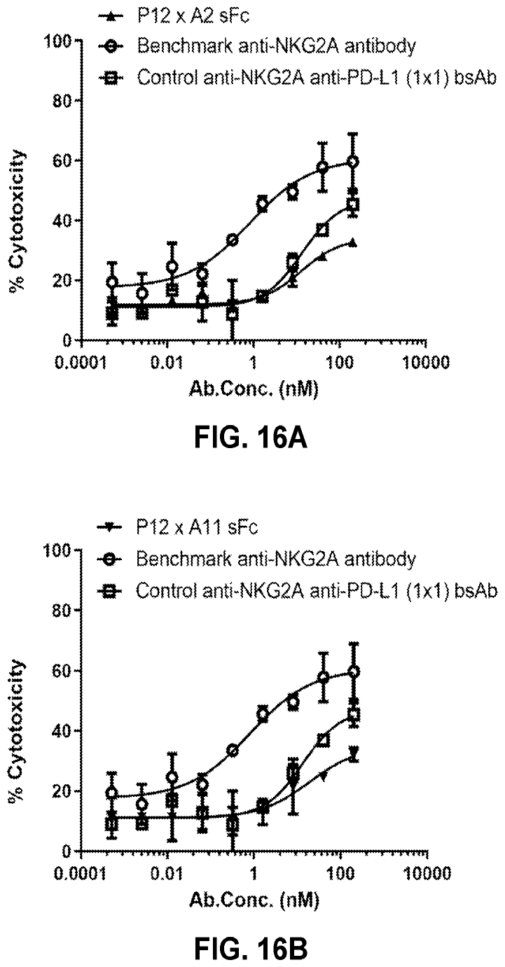

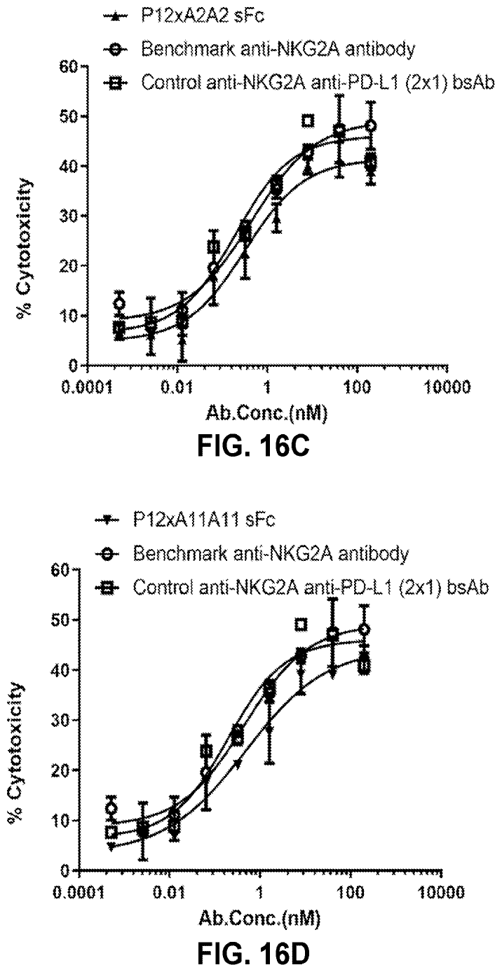

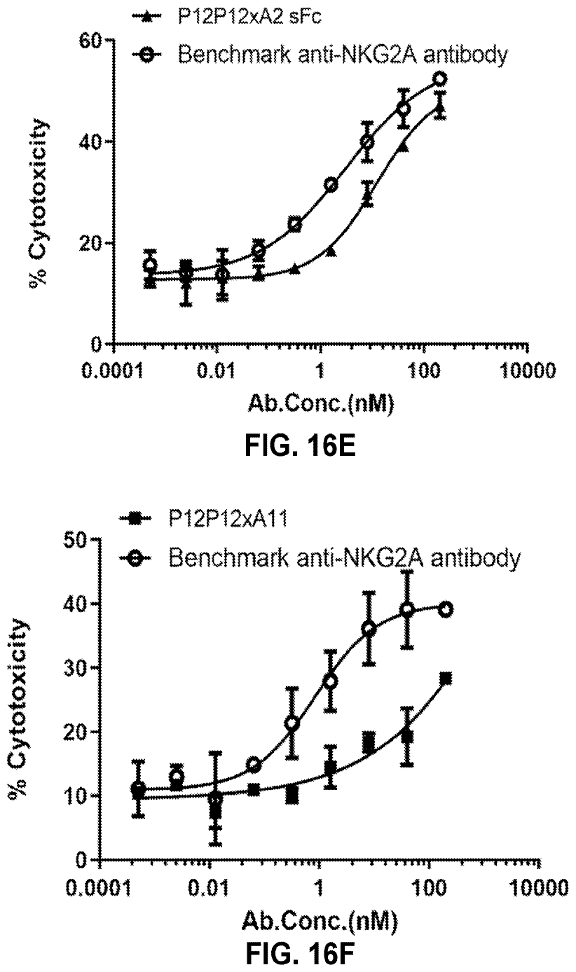

Exemplary results of the NK92 mediated cytotoxicity assays are set forth in FIGS. 16A-16F (benchmark anti-NKG2A antibody, CAS #1228763-95-8; and control anti-NKG2A anti-PD-L1 bsAb, benchmark anti-NKG2A antibody×benchmark anti-PD-L1 antibody-2). In this test, a starting concertation of 1000 nM was used for the 1×1 antibodies, while a starting concentration of 200 nM was used for the 1×2 antibodies. The EC₅₀ value of P12×A2 in cytotoxicity assays was 17.0±3.6 nM. In the same assays, the EC₅₀ value of P12×A11 was 21.7±2.9 nM; the EC₅₀ value of P12×A2A2 was 1.1±1.4 nM; the EC₅₀ value of P12×A11A11 was 0.9±0.7 nM; the EC₅₀ value of P12P12×A2 was 12.4 nM; and the EC₅₀ value of P12P12×A11 was >200 nM. Results indicate that the selected bispecific antibodies promoted NK92 mediated cytotoxicity to target cells.

### Example 12. In Vitro Functional Evaluation

### A. NK Cell Degranulation Assay

Human NK cells were purified from PBMCs of healthy donors (N=9) with an NK cell isolation kit and expanded in NK MACS Medium (Miltenyi) supplemented with IL-2 and IL-15. The phenotype of NK cells was analyzed on day 0 and at the end of the expansion on Day 14. Dead cells were excluded with Live/Dead stain and surface expression of CD56, PD-1, PD-L1 and NKG2A were measured by flow cytometry.

Purified human NK cells were co-cultured with the HLA-E+PD-L1+target cell line (LCL721.221-PD-L1) at an E:T (e.g., NK cells:LCL721.221-PD-L1) ratio of 10:1. The co-culture was performed in the presence of the test compounds (e.g., benchmark anti-NKG2A antibody, IgG4, CAS #1228763-95-8; benchmark anti-PD-L1 antibody, Fc silent IgG1, CAS #1428935-60-7; benchmark anti-PD-L1 antibody, Fc silent IgG1+benchmark anti-NKG2A antibody, IgG4, also referred to herein as the benchmark antibody or ab combination; P12×A2, silent Fc (sFc); P12×A11, sFc; P12×A2A2, sFc; P12×A11A11, sFc; IgG1 sFc isotype; and IgG4 isotype) at 3 or 6 concentrations (e.g., 67, 22, 7.4, 2.5, 0.8 and 0.3 nM). GOLGISTOP™ (5 g/ml) was added and the co-culture was incubated for 4 hours. After incubation, cells were labeled with a viability dye, and analyzed for CD56, CD107a and CD107b expression by flow cytometry to determine the percentage of degranulation within the CD56+ population.

### B. NK Cell Killing Assay

Human NK cells were purified from PBMCs of healthy donors (N=3), expanded and phenotyped as described above.

Purified human NK cells were co-cultured with fluorescent labeled HLA-E+PD-L1+target cell line (LCL721.221-PD-L1) at an E:T (e.g., NK cells:LCL721.221-PD-L1) ratio of 10:1. The co-culture was performed in the presence of the test compounds (e.g., benchmark anti-NKG2A antibody, IgG4, CAS #1228763-95-8; benchmark anti-PD-L1 antibody, Fc silent IgG1, CAS #1428935-60-7; benchmark anti-PD-L1 antibody, Fc silent IgG1+benchmark anti-NKG2A antibody, IgG4, also referred to herein as the benchmark antibody or ab combination; P12×A2, silent Fc (sFc); P12×A11, sFc; P12×A2A2, sFc; P12×A11A11, sFc; IgG1 sFc isotype; and IgG4 isotype) at 6 concentrations (i.e., 67, 22, 7.4, 2.5, 0.8 and 0.3 nM) and incubated for 4 hours. After incubation, cells were labeled with a viability dye and analyzed by flow cytometry to determine the percentage of target cell lysis.

### C. Results of NK Cell Degranulation Assay and Killing Assay

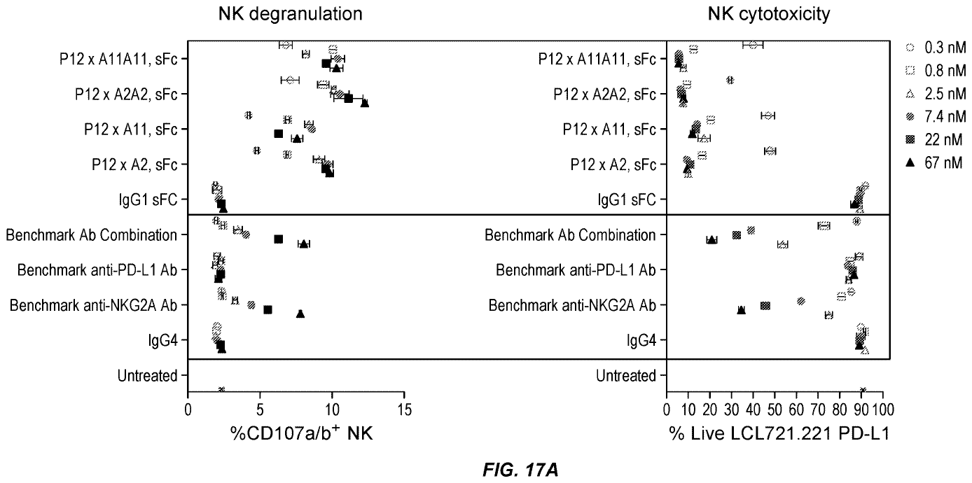

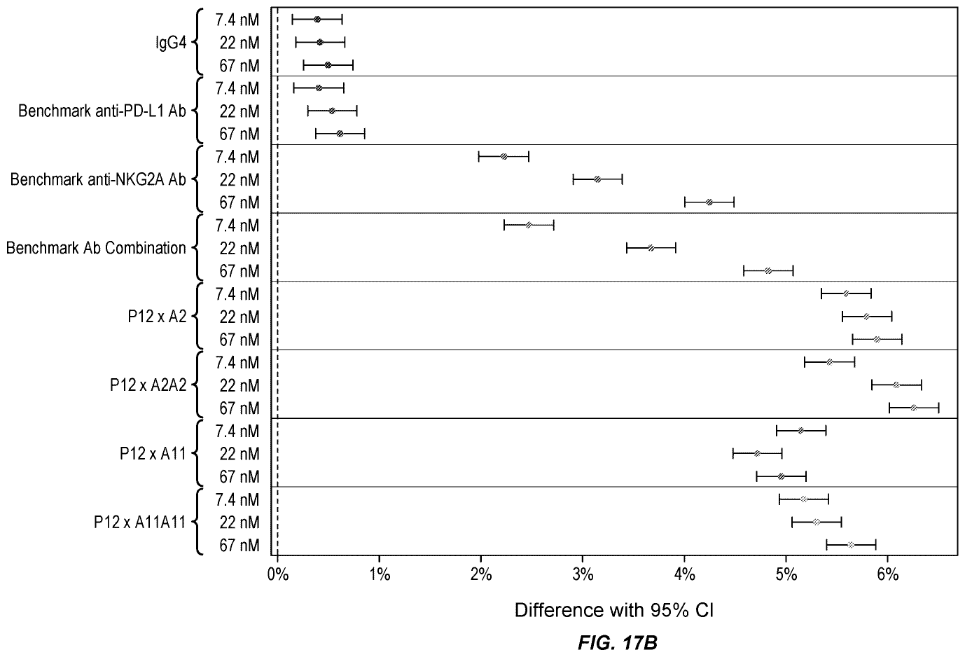

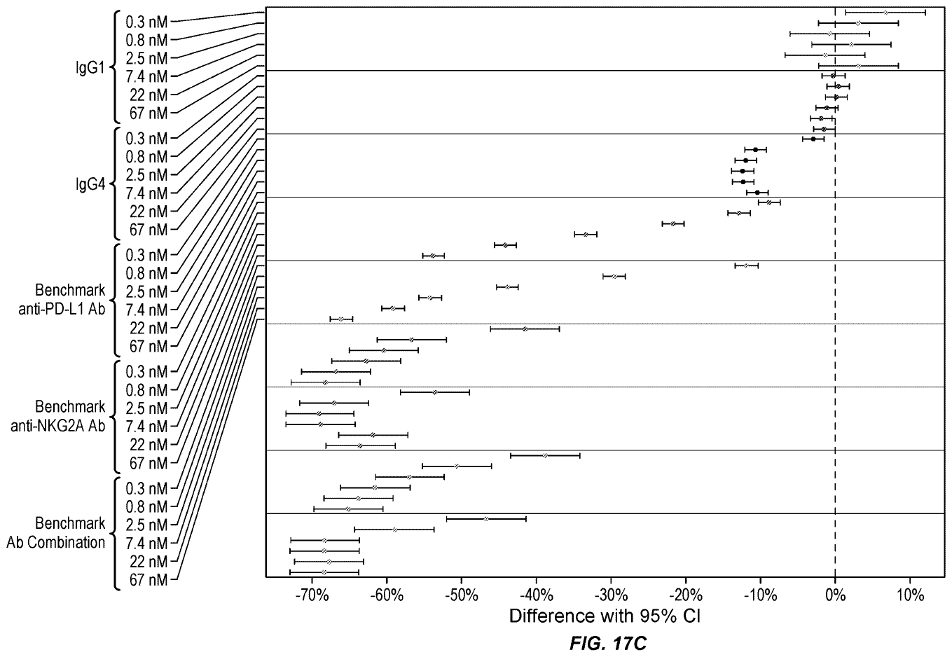

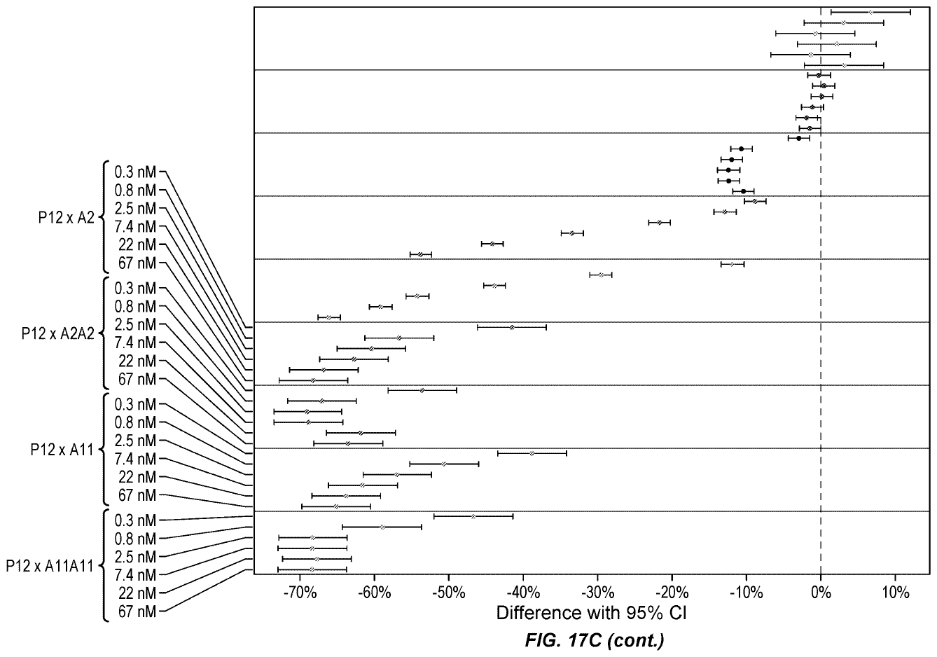

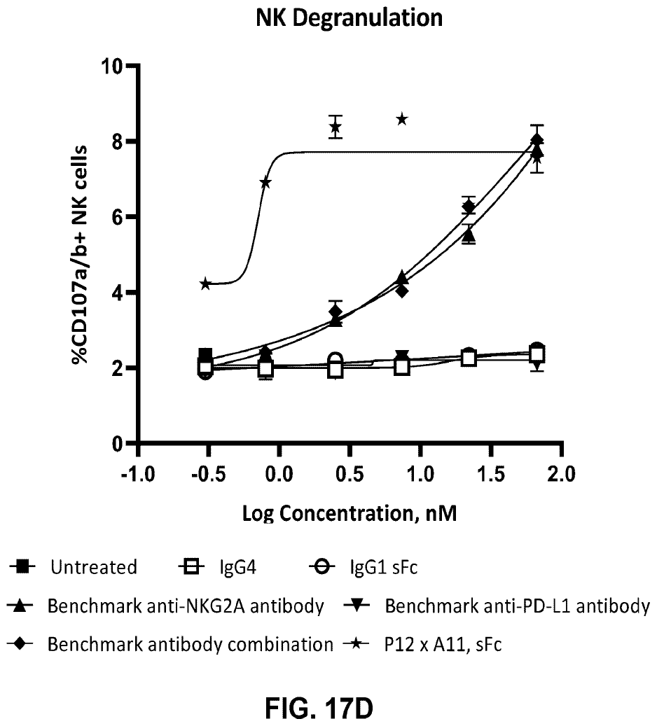

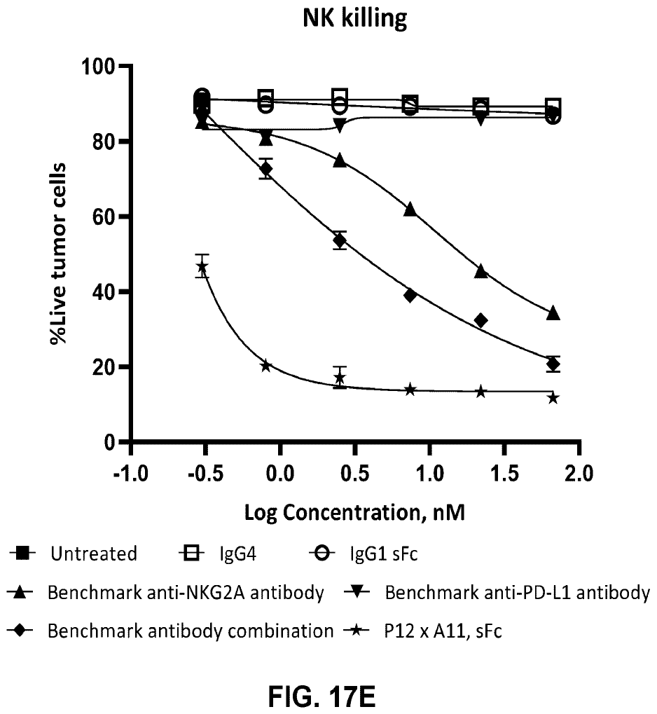

The benchmark anti-NKG2A antibody led to a concentration-dependent increase in degranulation, while the benchmark anti-PD-L1 antibody had a limited effect on NK degranulation. The benchmark antibody combination induced a concentration-dependent increase in degranulation and had a similar effect as the benchmark anti-NKG2A antibody alone (FIG. 17A).

The results also showed that NK degranulation correlated with NK killing, e.g., the higher percentage of degranulating NK cells detected, the lower percentage of live LCL721.221-PD-L1 target cells observed. Further, compared to the benchmark antibodies used alone or in combination, the tested antibodies, e.g., P12×A11A11, P12×A2A2, P12×A11 and P12×A2, resulted in stronger killing requiring a lower concentration to reach maximal cytotoxic activity. One representative donor set of degranulation and cytotoxicity result is shown in FIGS. 17A-17E, showing that P12×A11, sFc induced more pronounced NK degranulation and killing at lower concentration(s) compared to the benchmark antibody combination.

ANOVA statistical analysis of the NK degranulation and cytotoxicity data was performed. The results are shown in FIG. 17B (based on results of the tested samples from 9 donors) and FIG. 17C (based on results of the tested samples from 3 donors). It is noted that, while consistent with the observation described above, the benchmark antibody combination resulted in slightly higher increases in NK degranulation than the benchmark anti-NKG2A antibody alone. P12×A2 (and/or P12×A2A2) resulted in slightly higher increases than P12×A11 (and/or P12×A11A11) and P12×A2A2 and P12×A11A11 resulted in slightly higher increases than P12×A2 and P12×A11, respectively. For all test compounds, increases compared to the benchmark antibody combination were more pronounced for the lower doses.

### D. Flu-Stimulated CD8+ T Cell Assay

PBMC from six healthy donors were enriched for flu-peptide-stimulated CD8+ T cells by incubating PBMC samples with flu peptide (GILGFVFTL (SEQ ID NO:133)), IL-7, IL-2 and IL-15. After 7 days of culture, flu-peptide-stimulated CD8⁺ T cells were negatively isolated, rested overnight and then used in assay. MDA-MB-231-HLA-E-PD-L1 target cells (e.g., HLA-E+PD-L1+MDA-MB-231 cells) were pulsed with flu-peptide for 1 h and washed prior to use. The flu-specific CD8⁺ T cells were then co-cultured with flu peptide-pulsed target MDA-MB-231-HLA-E-PD-L1 cells at an effector to target ratio of 20:1 in the presence of test compounds (e.g., benchmark anti-NKG2A antibody, IgG4, CAS #1228763-95-8; benchmark anti-PD-L1 antibody, Fc silent IgG1, CAS #1428935-60-7; benchmark anti-PD-L1 antibody, Fc silent IgG1+benchmark anti-NKG2A antibody, IgG4, also referred to herein as the benchmark antibody or ab combination; P12×A2, sFc; P12×A11, sFc; P12×A2A2, sFc; P12×A11A11, sFc; IgG1 sFc isotype; and IgG4 isotype) at 3 and/or 6 test concentrations (i.e., 67, 22, 7.4 and/or 67, 22, 7.4, 2.5, 0.8 and 0.3 nM). After overnight incubation, granzyme and IFN-γ secretions of the CD8⁺ T cells were measured by FluoroSpot.

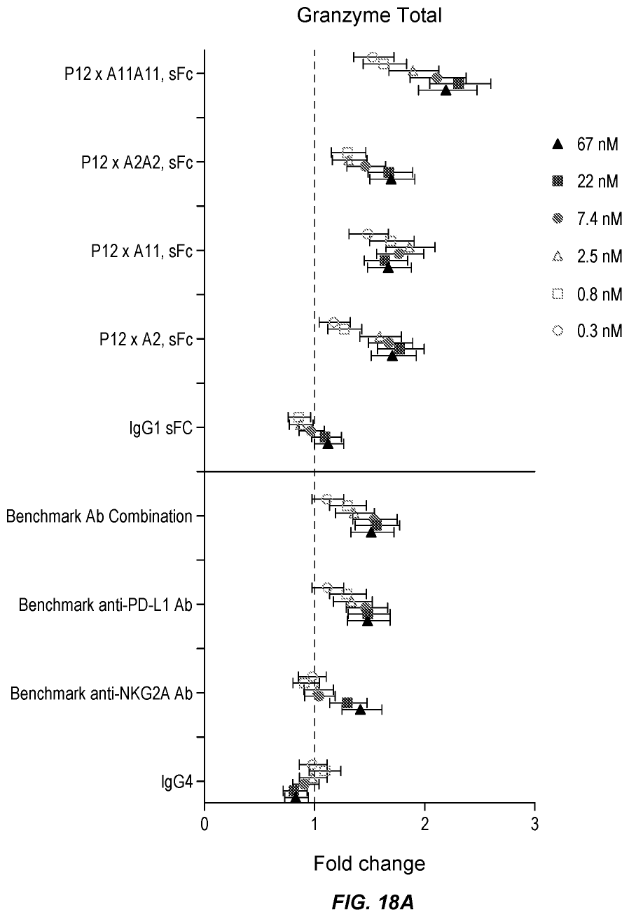

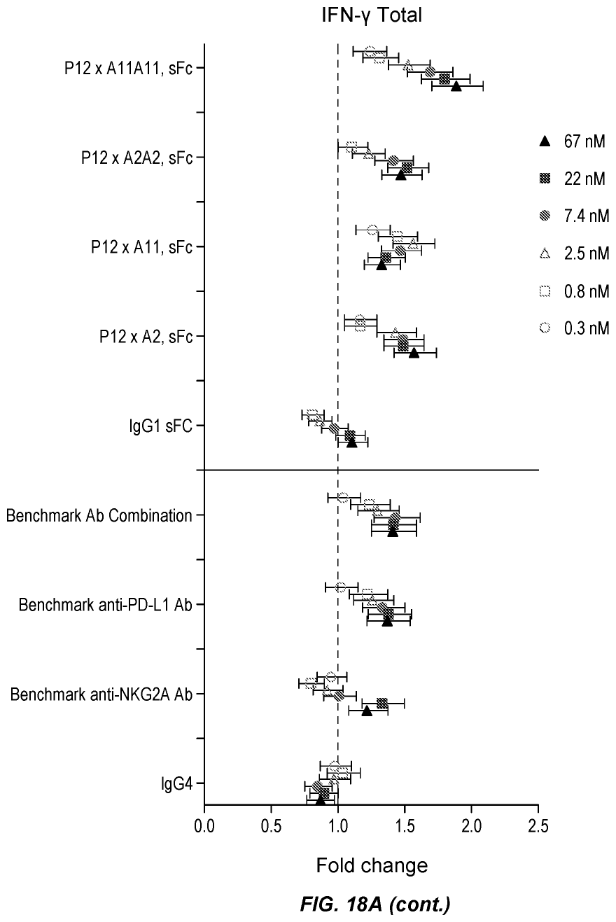

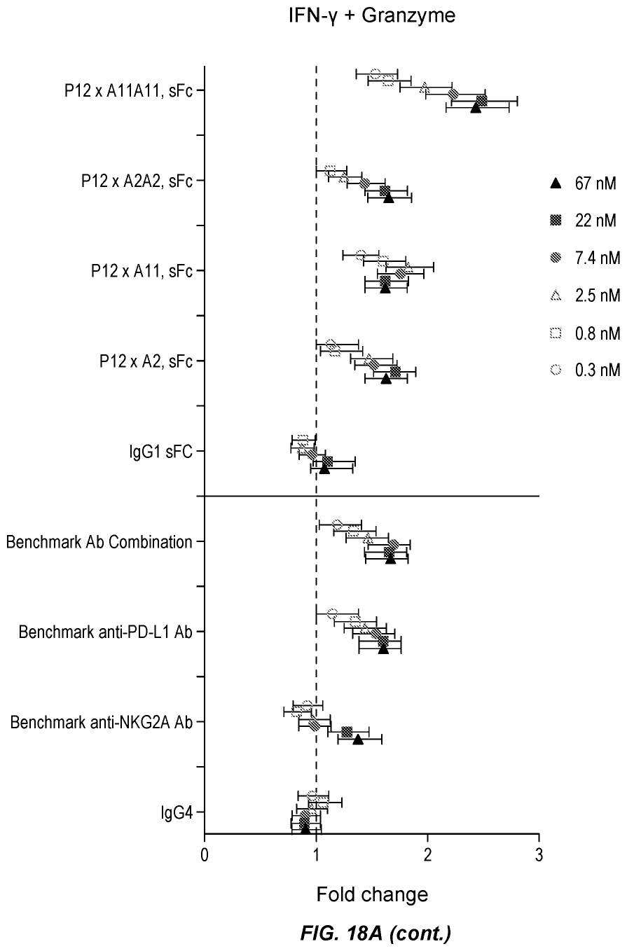

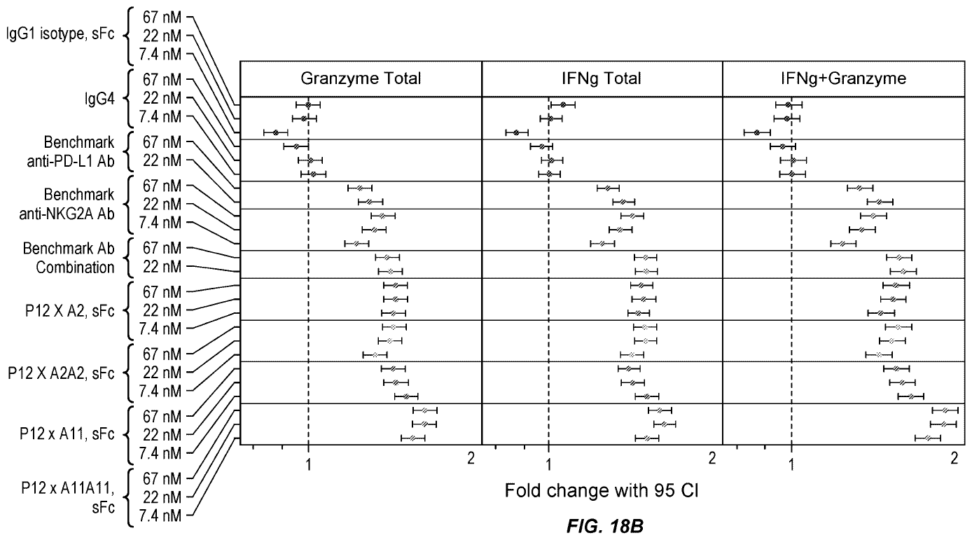

Results from one representative donor is shown in FIG. 18A. The tested antibodies, e.g., P12×A11A11, P12×A2A2, P12×A11 and P12×A2, showed a concentration-dependent response in granzyme and/or IFN-γ secretions, and resulted in a significant increase in granzyme and/or IFN-γ secretions that is greater than the isotype controls. P12×A11 and P12×A2 showed at least equivalent granzyme and/or IFN-γ activities as the benchmark antibody combination, while P12×A11A11 showed a better activity than the benchmark antibody combination.

An ANOVA analysis was performed across all six tested donors. The result is plotted in FIG. 18B. The tested antibodies, e.g., P12×A11A11, P12×A2A2, P12×A11 and P12×A2, showed a significant increase in granzyme and/or IFN-γ secretions that is greater than the isotype controls. Compared to the benchmark antibody combination, P12×A11A11 has superior activity, while the rest tested antibodies (e.g., P12×A2A2, P12×A11 and P12×A2) have at least equivalent activity.

### Example 13. Developability Assays

Bispecific antibodies in exemplary formats as described in Example 7 were evaluated in various developability methods. For example, various chromatographic methods, including size exclusion chromatography (SEC), hydrophobic interaction chromatography (HIC), and standup monolayer adsorption chromatography (SMAC) were employed to assess developability factors, such as monomer percentage, solubility, and antibody aggregation or precipitation.

Size exclusion chromatography (SEC) analysis was performed using a 7.8 mm ID×30 cm TSKgel G3000SWXL column (Tosoh Bioscience LLC, PN 08541) on an Agilent 1100 HPLC. The bispecific antibodies as described herein were normalized to 1 mg/ml concentration in Dulbecco's PBS (pH 7.4, without Ca²⁺/Mg²⁺) and clarified via centrifugation to pellet particulates while still retaining soluble aggregates. The mobile phase buffer was Dulbecco's PBS (pH 7.4, without Ca²⁺/Mg²⁺). 10 μL sample was loaded and isocratically eluted at 1.0 mL/min over 20 minutes. Absorbance was monitored at 280 nm. Chromatographic peaks were integrated to determine % homogeneity and retention time. The column stationary phase along with choice of mobile phase supports hydrophobic interaction in addition to molecular sizing (hydrophobic interaction much milder compared to SMAC). Data analysis was performed using Agilent ChemStation B.04.03.

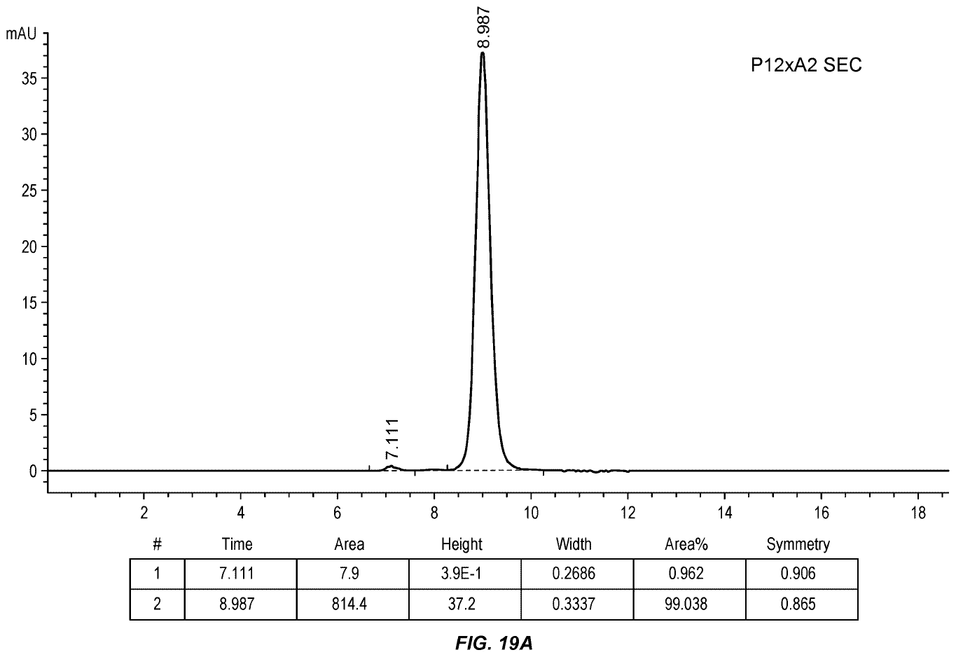

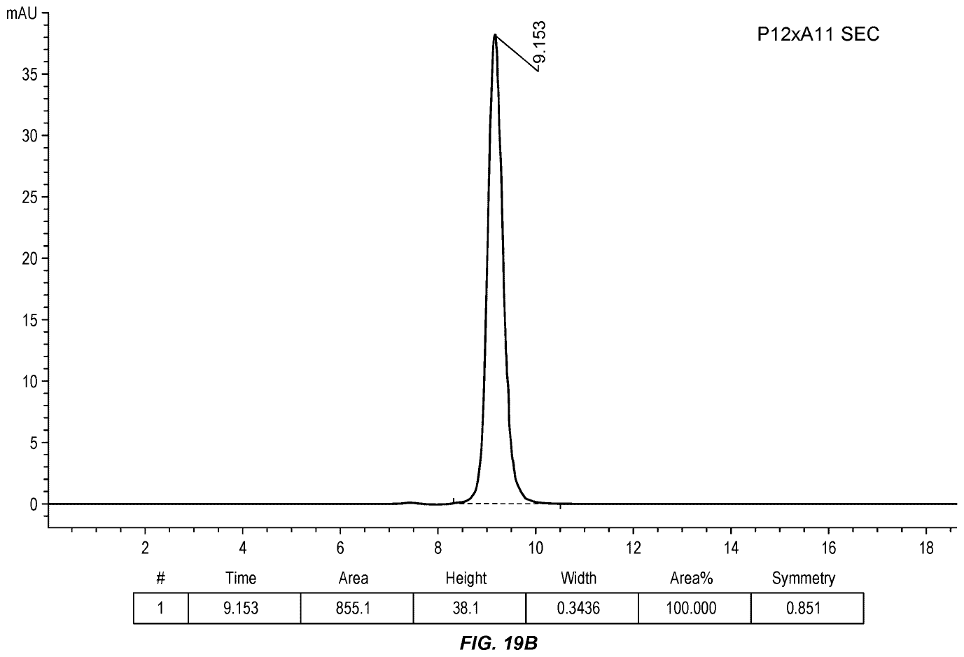

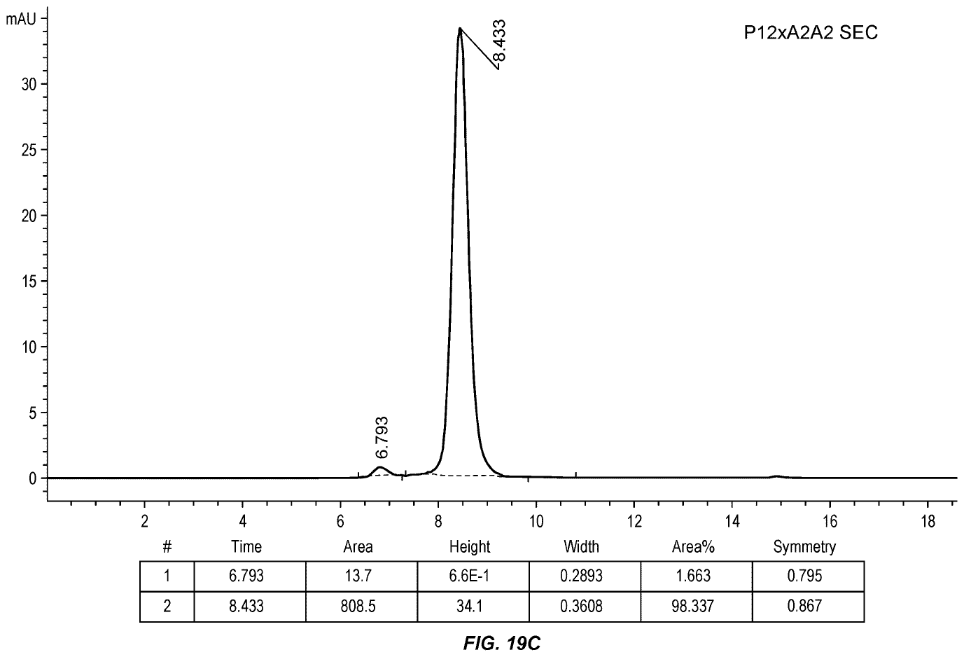

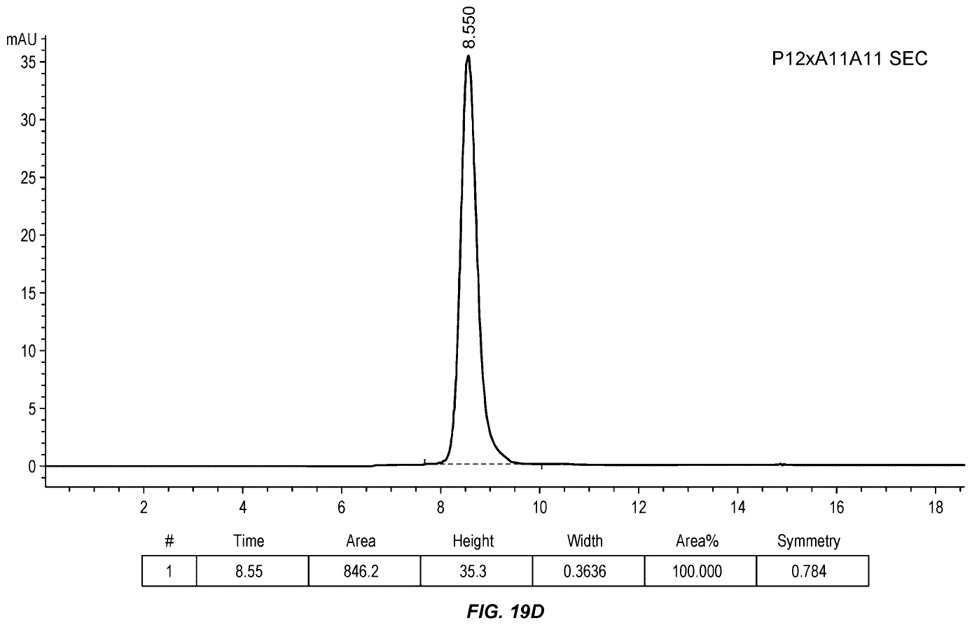

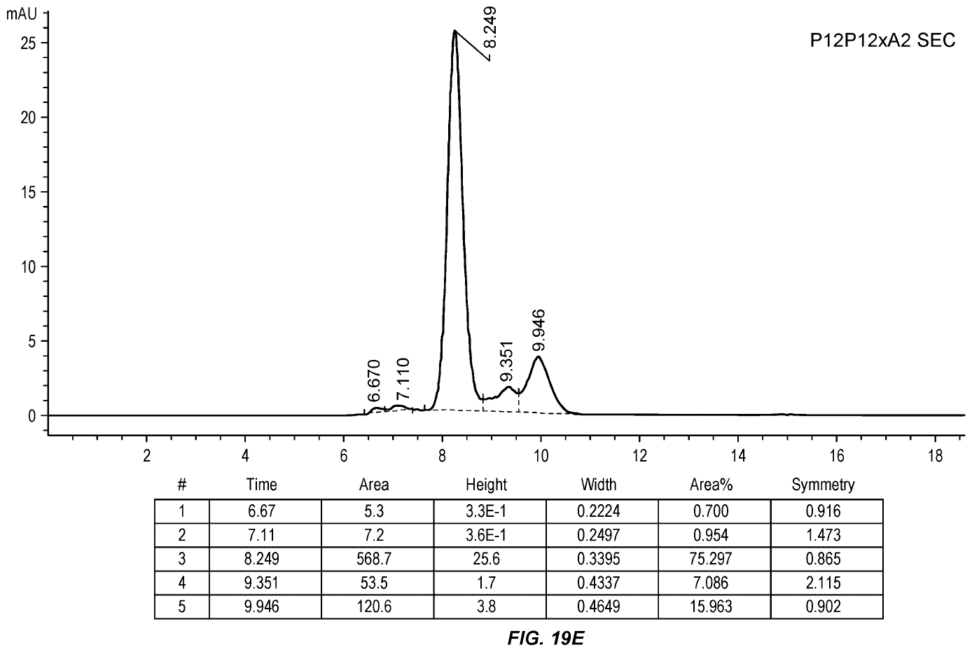

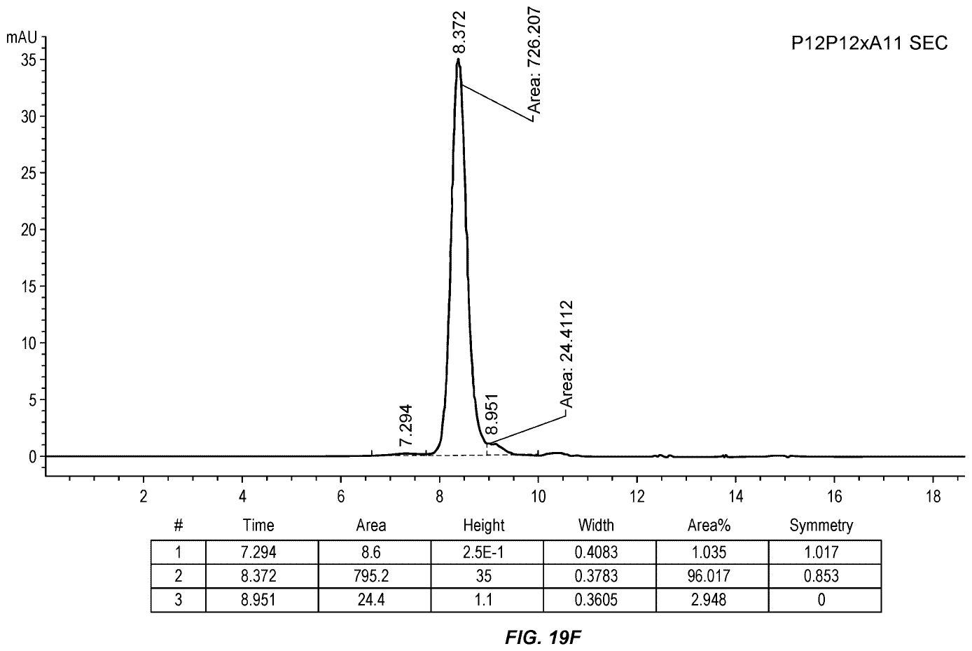

Exemplary SEC results of bispecific antibodies P12×A2, P12×A11, P12×A2A2, P12×A11A11, P12P12×A2, and P12P12×A11 are shown in FIGS. 19A-19F. Results indicate low bispecific antibody aggregation based on SEC.

Hydrophobic interaction chromatography (HIC) analysis was performed using a 4.6 mm ID×3.5 cm TSKgel Butyl-NPR column (Tosoh Bioscience LLC, PN 14947) on an Agilent 1100 HPLC. The bispecific antibodies as described herein were normalized to 2 mg/mL concentration in dPBS (pH 7.4) and then diluted with an equal volume of mobile phase buffer B to a final protein concentration of 1 mg/mL. The column was equilibrated with 100% mobile phase Buffer B (2 M ammonium sulfate/20 mM sodium phosphate, pH 7.0) at a flow rate of 1 mL/min. 10 μL sample was loaded and eluted using a gradient from 100% mobile phase buffer B to 100% mobile phase buffer A (20 mM sodium phosphate, pH 7.0) at 1.0 mL/min over 15 min, held at 100% A for 3 min to wash the column, and returned 100% B for 2 min for equilibration. Absorbance was monitored at 280 nm. Sample retention time was calculated and compared to a set of standard controls to identify antibodies with increased retention time (increased hydrophobicity).

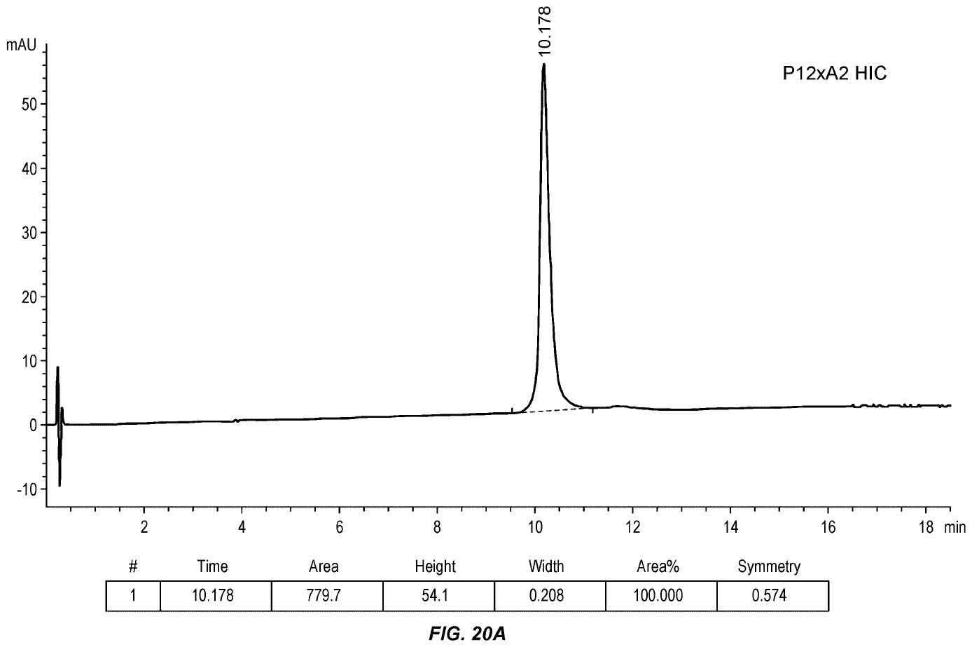

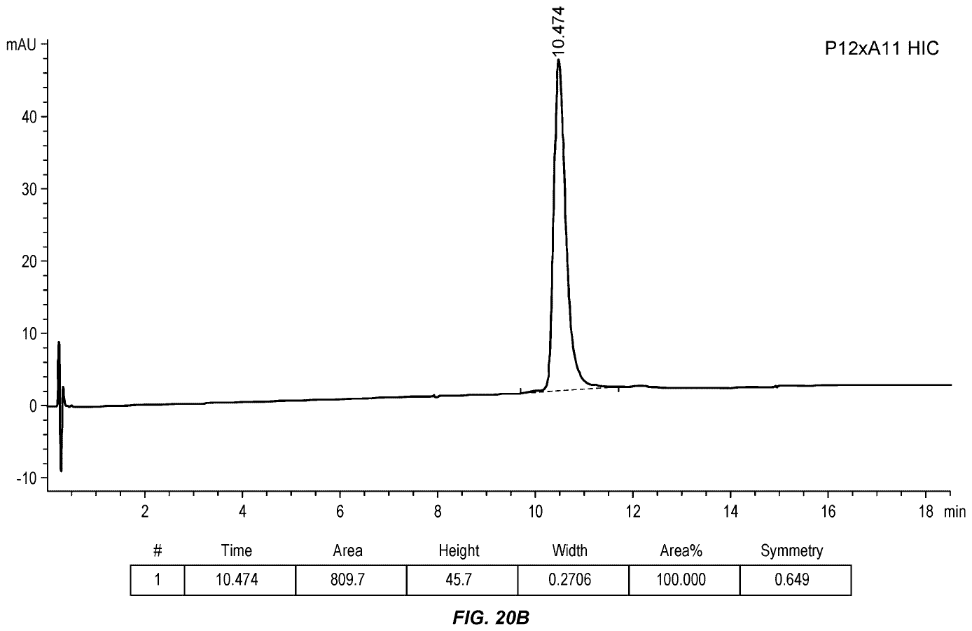

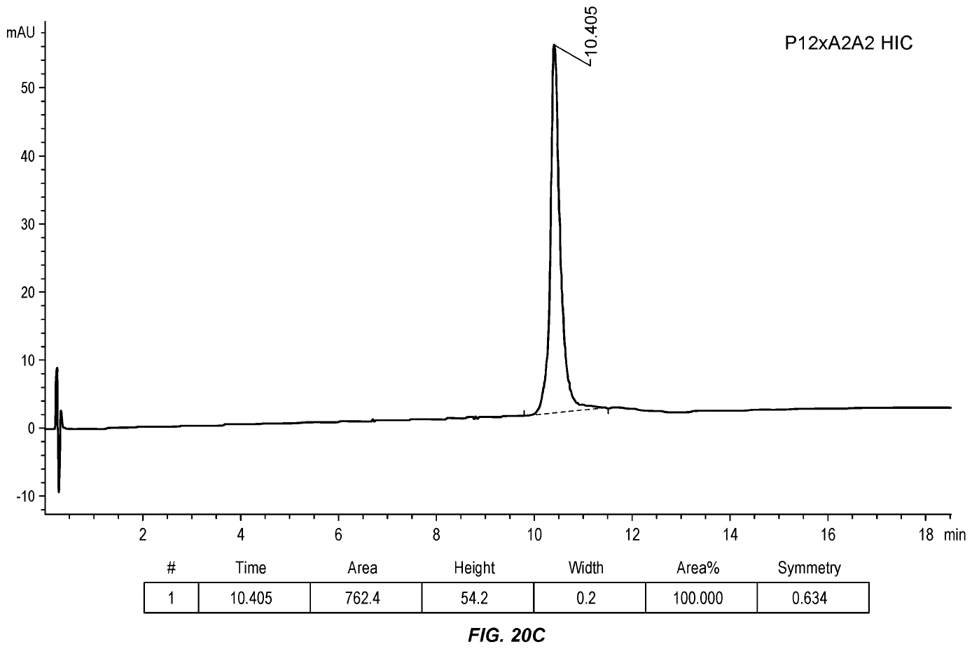

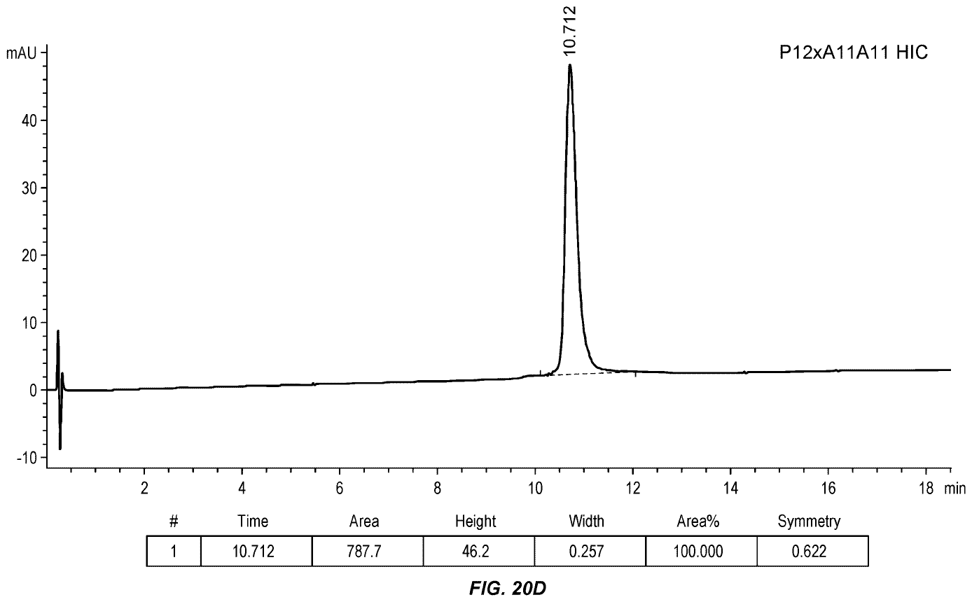

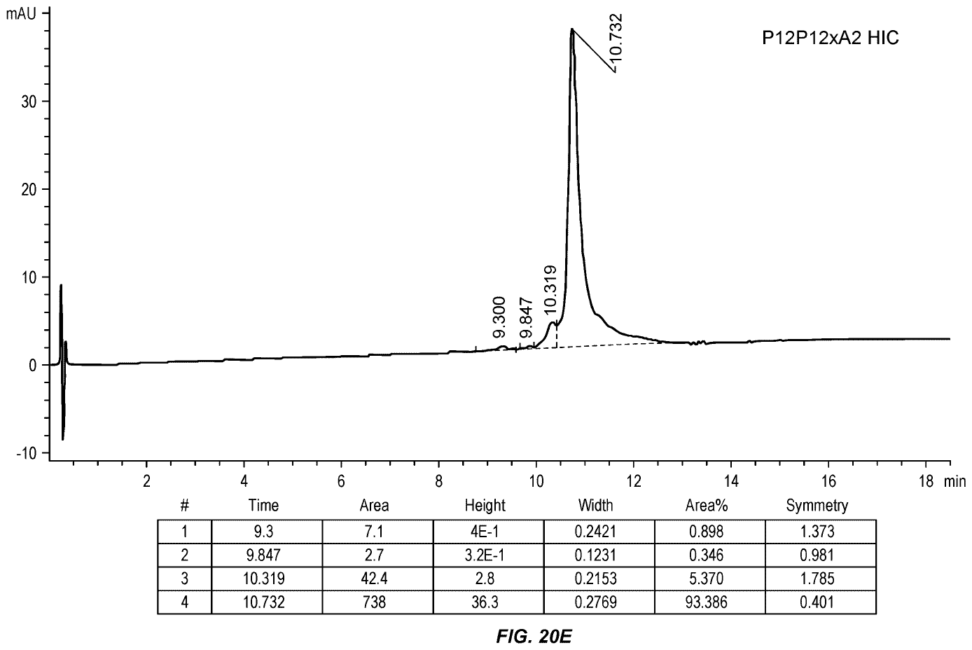

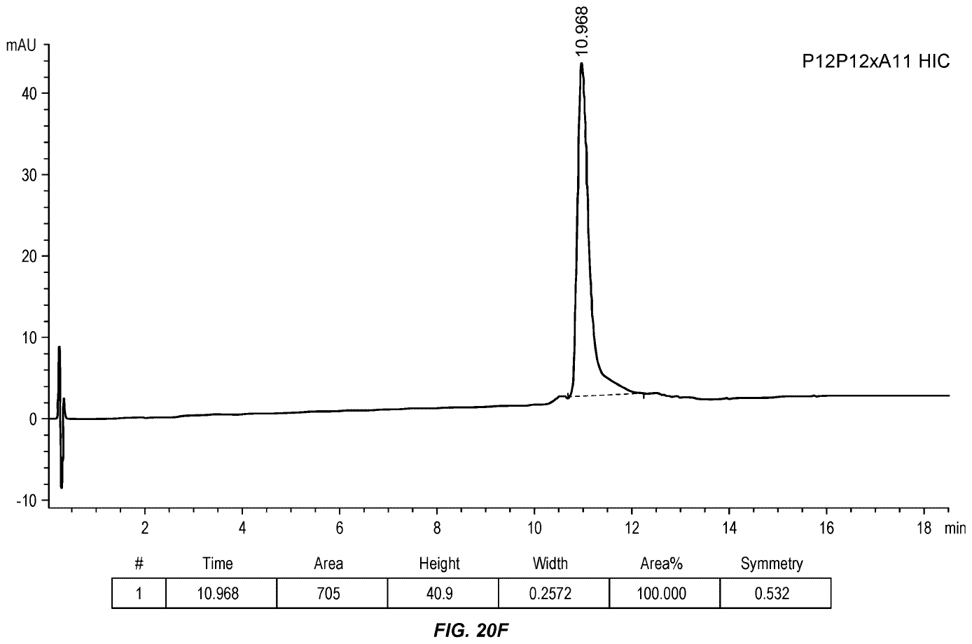

Exemplary HIC results of bispecific antibodies P12×A2, P12×A11, P12×A2A2, P12×A11A11, P12P12×A2, and P12P12×A11 are shown in FIGS. 20A-20F. Antibody hydrophobicity can impact antibody aggregation, solubility and viscosity. Results show similar retention times for the selected bispecific antibodies, and indicate a low propensity for aggregation and precipitation. The HIC elution profiles (e.g., sharpness of elution peak and uniform retention times) of the selected bispecific antibody suggested low propensities to aggregate and/or precipitate.

Standup monolayer adsorption chromatography (SMAC) analysis was performed using a 4.6 mm ID×300 mm Zenix SEC 300 column (Sepax Technologies, PN 213300P-4630) on an Agilent 1100 HPLC. The bispecific antibodies as described herein were normalized to 1 mg/mL concentration in dPBS (pH 7.4) and clarified via centrifugation to pellet particulates. The mobile phase buffer was dPBS (pH 7.4, without calcium and magnesium). 10 μL sample was loaded and isocratically eluted at 0.25 mL/min over 32 min. Absorbance was monitored at 280 nm. Sample retention time was calculated and compared to a set of standard controls to identify binders with increased retention time (increased propensity to form aggregates).

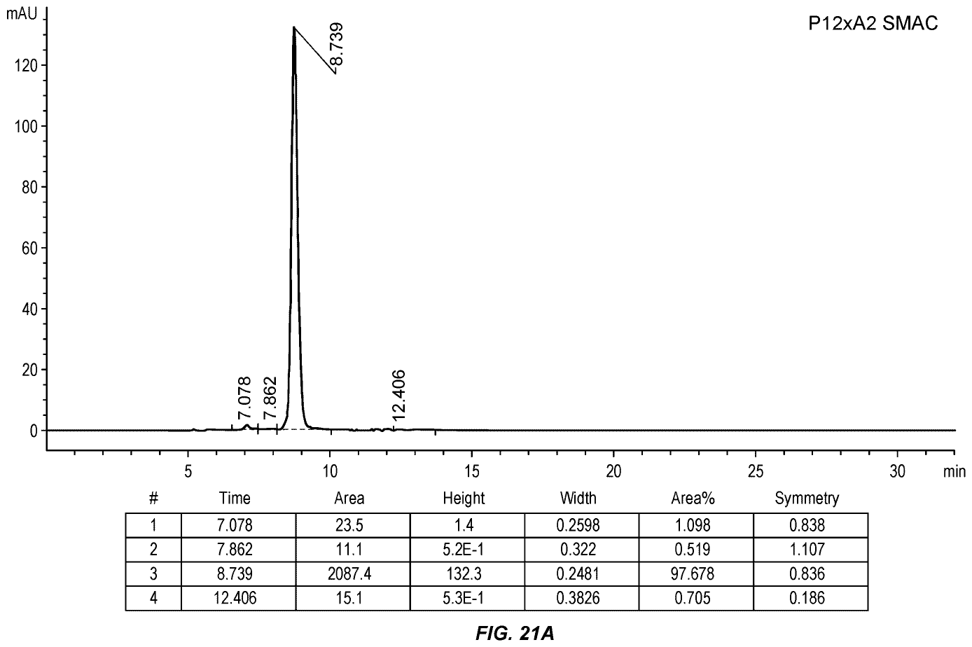

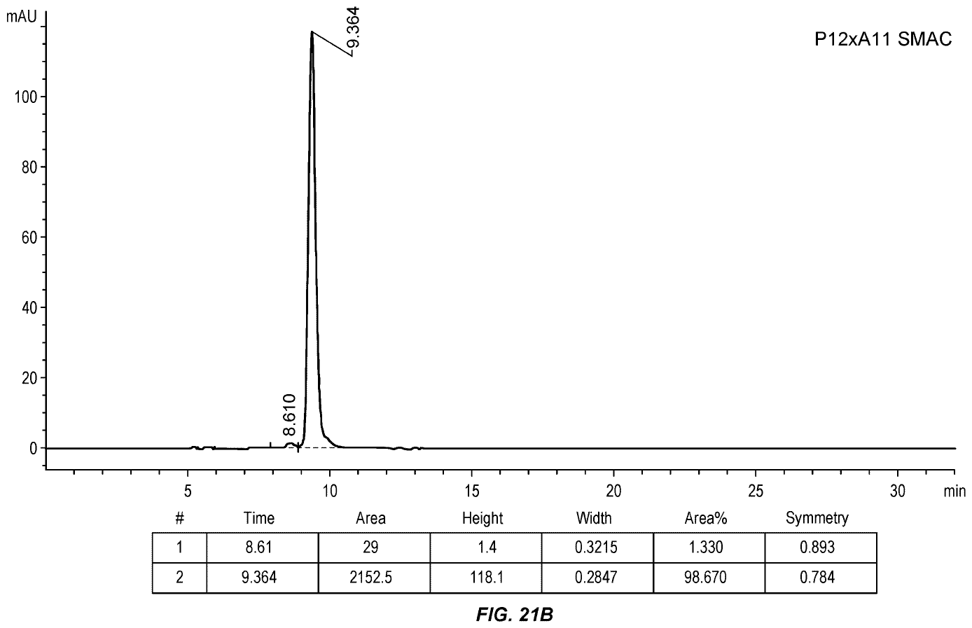

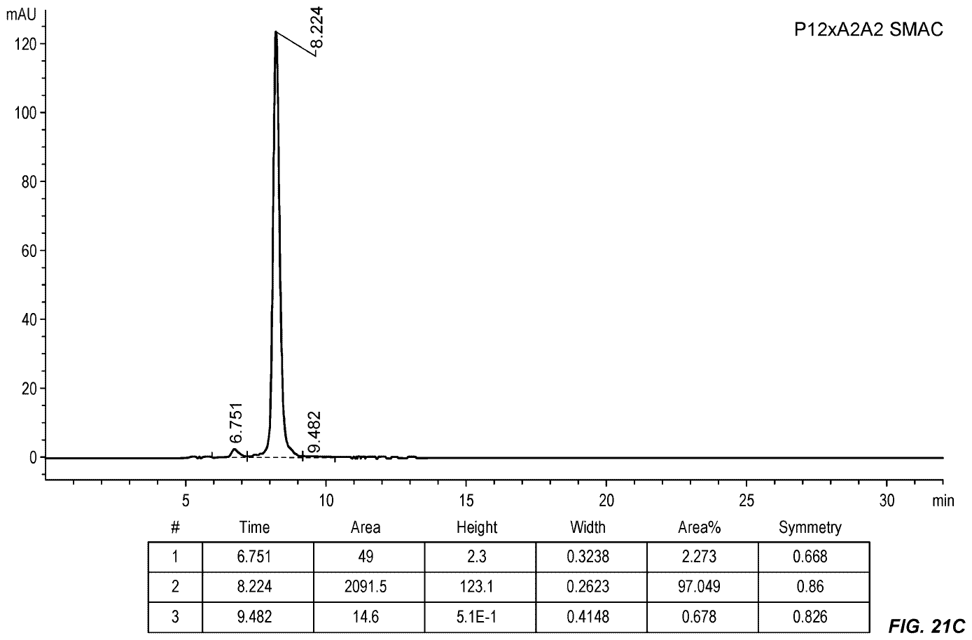

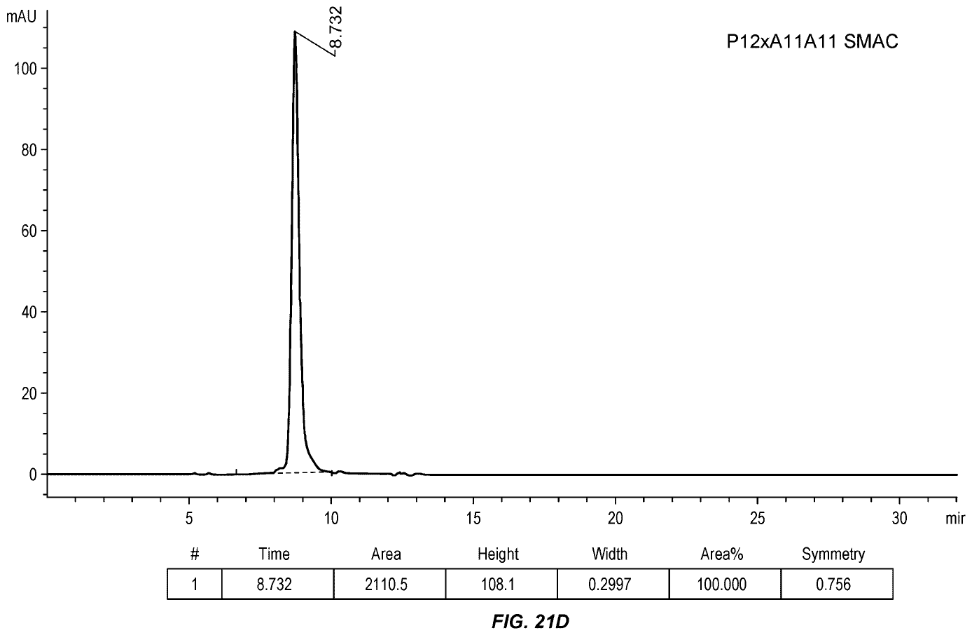

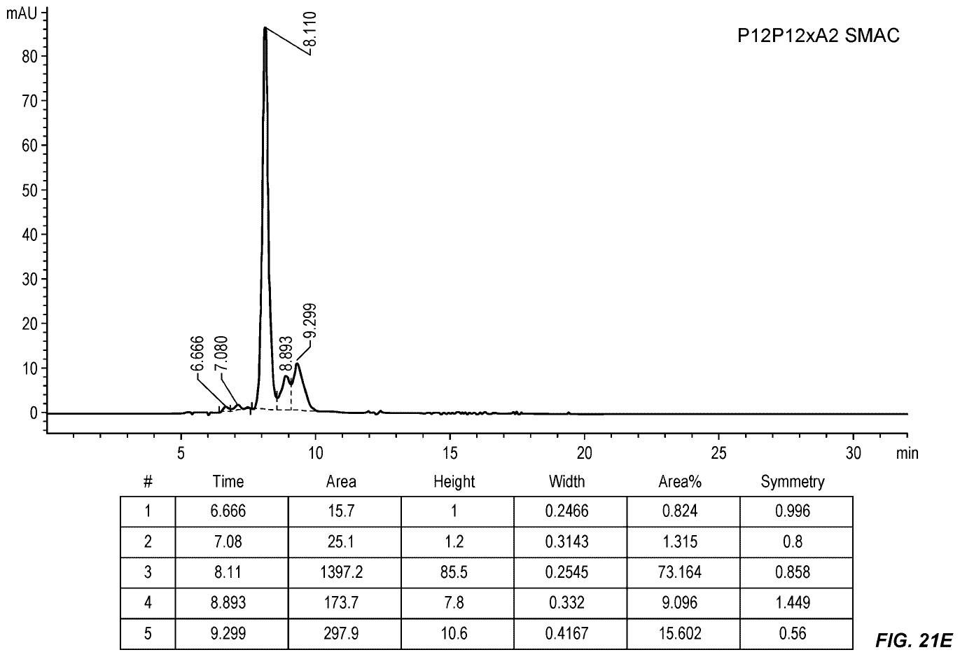

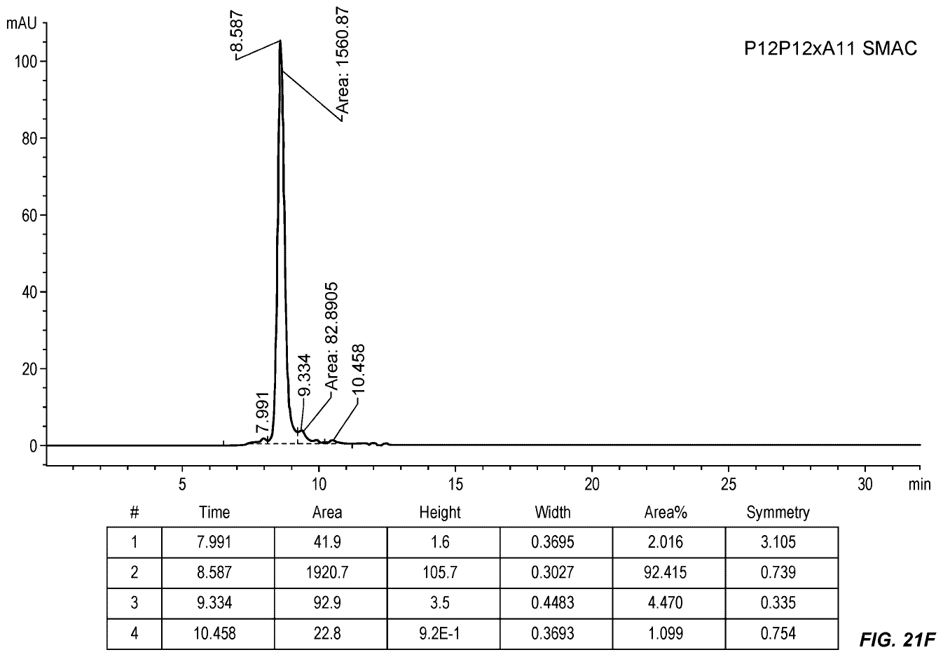

Exemplary SMAC results of bispecific antibodies P12×A2, P12×A11, P12×A2A2, P12×A11A11, P12P12×A2, and P12P12×A11 are shown in FIGS. 21A-21F. Results show that the selected bispecific antibodies had similar short retention times, indicating colloidal stability and low propensity to aggregate.

Overall, the results of the developability assays show that the developability criteria are met for the selected bispecific antibodies.

### Example 14. Co-Engagement of NK and Tumor Cells by the Disclosed Bispecific Antibodies

NKG2A×PD-L1 coupling experiment was performed to assess the engagement of NK and tumor cells in the presence of a bispecific antibody as disclosed here. Briefly, NKG2A expressing (NKG2A+) KHYG-1 effector cells (1×10⁵) were labeled with CellTrace Far Red (Thermo) per manufacturer's instructions, while the PD-L1 expressing (PD-L1+) LCL721.221-PD-L1 target cells (1×10⁵) were labeled with CellTrace Violet (Thermo) per manufacturer's instructions. Both cell types were mixed at 1:1 effector to target ratio in a 96-well V-bottom plate. Other effector to target ratios were also explored, including 25:1 and 50:1. Serial dilutions of the P12×A11, sFc antibody (0 to 2 nM) were added to the cells and incubated on ice for 2 hours. Several controls were also tested in parallel at 2 nM, including benchmark anti-NKG2A antibody, IgG4, CAS #1228763-95-8; benchmark anti-PD-L1 antibody, Fc silent IgG1, CAS #1428935-60-7; benchmark anti-PD-L1 antibody, Fc silent IgG1+benchmark anti-NKG2A antibody, IgG4, also referred to herein as the benchmark antibody or ab combination; benchmark anti-PD-L1 antibody, Fc silent IgG1+ an isotype anti-KLH arm, referred to herein as benchmark anti-PD-L1 ab×Dummy or PD-L1×Dummy; an isotype anti-KLH arm×benchmark anti-NKG2A antibody, IgG4, referred to herein Dummy×benchmark anti-NKG2A ab or NKG2A×Dummy; and an anti-KLH isotype). After the incubation, cells were spun down, resuspended in 100 μL 4% paraformaldehyde per well, and incubated for 10 minutes. The cells were then washed once with BD staining buffer. The cells were analyzed by flow cytometry on Cytoflex. All live cells were gated and analyzed accordingly.

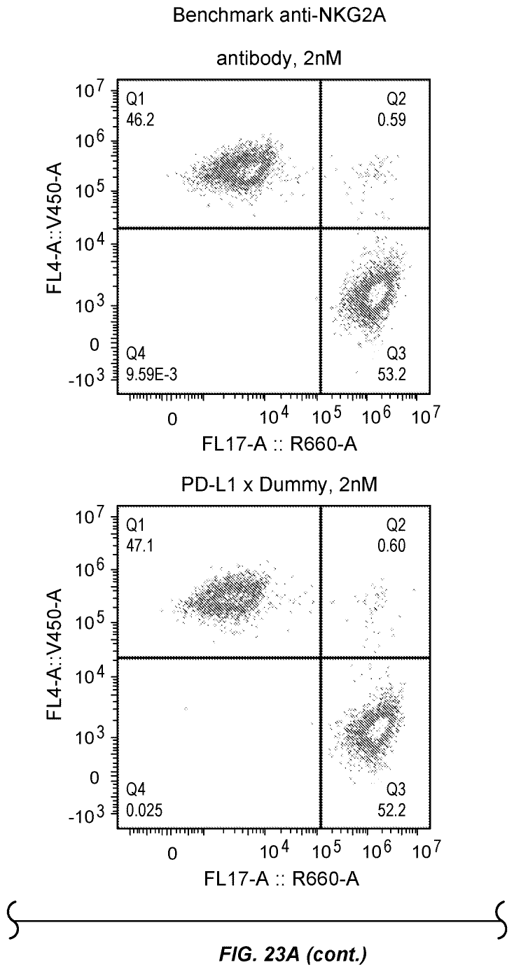

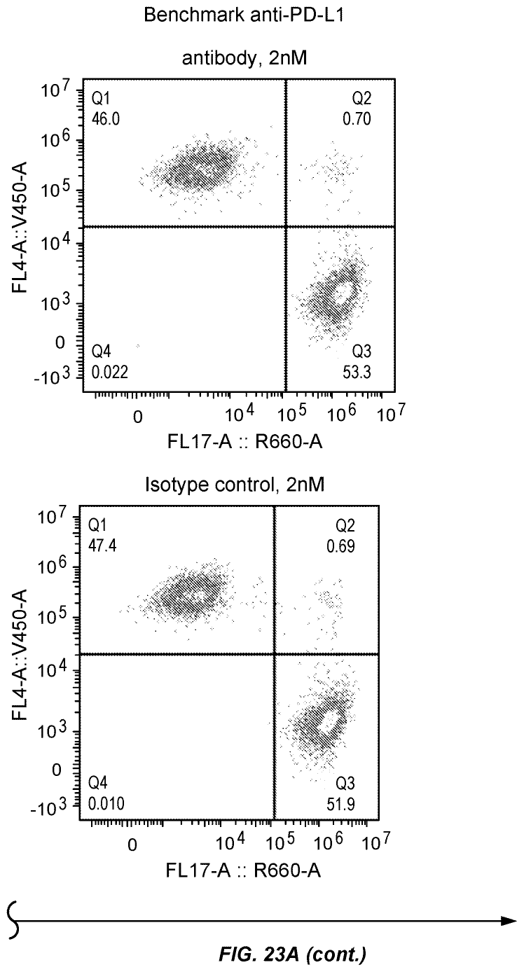

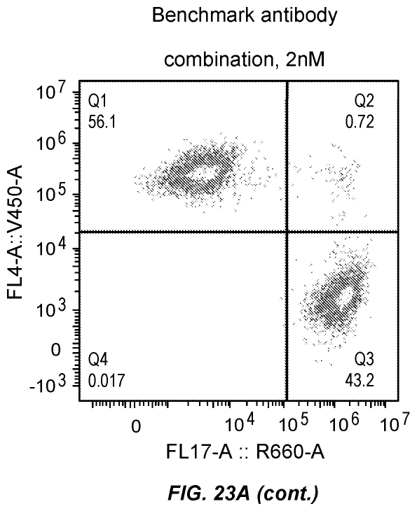

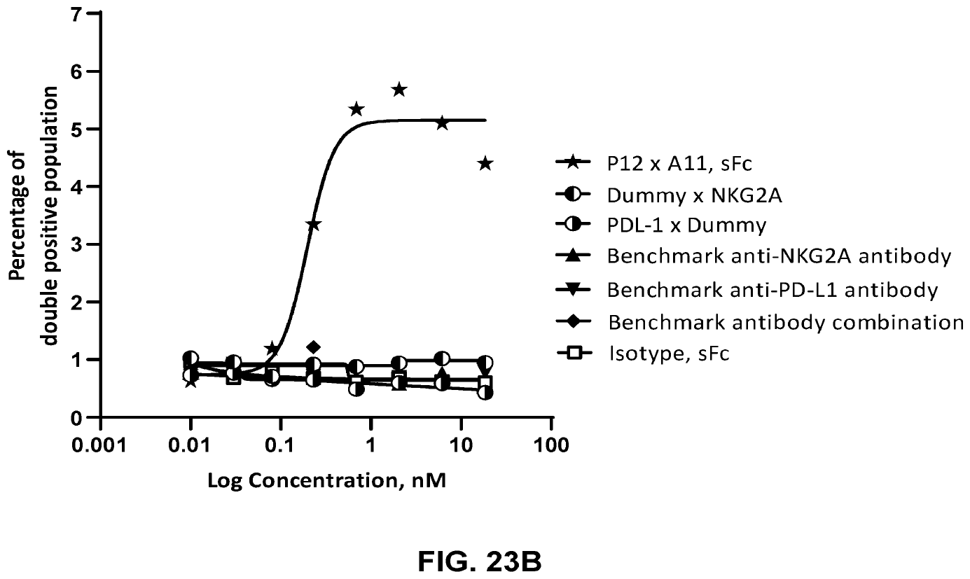

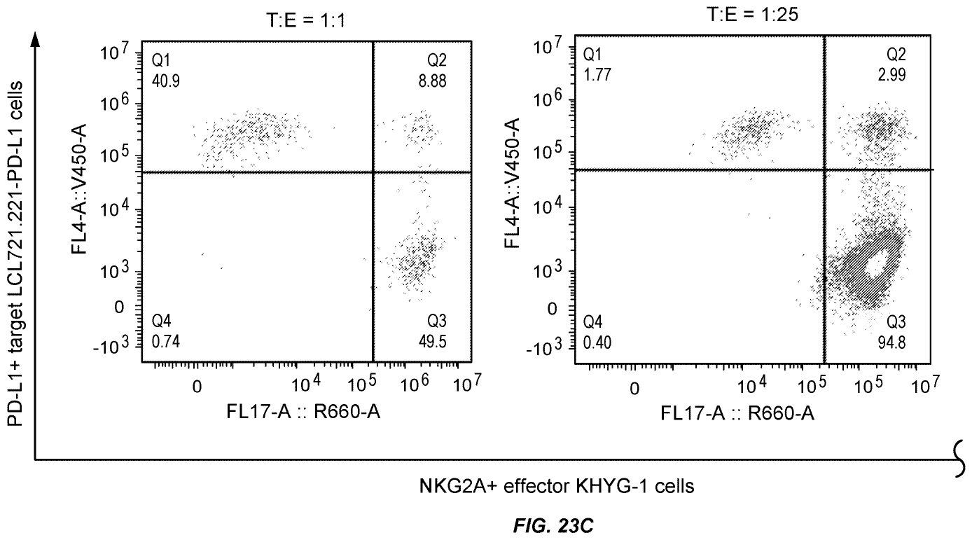

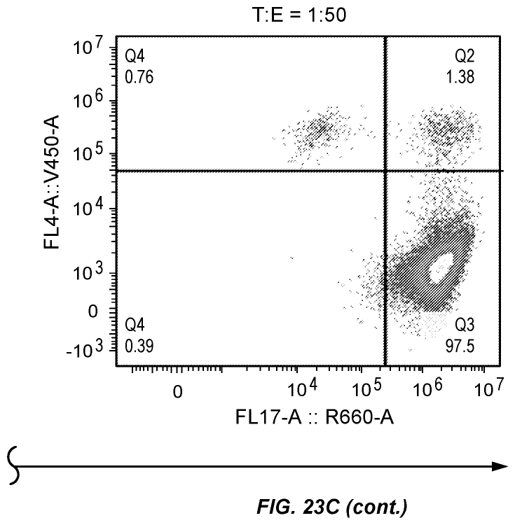

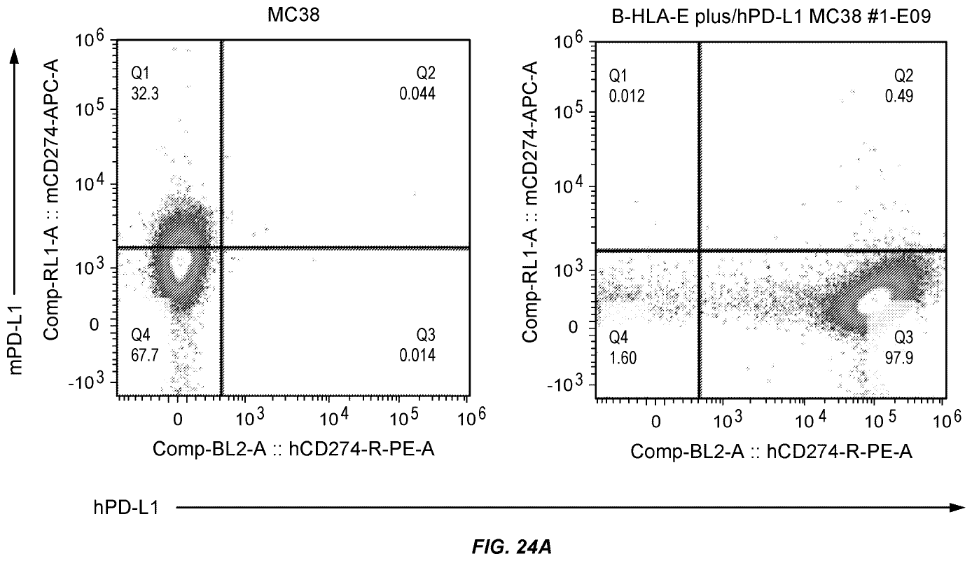

Briefly, a complex labeled with both CellTrace Far Red and CellTrace Violet was considered as comprising a tumor cell and an NK cell, and accordingly, the presence of such a double positive complex (also referred to herein as a doublet) indicated that the tested antibody brought a tumor cell and an NK cell together. An exemplary result is shown in FIG. 23A, and the corresponding measured percentages of double-positive complexes are provided in Table 10. It shows that, at the same tested concentration (2 nM), only P12×A11, sFc, but not any of the controls, brought the target tumor cells and the effector NK cells together.

TABLE 10
Percentages of double-positive complexes in cells
treated with 2 nM of P12 × A11, sFc or a control.

| | % double positive within |
|---|---|
| | total live cells |
| P12 × A11, sFc | 5.7 |
| benchmark anti-NKG2A antibody | <1 |
| benchmark anti-PD-L1 antibody | <1 |
| benchmark ab combination | <1 |
| Dummy × benchmark anti-NKG2A ab | <1 |
| benchmark anti-PD-L1 ab × Dummy | <1 |
| KLH isotype | <1 |

Further, the dose effect of P12×A11, sFc was evaluated. Percentages of double positive complexes in the serial-dilution groups were measured and the result is plotted in FIG. 23B, showing that P12×A11, sFc brought target tumor cells and effector NK cells together in a dose-dependent manner.

The effect of a target-to-effector ratio (T:E) used in the experiments was also explored, in view that an LCL721.221-PD-L1 (T) cell used herein had 550,000 copies of PD-L1 while a KHYG-1 (E) cell had 5,000 to 10,000 copies of NKG2A. Briefly, mixed cell populations having increasing NK concentrations (e.g., having a T:E of 1:1, 1:25 or 1:50) were treated with 2 nM of P12×A11, sFc and analyzed as indicated above. An exemplary result is shown in FIG. 23C, and the corresponding measured percentages of double-positive complexes are provided in Table 11. More doublets were found with increasing NK concentrations.

TABLE 11
Percentages of double-positive complexes in cells having
various T:Es and treated with 2 nM of P12 × A11, sFc.

| P12 × A11, sFc, 2 nM | T:E = 1:1 | T:E = 1:25 | T:E = 1:50 |
|---|---|---|---|
| % doublets within labeled | 19 | 62 | 64 |
| target cells | | | |

### Example 15. In Vivo Functional Evaluation

The in vivo efficacy of an NKG2A×PD-L1 bispecific as disclosed herein was tested in the treatment of subcutaneous B-hHLA-E plus/hPD-L1 MC38 in B-hPD-1/hPD-L1/hNKG2A/hCD94 mice.

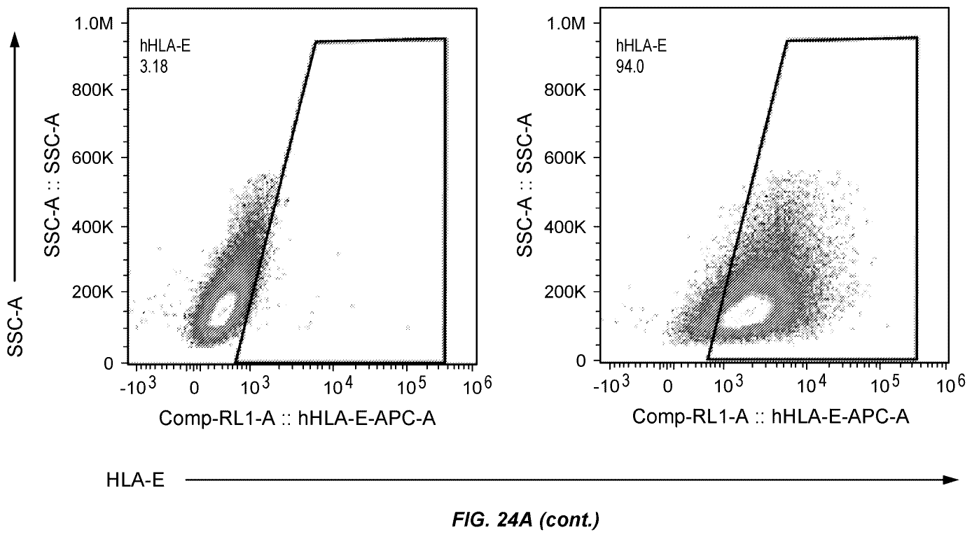

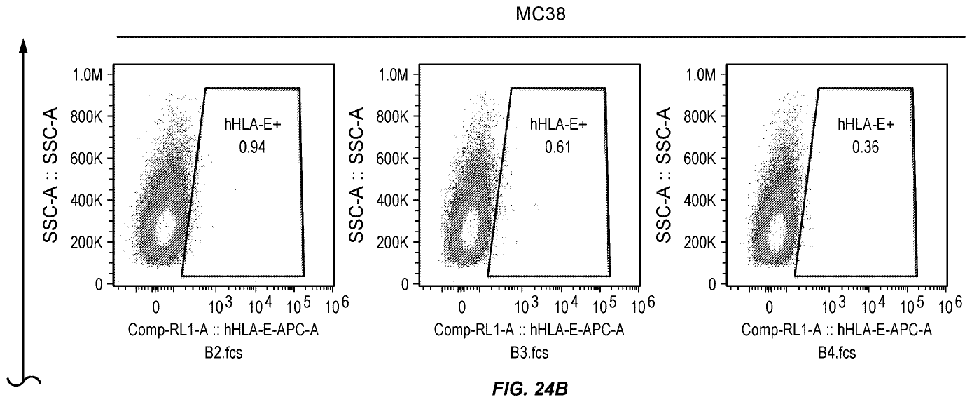

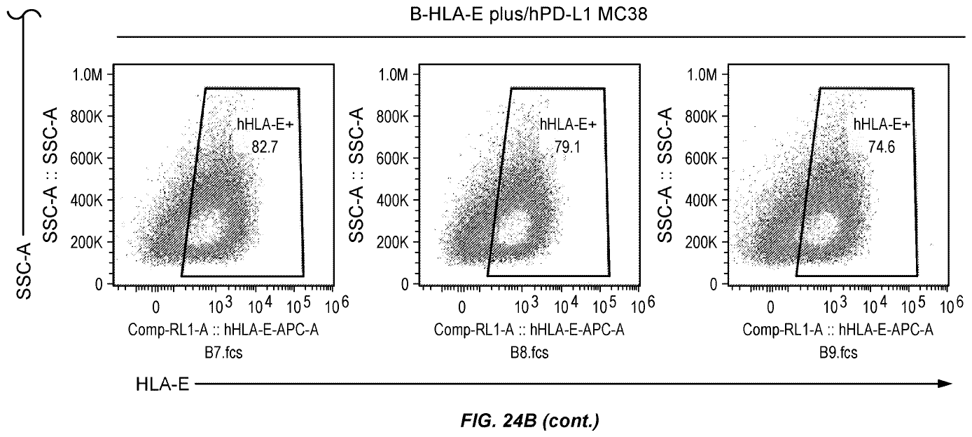

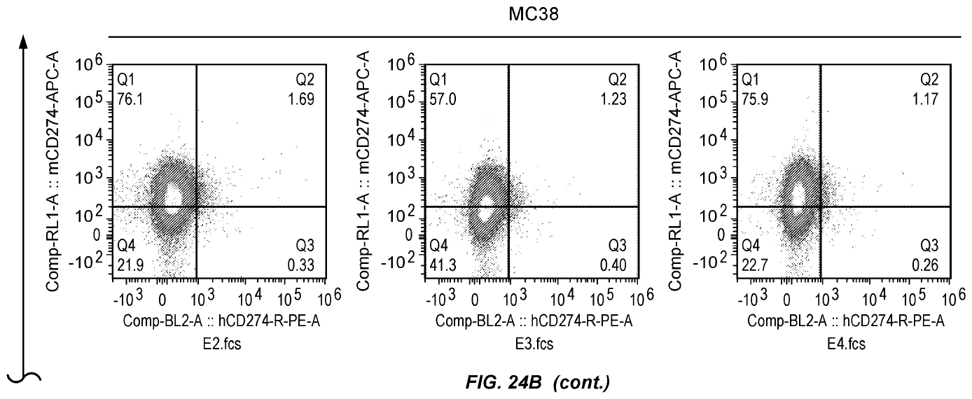

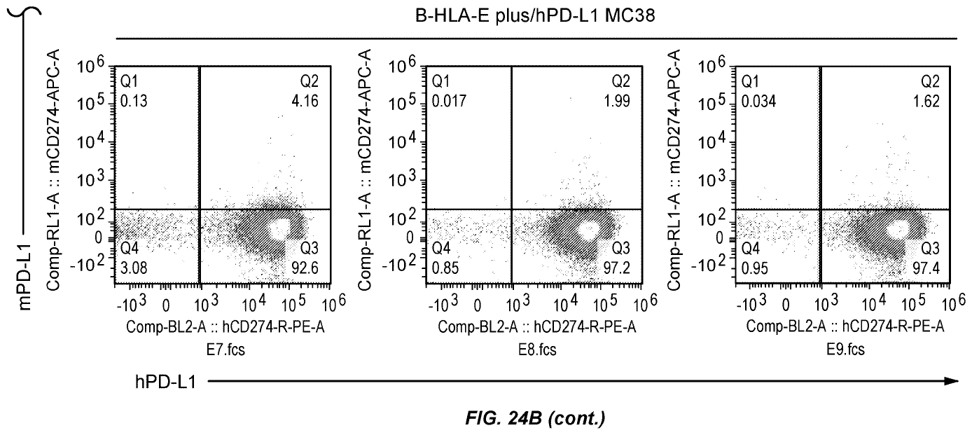

Good expressions of human HLA-E (hHLA-E) and human PD-L1 (hPD-L1) on the used tumor cells (B-hHLA-E plus/hPD-L1 MC38 cells) were confirmed in vitro via flow cytometry (FIG. 24A). The expressions of hHLA-E and hPD-L1 on B-hHLA-E plus/hPD-L1 MC38 cells were also confirmed ex vivo. Briefly, B-hHLA-E plus/hPD-L1 MC38 cells were subcutaneously transplanted into B-hPD-1/hPD-L1/hNKG2A/hCD94 mice (n=6), and on 31 days post inoculation, tumor cells were harvested and assessed for human PD-L1 and HLA-E expression by FACS. As shown in FIG. 24B, human HLA-E and PD-L1 were highly expressed by tumor cells. Therefore, B-hHLA-E plus/hPD-L1 MC38 cells is suitable for use in in vivo efficacy studies.

P12×A11, sFc antibody (0 to 2 nM) were then tested in B-hPD-1/hPD-L1/hNKG2A/hCD94 mice inoculated with B-hHLA-E plus/hPD-L1 MC38 cells. Briefly, B-hPD-1/hPD-L1/hNKG2A/hCD94 mice (a Biocytogen mouse model, C57BL/6 background, respective mouse genes replaced with human version) were subcutaneously injected with B-hHLA-E plus/hPD-L1 MC38 cells (C57BL/6 syngeneic tumor with respective mouse gene replaced with human version) (5×10⁵) in 0.1 mL PBS in the right front flank for tumor development. When the mean tumor volume reached 80-100 mm³, tumor-bearing animals were randomly enrolled into study groups based on the tumor volume and body weight. Each group consisted of 6 mice. Treatments were administrated on the same day of grouping i.p., which was then denoted as Day 0. The same treatments were repeated on Days 4, 7, 11, 14, and 18, i.e., the dosing frequency was biweekly, and the total number of the treatments was 6.

Two doses of P12×A11, sFc were tested (3 mg/kg and 10 mg/kg). Several controls were also tested in parallel, including (1) 3 mg/kg or 10 mg/kg of benchmark anti-NKG2A antibody, IgG4, CAS #1228763-95-8; (2) 3 mg/kg or 10 mg/kg of benchmark anti-PD-L1 antibody, Fc silent IgG1, CAS #1428935-60-7; 10 mg/kg of an isotype; and vehicle).

Tumor size was measured two times weekly in two dimensions using a caliper, and the volume was expressed in mm³ using the formula: V=0.5 a×b² where a and b are the long and short dimensions of the tumor, respectively. Animals were weighed before tumor inoculation and animal grouping, then two times per week during the experiment as surrogate indication for toxicity.

The tumor growth inhibition rate TGI_{TV} was calculated by the following equation: TGI_{TV}%=[1−(Tᵢ−T₀)/(Vᵢ−V₀)]×100%. (Tᵢ: means tumor volume of the treated groups at day i following treatment; T₀: means tumor volume of the treated groups at day 0; Vᵢ: means tumor volume of the vehicle groups at day i following treatment; Vₒ: means tumor volume of the vehicle groups at day 0).

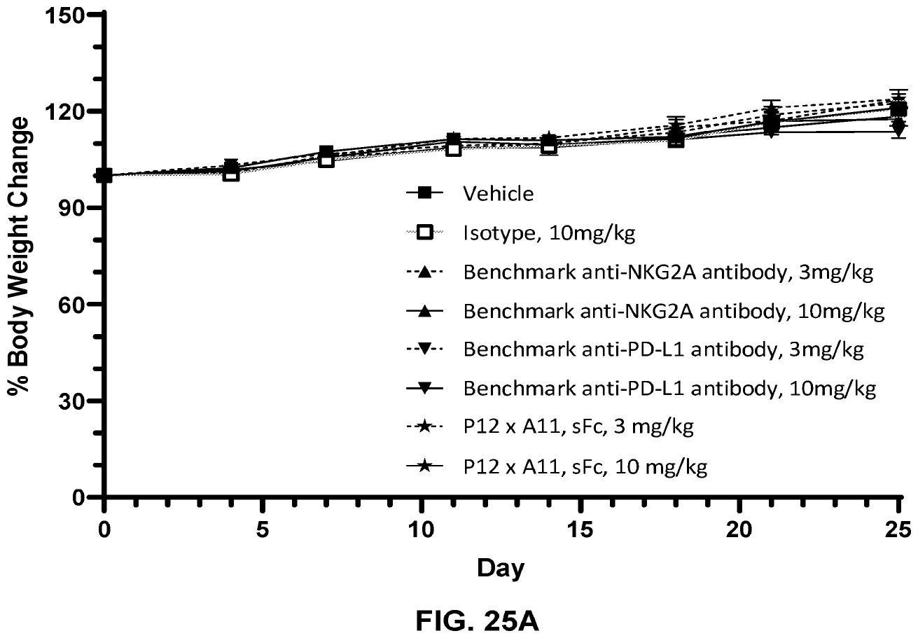

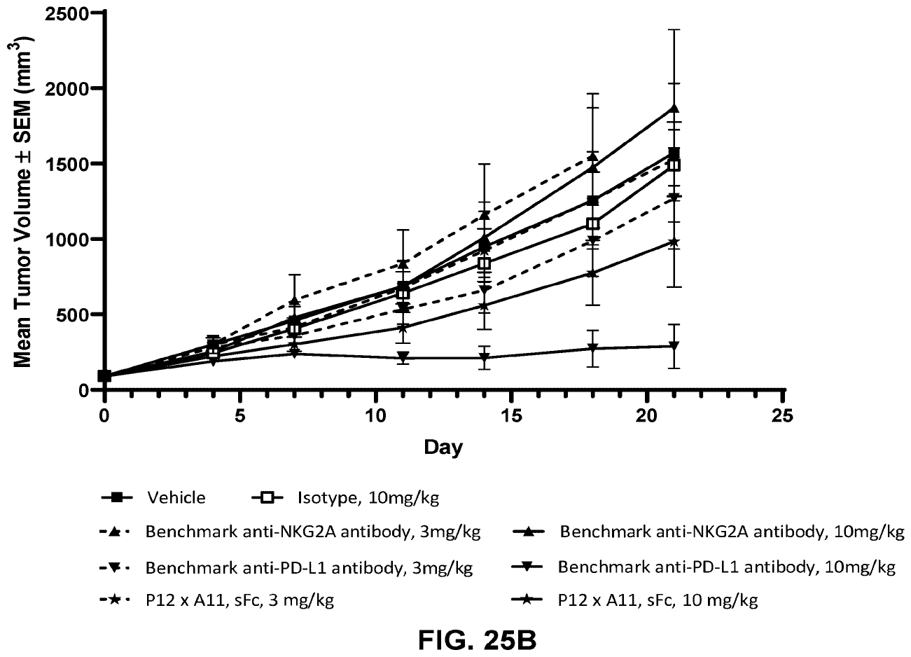

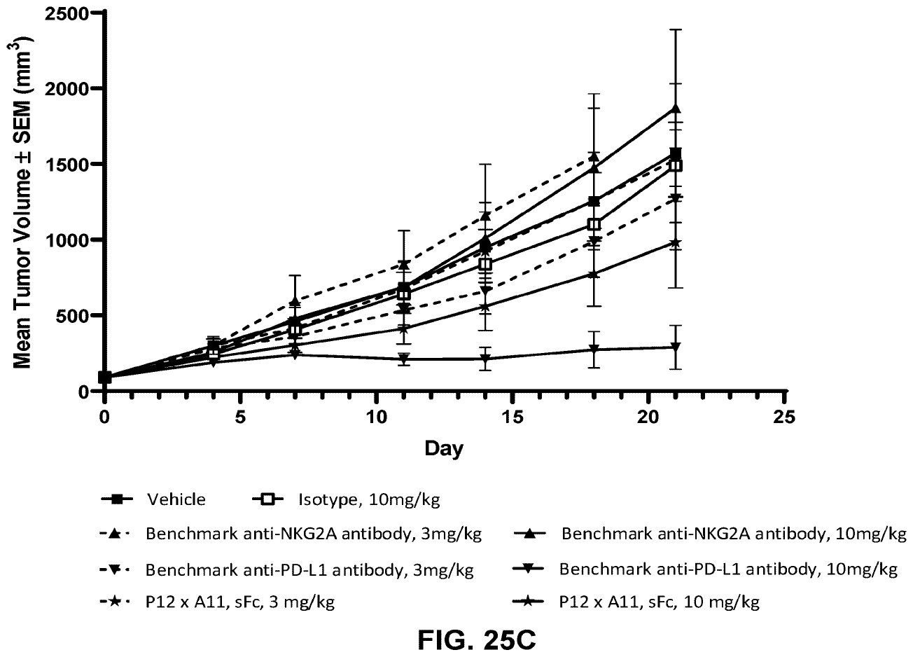

As shown in FIG. 25A, no significant change in mouse body weight was observed in any of the groups, indicating the test articles were well tolerated. Changes in tumor volumes of mice with the indicated treatments were plotted in FIGS. 25B-25C. As shown in FIG. 25B, P12×A11, sFc demonstrated a dose-dependent response. In addition, the efficacy of P12×A11, sFc observed was higher than that of the benchmark anti-NKG2A antibody, while the efficacy observed in the group treated with the benchmark anti-PD-L1 antibody was higher than in the two aforementioned groups, indicating this animal model was more sensitive to a treatment targeting the PD-L1/PD-1 axis compared to the one targeting NKG2A/HLA-E. The same relative efficacies (benchmark anti-PD-L1 antibody>P12×A11, sFc>benchmark anti-NKG2A antibody) and sensitivity were also observed in FIG. 25C and Table 12. Without wishing to be bound by the theory, the MC38 tumor (cell line and collected tumors) expresses higher levels of PD-L1 than HLA-E (for example, as stronger mean fluorescence intensity (MFI) shift was observed in PD-L1 vs. HLA-E). Quantitation on copy number of PD-L1 and HLA-E on the cell line and tumors is performed. Additionally or alternatively, without wishing to be bound by the theory, more T cells than NK cells are present in the tumor microenvironment (TME).

The efficacy of those treatments at 10 mg/kg shown by tumor volume changes was further compared and analyzed. See, FIG. 25C. In addition, the corresponding tumor growth inhibition rates (TGI_{TV}, also referred to herein as % TGI) on Day 21 (D21) were calculated and provided in Table 12.

TABLE 12
Tumor growth inhibition rates of various treatments at 10 mg/kg.

| Test article | % TGI, D21 |
|---|---|
| Isotype | 6 |
| benchmark anti-NKG2A antibody | −20 |
| P12 × A11, sFc | 40 |
| benchmark anti-PD-L1 antibody | 87 |

### Example 16. Epitope Mapping of CD94/NKG2A

Leap HDX auto sampler and Waters Cyclic IMS MS were used to map P12×A11 (sFc)'s epitope on the CD94 and NKG2A antigens. Two different Quench conditions were used for digestion to improve the coverage. Both Data sets 1 and 2 were collected for 3 different timepoints (2, 10, and 60 mins) in triplicate. D₂O Buffer used was 20 mM Phos, 150 mM NaCl, pH 7.4. The relative fractional uptake greater than 5% was considered significant in the analysis.

Specifically, the experiment was performed as follows:

Labeling with D₂O: Proteins (NKG2A/CD94 heterodimeric ectodomain complex and anti-NKG2A Mab P12×A11, sFc) were incubated in deuterated buffer (D₂O in 20 mM Phos, 150 mM NaCl, pH 7.4.) for a number of time points (2 min, 10 min, 60 min) in triplicate, allowing for the incorporation of deuterium into the protein backbone. A control arm with only the NKG2A-CD94 without the P12×A11, sFc Mab was run in parallel. Leap HDX auto sampler and Waters Cyclic IMS MS were the instruments used.

Quenching: The exchange reaction was quenched by a shift to acidic pH and a temperature drop (two proprietary conditions were used-potential variation in buffer formulation included the use of denaturants or reducing agents).

Digestion: Proteins were then digested by an acid-functional protease, such as pepsin.

LC/MS: The proteolytic peptides were desalted and separated using a chilled reversed-phase UHPLC system and eluted into a mass spectrometer, where they were ionized by electrospray and subjected to mass analysis to determine the increase in mass resulting from deuterium uptake.

During spectral analysis, the isotopic envelopes of peptides were visualized, and levels of deuteration were determined, typically through comparison of the average mass from the intensity-weighted centroid m/z value of the peptide.

Analysis: The deuterium uptake, resolved to individual peptide segments, was plotted across multiple time points.

Peptide uptake plots revealed the local HDX profile of individual protein regions.

Comparison: Peptide uptake plots obtained in an identical manner for multiple states of the protein, P12×A11, sFc bound and unbound to NKG2A-CD94 could be overlaid to enable comparison and detection of local differences in HDX between protein states. The relative fractional uptake greater than 5% was considered significant in the analysis.

The differences in HDX were then mapped on a three-dimensional representation and protein sequence to facilitate structural interpretation.

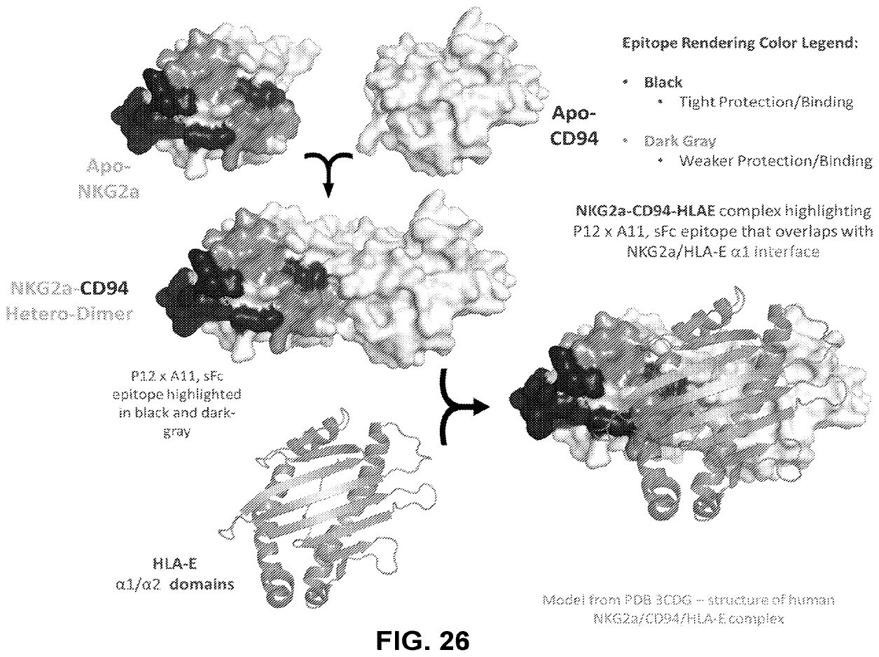

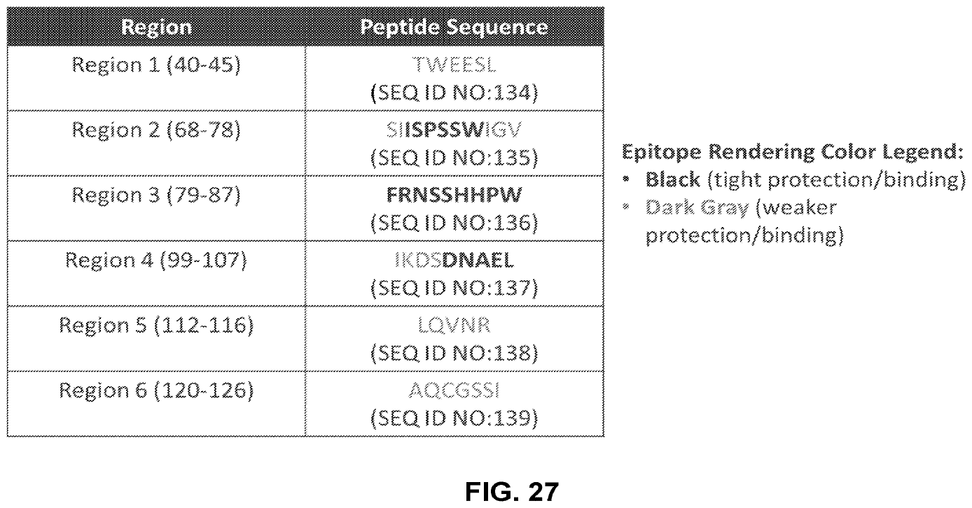

Both experimental results provided the same results and using two different conditions helped to get a good coverage of the protein. Specifically, the results indicate (1) the experimental results aligned with each other, and a better coverage of the protein was achieved by using two different conditions; (2) there was no change in deuterium (D) uptake observed for the CD94 protein, suggesting that P12×A11, sFc does not interact with CD94; and (3) the epitope was structural in nature due to the involvement of various regions of the NKG2A protein. See FIG. 26 for the epitope mapping result. A list of regions showed significant protection from Deuterium exchange was generated. See FIG. 27.

### EXEMPLARY SEQUENCES

| | Chain 1 | Chain 2 | Chain 3 | Chain 4 |
|---|---|---|---|---|
| Variant | HC (hole) | LC | HC (knob) | LC |
| P12 × A2 wtFc | SEQ ID | SEQ ID | SEQ ID | SEQ ID |
| | NO: 118 | NO: 120 | NO: 121 | NO: 122 |
| P12 × A2A2 wtFc | SEQ ID | SEQ ID | SEQ ID | SEQ ID |
| | NO: 118 | NO: 120 | NO: 123 | NO: 122 |
| P12P12 × A2 wtFc | SEQ ID | SEQ ID | SEQ ID | SEQ ID |
| | NO: 119 | NO: 120 | NO: 121 | NO: 122 |
| P12 × A11 wtFc | SEQ ID | SEQ ID | SEQ ID | SEQ ID |
| | NO: 118 | NO: 120 | NO: 125 | NO: 126 |
| P12 × A11A11 | SEQ ID | SEQ ID | SEQ ID | SEQ ID |
| wtFc | NO: 118 | NO: 120 | NO: 124 | NO: 126 |
| P12P12 × A11 wtFc | SEQ ID | SEQ ID | SEQ ID | SEQ ID |
| | NO: 119 | NO: 120 | NO: 125 | NO: 126 |
| P12 × A2 sFc | SEQ ID | SEQ ID | SEQ ID | SEQ ID |
| | NO: 127 | NO: 120 | NO: 129 | NO: 122 |
| P12 × A2A2 sFc | SEQ ID | SEQ ID | SEQ ID | SEQ ID |
| | NO: 127 | NO: 120 | NO: 130 | NO: 122 |
| P12P12 × A2 sFc | SEQ ID | SEQ ID | SEQ ID | SEQ ID |
| | NO: 128 | NO: 120 | NO: 129 | NO: 122 |
| P12 × A11 sFc | SEQ ID | SEQ ID | SEQ ID | SEQ ID |
| | NO: 127 | NO: 120 | NO: 132 | NO: 126 |
| P12 × A11A11 sFc | SEQ ID | SEQ ID | SEQ ID | SEQ ID |
| | NO: 127 | NO: 120 | NO: 131 | NO: 126 |
| P12P12 × A11 sFc | SEQ ID | SEQ ID | SEQ ID | SEQ ID |
| | NO: 128 | NO: 120 | NO: 132 | NO: 126 |

P12 Chain 1 (SEQ ID NO: 118): VL-CH3 (Y349C and K370R)-Hinge-CH2 (L234,
L235, P329, wt)-CH3 (D356E, L358M, T366S, L368A and Y407V, hole)
DIQMTQSPSSLSASVGDRVTITCRASQSVSSAVAWYQQKPGKAPKLLIYSASSLYSGVPSRFS
GSRSGTDFTLTISSLQPEDFATYYCQQFMLEPITFGQGTKVEIKGQPREPQVCTLPPSRDE
LTKNQVSLTCLVRGFYPSDIAVEWESNGQPENNYKTTPPVLDSDGSFFLYSKLTVDK
SRWQQGNVFSCSVMHEALHNHYTQKSLSLSPGKDKTHTCPPCPAPELLGGPSVFLF
PPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYT
LPPSREEMTKNQVSLSCAVKGFYPSDIAVEWESNGQPENNYKTTPPVLDSDGSFFLV
SKLTVDKSRWQQGNVFSCSVMHEALHNHYTQKSLSLSPGK
P12P12 Chain 1 (SEQ ID NO: 119): VL-CH3 (Y349C and K370R)-Linker-VL-CH3
(Y349C and K370R)-Hinge-CH2 (L234, L235, and P329, wt)-CH3 (D356E, L358M,
T366S, L368A and Y407V, hole)
DIQMTQSPSSLSASVGDRVTITCRASQSVSSAVAWYQQKPGKAPKLLIYSASSLYSGVPSRFS
GSRSGTDFTLTISSLQPEDFATYYCQQFMLEPITFGQGTKVEIKGQPREPQVCTLPPSRDE
LTKNQVSLTCLVRGFYPSDIAVEWESNGQPENNYKTTPPVLDSDGSFFLYSKLTVDK
SRWQQGNVFSCSVMHEALHNHYTQKSLSLSPGKTASSGGSSSGDIQMTQSPSSLSASV
GDRVTITCRASQSVSSAVAWYQQKPGKAPKLLIYSASSLYSGVPSRFSGSRSGTDFTLTISSL
QPEDFATYYCQQFMLEPITFGQGTKVEIKGQPREPQVCTLPPSRDELTKNQVSLTCLVR
GFYPSDIAVEWESNGQPENNYKTTPPVLDSDGSFFLYSKLTVDKSRWQQGNVFSCSV
MHEALHNHYTQKSLSLSPGKDKTHTCPPCPAPELLGGPSVFLFPPKPKDTLMISRT
PEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSREEMTKNQVS
LSCAVKGFYPSDIAVEWESNGQPENNYKTTPPVLDSDGSFFLVSKLTVDKSRWQQG
NVFSCSVMHEALHNHYTQKSLSLSPGK
P12 Chain 2 (SEQ ID NO: 120): VH-CH3 (G341A, S354C and E357W)
EVQLVESGGGLVQPGGSLRLSCAASGFTFSSYYIHWVRQAPGKGLEWVATINSRGGFTLYA
DSVKGRFTISADTSKNTAYLQMNSLRAEDTAVYYCARGYTLTPVLDYWGQGTLVTVSSAQP
REPQVYTLPPCRDWLTKNQVSLTCLVKGFYPSDIAVEWESNGQPENNYKTTPPVLDS
DGSFFLYSKLTVDKSRWQQGNVFSCSVMHEALHNHYTQKSLSLSPGK
A2 Chain 3 (SEQ ID NO: 121): VL-CL-Hinge-CH2 (L234, L235, and P329, wt)-
CH3 (T366W, knob)
DIQMTQSPSSLSASVGDRVTITCRASQSVSRAVAWYQQKPGKAPKLLIYSASSLHSGVPSRFS
GSRSGTDFTLTISSLQPEDFATYYCQQYGSSARTFGQGTKVEIKRTVAAPSVFIFPPSDEQ
LKSGTASVVCLLNNFYPREAKVQWKVDNALQSGNSQESVTEQDSKDSTYSLSSTLTLS
KADYEKHKVYACEVTHQGLSSPVTKSFNRGECDKTHTCPPCPAPELLGGPSVFLFPP
KPKDTLMISRTPEVTCVVVDVSHEDPEVKENWYVDGVEVHNAKTKPREEQYNS
TYRVVSVLTVLHQDWLNGKEYKCKVSNKALPAPIEKTISKAKGQPREPQVYTLP
PSRDELTKNQVSLWCLVKGFYPSDIAVEWESNGQPENNYKTTPPVLDSDGSFFLYSK
LTVDKSRWQQGNVFSCSVMHEALHNHYTQKSLSLSPGK
A2 Chain 4 (SEQ ID NO: 122): VH-CH1
EVQLVESGGGLVQPGGSLRLSCAASGFTFSSYYIHWVRQAPGKGLEWVASYVPEDGYTDY
ADSVKGRFTISADTSKNTAYLQMNSLRAEDTAVYYCARSSDFIPWTSSGGMDYWGQGTLVT
VSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSC
A2A2 Chain 3 (SEQ ID NO: 123): VL-CL-Linker-VL-CL-Hinge-CH2 (L234,
L235, and P329, wt)-CH3 (T366W, knob)
DIQMTQSPSSLSASVGDRVTITCRASQSVSRAVAWYQQKPGKAPKLLIYSASSLHSGVPSRFS
GSRSGTDFTLTISSLQPEDFATYYCQQYGSSARTFGQGTKVEIKRTVAAPSVFIFPPSDEQ
LKSGTASVVCLLNNFYPREAKVQWKVDNALQSGNSQESVTEQDSKDSTYSLSSTLTLS
KADYEKHKVYACEVTHQGLSSPVTKSFNRGECTASSGGSSSGDIQMTQSPSSLSASVG
DRVTITCRASQSVSRAVAWYQQKPGKAPKLLIYSASSLHSGVPSRFSGSRSGTDFTLTISSLQ
PEDFATYYCQQYGSSARTFGQGTKVEIKRTVAAPSVFIFPPSDEQLKSGTASVVCLLNN
FYPREAKVQWKVDNALQSGNSQESVTEQDSKDSTYSLSSTLTLSKADYEKHKVYACE
VTHQGLSSPVTKSFNRGECDKTHTCPPCPAPELLGGPSVFLFPPKPKDTLMISRTPE
VTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSRDELTKNQVSL
WCLVKGFYPSDIAVEWESNGQPENNYKTTPPVLDSDGSFFLYSKLTVDKSRWQQGN
VFSCSVMHEALHNHYTQKSLSLSPGK
A11A11 Chain 3 (SEQ ID NO: 124): VL-CL-Linker-VL-CL-Hinge-CH2 (L234,
L235, and P329, wt)-CH3 (T366W, knob)
DIQMTQSPSSLSASVGDRVTITCRASKSVSSALAWYQQKPGKAPKLLIYVGSSRYSGVPSRFS
GSRSGTDFTLTISSLQPEDFATYYCQQYYSSPYTFGQGTKVEIKRTVAAPSVFIFPPSDEQL
KSGTASVVCLLNNFYPREAKVQWKVDNALQSGNSQESVTEQDSKDSTYSLSSTLTLS
KADYEKHKVYACEVTHQGLSSPVTKSFNRGECTASSGGSSSGDIQMTQSPSSLSASVG
DRVTITCRASKSVSSALAWYQQKPGKAPKLLIYVGSSRYSGVPSRFSGSRSGTDFTLTISSLQ
PEDFATYYCQQYYSSPYTFGQGTKVEIKRTVAAPSVFIFPPSDEQLKSGTASVVCLLNNF
YPREAKVQWKVDNALQSGNSQESVTEQDSKDSTYSLSSTLTLSKADYEKHKVYACEV
THQGLSSPVTKSFNRGECDKTHTCPPCPAPELLGGPSVFLFPPKPKDTLMISRTPEV
TCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLW
CLVKGFYPSDIAVEWESNGQPENNYKTTPPVLDSDGSFFLYSKLTVDKSRWQQGNV
FSCSVMHEALHNHYTQKSLSLSPGK
A11 Chain 3 (SEQ ID NO: 125): VL-CL-Hinge-CH2 (L234, L235, and P329, wt)-
CH3 (T366W, knob)
DIQMTQSPSSLSASVGDRVTITCRASKSVSSALAWYQQKPGKAPKLLIYVGSSRYSGVPSRFS
GSRSGTDFTLTISSLQPEDFATYYCQQYYSSPYTFGQGTKVEIKRTVAAPSVFIFPPSDEQL
KSGTASVVCLLNNFYPREAKVQWKVDNALQSGNSQESVTEQDSKDSTYSLSSTLTLS
KADYEKHKVYACEVTHQGLSSPVTKSFNRGECDKTHTCPPCPAPELLGGPSVFLFPP
KPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLP
PSRDELTKNQVSLWCLVKGFYPSDIAVEWESNGQPENNYKTTPPVLDSDGSFFLYSK
LTVDKSRWQQGNVFSCSVMHEALHNHYTQKSLSLSPGK
A11 Chain 4 (SEQ ID NO: 126): VH-CH1
EVQLVESGGGLVQPGGSLRLSCAASGFTFSWYYIHWVRQAPGKGLEWVAVIDPISSYTYYA
DSVKGRFTISADTSKNTAYLQMNSLRAEDTAVYYCARGFDRAMDYWGQGTLVTVSSASTK
GPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSC
P12 Chain 1 (SEQ ID NO: 127): VL-CH3 (Y349C and K370R)-Hinge-CH2 (L234A,
L235A, and P329K, silent)-CH3 (D356E, L358M, T366S, L368A and Y407V, hole)
DIQMTQSPSSLSASVGDRVTITCRASQSVSSAVAWYQQKPGKAPKLLIYSASSLYSGVPSRES
GSRSGTDFTLTISSLQPEDFATYYCQQFMLEPITFGQGTKVEIKGQPREPQVCTLPPSRDE
LTKNQVSLTCLVRGFYPSDIAVEWESNGQPENNYKTTPPVLDSDGSFFLYSKLTVDK
SRWQQGNVFSCSVMHEALHNHYTQKSLSLSPGKDKTHTCPPCPAPEAAGGPSVFLF
PPKPKDTLMISRTPEVTCVVVDVSHEDPEVKFNWYVDGVEVHNAKTKPREEQY
NSTYRVVSVLTVLHQDWLNGKEYKCKVSNKALKAPIEKTISKAKGQPREPQVYT
LPPSREEMTKNQVSLSCAVKGFYPSDIAVEWESNGQPENNYKTTPPVLDSDGSFFLV
SKLTVDKSRWQQGNVFSCSVMHEALHNHYTQKSLSLSPGK
P12P12 chain 1 (SEQ ID NO: 128): VL-CH3 (Y349C and K370R)-Linker-VL-CH3
(Y349C and K370R)-Hinge-CH2 (L234A, L235A, and P329K, silent)-CH3 (D356E,
L358M, T366S, L368A and Y407V, hole)
DIQMTQSPSSLSASVGDRVTITCRASQSVSSAVAWYQQKPGKAPKLLIYSASSLYSGVPSRES
GSRSGTDFTLTISSLQPEDFATYYCQQFMLEPITFGQGTKVEIKGQPREPQVCTLPPSRDE
LTKNQVSLTCLVRGFYPSDIAVEWESNGQPENNYKTTPPVLDSDGSFFLYSKLTVDK
SRWQQGNVFSCSVMHEALHNHYTQKSLSLSPGKTASSGGSSSGDIQMTQSPSSLSASV
GDRVTITCRASQSVSSAVAWYQQKPGKAPKLLIYSASSLYSGVPSRFSGSRSGTDFTLTISSL
QPEDFATYYCQQFMLEPITFGQGTKVEIKGQPREPQVCTLPPSRDELTKNQVSLTCLVR
GFYPSDIAVEWESNGQPENNYKTTPPVLDSDGSFFLYSKLTVDKSRWQQGNVFSCSV
MHEALHNHYTQKSLSLSPGKDKTHTCPPCPAPEAAGGPSVFLFPPKPKDTLMISRT
PEVTCVVVDVSHEDPEVKFNWYVDGVEVHNAKTKPREEQYNSTYRVVSVLTVL
HQDWLNGKEYKCKVSNKALKAPIEKTISKAKGQPREPQVYTLPPSREEMTKNQVS
LSCAVKGFYPSDIAVEWESNGQPENNYKTTPPVLDSDGSFFLVSKLTVDKSRWQQG
NVFSCSVMHEALHNHYTQKSLSLSPGK
A2 Chain 3 (SEQ ID NO: 129): VL-CL-Hinge-CH2 (L234A, L235A, and P329K,
silent)-CH3 (T366W, knob)
DIQMTQSPSSLSASVGDRVTITCRASQSVSRAVAWYQQKPGKAPKLLIYSASSLHSGVPSRFS
GSRSGTDFTLTISSLQPEDFATYYCQQYGSSARTFGQGTKVEIKRTVAAPSVFIFPPSDEQ
LKSGTASVVCLLNNFYPREAKVQWKVDNALQSGNSQESVTEQDSKDSTYSLSSTLTLS
KADYEKHKVYACEVTHQGLSSPVTKSFNRGECDKTHTCPPCPAPEAAGGPSVFLFPP
KPKDTLMISRTPEVTCVVVDVSHEDPEVKFNWYVDGVEVHNAKTKPREEQYNS
TYRVVSVLTVLHQDWLNGKEYKCKVSNKALKAPIEKTISKAKGQPREPQVYTLP
PSRDELTKNQVSLWCLVKGFYPSDIAVEWESNGQPENNYKTTPPVLDSDGSFFLYSK
LTVDKSRWQQGNVFSCSVMHEALHNHYTQKSLSLSPGK
A2A2 Chain 3 (SEQ ID NO: 130): VL-CL-Linker-VL-CL-Hinge-CH2 (L234A,
L235A, and P329K, silent)-CH3 (T366W, knob)
DIQMTQSPSSLSASVGDRVTITCRASQSVSRAVAWYQQKPGKAPKLLIYSASSLHSGVPSRFS
GSRSGTDFTLTISSLQPEDFATYYCQQYGSSARTFGQGTKVEIKRTVAAPSVFIFPPSDEQ
LKSGTASVVCLLNNFYPREAKVQWKVDNALQSGNSQESVTEQDSKDSTYSLSSTLTLS
KADYEKHKVYACEVTHQGLSSPVTKSFNRGECTASSGGSSSGDIQMTQSPSSLSASVG
DRVTITCRASQSVSRAVAWYQQKPGKAPKLLIYSASSLHSGVPSRFSGSRSGTDFTLTISSLQ
PEDFATYYCQQYGSSARTFGQGTKVEIKRTVAAPSVFIFPPSDEQLKSGTASVVCLLNN
FYPREAKVQWKVDNALQSGNSQESVTEQDSKDSTYSLSSTLTLSKADYEKHKVYACE
VTHQGLSSPVTKSFNRGECDKTHTCPPCPAPEAAGGPSVFLFPPKPKDTLMISRTPE
VTCVVVDVSHEDPEVKFNWYVDGVEVHNAKTKPREEQYNSTYRVVSVLTVLH
QDWLNGKEYKCKVSNKALKAPIEKTISKAKGQPREPQVYTLPPSRDELTKNQVSL
WCLVKGFYPSDIAVEWESNGQPENNYKTTPPVLDSDGSFFLYSKLTVDKSRWQQGN
VFSCSVMHEALHNHYTQKSLSLSPGK
A11A11 Chain 3 (SEQ ID NO: 131): VL-CL-Linker-VL-CL-Hinge-CH2 (L234A,
L235A, and P329K, silent)-CH3 (T366W, knob)
DIQMTQSPSSLSASVGDRVTITCRASKSVSSALAWYQQKPGKAPKLLIYVGSSRYSGVPSRFS
GSRSGTDFTLTISSLQPEDFATYYCQQYYSSPYTFGQGTKVEIKRTVAAPSVFIFPPSDEQL
KSGTASVVCLLNNFYPREAKVQWKVDNALQSGNSQESVTEQDSKDSTYSLSSTLTLS
KADYEKHKVYACEVTHQGLSSPVTKSFNRGECTASSGGSSSGDIQMTQSPSSLSASVG
DRVTITCRASKSVSSALAWYQQKPGKAPKLLIYVGSSRYSGVPSRFSGSRSGTDFTLTISSLQ
PEDFATYYCQQYYSSPYTFGQGTKVEIKRTVAAPSVFIFPPSDEQLKSGTASVVCLLNNF
YPREAKVQWKVDNALQSGNSQESVTEQDSKDSTYSLSSTLTLSKADYEKHKVYACEV
THQGLSSPVTKSFNRGECDKTHTCPPCPAPEAAGGPSVFLFPPKPKDTLMISRTPEV
TCVVVDVSHEDPEVKFNWYVDGVEVHNAKTKPREEQYNSTYRVVSVLTVLHQ
DWLNGKEYKCKVSNKALKAPIEKTISKAKGQPREPQVYTLPPSRDELTKNQVSLW
CLVKGFYPSDIAVEWESNGQPENNYKTTPPVLDSDGSFFLYSKLTVDKSRWQQGNV
FSCSVMHEALHNHYTQKSLSLSPGK
A11 Chain 3 (SEQ ID NO: 132): VL-CL-Hinge-CH2 (L234A, L235A, and P329K,
silent)-CH3 (T366W, knob)
DIQMTQSPSSLSASVGDRVTITCRASKSVSSALAWYQQKPGKAPKLLIYVGSSRYSGVPSRFS
GSRSGTDFTLTISSLQPEDFATYYCQQYYSSPYTFGQGTKVEIKRTVAAPSVFIFPPSDEQL
KSGTASVVCLLNNFYPREAKVQWKVDNALQSGNSQESVTEQDSKDSTYSLSSTLTLS
KADYEKHKVYACEVTHQGLSSPVTKSFNRGECDKTHTCPPCPAPEAAGGPSVFLFPP
KPKDTLMISRTPEVTCVVVDVSHEDPEVKFNWYVDGVEVHNAKTKPREEQYNS
TYRVVSVLTVLHQDWLNGKEYKCKVSNKALKAPIEKTISKAKGQPREPQVYTLP
PSRDELTKNQVSLWCLVKGFYPSDIAVEWESNGQPENNYKTTPPVLDSDGSFFLYSK
LTVDKSRWQQGNVFSCSVMHEALHNHYTQKSLSLSPGK
******

Throughout this application various publications, patents, patent applications and other documents have been referenced. The disclosures of these publications, patents, patent applications and other documents in their entireties are hereby incorporated by reference in this application for all purposes, including in order to more fully describe the state of the art to which this the subject matter disclosed herein pertains. Although the disclosed subject matter has been described with reference to the examples provided above, it should be understood that various modifications can be made without departing from the spirit of the disclosed subject matter. Many variations will become apparent to those skilled in the art upon review of this specification.

What is claimed is:

1. A multispecific antibody or fragment thereof comprising a first binding domain that binds to NKG2A and a second binding domain that binds to PD-L1, wherein the first binding domain comprises any one or more of (i)-(iv):
(i) a VH CDR1, a VH CDR2, and a VH CDR3 as set forth in a VH comprising the amino acid sequence of SEQ ID NO:25 and a VL CDR1, a VL CDR2, and a VL CDR3 as set forth in a VL comprising the amino acid sequence of SEQ ID NO:26;
(ii) a VH CDR1, a VH CDR2, and a VH CDR3 as set forth in a VH comprising the amino acid sequence of SEQ ID NO:45 and a VL CDR1, a VL CDR2, and a VL CDR3 as set forth in a VL comprising the amino acid sequence of SEQ ID NO:46;
(iii) a VH CDR1, a VH CDR2, and a VH CDR3 as set forth in a VH comprising the amino acid sequence of SEQ ID NO:64 and a VL CDR1, a VL CDR2, and a VL CDR3 as set forth in a VL comprising the amino acid sequence of SEQ ID NO:65; or
(iv) a VH CDR1, a VH CDR2, and a VH CDR3 as set forth in a VH comprising the amino acid sequence of SEQ ID NO:64 and a VL CDR1, a VL CDR2, and a VL CDR3 as set forth in a VL comprising the amino acid sequence of SEQ ID NO:73.

2. The multispecific antibody or fragment thereof of claim 1, wherein the first binding domain comprises
(a) a VH region comprising:
(1) a VH CDR1 having an amino acid sequence selected from the group consisting of SEQ ID NOs: 1, 7, 12, 13, and 18;
(2) a VH CDR2 having an amino acid sequence selected from the group consisting of SEQ ID NOs: 2, 8, 14, 19, and 24; and
(3) a VH CDR3 having an amino acid sequence selected from the group consisting of SEQ ID NOs: 3, 9, 15, and 20;
and
(b) a VL region comprising:
(1) a VL CDR1 having an amino acid sequence selected from the group consisting of SEQ ID NOs: 4, 10, 16, and 21;
(2) a VL CDR2 having an amino acid sequence selected from the group consisting of SEQ ID NOs: 5, 11, and 22; and
(3) a VL CDR3 having an amino acid sequence selected from the group consisting of SEQ ID NOs: 6, 17, and 23.

3. The multispecific antibody or fragment thereof of claim 1, wherein the first binding domain comprises any one or more of (i)-(vi):
(i) a VH region comprising a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6;
(ii) a VH region comprising a VH CDR1 comprising the amino acid sequence of SEQ ID NO:7, a VH CDR2 comprising the amino acid sequence of SEQ ID NO:8, a VH CDR3 comprising the amino acid sequence of SEQ ID NO:9; and a VL region comprising a VL CDR1 comprising the amino acid sequence of SEQ ID NO:10, a VL CDR2 comprising the amino acid sequence of SEQ ID NO:11, and a VL CDR3 comprising the amino acid sequence of SEQ ID NO:6;
(iii) a VH region comprising a VH CDR1 comprising the amino acid sequence of SEQ ID NO:12, a VH CDR2 comprising the amino acid sequence of SEQ ID NO:2, a VH CDR3 comprising the amino acid sequence of SEQ ID NO:3; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6;
(iv) a VH region comprising a VH CDR1 comprising the amino acid sequence of SEQ ID NO:13, a VH CDR2 comprising the amino acid sequence of SEQ ID NO:14, a VH CDR3 comprising the amino acid sequence of SEQ ID NO:15; and a VL region comprising a VL CDR1 comprising the amino acid sequence of SEQ ID NO:16, a VL CDR2 comprising the amino acid sequence of SEQ ID NO:11, and a VL CDR3 comprising the amino acid sequence of SEQ ID NO:17;
(v) a VH region comprising a VH CDR1 comprising the amino acid sequence of SEQ ID NO:18, a VH CDR2 comprising the amino acid sequence of SEQ ID NO:19, a VH CDR3 comprising the amino acid sequence of SEQ ID NO:20; and a VL region comprising a VL CDR1 comprising the amino acid sequence of SEQ ID NO:21, a VL CDR2 comprising the amino acid sequence of SEQ ID NO:22, and a VL CDR3 comprising the amino acid sequence of SEQ ID NO:23; or
(vi) a VH region comprising a VH CDR1 comprising the amino acid sequence of SEQ ID NO:1, a VH CDR2 comprising the amino acid sequence of SEQ ID NO:24, a VH CDR3 comprising the amino acid sequence of SEQ ID NO:3; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6.

4. The multispecific antibody or fragment thereof of claim 1, wherein the first binding domain comprises
(a) a VH region comprising:
(1) a VH CDR1 having an amino acid sequence selected from the group consisting of SEQ ID NOs: 1, 7, 12, 13, and 18;
(2) a VH CDR2 having an amino acid sequence selected from the group consisting of SEQ ID NOs: 27, 32, 35, 39, and 44; and
(3) a VH CDR3 having an amino acid sequence selected from the group consisting of SEQ ID NOs: 28, 33, 36, and 40;
and
(b) a VL region comprising:
(1) a VL CDR1 having an amino acid sequence selected from the group consisting of SEQ ID NOs: 29, 34, 37, and 41;
(2) a VL CDR2 having an amino acid sequence selected from the group consisting of SEQ ID NOs: 30, 11, and 42; and
(3) a VL CDR3 having an amino acid sequence selected from the group consisting of SEQ ID NOs: 31, 38, and 43.

5. The multispecific antibody or fragment thereof of claim 1, wherein the first binding domain comprises any one or more of (i)-(vi):
(i) a VH region comprising a VH CDR1 comprising the amino acid sequence of SEQ ID NO:1, a VH CDR2 comprising the amino acid sequence of SEQ ID NO:27, a VH CDR3 comprising the amino acid sequence of SEQ ID NO:28; and a VL region comprising a VL CDR1 comprising the amino acid sequence of SEQ ID NO:29, a VL CDR2 comprising the amino acid sequence of SEQ ID NO:30, and a VL CDR3 comprising the amino acid sequence of SEQ ID NO:31;
(ii) a VH region comprising a VH CDR1 comprising the amino acid sequence of SEQ ID NO:7, a VH CDR2 comprising the amino acid sequence of SEQ ID NO:32, a VH CDR3 comprising the amino acid sequence of SEQ ID NO:33; and a VL region comprising a VL CDR1 comprising the amino acid sequence of SEQ ID NO:34, a VL CDR2 comprising the amino acid sequence of SEQ ID NO:11, and a VL CDR3 comprising the amino acid sequence of SEQ ID NO:31;
(iii) a VH region comprising a VH CDR1 comprising the amino acid sequence of SEQ ID NO:12, a VH CDR2 comprising the amino acid sequence of SEQ ID NO:27, a VH CDR3 comprising the amino acid sequence of SEQ ID NO:28; and a VL region comprising a VL CDR1 comprising the amino acid sequence of SEQ ID NO:29, a VL CDR2 comprising the amino acid sequence of SEQ ID NO:30, and a VL CDR3 comprising the amino acid sequence of SEQ ID NO:31;
(iv) a VH region comprising a VH CDR1 comprising the amino acid sequence of SEQ ID NO:13, a VH CDR2 comprising the amino acid sequence of SEQ ID NO:35, a VH CDR3 comprising the amino acid sequence of SEQ ID NO:36; and a VL region comprising a VL CDR1 comprising the amino acid sequence of SEQ ID NO:37, a VL CDR2 comprising the amino acid sequence of SEQ ID NO:11, and a VL CDR3 comprising the amino acid sequence of SEQ ID NO:38;
(v) a VH region comprising a VH CDR1 comprising the amino acid sequence of SEQ ID NO:18, a VH CDR2 comprising the amino acid sequence of SEQ ID NO:39, a VH CDR3 comprising the amino acid sequence of SEQ ID NO:40; and a VL region comprising a VL CDR1 comprising the amino acid sequence of SEQ ID NO:41, a VL CDR2 comprising the amino acid sequence of SEQ ID NO:42, and a VL CDR3 comprising the amino acid sequence of SEQ ID NO:43; or
(vi) a VH region comprising a VH CDR1 comprising the amino acid sequence of SEQ ID NO:1, a VH CDR2 comprising the amino acid sequence of SEQ ID NO:44, a VH CDR3 comprising the amino acid sequence of SEQ ID NO:28; and a VL region comprising a VL CDR1 comprising the amino acid sequence of SEQ ID NO:29, a VL CDR2 comprising the amino acid sequence of SEQ ID NO:30, and a VL CDR3 comprising the amino acid sequence of SEQ ID NO:31.

6. The multispecific antibody or fragment thereof of claim 1, wherein the first binding domain comprises
(a) a VH region comprising:
(1) a VH CDR1 having an amino acid sequence selected from the group consisting of SEQ ID NOs: 47, 51, 54, 55, and 59;
(2) a VH CDR2 having an amino acid sequence selected from the group consisting of SEQ ID NOs: 48, 52, 56, 60, and 63; and
(3) a VH CDR3 having an amino acid sequence selected from the group consisting of SEQ ID NOs: 49, 53, 57, and 61;
and
(b) a VL region comprising:
(1) a VL CDR1 having an amino acid sequence selected from the group consisting of SEQ ID NOs: 4, 10, 16, and 21;
(2) a VL CDR2 having an amino acid sequence selected from the group consisting of SEQ ID NOs: 5, 11, and 22; and
(3) a VL CDR3 having an amino acid sequence selected from the group consisting of SEQ ID NOs: 50, 58, and 62.

7. The multispecific antibody or fragment thereof of claim 1, wherein the first binding domain comprises any one or more of (i)-(vi):
(i) a VH region comprising a VH CDR1 comprising the amino acid sequence of SEQ ID NO:47, a VH CDR2 comprising the amino acid sequence of SEQ ID NO:48, a VH CDR3 comprising the amino acid sequence of SEQ ID NO:49; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:50;
(ii) a VH region comprising a VH CDR1 comprising the amino acid sequence of SEQ ID NO:51, a VH CDR2 comprising the amino acid sequence of SEQ ID NO:52, a VH CDR3 comprising the amino acid sequence of SEQ ID NO:53; and a VL region comprising a VL CDR1 comprising the amino acid sequence of SEQ ID NO:10, a VL CDR2 comprising the amino acid sequence of SEQ ID NO:11, and a VL CDR3 comprising the amino acid sequence of SEQ ID NO:50;
(iii) a VH region comprising a VH CDR1 comprising the amino acid sequence of SEQ ID NO:54, a VH CDR2 comprising the amino acid sequence of SEQ ID NO:48, a VH CDR3 comprising the amino acid sequence of SEQ ID NO:49; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:50;
(iv) a VH region comprising a VH CDR1 comprising the amino acid sequence of SEQ ID NO:55, a VH CDR2 comprising the amino acid sequence of SEQ ID NO:56, a VH CDR3 comprising the amino acid sequence of SEQ ID NO:57; and a VL region comprising a VL CDR1 comprising the amino acid sequence of SEQ ID NO:16, a VL CDR2 comprising the amino acid sequence of SEQ ID NO:11, and a VL CDR3 comprising the amino acid sequence of SEQ ID NO:58;
(v) a VH region comprising a VH CDR1 comprising the amino acid sequence of SEQ ID NO:59, a VH CDR2 comprising the amino acid sequence of SEQ ID NO:60, a VH CDR3 comprising the amino acid sequence of SEQ ID NO:61; and a VL region comprising a VL CDR1 comprising the amino acid sequence of SEQ ID NO:21, a VL CDR2 comprising the amino acid sequence of SEQ ID NO:22, and a VL CDR3 comprising the amino acid sequence of SEQ ID NO:62; or
(vi) a VH region comprising a VH CDR1 comprising the amino acid sequence of SEQ ID NO:47, a VH CDR2 comprising the amino acid sequence of SEQ ID NO:63, a VH CDR3 comprising the amino acid sequence of SEQ ID NO:49; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:50.

8. The multispecific antibody or fragment thereof of claim 1, wherein the first binding domain comprises
(a) a VH region comprising:
(1) a VH CDR1 having an amino acid sequence selected from the group consisting of SEQ ID NOs: 47, 51, 54, 55, and 59;
(2) a VH CDR2 having an amino acid sequence selected from the group consisting of SEQ ID NOs: 48, 52, 56, 60, and 63; and
(3) a VH CDR3 having an amino acid sequence selected from the group consisting of SEQ ID NOs: 49, 53, 57, and 61;
and
(b) a VL region comprising:
(1) a VL CDR1 having an amino acid sequence selected from the group consisting of SEQ ID NOs: 66, 68, 70, and 71;
(2) a VL CDR2 having an amino acid sequence selected from the group consisting of SEQ ID NOs: 67, 69, and 72; and
(3) a VL CDR3 having an amino acid sequence selected from the group consisting of SEQ ID NOs: 50, 58, and 62.

9. The multispecific antibody or fragment thereof of claim 1, wherein the first binding domain comprises any one or more of (i)-(vi):
(i) a VH region comprising a VH CDR1 comprising the amino acid sequence of SEQ ID NO:47, a VH CDR2 comprising the amino acid sequence of SEQ ID NO:48, a VH CDR3 comprising the amino acid sequence of SEQ ID NO:49; and a VL region comprising a VL CDR1 comprising the amino acid sequence of SEQ ID NO:66, a VL CDR2 comprising the amino acid sequence of SEQ ID NO:67, and a VL CDR3 comprising the amino acid sequence of SEQ ID NO:50;
(ii) a VH region comprising a VH CDR1 comprising the amino acid sequence of SEQ ID NO:51, a VH CDR2 comprising the amino acid sequence of SEQ ID NO:52, a VH CDR3 comprising the amino acid sequence of SEQ ID NO:53; and a VL region comprising a VL CDR1 comprising the amino acid sequence of SEQ ID NO:68, a VL CDR2 comprising the amino acid sequence of SEQ ID NO:69, and a VL CDR3 comprising the amino acid sequence of SEQ ID NO:50;
(iii) a VH region comprising a VH CDR1 comprising the amino acid sequence of SEQ ID NO:54, a VH CDR2 comprising the amino acid sequence of SEQ ID NO:48, a VH CDR3 comprising the amino acid sequence of SEQ ID NO:49; and a VL region comprising a VL CDR1 comprising the amino acid sequence of SEQ ID NO:66, a VL CDR2 comprising the amino acid sequence of SEQ ID NO:67, and a VL CDR3 comprising the amino acid sequence of SEQ ID NO:50;
(iv) a VH region comprising a VH CDR1 comprising the amino acid sequence of SEQ ID NO:55, a VH CDR2 comprising the amino acid sequence of SEQ ID NO:56, a VH CDR3 comprising the amino acid sequence of SEQ ID NO:57; and a VL region comprising a VL CDR1 comprising the amino acid sequence of SEQ ID NO:70, a VL CDR2 comprising the amino acid sequence of SEQ ID NO:69, and a VL CDR3 comprising the amino acid sequence of SEQ ID NO:58;
(v) a VH region comprising a VH CDR1 comprising the amino acid sequence of SEQ ID NO:59, a VH CDR2 comprising the amino acid sequence of SEQ ID NO:60, a VH CDR3 comprising the amino acid sequence of SEQ ID NO:61; and a VL region comprising a VL CDR1 comprising the amino acid sequence of SEQ ID NO:71, a VL CDR2 comprising the amino acid sequence of SEQ ID NO:72, and a VL CDR3 comprising the amino acid sequence of SEQ ID NO:62; or
(vi) a VH region comprising a VH CDR1 comprising the amino acid sequence of SEQ ID NO:47, a VH CDR2 comprising the amino acid sequence of SEQ ID NO:63, a VH CDR3 comprising the amino acid sequence of SEQ ID NO:49; and a VL region comprising a VL CDR1 comprising the amino acid sequence of SEQ ID NO:66, a VL CDR2 comprising the amino acid sequence of SEQ ID NO:67, and a VL CDR3 comprising the amino acid sequence of SEQ ID NO:50.

10. The multispecific antibody or fragment thereof of any one of claims 1-9, wherein the first binding domain further comprises a framework 1 (FR1), a framework 2 (FR2), a framework 3 (FR3) and/or a framework 4 (FR4) sequence, optionally as set forth in any one of SEQ ID NOs: 25, 26, 45, 46, 64, 65, and 73.

11. The multispecific antibody or fragment thereof of any one of claims 1-10, wherein the first binding domain further comprises human framework sequences.

12. The multispecific antibody or fragment thereof of any one of claims 1-11, wherein the first binding domain comprises:
(i) a VH comprising the amino acid sequence of SEQ ID NO:25 and a VL comprising the amino acid sequence of SEQ ID NO:26;
(ii) a VH comprising the amino acid sequence of SEQ ID NO:45 and a VL comprising the amino acid sequence of SEQ ID NO:46;
(iii) a VH comprising the amino acid sequence of SEQ ID NO:64 and a VL comprising the amino acid sequence of SEQ ID NO:65; or
(iv) a VH comprising the amino acid sequence of SEQ ID NO:64 and a VL comprising the amino acid sequence of SEQ ID NO:73.

13. The multispecific antibody or fragment thereof of any one of claims 1-12, wherein the second binding domain comprises a VH CDR1, a VH CDR2, and a VH CDR3 as set forth in a VH comprising the amino acid sequence of SEQ ID NO:98 and a VL CDR1, a VL CDR2, and a VL CDR3 as set forth in a VL comprising the amino acid sequence of SEQ ID NO: 99;
wherein the first binding domain binds to a complex comprising NKG2A and CD94 or extracellular domains of each thereof;
wherein the first binding domain does not bind to NKG2C, or a complex comprising NKG2C and CD94 or extracellular domains of each thereof;
wherein the first binding domain also binds to cyno NKG2A; and/or
wherein the first binding domain does not bind to cyno NKG2A.

14. A multispecific antibody or fragment thereof comprising a first binding domain that binds to NKG2A and a second binding domain that binds to PD-L1, wherein the second binding domain comprises a VH CDR1, a VH CDR2, and a VH CDR3 as set forth in a VH comprising the amino acid sequence of SEQ ID NO:98 and a VL CDR1, a VL CDR2, and a VL CDR3 as set forth in a VL comprising the amino acid sequence of SEQ ID NO:99.

15. The multispecific antibody or fragment thereof of claim 13 or 14, wherein the second binding domain comprises
(a) a VH region comprising:
(1) a VH CDR1 having an amino acid sequence selected from the group consisting of SEQ ID NOs: 1, 7, 12, 13, and 18;
(2) a VH CDR2 having an amino acid sequence selected from the group consisting of SEQ ID NOs: 86, 89, 91, 94, and 97; and
(3) a VH CDR3 having an amino acid sequence selected from the group consisting of SEQ ID NOs: 87, 90. 92, and 95; and
(b) a VL region comprising:
(1) a VL CDR1 having an amino acid sequence selected from the group consisting of SEQ ID NOs: 4, 10, 16, and 21;
(2) a VL CDR2 having an amino acid sequence selected from the group consisting of SEQ ID NOs: 5, 11, and 22; and
(3) a VL CDR3 having an amino acid sequence selected from the group consisting of SEQ ID NOs: 88, 93, and 96.

16. The multispecific antibody or fragment thereof of claim 13 or 14, wherein the second binding domain comprises any one or more of (i)-(vi):
(i) a VH region comprising a VH CDR1 comprising the amino acid sequence of SEQ ID NO:1, a VH CDR2 comprising the amino acid sequence of SEQ ID NO:86, a VH CDR3 comprising the amino acid sequence of SEQ ID NO:87; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:88;
(ii) a VH region comprising a VH CDR1 comprising the amino acid sequence of SEQ ID NO:7, a VH CDR2 comprising the amino acid sequence of SEQ ID NO:89, a VH CDR3 comprising the amino acid sequence of SEQ ID NO:90; and a VL region comprising a VL CDR1 comprising the amino acid sequence of SEQ ID NO:10, a VL CDR2 comprising the amino acid sequence of SEQ ID NO:11, and a VL CDR3 comprising the amino acid sequence of SEQ ID NO:88;
(iii) a VH region comprising a VH CDR1 comprising the amino acid sequence of SEQ ID NO:12, a VH CDR2 comprising the amino acid sequence of SEQ ID NO:86, a VH CDR3 comprising the amino acid sequence of SEQ ID NO:87; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:88;
(iv) a VH region comprising a VH CDR1 comprising the amino acid sequence of SEQ ID NO:13, a VH CDR2 comprising the amino acid sequence of SEQ ID NO:91, a VH CDR3 comprising the amino acid sequence of SEQ ID NO:92; and a VL region comprising a VL CDR1 comprising the amino acid sequence of SEQ ID NO:16, a VL CDR2 comprising the amino acid sequence of SEQ ID NO:11, and a VL CDR3 comprising the amino acid sequence of SEQ ID NO:93;
(v) a VH region comprising a VH CDR1 comprising the amino acid sequence of SEQ ID NO:18, a VH CDR2 comprising the amino acid sequence of SEQ ID NO:94, a VH CDR3 comprising the amino acid sequence of SEQ ID NO:95; and a VL region comprising a VL CDR1 comprising the amino acid sequence of SEQ ID NO:21, a VL CDR2 comprising the amino acid sequence of SEQ ID NO:22, and a VL CDR3 comprising the amino acid sequence of SEQ ID NO:96; or
(vi) a VH region comprising a VH CDR1 comprising the amino acid sequence of SEQ ID NO:1, a VH CDR2 comprising the amino acid sequence of SEQ ID NO:97, a VH CDR3 comprising the amino acid sequence of SEQ ID NO:87; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:88.

17. The multispecific antibody or fragment thereof of claim 13 or 14, wherein the second binding domain comprises VH region comprising the amino acid sequence of SEQ ID NO: 98 and a VL region comprising the amino acid sequence of SEQ ID NO:99.

18. The multispecific antibody or fragment thereof of any one of claims 1-17, wherein the multispecific antibody or fragment thereof comprises:
(i) a first polypeptide comprising from N-terminus to C-terminus: a first VL, a first CH3, a first CH2, and a second CH3;
(ii) a second polypeptide comprising from N-terminus to C-terminus: a first VH and a third CH3;
(iii) a third polypeptide comprising from N-terminus to C-terminus: a second VL, a CL, a second CH2, and a fourth CH3;
(iv) a fourth polypeptide comprising from N-terminus to C-terminus: a second VH and a CH1;
wherein the first polypeptide and the second polypeptide form the second binding domain that binds to PD-L1, and the third polypeptide and the fourth polypeptide form the first binding domain that binds to NKG2A,
wherein optionally:
(1) the first polypeptide comprises the amino acid sequence as set forth in SEQ ID NO: 118, the second polypeptide comprises the amino acid sequence as set forth in SEQ ID NO:120, the third polypeptide comprises the amino acid sequence as set forth in SEQ ID NO:121, and the fourth polypeptide comprises the amino acid sequence as set forth in SEQ ID NO:122;
(2) the first polypeptide comprises the amino acid sequence as set forth in SEQ ID NO: 118, the second polypeptide comprises the amino acid sequence as set forth in SEQ ID NO:120, the third polypeptide comprises the amino acid sequence as set forth in SEQ ID NO:123, and the fourth polypeptide comprises the amino acid sequence as set forth in SEQ ID NO:122;
(3) the first polypeptide comprises the amino acid sequence as set forth in SEQ ID NO: 119, the second polypeptide comprises the amino acid sequence as set forth in SEQ ID NO:120, the third polypeptide comprises the amino acid sequence as set forth in SEQ ID NO:121, and the fourth polypeptide comprises the amino acid sequence as set forth in SEQ ID NO:122;
(4) the first polypeptide comprises the amino acid sequence as set forth in SEQ ID NO: 118, the second polypeptide comprises the amino acid sequence as set forth in SEQ ID NO:120, the third polypeptide comprises the amino acid sequence as set forth in SEQ ID NO:125, and the fourth polypeptide comprises the amino acid sequence as set forth in SEQ ID NO:126;
(5) the first polypeptide comprises the amino acid sequence as set forth in SEQ ID NO: 118, the second polypeptide comprises the amino acid sequence as set forth in SEQ ID NO:120, the third polypeptide comprises the amino acid sequence as set forth in SEQ ID NO:124, and the fourth polypeptide comprises the amino acid sequence as set forth in SEQ ID NO:126;
(6) the first polypeptide comprises the amino acid sequence as set forth in SEQ ID NO: 119, the second polypeptide comprises the amino acid sequence as set forth in SEQ ID NO:120, the third polypeptide comprises the amino acid sequence as set forth in SEQ ID NO:125, and the fourth polypeptide comprises the amino acid sequence as set forth in SEQ ID NO:126;
(7) the first polypeptide comprises the amino acid sequence as set forth in SEQ ID NO: 127, the second polypeptide comprises the amino acid sequence as set forth in SEQ ID NO:120, the third polypeptide comprises the amino acid sequence as set forth in SEQ ID NO: 129, and the fourth polypeptide comprises the amino acid sequence as set forth in SEQ ID NO:122;
(8) the first polypeptide comprises the amino acid sequence as set forth in SEQ ID NO: 127, the second polypeptide comprises the amino acid sequence as set forth in SEQ ID NO:120, the third polypeptide comprises the amino acid sequence as set forth in SEQ ID NO:130, and the fourth polypeptide comprises the amino acid sequence as set forth in SEQ ID NO:122;
(9) the first polypeptide comprises the amino acid sequence as set forth in SEQ ID NO: 128, the second polypeptide comprises the amino acid sequence as set forth in SEQ ID NO:120, the third polypeptide comprises the amino acid sequence as set forth in SEQ ID NO:129, and the fourth polypeptide comprises the amino acid sequence as set forth in SEQ ID NO:122;
(10) the first polypeptide comprises the amino acid sequence as set forth in SEQ ID NO: 127, the second polypeptide comprises the amino acid sequence as set forth in SEQ ID NO:120, the third polypeptide comprises the amino acid sequence as set forth in SEQ ID NO:132, and the fourth polypeptide comprises the amino acid sequence as set forth in SEQ ID NO:126;
(11) the first polypeptide comprises the amino acid sequence as set forth in SEQ ID NO: 127, the second polypeptide comprises the amino acid sequence as set forth in SEQ ID NO:120, the third polypeptide comprises the amino acid sequence as set forth in SEQ ID NO:131, and the fourth polypeptide comprises the amino acid sequence as set forth in SEQ ID NO:126; or
(12) the first polypeptide comprises the amino acid sequence as set forth in SEQ ID NO: 128, the second polypeptide comprises the amino acid sequence as set forth in SEQ ID NO:120, the third polypeptide comprises the amino acid sequence as set forth in SEQ ID NO:132, and the fourth polypeptide comprises the amino acid sequence as set forth in SEQ ID NO:126.

19. The multispecific antibody or fragment thereof of any one of claims 1-18, wherein the multispecific antibody or fragment thereof further comprises a third binding domain.

20. The multispecific antibody or fragment thereof of claim 19, wherein the third binding domain binds to NKG2A.

21. The multispecific antibody or fragment thereof of claim 19, wherein the third binding domain is same as the first binding domain.

22. The multispecific antibody or fragment thereof of claim 20, wherein the multispecific antibody or fragment thereof comprises:
(i) a first polypeptide comprising from N-terminus to C-terminus: a first VL, a first CH3, a first CH2, and a second CH3;
(ii) a second polypeptide comprising from N-terminus to C-terminus: a first VH and a third CH3;
(iii) a third polypeptide comprising from N-terminus to C-terminus: a second VL, a first CL, a third VL, a second CL, a second CH2, and a fourth CH3, wherein the second VL and the third VL are the same, and the first CL and the second CL are the same;
(iv) a fourth polypeptide comprising from N-terminus to C-terminus: a second VH and a first CH1;
(v) a fifth polypeptide comprising from N-terminus to C-terminus: a third VH and a second CH1;
wherein the second VH and the third VH are the same, and the first CH1 and the second CH1 are the same; and
wherein the first polypeptide and the second polypeptide form the second binding domain that binds to PD-L1, the third polypeptide and the fourth polypeptide form the first binding domain that binds to NKG2A, and the third polypeptide and the fifth polypeptide form the third binding domain that binds to NKG2A.

23. The multispecific antibody or fragment thereof of claim 19, wherein the third binding domain binds to PD-L1.

24. The multispecific antibody or fragment thereof of claim 23, wherein the third binding domain is same as the second binding domain.

25. The multispecific antibody or fragment thereof of claim 23, wherein the multispecific antibody or fragment thereof comprises:
(i) a first polypeptide comprising from N-terminus to C-terminus: a first VL, a first CH3, a second VL, a second CH3, a first CH2, and a third CH3, wherein the first VL and the second VL are the same, and the first CH3 and the second CH3 are the same;
(ii) a second polypeptide comprising from N-terminus to C-terminus: a first VH and a fourth CH3;
(iii) a third polypeptide comprising from N-terminus to C-terminus: a second VH and a fifth CH3;
(iv) a fourth polypeptide comprising from N-terminus to C-terminus: a third VL, a first CL, a second CH2 and a sixth CH3;
(v) a fifth polypeptide comprising from N-terminus to C-terminus: a third VH and a CH1;
wherein the first VH and the second VH are the same, and the fourth CH3 and the fifth CH3 are the same; and
wherein the first polypeptide and the second polypeptide form the second binding domain that binds to PD-L1, the first polypeptide and the third polypeptide form the third binding domain that binds to PD-L1, and the fourth polypeptide and the fifth polypeptide form the first binding domain that binds to NKG2A.

26. A polynucleotide encoding the multispecific antibody or fragment thereof of any one of claims 1-25.

27. One or more vectors comprising one or more polynucleotides of claim 26 or a complementary polynucleotide thereto.

28. A cell comprising any one or more of: the multispecific antibody or fragment thereof of any one of claims 1-25, the polynucleotide of claim 26, or the one or more vectors of claim 27.

29. A pharmaceutical composition that comprises a pharmaceutically acceptable excipient and any one or more of: the multispecific antibody or fragment thereof of any one of claims 1-25, the polynucleotide of claim 26, the one or more vectors of claim 27, or the cell of claim 28.

30. A method of inhibiting interaction between HLA-E and NKG2A (or a complex comprising NKG2A and CD94 or extracellular domain of each thereof) and/or inhibiting interaction between PD-1 and PD-L1, comprising contacting the NKG2A (or the complex comprising NKG2A and CD94) and/or PD-L1 with the multispecific antibody or fragment thereof of any one of claims 1-25 or the pharmaceutical composition of claim 29.

31. The method of claim 30, wherein the NKG2A cell is expressed on an immune cell.

32. The method of claim 31, wherein the immune cell is an NK cell or a T cell, optionally wherein the T cell is a CD8⁺ T cell.

33. The method of any one of claims 30-32, wherein the HLA-E is expressed on a cancer cell.

34. A method of inhibiting interaction between PD-1 and PD-L1, comprising contacting the PD-L1 with the multispecific antibody or fragment thereof of any one of claims 1-25 or the pharmaceutical composition of claim 29.

35. The method of any one of claims 30-34, wherein the PD-L1 is on an immune cell or a cancer cell.

36. The method of any one of claims 30-35, wherein the PD-1 is expressed on an immune cell, optionally an NK cell or a T cell, further optionally a CD8⁺ T cell.

37. A method of preventing suppression of an immune cell or activating a response mediated by an immune cell, comprising contacting the immune cell with the multispecific antibody or fragment thereof of any one of claims 1-25 or the pharmaceutical composition of claim 29.

38. The method of claim 37, wherein the immune cell is an NK cell or a T cell.

39. The method of claim 38, wherein the T cell is a CD8⁺ T cell.

40. The method of any one of claims 37-39, wherein the immune cell expresses NKG2A and/or PD-1.

41. The method of any one of claims 37-40, wherein the response mediated by the immune cell is an anti-tumor response, optionally wherein the tumor cell expresses HLA-E and/or PD-L1.

42. A method for treating a disease or disorder in a subject comprising administering to the subject the multispecific antibody or fragment thereof of any one of claims 1-25, or the pharmaceutical composition of claim 29.

43. The method of claim 42, wherein the disease or disorder is a cancer, optionally the cancer expresses HLA-E and/or PD-L1.

44. The method of claim 42, wherein the disease or disorder is an autoimmune and inflammatory disease.

45. The method of any one of claims 42-44, wherein the subject is a human subject.

46. An antibody or fragment thereof that binds to PD-L1, comprising a VH CDR1, a VH CDR2, and a VH CDR3 as set forth in a VH comprising the amino acid sequence of SEQ ID NO:98 and a VL CDR1, a VL CDR2, and a VL CDR3 as set forth in a VL comprising the amino acid sequence of SEQ ID NO:99.

47. The antibody or fragment thereof of claim 46, comprising
(a) a VH region comprising:
(1) a VH CDR1 having an amino acid sequence selected from the group consisting of SEQ ID NOs: 1, 7, 12, 13, and 18;
(2) a VH CDR2 having an amino acid sequence selected from the group consisting of SEQ ID NOs: 86, 89, 91, 94, and 97; and
(3) a VH CDR3 having an amino acid sequence selected from the group consisting of SEQ ID NOs: 87, 90. 92, and 95;
and
(b) a VL region comprising:
(1) a VL CDR1 having an amino acid sequence selected from the group consisting of SEQ ID NOs: 4, 10, 16, and 21;
(2) a VL CDR2 having an amino acid sequence selected from the group consisting of SEQ ID NOs: 5, 11, and 22; and
(3) a VL CDR3 having an amino acid sequence selected from the group consisting of SEQ ID NOs: 88, 93, and 96.

48. The antibody or fragment thereof of claim 46 or 47, comprising any one or more of (i)-(vi):
(i) a VH region comprising a VH CDR1 comprising the amino acid sequence of SEQ ID NO:1, a VH CDR2 comprising the amino acid sequence of SEQ ID NO:86, a VH CDR3 comprising the amino acid sequence of SEQ ID NO:87; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:88;
(ii) a VH region comprising a VH CDR1 comprising the amino acid sequence of SEQ ID NO:7, a VH CDR2 comprising the amino acid sequence of SEQ ID NO:89, a VH CDR3 comprising the amino acid sequence of SEQ ID NO:90; and a VL region comprising a VL CDR1 comprising the amino acid sequence of SEQ ID NO:10, a VL CDR2 comprising the amino acid sequence of SEQ ID NO:11, and a VL CDR3 comprising the amino acid sequence of SEQ ID NO:88;
(iii) a VH region comprising a VH CDR1 comprising the amino acid sequence of SEQ ID NO:12, a VH CDR2 comprising the amino acid sequence of SEQ ID NO:86, a VH CDR3 comprising the amino acid sequence of SEQ ID NO:87; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:88;
(iv) a VH region comprising a VH CDR1 comprising the amino acid sequence of SEQ ID NO:13, a VH CDR2 comprising the amino acid sequence of SEQ ID NO:91, a VH CDR3 comprising the amino acid sequence of SEQ ID NO:92; and a VL region comprising a VL CDR1 comprising the amino acid sequence of SEQ ID NO:16, a VL CDR2 comprising the amino acid sequence of SEQ ID NO:11, and a VL CDR3 comprising the amino acid sequence of SEQ ID NO:93;
(v) a VH region comprising a VH CDR1 comprising the amino acid sequence of SEQ ID NO:18, a VH CDR2 comprising the amino acid sequence of SEQ ID NO:94, a VH CDR3 comprising the amino acid sequence of SEQ ID NO:95; and a VL region comprising a VL CDR1 comprising the amino acid sequence of SEQ ID NO:21, a VL CDR2 comprising the amino acid sequence of SEQ ID NO:22, and a VL CDR3 comprising the amino acid sequence of SEQ ID NO:96; or
(vi) a VH region comprising a VH CDR1 comprising the amino acid sequence of SEQ ID NO:1, a VH CDR2 comprising the amino acid sequence of SEQ ID NO:97, a VH CDR3 comprising the amino acid sequence of SEQ ID NO:87; and a VL region comprising a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:88.

49. The antibody or fragment thereof of any one of claims 46-48, wherein the VH region comprises the amino acid sequence of SEQ ID NO:98 and the VL region comprises the amino acid sequence of SEQ ID NO:99.

50. A polynucleotide encoding the antibody or fragment thereof of any one of claims 46-49.

51. One or more vectors comprising one or more polynucleotides of claim 50 or a complementary polynucleotide thereto.

52. A cell comprising any one or more of: the antibody or fragment thereof of any one of claims 46-49, the polynucleotide of claim 50, or the one or more vectors of claim 51.

53. A pharmaceutical composition that comprises a pharmaceutically acceptable excipient and any one or more of: the antibody or fragment thereof of any one of claims 46-49, the polynucleotide of claim 50, the one or more vectors of claim 51, or the cell of claim 52.

54. A method of inhibiting interaction between PD-1 and PD-L1, comprising contacting the PD-L1 with the antibody or fragment thereof of any one of claims 46-49 or the pharmaceutical composition of claim 53.

55. A method of preventing suppression of an immune cell or activating a response mediated by an immune cell, comprising contacting the immune cell with the antibody or fragment thereof of any one of claims 46-49 or the pharmaceutical composition of claim 53.

56. A method for treating a disease or disorder in a subject comprising administering to the subject the antibody or fragment thereof of any one of claims 46-49 or the pharmaceutical composition of claim 53.
